SEQUENCE LISTING (163 sequences)

SEQ ID NO: 1 - DNA, 816 bp, Artificial Sequence
  scFv#2 single chain antibody
caggctgtgctgactcagccgtcctccgtgtccgggtccccgggccnnanggtctccatcacctgctctggaagcaggagtaacgttggcacatatggtgtaggttggttccaacagctcccaggatcgggcctcagaaccatcatctattataatgacaaacgaccctcaggggtccccgaccgattctctgcctccaaatcgggcaacacagccaccctgatcatcagctcgctccaggctgaggatgaggccgattatttctgtggaagtgccgacggtagtagttatggtattttcggcagtgggaccagactgaccgtcctgggtcagcccgcggccgctggtggaggcggttcaggcggaggtggctctggcggtggcggatcggcgcgccaggtggggctgcaggagtcgggacccagcctggtgaagccctcacagaccctctccctcacctgcacggtctctggattctcattgtccaacagtgttgtaggctgggtccgccaggctccaggaaaggtgccggagtggcttggtagtatagacagtggtggttacacagtcgctgacccggccctgaaatcccgactcagcatcacaagggacacttccaagagccaagtctccctgtcactgaacagcgtgacaactgaggacacggccgtgtactactgtacaagggcttatagtattacttattatgcgtatcccccctatatcgactactggggcccaggactcctggtcaccgtctcctcaactagtggtgcgccggtgccgtatccggatccgctggaaccgcgtgccgca

SEQ ID NO: 2 - protein, 272 aa, Artificial Sequence
  scFv#2 single chain antibody
QAVLTQPSSVSGSPGXXVSITCSGSRSNVGTYGVGWFQQLPGSGLRTIIYYNDKRPSGVPDRFSASKSGNTATLIISSLQAEDEADYFCGSADGSSYGIFGSGTRLTVLGQPAAAGGGGSGGGGSGGGGSARQVGLQESGPSLVKPSQTLSLTCTVSGFSLSNSVVGWVRQAPGKVPEWLGSIDSGGYTVADPALKSRLSITRDTSKSQVSLSLNSVTTEDTAVYYCTRAYSITYYAYPPYIDYWGPGLLVTVSSTSGAPVPYPDPLEPRAA

SEQ ID NO: 3 - DNA, 826 bp, Artificial Sequence
  scFv#3 Single Chain Antibody
caggctgtgctgactcagccgtcctccgtgtccaggtccctgggccagagtgtctccatcacctgctctggaagcagcagcaacgttggatatggtgattatgtgggctggttccaacgggtcccaggatcagcccccaaactcctcatctatggtgcaaccactcgagcctcgggggtccccgaccgattctccggctccaggtctggcaacacagcgactctgaccatcagctcgctccaggctgaggacgaggccgattattactgttcatcttacgacagtagtcactatagtattttcggcagtgggaccagcctgaccgtcctgggtcagcccgcggccgctggtggaggcggttcaggcggaggtggctctggcggtggcggatcggcgcgccaggtggagctgcaggagtcgggacccagcctggtgaagccctcacagaccctctccctcacctgcacggtctctggattctcattaagtagcaatgctgtaggctgggtccgccaggctccaggaaaggcgccggagtgggttggtggtatagatatagatggaaggccggtctataaaccaggccttaagtcccggctcagcatcaccagggacacctccaacgctcaagtctccctgtcactgagcagcgtgacaactgaggacacggccgtgtacttctgtgcaagttattatggtggttatctttataattatgcccctggggcatatatcgagcacttgagcccaggactcctgatcaccgtctcctcaactagtggtgcgccggtgccgtatccggatccgctggaaaccgcgtgccgca

SEQ ID NO: 4 - protein, 275 aa, Artificial Sequence
  scFv#3 single chain antibody
QAVLTQPSSVSRSLGQSVSITCSGSSSNVGYGDYVGWFQRVPGSAPKLLIYGATTRASGVPDRFSGSRSGNTATLTISSLQAEDEADYYCSSYDSSHYSIFGSGTSLTVLGQPAAAGGGGSGGGGSGGGGSARQVELQESGPSLVKPSQTLSLTCTVSGFSLSSNAVGWVRQAPGKAPEWVGGIDIDGRPVYKPGLKSRLSITRDTSNAQVSLSLSSVTTEDTAVYFCASYYGGYLYNYAPGAYIEHLSPGLLITVSSTSGAPVPYPDPLEPRAA

SEQ ID NO: 5 - DNA, 816 bp, Artificial Sequence
  scFv#7 Single Chain Antibody
tcctatgaactgacccagccgccttcaatgtcggtggccttgggacagacggccaaggtcacctgccagggagacaacttagaaaacttttatgttcagtggcaccagcagaagccgggccaggcccctgtgacggtcatttttcaggataataagaggccctcggggatccctgaccggttctctggctccaactcggggaacacggccaccctgaccatcagcggggcccggaccgaggacgaggccgactattactgtcagtcaggccacagcagtatcggtggtgttttcggcagcgggaccagcctgaccgtcctgggtcagcccgcggccgctggtggaggcggttcaggcggaggtggctctggcggtggcggatcggcgcgccaggtgcagctgcaggagtcgggacccagcctggtgaagccctcacagaccctctccctcacctgcacggtctctggcttctcattaacgggaaattctgtaacctgggtccgccaggctccaggaaacgtgccggagtggcttggtggtataagccgcggtggacgcacatactatgatacggccctgaagtcccggctcagcatcaccagggacacctccaagaggcaagtctccctatcactgagcagcgtgacgactgaggacacggccatgtacttctgtgcaagatcggcatatagtactctttatgattatgagtatgccgctgatatctacgactggggcccaggactcctggtcaccgtctcctcaactagtggtgcgccggtgccgtatccggatccgctggaaccgcgtgccgca

SEQ ID NO: 6 - protein, 272 aa, Artificial Sequence
  scFv#7 Single Chain Antibody
SYELTQPPSMSVALGQTAKVTCQGDNLENFYVQWHQQKPGQAPVTVIFQDNKRPSGIPDRFSGSNSGNTATLTISGARTEDEADYYCQSGHSSIGGVFGSGTSLTVLGQPAAAGGGGSGGGGSGGGGSARQVQLQESGPSLVKPSQTLSLTCTVSGFSLTGNSVTWVRQAPGNVPEWLGGISRGGRTYYDTALKSRLSITRDTSKRQVSLSLSSVTTEDTAMYFCARSAYSTLYDYEYAADIYDWGPGLLVTVSSTSGAPVPYPDPLEPRAA

SEQ ID NO: 7 - DNA, 795 bp, Artificial Sequence
  scFv#8 Single Chain Antibody
tcctatgaactgacccagccgccttcagtgtcggtggtttggggncnganggccgagatcacctgccagggagacctactggataaaaaatatacagcttggtaccagcagaagccgggccaggctcctatgaaaatcattaataaagacagtgagcggccttcagggatccgggaccggttctcgggctccagctcaggcaaaacagccaccctaaccatcaacggggcccggcctgaggacgaggccgactattactgtttatcaggtgacagcaataataatggtgtcttcggcagcgggaccagcctgaccgtcctgggtcagcccgcggccgctggtggaggcggttcaggcggaggtggctctggcggtggcggatcggcgcgccaggtggagctgcaggggtcgggacccagcctggtgaagccctcgcagaccctctccctcacctgcacggtctctggattctcatggcccaacaatgctgtggattgggtccgccaggctccaggaaaggcgccggagtggcttggtggtattgccgataatggaagaacaaactacaacacggccctaaaagcccggctcagcatcactagggacaccgccaagagccatgtctccctatcgctgagcagcgtgacagctgaggatacggccgtttactattgtacagcgggggttatggtcatgcacgccactgactactggggcccgggactcctggtcaccgtctcctcaactagtggtgcgccggtgccgtatccggatccgctggaaccgcgtgccgca

SEQ ID NO: 8 - protein, 265 aa, Artificial Sequence
  scFv#8 Single Chain Antibody
SYELTQPPSVSVVWGXXAEITCQGDLLDKKYTAWYQQKPGQAPMKIINKDSERPSGIRDRFSGSSSGKTATLTINGARPEDEADYYCLSGDSNNNGVFGSGTSLTVLGQPAAAGGGGSGGGGSGGGGSARQVELQGSGPSLVKPSQTLSLTCTVSGFSWPNNAVDWVRQAPGKAPEWLGGIADNGRTNYNTALKARLSITRDTAKSHVSLSLSSVTAEDTAVYYCTAGVMVMHATDYWGPGLLVTVSSTSGAPVPYPDPLEPRAA

SEQ ID NO: 9 - DNA, 810 bp, Artificial Sequence
  scFv#21 Single Chain Antibody
caggctgtggtgactcagccgtcctccgtgtccgggtccccgggccnnanagtctccatcacctgctctggaagcagcagcaacgttggtagatatgctgtaggctggttccaacagctcccaggatcgggcctcagaaccgtcatctattataatagcaatcgaccctcaggggtccccgaccgattctctggctccaaatcgggcaacacagccaccctgaccatcagctcgctccaggctgaggatgaggccgattatttctgtggaagttatgacagtagtatctatggtgttttcggcagcgggaccaggctgaccgtcctgggtcagcccgcggccgctggtggaggcggttcaggcggaggtggctctggcggtggcggatcggcgcgccaggtgcagctgcaggagtcgggacccagcctggtgaggccctcacagaccctctccctcacctgcacgatctctggattctctttaagagagtatggtgtaggttgggtccgccaggctccaggaaaggcgttggagtggcttgggcgaatagatgattctggatacacattacataatcctgcccttaagtcccggctcaccataactagggacatctccaagagccaagtctccctgtcactgagcagcgtgacacttgaggacacggccgaatattactgcgtatatgctagtcgtggtactgcttggttgggagacatcgatgtctggggcccaggactcctgctcactgtctcctcaactagtggtgcgccggtgccgtatccggatccgctggaaccgcgtgccgca

SEQ ID NO: 10 - protein, 270 aa, Artificial Sequence
  scFv#21 Single Chain Antibody
QAVVTQPSSVSGSPGXXVSITCSGSSSNVGRYAVGWFQQLPGSGLRTVIYYNSNRPSGVPDRFSGSKSGNTATLTISSLQAEDEADYFCGSYDSSIYGVFGSGTRLTVLGQPAAAGGGGSGGGGSGGGGSARQVQLQESGPSLVRPSQTLSLTCTISGFSLREYGVGWVRQAPGKALEWLGRIDDSGYTLHNPALKSRLTITRDISKSQVSLSLSSVTLEDTAEYYCVYASRGTAWLGDIDVWGPGLLLTVSSTSGAPVPYPDPLEPRAA

SEQ ID NO: 11 - DNA, 816 bp, Artificial Sequence
  scFv#E Single Chain Antibody
caggctgtgctgactcagccgtcctccgtgtccaggtccctgggccnnantgtctcgatcacctgctctggaggcagcagcaacgttggacaaggtgattatgtggcctggttccaacaggtcccaggatcagcccccaaactcctcatctatgatgcgacgaatcgagcctcgggggtccccgaccgattcgtcggctccagatatggcaactcagcgactctgatcatcacctcggtccaggctgaggacgaggccgattattattgtgcatcttatgacagtagtatgtatacgattttcggcagcgggaccagcctgaccgtcctgggtcagcccgcggccgctggtggaggcggttcaggcggaggtggctctggcggtggcggatcggcgcgccaggtggagctgcaggggtcgggacccagccaggtgaagccctcacagaccctctccctcatctgcacgatctctggattctcattaaccagcaataatgtagcctgggtccgccaggctccaggaaagggactggagtgggttggtgtcataagtgatggtggaactccatactataactcggccctgaaatcccggctcagcatcaccagggacacctccaagagccaggtctccctgtcactgagcagcgtgacaactgaggacacggccgtgtactactgtgcacggacgttggattatagtcatatttggttgtactccgccgaccaatggggcccaggactcctggtcaccgtctcctcaactagtggtgcgccggtgccgtatccggatccgctggaaccgcgtgccgca

SEQ ID NO: 12 - protein, 272 aa, Artificial Sequence
  scFv#E Single Chain Antibody
QAVLTQPSSVSRSLGXXVSITCSGGSSNVGQGDYVAWFQQVPGSAPKLLIYDATNRASGVPDRFVGSRYGNSATLIITSVQAEDEADYYCASYDSSMYTIFGSGTSLTVLGQPAAAGGGGSGGGGSGGGGSARQVELQGSGPSQVKPSQTLSLICTISGFSLTSNNVAWVRQAPGKGLEWVGVISDGGTPYYNSALKSRLSITRDTSKSQVSLSLSSVTTEDTAVYYCARTLDYSHIWLYSADQWGPGLLVTVSSTSGAPVPYPDPLEPRAA

SEQ ID NO: 13 - DNA, 861 bp, Artificial Sequence
  scFv#7-2E Single Chain Antibody
ggtgcgccggtgccgtatccggatccgctcgagccgcgtgccggctcctatgaactgacccagccgccttcaatgtcggtggccttgggacagacggccaaggtcacctgccagggagacaacttagaaaacttttatgttcagtggcaccagcagaagccgggccaggcccctgtgacggtcatttttcaggataataagaggccctcggggatccctgaccggttctctggctccaactcggggaacacggccaccctgaccatcagcggggcccggaccgaggacgaggccgactattactgtcagtcaggccacagcagtatcggtggtgttttcggcagcgggaccagcctgaccgtcctgggtcagcccgcggccgctggtggaggcggttcaggcggaggtggctctggcggtggcggatcggcgcgccaggtgcagctgcaggagtcgggacccagcctggtgaagccctcacagaccctctccctcacctgcacggtctctggcttctcattaacgggaaattctgtaacctgggtccgccaggctccaggaaacgtgccggagtggcttggtggtataagccgcggtggacgcacatactatgatacggccctgaagtcccggctcagcatcaccagggacacctccaagaggcaagtctccctatcactgagcagcgtgacgactgaggacacggccatgtacttctgtgcaagatcggcatatagtactctttatgattatgagtatgccgctgatatctacgactggggcccaggactcctggtcaccgtctcctcaactagtggtgcgccggtgccgtatccggatccgctggaaccgcgtgccgca

SEQ ID NO: 14 - protein, 287 aa, Artificial Sequence
  scFv#7-2E Single Chain Antibody
GAPVPYPDPLEPRAGSYELTQPPSMSVALGQTAKVTCQGDNLENFYVQWHQQKPGQAPVTVIFQDNKRPSGIPDRFSGSNSGNTATLTISGARTEDEADYYCQSGHSSIGGVFGSGTSLTVLGQPAAAGGGGSGGGGSGGGGSARQVQLQESGPSLVKPSQTLSLTCTVSGFSLTGNSVTWVRQAPGNVPEWLGGISRGGRTYYDTALKSRLSITRDTSKRQVSLSLSSVTTEDTAMYFCARSAYSTLYDYEYAADIYDWGPGLLVTVSSTSGAPVPYPDPLEPRAA

SEQ ID NO: 15 - protein, 13 aa, Artificial Sequence
  Epitope Tag
GAPVPYPDPLEPR

SEQ ID NO: 16 - protein, 9 aa, Artificial Sequence
  VHH binding agents
QVQLVESGG

SEQ ID NO: 17 - protein, 8 aa, Artificial Sequence
  VHH binding agents
AHHSEDPS

SEQ ID NO: 18 - protein, 8 aa, Artificial Sequence
  VHH binding agents
EPKTPKPQ

SEQ ID NO: 19 - DNA, 396 bp, Artificial Sequence
  VHH binding agents specific to BoNT/A holotoxin
caggtgcagctcgtggagtcaggaggaggcttggtgcagcctgggggatctctgagactctcgtgtgtagtctctggaagtgacttcaatacctatatcatgggctggtaccgccaggttccagggaagccgcgcgagttggtcgcagatattactactgaaggaaaaacaaactatggcggctccgtaaagggacgattcaccatctccagagacaacgccaaaaacacggtgtatctgcaaatgttcggcctgaaacctgaggacgcgggtaattatgtctgtaacgcagactggaagatgggtgcatggaccgcgggggactacggtatcgactactggggcaaagggaccctggtcaccgtctcctcagaacccaagacaccaaaaccacaa

SEQ ID NO: 20 - protein, 132 aa, Artificial Sequence
  VHH binding agents specific to BoNT/A holotoxin
QVQLVESGGGLVQPGGSLRLSCVVSGSDFNTYIMGWYRQVPGKPRELVADITTEGKTNYGGSVKGRFTISRDNAKNTVYLQMFGLKPEDAGNYVCNADWKMGAWTAGDYGIDYWGKGTLVTVSSEPKTPKPQ

SEQ ID NO: 21 - DNA, 408 bp, Artificial Sequence
  VHH binding agents specific to BoNT/A holotoxin
caggtgcagctcgtggagtccggtggaggcttggtgcagcctggggggtctctgagactctcctgtgcagcctctgcaggcaatctggattattatgccataggctggttccgccaggccccagggaaggagcgcgagggggtctcatgtattagtagtagtgatggtagcactgtctatacagactccgtgaagggccgattcaccatctccagagacaataccaagaacacggtagatctgcaaatggacaatttgaaacctgaggacacggccgtttattactgtgcgacagtcgttaactactactgcacagccggtgggtccattcacgcgagcccgtatgaaatctggggccaggggacccaggtcaccgtctcctcagcgcaccacagcgaagacccctcg

SEQ ID NO: 22 - protein, 136 aa, Artificial Sequence
  VHH binding agents specific to BoNT/A holotoxin
QVQLVESGGGLVQPGGSLRLSCAASAGNLDYYAIGWFRQAPGKEREGVSCISSSDGSTVYTDSVKGRFTISRDNTKNTVDLQMDNLKPEDTAVYYCATVVNYYCTAGGSIHASPYEIWGQGTQVTVSSAHHSEDPS

SEQ ID NO: 23 - DNA, 375 bp, Artificial Sequence
  VHH binding agents specific to BoNT/A holotoxin
caggtgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaa

SEQ ID NO: 24 - protein, 133 aa, Artificial Sequence
  VHH binding agents specific to BoNT/A holotoxin
QVQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQEPKTPKPQ

SEQ ID NO: 25 - DNA, 378 bp, Artificial Sequence
  VHH binding agents specific to BoNT/A holotoxin
caggtgcagctcgtggagtctggtggaggcttggcgcagcctggggggtctctgagactctcctgtgaagcgtctggttttgggacatggttcaggttcgatgagaacaccgtgaactggtaccgccagcctccaggaaagtcgcgcgagttcgacgagttggtcgctcgttacccaaaaagtggcatcgtaacctatttagactccgtgaagggccgattcacgatctccagagacaacgccaaaaaaatggcgtttctgcaaatggacaacctgaaacctgaggacacggccgtctattattgcaatgtcggtgaattttggggccaggggacccaggtcacgatctcctcagaacccaagacaccaaaaccacaa

SEQ ID NO: 26 - protein, 126 aa, Artificial Sequence
  VHH binding agents specific to BoNT/A holotoxin
QVQLVESGGGLAQPGGSLRLSCEASGFGTWFRFDENTVNWYRQPPGKSREFDELVARYPKSGIVTYLDSVKGRFTISRDNAKKMAFLQMDNLKPEDTAVYYCNVGEFWGQGTQVTISSEPKTPKPQ

SEQ ID NO: 27 - DNA, 384 bp, Artificial Sequence
  VHH binding agents specific to BoNT/A holotoxin
caggtgcagctcgtggagtcggggggaggcttggtgcagcctggggggtctctgagactctcctgtgcagcctctggattcaccctagggtcgcgttacatgagctgggtccgccaggctccaggagaggggttcgagtgggtctcaagtattgaaccctctggtaccgcatgggatggagactccgcgaagggacgattcaccacttccagagacgacgccaagaacacgctttatctgcaaatgagcaacctgcaacccgaggacacgggtgtttattactgtgcaacagggtatcggacggacacgaggattccgggtggctcgtggggccaggggacccaggtcaccgtctcctcagaacccaagacaccaaaaccacaa

SEQ ID NO: 28 - protein, 128 aa, Artificial Sequence
  VHH binding agents specific to BoNT/A holotoxin
QVQLVESGGGLVQPGGSLRLSCAASGFTLGSRYMSWVRQAPGEGFEWVSSIEPSGTAWDGDSAKGRFTTSRDDAKNTLYLQMSNLQPEDTGVYYCATGYRTDTRIPGGSWGQGTQVTVSSEPKTPKPQ

SEQ ID NO: 29 - DNA, 360 bp, Artificial Sequence
  VHH binding agents specific to BoNT/A holotoxin
caggtgcagctcgtggagtctggaggaggcttggtgcagcctggggggtctctgagactctcctgtcaagtctctggattcaccttcggtgactgggtcatgagctggttccgccaggctccggggaaggagcgcgaattcgtcgcaagtattacggctactagtagtctaaagtattatgcagactccgtgaagggccgattcaccatctccagagacaatgtcaacaacacactgtttctgcaaatggatcgcctgaaatctgaggacacggccgtttattactgtcggtcccccaactactggggccaggggacccaggtcaccgtctccgccgaacccaagacaccaaaaccacaa

SEQ ID NO: 30 - protein, 120 aa, Artificial Sequence
  VHH binding agents specific to BoNT/A holotoxin
QVQLVESGGGLVQPGGSLRLSCQVSGFTFGDWVMSWFRQAPGKEREFVASITATSSLKYYADSVKGRFTISRDNVNNTLFLQMDRLKSEDTAVYYCRSPNYWGQGTQVTVSAEPKTPKPQ

SEQ ID NO: 31 - DNA, 366 bp, Artificial Sequence
  VHH binding agents specific to BoNT/A holotoxin
caggtgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagacccctcg

SEQ ID NO: 32 - protein, 122 aa, Artificial Sequence
  VHH binding agents specific to BoNT/A holotoxin
QVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPS

SEQ ID NO: 33 - DNA, 411 bp, Artificial Sequence
  VHH binding agents specific to BoNT/B holotoxin
caggtgcagctcgtggagtcaggcggaggcttggtgcagcctggggggtctctgaaactctcctgtgcagcctctggattcactttgggacaccatcgcgttggctggttccgccaggccccaggaaagaagcgtgagggggtcgcgtgtattagcgccactggtcttagcacacactattcagactccgtgaccggccgatttaccgtctccagagacaacctcaacaacgtggcgtatctgcagctgaacagcctgaaacctgaggacgcaggtgtttattactgtgcaagcagattctcccttaattcggtcgatgcgaatatgtgcctttcagagcctcagtatgacaactggggccaggggacccaggtcagaatctcctcagaacccaagacaccaaaaccacaa

SEQ ID NO: 34 - protein, 137 aa, Artificial Sequence
  VHH binding agents specific to BoNT/B holotoxin
QVQLVESGGGLVQPGGSLKLSCAASGFTLGHHRVGWFRQAPGKKREGVACISATGLSTHYSDSVTGRFTVSRDNLNNVAYLQLNSLKPEDAGVYYCASRFSLNSVDANMCLSEPQYDNWGQGTQVRISSEPKTPKPQ

SEQ ID NO: 35 - DNA, 399 bp, Artificial Sequence
  VHH binding agents specific to BoNT/B holotoxin
caggtgcagctcgtggagacgggtggaggattggtgcaggccgggggctctctgagactctcctgcgcaggctctggacgctccttcagcgccgctgtcatgggctggttccgccaggcgccagggaaggagcgagaattcgtagcagcacttagacaaattattggtagcacacactatgcagactccgtgaagggccgattcaccatctccagagacaacgccaagaacatgttgtatctcgacatgaacagcctgaaacctacggacacggccgcgtattactgcacagcgggacctccgactatgctggacgtttctaccgaccgggagtatgacacctggggtcaggggactcaggtcaccgtctcctcagcgcaccacagcgaagacccctcg

SEQ ID NO: 36 - protein, 133 aa, Artificial Sequence
  VHH binding agents specific to BoNT/B holotoxin
QVQLVESGGGLVQAGGSLRLSCAGSGRSFSAAVMGWFRQAPGKEREFVAALRQIIGSTHYADSVKGRFTISRDNAKNMLYLDMNSLKPTDTAAYYCTAGPPTMLDVSTDREYDTWGQGTQVTVSSAHHSEDPS

SEQ ID NO: 37 - DNA, 363 bp, Artificial Sequence
  VHH binding agent
caggtgcagctcgtggagtccggaggaggcttggtgcgacctggggggtctctgagactctcttgtgtagtctctggattcgcctacgaaatgcccatgatgggctggtaccgccaggctccagggaatcagcgcgagttggtcgcaactattggtacaggtggtaggatgaactatgcagactccgtgaagggccgattcaccatctccagagacaacgccaagaacacggtgtatctgcaaatgaacagcctgaaacctgaggacacagccgcctattactgtaaaatcgagtttacaaattactggggccaggggacccaagtcaccgtctcctcagaacccaagacaccaaaaccacaa

SEQ ID NO: 38 - protein, 121 aa, Artificial Sequence
  VHH binding agent
QVQLVESGGGLVRPGGSLRLSCVVSGFAYEMPMMGWYRQAPGNQRELVATIGTGGRMNYADSVKGRFTISRDNAKNTVYLQMNSLKPEDTAAYYCKIEFTNYWGQGTQVTVSSEPKTPKPQ

SEQ ID NO: 39 - DNA, 366 bp, Artificial Sequence
  VHH binding agent
caggtgcagctcgtggagtcaggtggaggcttggtgcagccggggggatctctgagactgtcctgtacagtctctggaagcatcttcgatctacctggaatgaactggtatcgccaggctccaggggcgcagcgcgagttggtcgcagatattagtagtgatggtaggaggacaaactatgcagactccgtgaagggccgattcaccatgtccagagacaatgccaagaaaacggtgtatctgcaaatggacagcctgaaacctgacgacacggccgtctattactgtaatgtgaaatttactcaccactggggccaggggatccaggtcaccgtctcctcagaacccaagacaccaaaaccacaa

SEQ ID NO: 40 - protein, 122 aa, Artificial Sequence
  VHH binding agent
QVQLVESGGGLVQPGGSLRLSCTVSGSIFDLPGMNWYRQAPGAQRELVADISSDGRRTNYADSVKGRFTMSRDNAKKTVYLQMDSLKPDDTAVYYCNVKFTHHWGQGIQVTVSSEPKTPKPQ

SEQ ID NO: 41 - DNA, 366 bp, Artificial Sequence
  VHH binding agent
caggtgcagctcgtggagtcaggcggaggcttggtgcagccggggggatctctgaggctgtcctgtacggtctctggaagcatcttcggcctacctggcatgagctggtatcgccaggctccaggggcgcagcgcgagttggtcgcagatattagtagtgatggtgggaggacgcactatgcagactccgtgaagggccgcttcaccatctccagagacaatgacaagaaaacggtgtatctgcagatggacagcctgaaacctgacgacacggccgtctattactgtaatgtgaaatttactcaccactggggccaggggatccaggtcaccgtctcctcagaacccaagacaccaaaaccacaa

SEQ ID NO: 42 - protein, 122 aa, Artificial Sequence
  VHH binding agent
QVQLVESGGGLVQPGGSLRLSCTVSGSIFGLPGMSWYRQAPGAQRELVADISSDGGRTHYADSVKGRFTISRDNDKKTVYLQMDSLKPDDTAVYYCNVKFTHHWGQGIQVTVSSEPKTPKPQ

SEQ ID NO: 43 - DNA, 366 bp, Artificial Sequence
  VHH binding agent
caggtgcagctcgtggagtctgggggaggcttggtgcaggatggggggtctctgaggctctcctgcacaacatctggaagtatcgacagtttcaatgccatagagtggtaccgccaggctccagggaagcagcgcgaattggtcgcaagtataagtagtgatggtcgtcgcacaaactatgcagactccgtgaagggccgattcaccatctccggagacaacgccaagaacacggtgtatctgcaaatgaacagcctgaaacctgaggacacagccgtgtattactgtcatagaccttttacccactactggggccaggggacccaggtcaccgtctcctcagaacccaagacaccaaaaccacaa

SEQ ID NO: 44 - protein, 122 aa, Artificial Sequence
  VHH binding agent
QVQLVESGGGLVQDGGSLRLSCTTSGSIDSFNAIEWYRQAPGKQRELVASISSDGRRTNYADSVKGRFTISGDNAKNTVYLQMNSLKPEDTAVYYCHRPFTHYWGQGTQVTVSSEPKTPKPQ

SEQ ID NO: 45 - DNA, 912 bp, Artificial Sequence
  VHH binding agent with tag
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgatatcggatccgaattcccaggtgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagaccccactagtggtgcgccggtgccgtatccggatccgctggaaccgcgttaa

SEQ ID NO: 46 - protein, 303 aa, Artificial Sequence
  VHH binding agent with tag
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSQVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSGAPVPYPDPLEPR

SEQ ID NO: 47 - DNA, 924 bp, Artificial Sequence
  VHH binding agent with tag
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgatatcggatccgaattcccaggtgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaaactagtggtgcgccggtgccgtatccggatccgctggaaccgcgttaa

SEQ ID NO: 48 - protein, 307 aa, Artificial Sequence
  VHH binding agent with tag
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSQVQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQTSGAPVPYPDPLEPR

SEQ ID NO: 49 - DNA, 1350 bp, Artificial Sequence
  VHH dimer binding agent with tag
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcggccgctcaggtgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagaccccactagtgcgatcgctggtggaggcggttcaggcggaggtggctctggcggtggcggttccctgcagggtcagttgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaaccggcgcgccagggtgcgccggtgccgtatccggacccgctggaaccgcgttaa

SEQ ID NO: 50 - protein, 449 aa, Artificial Sequence
  VHH dimer binding agent with tag
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAAAQVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQPARQGAPVPYPDPLEPR

SEQ ID NO: 51 - DNA, 1410 bp, Artificial Sequence
  VHH dimer binding agent with two tags
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgatatcggatccgaattctggcgcacctgtcccatacccagaccctctggaaccacgagcggccgctcaggtgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagaccccactagtgcgatcgctggtggaggcggttcaggcggaggtggctctggcggtggcggttccctgcagggtcagttgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaaccggcgcgccagggtgcgccggtgccgtatccggacccgctggaaccgcgttaa

SEQ ID NO: 52 - protein, 469 aa, Artificial Sequence
  VHH dimer binding agent with two tags
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSGAPVPYPDPLEPRAAAQVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQPARQGAPVPYPDPLEPR

SEQ ID NO: 53 - protein, 109 aa, Clostridium botulinum
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 54 - protein, 5 aa, Artificial Sequence
  The sequence has been designed and synthesized
GGGGS

SEQ ID NO: 55 - protein, 15 aa, Artificial Sequence
  The sequence has been designed and synthesized
GGGGSGGGGSGGGGS

SEQ ID NO: 56 - protein, 111 aa, Artificial Sequence
  The sequence has been designed and synthesized
LVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDP

SEQ ID NO: 57 - protein, 115 aa, Artificial Sequence
  The sequence has been designed and synthesized
LVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQ

SEQ ID NO: 58 - protein, 269 aa, Artificial Sequence
  The sequence has been designed and synthesized
QVQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQ

SEQ ID NO: 59 - protein, 135 aa, Artificial Sequence
  The sequence has been designed and synthesized
QVQLVETGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSEPKTPKPQP

SEQ ID NO: 60 - protein, 130 aa, Artificial Sequence
  The sequence has been designed and synthesized
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSEPKTPKPQP

SEQ ID NO: 61 - protein, 137 aa, Artificial Sequence
  The sequence has been designed and synthesized
QVQLVESGGGLVQPGGSLRLSCAASGFTLDYYAIGWFRQAPGKEREGVSGISSVDGSTYYADSVRGRFTISRDNAKNTVYLQMNSLKPEDTAVYYCAADQSPIPIHYSRTYSGPYGMDYWGKGTLVTVSSAHHSEDP

SEQ ID NO: 62 - protein, 139 aa, Artificial Sequence
  The sequence has been designed and synthesized
QLQLVESGGGLVQPGGSLRLSCAASGFTLDYYAIGWFRQAPGKEREGVSGISFVDGSTYYADSVKGRFAISRGNAKNTVYLQMNSLKPEDTAVYYCAADQSSIPMHYSSTYSGPSGMDYWGKGTLVTVSSEPKTPKPQP

SEQ ID NO: 63 - protein, 136 aa, Artificial Sequence
  The sequence has been designed and synthesized
QLQLVETGGGLVQAGGSLRLSCAASGRTLSNYPMGWFRQAPGKEREFVAAIRRIADGTYYADSVKGRFTISRDNAWNTLYLQMNGLKPEDTAVYFCATGPGAFPGMVVTNPSAYPYWGQGTQVTVSSEPKTPKPQP

SEQ ID NO: 64 - protein, 139 aa, Artificial Sequence
  The sequence has been designed and synthesized
QLQLVESGGGLVQPGGSLRLSCAASGFTLDYYAIGWFRQAPGKEREGVSGISSSDGSTYYADSVKGRFTISRDNATNTVYLQMNSLKPEDTAVYYCAADQAAIPMHYSASYSGPRGMDYWGKGTLVTVSSEPKTPKPQP

SEQ ID NO: 65 - protein, 135 aa, Artificial Sequence
  The sequence has been designed and synthesized
QVQLVESGGGLVQPGGSLRLSCAASGFSLDYYGIGWFRQAPGKERQEVSYISASAKTKLYSDSVKGRFTISRDNAKNAVYLEMNSLKREDTAVYYCARRRFDASASNRWLAADYDYWGQGTQVTVSSEPKTPKPQ

SEQ ID NO: 66 - protein, 123 aa, Artificial Sequence
  The sequence has been designed and synthesized
QVQLVESGGGLVQAGGSLRLSCVSSERNPGINAMGWYRQAPGSQRELVAIWQTGGSLNYADSVKGRFTISRDNLKNTVYLQMNSLKPEDTAVYYCYLKKWRDQYWGQGTQVTVSSEPKTPKPQ

SEQ ID NO: 67 - protein, 135 aa, Artificial Sequence
  The sequence has been designed and synthesized
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQ

SEQ ID NO: 68 - protein, 119 aa, Artificial Sequence
  The sequence has been designed and synthesized
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQ

SEQ ID NO: 69 - protein, 124 aa, Artificial Sequence
  The sequence has been designed and synthesized
QVQLVESGGGLVEAGGSLRLSCVVTGSSFSTSTMAWYRQPPGKQREWVASFTSGGAIKYTDSVKGRFTMSRDNAKKMTYLQMENLKPEDTAVYYCALHNAVSGSSWGRGTQVTVSSEPKTPKPQ

SEQ ID NO: 70 - protein, 123 aa, Artificial Sequence
  The sequence has been designed and synthesized
VQLVESGGGLVQAGGSLRLSCAASGLMFGAMTMGWYRQAPGKEREMVAYITAGGTESYSESVKGRFTISRINANNMVYLQMTNLKVEDTAVYYCNAHNFWRTSRNWGQGTQVTVSSEPKTPKP

SEQ ID NO: 71 - protein, 131 aa, Artificial Sequence
  The sequence has been designed and synthesized
VQLVESGGGLVQAGDSLTLSCAASESTFNTFSMAWFRQAPGKEREYVAAFSRSGGTTNYADSVKGRATISTDNAKNTVYLHMNSLKPEDTAVYFCAADRPAGRAYFQSRSYNYWGQGTQVTVSSAHHSEDP

SEQ ID NO: 72 - protein, 125 aa, Artificial Sequence
  The sequence has been designed and synthesized
VQLVESGGGSVQIGGSLRLSCVASGFTFSKNIMSWARQAPGKGLEWVSTISIGGAATSYADSVKGRFTISRDNANDTLYLQMNNLKPEDTAVYYCSRGPRTYINTASRGQGTQVTVSSEPKTPKP

SEQ ID NO: 73 - protein, 121 aa, Artificial Sequence
  The sequence has been designed and synthesized
VQLVESGGGLVQAGGSLRLSCVGSGRNPGINAMGWYRQAPGSQRELVAVWQTGGSTNYADSVKGRFTISRDNLKNTVYLQMNSLKPEDTAVYYCYLKKWRDEYWGQGTQVTVSSAHHSEDP

SEQ ID NO: 74 - protein, 124 aa, Artificial Sequence
  The sequence has been designed and synthesized
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTPKP

SEQ ID NO: 75 - protein, 122 aa, Artificial Sequence
  The sequence has been designed and synthesized
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTP

SEQ ID NO: 76 - protein, 129 aa, Artificial Sequence
  The sequence has been designed and synthesized
VQLVESGGGLVQAGGSLRLSCAAPGLTFTSYRMGWFRQAPGKEREYVAAITGAGATNYADSAKGRFTISKNNTASTVHLQMNSLKPEDTAVYYCAASNRAGGYWRASQYDYWGQGTQVTVSSAHHSEDP

SEQ ID NO: 77 - protein, 129 aa, Artificial Sequence
  The sequence has been designed and synthesized
QVQLVETGGGLAQAGDSLRLSCVEPGRTLDMYAMGWIRQAPGEEREFVASISGVGGSPRYADSVKGRFTISKDNTKSTIWLQMNSLKPEDTAVYYCAAGGDIYYGGSPQWRGQGTRVTVSSEPKTPKPQ

SEQ ID NO: 78 - protein, 132 aa, Artificial Sequence
  The sequence has been designed and synthesized
QVQLVESGGGLVQAGGSLRLSCAASGRINGDYAMGWFRQAPGEEREFVAVNSWIGGSTYYTDSVKGRFTLSRDNAKNTLSLQMNSLKPEDTAVYYCAAGHYTDFPTYFKEYDYWGQGTQVTVSSEPKTPKPQ

SEQ ID NO: 79 - protein, 132 aa, Artificial Sequence
  The sequence has been designed and synthesized
QVQLVETGGLVQAGGSLRLSCAASGVPFSDYTMAWFRQAPGKEREVVARITWRGGGPYYGNSGNGRFAISRDIAKSMVYLHMDSLKPEDTAVYYCAASRLRPALASMASDYDYWGQGTQVSVSSEPKTPKPQ

SEQ ID NO: 80 - protein, 124 aa, Artificial Sequence
  The sequence has been designed and synthesized
QVQLVESGGGLVQPGESLRLSCVASASTFSTSLMGWVRQAPGKGLESVAEVRTTGGTFYAKSVAGRFTISRDNAKNTLYLQMNSLKAEDTGVYYCTAGAGPIATRYRGQGTQVTVSSAHHSEDP

SEQ ID NO: 81 - protein, 137 aa, Artificial Sequence
  The sequence has been designed and synthesized
QVQLVESGGGLVQPGGSLKLSCAASGFTLADYVTVWFRQAPGKSREGVSCISSSRGTPNYADSVKGRATVSRNNANNTVYLQMNGLKPDDTAIYYCAAIRPARLRAYRECLSSQAEYDYWGQGTQVTVSSAHHSEDP

SEQ ID NO: 82 - protein, 134 aa, Artificial Sequence
  The sequence has been designed and synthesized
QVQLVESGGGLVQPGGSLGLSCAMSGTTQDYSAVGWFRQAPGKEREGVSCISRSGRRTNYADSVRGRFTISRDNAKDTVYLQMNSLKPDDTAVYYCAARKTDMSDPYYVGCNGMDYWGKGTLVTVSSAHHSEDP

SEQ ID NO: 83 - protein, 130 aa, Artificial Sequence
  The sequence has been designed and synthesized
QVQLVESGGGLVQPGGSLTLSCTASGFTLNSYKIGWFRQAPGKEREGVSCINSGGNLRSVEGRFTISRDNTKNTVSLHMDSLKPEDTGVYHCAAAPALNVFSPCVLAPRYDYWGQGTQVTVSSAHHSEDP

SEQ ID NO: 84 - protein, 134 aa, Artificial Sequence
  The sequence has been designed and synthesized
QVQLVESGGGLVQPGGSLRLSCAASGFTLGSYHIGWFRHPPGKEREGTSCLSSRGDYTKYAEAVKGRFTISRDNTKSTVYLQMNNLKPEDTGIYVCAAIRPVLSDSHCTLAARYNYWGQGTQVTVSSAHHSEDP

SEQ ID NO: 85 - protein, 133 aa, Artificial Sequence
  The sequence has been designed and synthesized
QVQLVESGGGLVQPGGSLRLSCAALEFTLEDYAIAWFRQAPGKEREGVSCISKSGVTKYTDSVKGRFTVARDNAKSTVILQMNNLRPEDTAVYNCAAVRPVFVDSVCTLATRYTYWGEGTQVTVSSAHHSEDP

SEQ ID NO: 86 - protein, 134 aa, Artificial Sequence
  The sequence has been designed and synthesized
QVQLVETGGGLVQPGGSLKLSCAASEFTLDDYHIGWFRQAPGKEREGVSCINKRGDYINYKDSVKGRFTISRDGAKSTVFLQMNNLRPEDTAVYYCAAVNPVFPDSRCTLATRYTHWGQGTQVTVSSAHHSEDP

SEQ ID NO: 87 - protein, 453 aa, Artificial Sequence
  The sequence has been designed and synthesized
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQTSAIAGGGGSGGGGSGGGGSLQAMAAASQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQPARR

SEQ ID NO: 88 - protein, 6 aa, Artificial Sequence
  The sequence has been designed and synthesized
QLQLVE

SEQ ID NO: 89 - protein, 6 aa, Artificial Sequence
  The sequence has been designed and synthesized
QVQLVE

SEQ ID NO: 90 - DNA, 22 bp, Artificial Sequence
  The sequence has been designed and synthesized
tttgtttatccaccgaactaag

SEQ ID NO: 91 - DNA, 22 bp, Artificial Sequence
  The sequence has been designed and synthesized
tcttcagaaagggatccaccag

SEQ ID NO: 92 - DNA, 22 bp, Artificial Sequence
  The sequence has been designed and synthesized
tggtggatccctttctgaagac

SEQ ID NO: 93 - DNA, 18 bp, Artificial Sequence
  The sequence has been designed and synthesized
actgctccagtttcccac

SEQ ID NO: 94 - protein, 43 aa, Artificial Sequence
  The sequence has been designed and synthesized
TSPSTVRLESRVRELEDRLEELRDELERAERRANEMSIQLDEC

SEQ ID NO: 95 - protein, 505 aa, Artificial Sequence
  The sequence has been designed and synthesized
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSQVQLVETGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSEPKTPKPQPTSAIAGGGGSGGGGSGGGGSLQAMAAAQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSEPKTPKPQPARQTSPSTVRLESRVRELEDRLEELRDELERAERRANEMSIQLDEC

SEQ ID NO: 96 - protein, 133 aa, Artificial Sequence
  The sequence has been designed and synthesized. The antibody is a camelid (VHH)
QVQLVETGGGLVQAGDPLRLSCVASGRTVSRYDKAWFRQAPGKEREFVAGISWNGDTKIYADSVKGRFTISRENSRDTLDLQIDNLKPEDTAAYYCAVGIAGVQSMARMLGVRYWGQGTQVTVSSEPKTPKPQ

SEQ ID NO: 97 - DNA, 399 bp, Artificial Sequence
  The sequence has been designed and synthesized. The antibody is a camelid (VHH)
caggtgcagctcgtggagacggggggaggattggtgcaggctggggaccctctgagactctcctgtgtagcctctggacgcaccgtcagtcgctatgacaaggcctggttccgccaggctccagggaaggagcgtgagtttgtagcaggaattagctggaacggcgatacaaaaatttatgcagactccgtgaagggccgattcaccatctccagagagaactccagggatacactggatctgcaaattgacaacctgaaacctgaggacacggccgcgtattactgtgcggtcggaattgcgggtgttcagagtatggcgcgtatgctcggagtgcgctactggggccaggggacccaggtcaccgtctcctcagaacccaagacaccaaaaccacaa

SEQ ID NO: 98 - protein, 138 aa, Artificial Sequence
  The sequence has been designed and synthesized. The antibody is a camelid (VHH)
QVQLVETGGGLVQPGGSLRLSCAASGFSLDPYVIGWFRQAPGKEREGVSCITSRAASRTSVDSVNERFTISRDNAKNTVDLHINNLKPEDSGVYYCAAVPPAKLPLFSLCRSLPAKYDYWGQGTQVTVSSAHHSEDPS

SEQ ID NO: 99 - DNA, 414 bp, Artificial Sequence
  The sequence has been designed and synthesized. The antibody is a camelid (VHH)
caggtgcagctcgtggagacggggggaggcttggtgcagcctggggggtctctgagactctcctgtgcagcctctggtttcagtttggacccttatgtgataggatggttccggcaggccccagggaaggagcgtgagggggtctcatgtattacgagtagggctgctagtcgaacgtctgtagactccgtgaacgagcgattcaccatctccagagacaacgccaagaatacggtcgatctacacatcaataacctgaaacctgaggactcgggcgtttattactgtgcagcggtcccccctgccaaattaccacttttcagcctatgtcgctccctgccagcaaagtatgactactggggccaggggacccaggtcaccgtctcctcagcgcaccacagcgaagacccctcg

SEQ ID NO: 100 - protein, 125 aa, Artificial Sequence
  The sequence has been designed and synthesized. The antibody is a camelid (VHH)
QVQLVESGGGLVQPGGSLRLSCAASGSSFSRYAMRWYRQAPGKQRELVANINSRGTSNYADSVKGRFTISRDNAKNTVYLQMNSLKPEDTAVYYCNAEWLGRSEPSWGQGTQVTVSSEPKTPKPQ

SEQ ID NO: 101 - DNA, 375 bp, Artificial Sequence
  The sequence has been designed and synthesized. The antibody is a camelid (VHH)
caggtgcagctcgtggagtcggggggaggcttggtgcagcctggggggtctctgagactctcctgtgcagcctctggaagtagcttcagtagatatgccatgcgctggtaccgccaggctccagggaagcagcgcgagttggtcgcaaacattaatagtcgtggtacctcaaactatgcagactccgtgaagggccgattcaccatctccagagacaacgccaagaacacggtgtatctgcaaatgaacagcctgaaacctgaagacacggccgtctattattgtaatgcagagtggttgggacgatcggagccttcctggggccaggggacccaggtcaccgtctcctcggaacccaagacaccaaaaccacaa

SEQ ID NO: 102 - protein, 126 aa, Artificial Sequence
  The sequence has been designed and synthesized. The antibody is a camelid (VHH)
QVQLVESGGGLVQPGGSLRLSCAASGFIFSLYTMRWHRQAPGKERELVATITSATGITNYADSVKGRFIISRDDAKKTGYLQMNSLKPEDTAVYYCNAVRTTVSRDYWGQGTQVTVSSEPKTPKPQ

SEQ ID NO: 103 - DNA, 378 bp, Artificial Sequence
  The sequence has been designed and synthesized. The antibody is a camelid (VHH)
caggtgcagctcgtggagtcaggaggaggcttggtgcagcctggggggtctctgagactctcctgtgcagcctctggattcattttcagtctttataccatgaggtggcaccgccaggctccagggaaggagcgcgagttggtcgcgactattactagtgctactggtattacaaactatgcagactccgtgaagggccgattcatcatctccagagacgatgccaagaagacggggtatctgcaaatgaacagcctgaaacctgaggacacggccgtgtattactgtaatgcagtccgcactaccgtgtcacgagactactggggccaggggacccaggtcaccgtctcctcagaacccaagacaccaaaaccacaa

SEQ ID NO: 104 - protein, 125 aa, Artificial Sequence
  The sequence has been designed and synthesized. The antibody is a camelid (VHH)
QVQLVESGGGLVQPGGSLRLSCAASGIIFSIYTMGWYRQAPGKQRELVAAIPSGPSANATDSVGGRFTITRDNAENTVYLQMNDLKPEDTAVYYCNARRGPGIKNYWGQGTQVTVSSEPKTPKPQ

SEQ ID NO: 105 - DNA, 375 bp, Artificial Sequence
  The sequence has been designed and synthesized. The antibody is a camelid (VHH)
caggtgcagctcgtggagtctgggggaggcttggtgcagcctggggggtctctgagactctcctgtgcagcctctggaatcatcttcagtatctataccatgggctggtaccgccaggctccagggaagcagcgcgaattggtcgcagctatacctagtggtcctagcgcaaacgctacagactccgtggggggccgattcaccatcaccagagacaacgccgagaacacggtgtatctgcaaatgaacgacctgaaacctgaggacacggccgtctattactgtaatgctcggcggggtccgggtatcaaaaactactggggccaggggacccaggtcaccgtctcctcagaacccaagacaccaaaaccacaa

SEQ ID NO: 106 - protein, 130 aa, Artificial Sequence
  The sequence has been designed and synthesized. The antibody is a camelid (VHH)
QVQLVESGGGLVQPGGSLSVSCAASGSIARPGAMAWYRQAPGKERELVASITPGGLTNYADSVTGRFTISRDNAKRTVYLQMNSLQPEDTAVYYCHARIIPLGLGSEYRDHWGQGTQVTVSSAHHSEDPS

SEQ ID NO: 107 - DNA, 390 bp, Artificial Sequence
  The sequence has been designed and synthesized. The antibody is a camelid (VHH)
caggtgcagctcgtggagtccgggggcggcttggtgcagcccggggggtctctgagtgtctcctgtgcagcctctggaagcatcgcaagaccaggtgccatggcctggtaccgccaggctccagggaaggagcgcgagttggtcgcgtctattacgcctggtggtcttacaaactatgcggactccgtgacgggccgattcaccatttccagagacaacgccaagaggacggtgtatctgcagatgaacagcctccaacccgaggacacggccgtctattactgtcatgcacgaataattcccctaggacttgggtccgaatacagggaccactggggccaggggactcaggtcaccgtctcctcagcgcaccacagcgaagacccctcg

SEQ ID NO: 108 - protein, 123 aa, Artificial Sequence
  The sequence has been designed and synthesized. The antibody is a camelid (VHH)
QVQLVETGGGLVQPGGSLGLSCVVASGRSINNYGMGWYRQAPGKQRELVAQISSGGTTNYAGSVEGRFTISRDNVKKMVYLQMNSLKPEDTAVYYCNSLLRTFSWGQGTQVTVSSAHHSEDPS

SEQ ID NO: 109 - DNA, 369 bp, Artificial Sequence
  The sequence has been designed and synthesized. The antibody is a camelid (VHH)
caggtgcagctcgtggagacggggggaggcttggtgcagcctggggggtctctgggactctcctgtgtagtcgcctctggaagaagcatcaataattatggcatgggctggtaccgccaggctccagggaagcagcgcgagttggtcgcgcaaattagtagtggtggtaccacaaattatgcaggctccgtagagggccgattcaccatctccagagacaacgtcaagaaaatggtgtatcttcaaatgaacagcctgaaacctgaggacacggccgtctattactgtaattcactgctccgaactttttcctggggccaggggacccaggtcaccgtctcctcggcgcaccacagcgaagacccctcg

SEQ ID NO: 110 - protein, 133 aa, Artificial Sequence
  The sequence has been designed and synthesized. The antibody is a camelid (VHH)
QVQLVETGGLVQPGGSLRLSCAASGLTFSSTAMAWFRQAPGKEREFVARISGAGITIYYSDSVKDRFTISRNNVENTVYLQMNSLKTEDTAVYYCAARRNTYTSDYNIPARYPYWGQGTQVTVSSEPKTPKPQ

SEQ ID NO: 111 - DNA, 399 bp, Artificial Sequence
  The sequence has been designed and synthesized. The antibody is a camelid (VHH)
caggtgcagctcgtggagaccggggggttggtgcagcctgggggctccctgcgactctcctgtgcagcctccggactcaccttcagtagcactgccatggcctggttccgccaggctccagggaaggagcgtgagtttgtagcacgtattagcggggctggtattacgatctactattcggactccgtgaaggaccgattcaccatctccagaaacaacgtcgagaacacggtgtatttgcaaatgaacagcctgaaaactgaggacacggccgtttactactgtgcagcaagacggaatacttacactagcgactataacatacccgcccggtatccctactggggccaggggacccaggtcaccgtctcctcagaacccaagacaccaaaaccacaa

SEQ ID NO: 112 - protein, 126 aa, Artificial Sequence
  The sequence has been designed and synthesized. The antibody is a camelid (VHH)
QVQLVETGGLVQPGGSLRLSCAASRSTTATIYSMNWYRQAPGKQRELVAGMTSDGQTNYATSVKGRFTISRDNAKNTVYLLMNSLKLEDTAVYYCYVKPWRLQGWDYWGQGTQVTVSSEPKTPKPQ

SEQ ID NO: 113 - DNA, 378 bp, Artificial Sequence
  The sequence has been designed and synthesized. The antibody is a camelid (VHH)
caggtgcagctcgtggagacggggggcttggtgcagcctggggggtctctgagactctcctgtgcagcctctagaagcacgacggccacaatttatagtatgaactggtaccgccaggctccagggaagcagcgcgagttggtcgcgggtatgactagtgatggtcagacaaactatgcaacctccgtgaagggccgattcaccatctccagagacaacgccaagaacacggtatatttgctaatgaacagcctgaaacttgaggacacggccgtctattattgttatgtaaaaccatggagactacaaggttgggactactggggccaggggacccaggtcaccgtctcctcagaacccaagacaccaaaaccacaa

SEQ ID NO: 114 - protein, 122 aa, Artificial Sequence
  The sequence has been designed and synthesized. The antibody is a camelid (VHH)
QVQLVESGGGLVQPGGSLRLSCAAPESIVNSRTMAWYRQAPGKQRERVATITTAGSPNYADSVKGRFAISRDNAKNTVYLQMNSLKPEDTAVYYCNTLLSTLPYGQGTQVTVSSAHHSEDPS

SEQ ID NO: 115 - DNA, 366 bp, Artificial Sequence
  The sequence has been designed and synthesized. The antibody is a camelid (VHH)
caggtgcagctcgtggagtcgggcggcggcttggtgcagcctggggggtctctgagactctcctgtgcagcccctgaaagcatcgtcaatagcagaaccatggcctggtaccgccaggctccaggaaagcagcgcgaaagggtcgccactattactactgctggtagcccaaattatgcagactctgtgaagggccgattcgccatctccagagacaacgccaagaacacggtatatctgcaaatgaacagcctgaaacctgaggacacggccgtctattactgcaatacacttctcagcacccttccctatggccaggggacccaggtcaccgtctcctcggcgcaccacagcgaagacccctcg

SEQ ID NO: 116 - protein, 123 aa, Artificial Sequence
  The sequence has been designed and synthesized. The antibody is a camelid (VHH)
QVQLVESGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSSEPKTPKPQ

SEQ ID NO: 117 - DNA, 369 bp, Artificial Sequence
  The sequence has been designed and synthesized. The antibody is a camelid (VHH)
caggtgcagctcgtggagtcgggcggaggcttggtgcagcctggggggtctctgggactctcctgtgtagtcgcctctgaaagaagcatcaataattatggcatgggctggtaccgccaggctccagggaagcagcgcgagttggtcgcgcaaattagtagtggtggtaccacaaattatgcagactccgtagagggccgattcaccatctccagagacaacgtcaagaaaatggtgcatcttcaagtgaacagcctgaaacctgaggacacggccgtctattactgtaattcgctactccgaactttttcctggggccaggggacccaggtcaccgtctcctcggaacccaagacaccaaaaccacaa

SEQ ID NO: 118 - protein, 126 aa, Artificial Sequence
  The sequence has been designed and synthesized. The antibody is a camelid (VHH)
QVQLVETGGGLVQPGGSLRLSCAASGFTFSSYRMSWYRQAAGKERDVVATITANGVPTGYADSVMGRFTISRDNAKNTVYLEMNSLNPEDTAVYYCNAPRLHTSVGYWGQGTQVTVSSEPKTPKPQ

SEQ ID NO: 119 - DNA, 378 bp, Artificial Sequence
  The sequence has been designed and synthesized. The antibody is a camelid (VHH)
caggtgcagctcgtggagacgggaggaggcttggtgcagcctggggggtctctgagactctcctgtgcagcctctggattcaccttcagtagttatcgcatgagctggtaccggcaggctgcagggaaggagcgcgacgtggtcgcaacaattactgctaatggtgttcccacaggctatgcagactccgtgatgggccgattcaccatttccagagacaatgccaagaacacggtgtatctggaaatgaacagcctgaatcctgaggacacggccgtgtattactgtaacgcgccccgtttgcatacatctgtaggctactggggccaggggacccaggtcaccgtctcctcagaacccaagacaccaaaaccacaa

SEQ ID NO: 120 - protein, 135 aa, Artificial Sequence
  The sequence has been designed and synthesized. The antibody is a camelid (VHH)
QVQLVESGGGLVQAGNSLRLSCTASGVIFSIYTMGWFRQAPGKEREFVAAIGVADGTALVADSVTGRFTISRDNAKNTVYLHMNSLKPEDTAVYSCAAYLSPRVQSPYITDSRYQLWGQGTQVTVSSEPKTPKPQ

SEQ ID NO: 121 - DNA, 405 bp, Artificial Sequence
  The sequence has been designed and synthesized. The antibody is a camelid (VHH)
caggtgcagctcgtggagtcgggaggaggattggtgcaggctgggaactctctgagactctcctgtacggcctctggtgtgatcttctctatctataccatgggctggttccgccaggctccagggaaggagcgtgagtttgtagcagcgataggggtggctgatggtaccgcacttgtggcagactccgtgacgggccgattcaccatctccagagacaacgccaagaacaccgtttatctgcatatgaacagcctgaagcctgaggacacggccgtctattcctgtgcagcgtatcttagcccccgtgtccaatccccctacataactgactcccggtatcaactctggggccaggggacccaggtcaccgtctcctcagaacccaagacaccaaaaccacaa

SEQ ID NO: 122 - protein, 119 aa, Artificial Sequence
  The sequence has been designed and synthesized. The antibody is a camelid (VHH)
TGGGLVQAGGSLRLSCAASGRYAMGWFRQAPGKEREFVATISRSGAIREYADSVKGRFTISRDGAENTVYLEMNSLKPDDTAIYVCAEGRGATFNPEYAYWGQGTQVTVSSAHHSEDPS

SEQ ID NO: 123 - DNA, 375 bp, Artificial Sequence
  The sequence has been designed and synthesized. The antibody is a camelid (VHH)
caggtgcagctcgtggagactgggggaggattggtgcaggctgggggctctctgaggctctcctgtgcagcctctggacgctatgccatgggctggttccgccaggctccagggaaggagcgtgaatttgtagcgactattagccggagtggtgctatcagagagtatgcagactccgtgaagggccgattcaccatctccagagacggcgccgagaacacggtgtatctggaaatgaacagcctgaaacctgacgacacggccatttatgtctgtgcagaaggacgaggggcgacattcaaccccgagtatgcttactggggccaggggacccaggtcaccgtctcctcagcgcaccacagcgaagacccctcg

SEQ ID NO: 124 - protein, 127 aa, Artificial Sequence
  The sequence has been designed and synthesized. The antibody is a camelid (VHH)
QVQLVESGGGLVQPGGSLRLSCAASGFTLDDYAIGWFRQVPGKEREGVACVKDGSTYYADSVKGRFTISRDNGAVYLQMNSLKPEDTAVYYCASRPCFLGVPLIDFGSWGQGTQVTVSSEPKTPKPQ

SEQ ID NO: 125 - DNA, 381 bp, Artificial Sequence
  The sequence has been designed and synthesized. The antibody is a camelid (VHH)
caggtgcagctcgtggagtcgggcggaggcttggtgcagcctggggggtctctgagactctcctgtgcagcctctggattcactttggatgattatgccataggctggttccgccaggtcccagggaaggagcgtgagggggtcgcatgtgttaaagatggtagtacatactatgcagactccgtgaagggccgattcaccatctccagagacaacggcgcggtgtatctgcaaatgaacagcctgaaacctgaggacacagccgtttattactgtgcatccaggccctgctttttgggtgtaccacttattgactttggttcctggggccaggggacccaggtcaccgtctcctcggaacccaagacaccaaaaccacaa

SEQ ID NO: 126 - protein, 135 aa, Artificial Sequence
  The sequence has been designed and synthesized. The antibody is a camelid (VHH)
QVQLVESGGGLVQAGGSLRLSCATSGGTFSDYGMGWFRQAPGKEREFVAAIRRNGNGGNGIEYADSVKGRFTISRDNAKNTVHLQMNSLTPEDTAVYYCAASISGYAYNTIERYNYWGQGTQVTVSSEPKTPKPQ

SEQ ID NO: 127 - DNA, 406 bp, Artificial Sequence
  The sequence has been designed and synthesized. The antibody is a camelid (VHH)
caggtgcagctcgtggagtcagggggaggattggtgcaggctgggggctctctgagactctcctgcgcaacctctggcggcaccttcagtgactatggaatgggctggttccgccaggctccagggaaggagcgtgagtttgtagcagctattaggcggaatggtaatggcggtaatggcattgaatatgcagactccgtgaagggccgattcaccatctccagagacaacgccaagaacacggtgcatctacaaatgaacagcctgacacctgaggacacggccgtttattactgtgcagcgtcaatatcgggatacgcttataacacaattgaaagatataactactggggccagggaacccaggtcaccgtctcctcaggaacccaagacaccaaaaccacaa

SEQ ID NO: 128 - protein, 137 aa, Artificial Sequence
  The sequence has been designed and synthesized. The antibody is a camelid (VHH)
QVQLVESGGGLVQAGGSLSLSCAASGGDFSRNAMAWFRQAPGKEREFVASINWTGSGTYYLDSVKGRFTISRDNAKNALYLQMNNLKPEDTAVYYCARSTVFAEITGLAGYQSGSYDYWGQGTQVTVSSEPKTPKPQ

SEQ ID NO: 129 - DNA, 411 bp, Artificial Sequence
  The sequence has been designed and synthesized. The antibody is a camelid (VHH)
caggtgcagctcgtggagtccggcggaggattggtgcaggcggggggctctctgagtctctcctgtgcagcctctggaggtgacttcagtaggaatgccatggcctggttccgtcaggctccagggaaggagcgtgaatttgtagcatctattaactggactggtagtggcacatattatctagactccgtgaagggccgattcaccatctccagagacaacgccaagaacgccctgtatctgcaaatgaacaacctgaaacctgaggacacggccgtttattactgtgcacgctccacggtgtttgccgaaattacaggcttagcaggctaccagtcgggatcgtatgactactggggccaggggacccaggtcaccgtctcctcagaacccaagacaccaaaaccacaa

SEQ ID NO: 130 - protein, 137 aa, Artificial Sequence
  The sequence has been designed and synthesized. The antibody is a camelid (VHH)
QVQLVETGGGTVQTGGSLRLSCSASGGSFSRNAMGWFRQAPGKEREFVAAINWSASSTYYRDSVKGRFTVSRDNAKNTVYLHLNSLKLEDTAAYYCAGSSVYAEMPYADSVKATSYNYWGQGTQVTVSSEPKTPKPQ

SEQ ID NO: 131 - DNA, 411 bp, Artificial Sequence
  The sequence has been designed and synthesized. The antibody is a camelid (VHH)
caggtgcagctcgtggagaccggcggaggaacggtgcanactgggggctctctgagactctcctgttcagcctctggcggctccttcagtaggaatgccatgggctggttccgccaggctccagggaaggagcgtgaatttgtagcagctattaactggagtgcctctagtacttattatagagactccgtgaagggacgattcaccgtctccagagacaacgccaagaacacggtgtatctgcatttgaacagcctgaaacttgaggacacggccgcgtattactgtgctggaagctcggtgtatgcagaaatgccgtacgccgactctgtcaaggcaacttcctataactactggggccaggggacccaggtcaccgtctcctcagaacccaagacaccaaaaccacaa

SEQ ID NO: 132 - protein, 131 aa, Artificial Sequence
  The sequence has been designed and synthesized. The antibody is a camelid (VHH)
QVQLVETGGGLVQAGGSLRLPCSFSGFPFDNYFVGWFRQAPGKEREGVSCISSSDGSTYYADSVKGRFTISRDNAKNTVYLQMNSLKPEDTAVYYCGADFLTPHRCPALYDYWGQGTQVTVSSAHHSEDPS

SEQ ID NO: 133 - DNA, 393 bp, Artificial Sequence
  The sequence has been designed and synthesized. The antibody is a camelid (VHH)
caggtgcagctcgtggagaccgggggaggcttggtgcaggctggggggtctctgagactcccctgttcattctctggattccctttcgataattatttcgtaggctggttccgccaggccccagggaaggagcgtgagggggtctcatgtattagtagtagtgatggtagcacatactatgcagactccgtgaagggccggttcaccatctccagagacaacgccaagaacacggtgtatctgcaaatgaacagtctgaaacctgaggatacggccgtttattactgtggagcagatttcctcaccccacataggtgtccagccttatatgactactggggccaggggacccaggtcaccgtctcctcagcgcaccacagcgaagacccctcg

SEQ ID NO: 134 - protein, 127 aa, Artificial Sequence
  The sequence has been designed and synthesized. The antibody is a camelid (VHH)
QVQLVESGGGLVQPGGSLRLHCAASGSIASIYRTCWYRQGTGKQRELVAAITSGGNTYYADSVKGRFTISRDNAKNTIDLQMNSLKPEDTAVYYCNADEAGIGGFNDYWGQGTQVTVSSAHHSEDPS

SEQ ID NO: 135 - DNA, 381 bp, Artificial Sequence
  The sequence has been designed and synthesized. The antibody is a camelid (VHH)
caggtgcagctcgtggagtctggtggaggcttggtgcagcctggggggtctctgagactccactgtgcagcctctggaagcatcgccagtatctatcgcacgtgctggtaccgccagggcacagggaagcagcgcgagttggtcgcagccattactagtggtggtaacacatactatgcggactccgttaagggccgattcaccatctccagagacaacgccaaaaacacaatcgatctgcaaatgaacagcctgaaacctgaggacacggccgtctattactgtaatgcagacgaggcggggatcgggggatttaatgactactggggccaggggacccaggtcaccgtctcctcagcgcaccacagcgaagacccctcg

SEQ ID NO: 136 - protein, 134 aa, Artificial Sequence
  The sequence has been designed and synthesized. The antibody is a camelid (VHH)
QVQLVESGGGLVQAGGSLRLSCAASGRTFSRSSMGWFRQAPGKEREFVASIVWADGTTLYGDSVKGRFTVSRDNVKNMVYLQMNNLKPEDTALYYCADNKFVRGLVAVRAIDYDYWGQGTQVTVSSEPKTPKPQ

SEQ ID NO: 137 - DNA, 402 bp, Artificial Sequence
  The sequence has been designed and synthesized. The antibody is a camelid (VHH)
caggtgcagctcgtggagtcggggggaggattggtgcaggctgggggctctctgagactctcctgtgcagcctctggacgcaccttcagtcgcagttccatgggctggttccgccaggctccagggaaggagcgtgaattcgttgcgtccattgtctgggctgatggtacgacgttgtatggagactccgtaaagggccgattcaccgtctccagggacaacgtcaagaacatggtgtatctacaaatgaacaacctgaaacctgaggacacggccctttattactgtgcggacaataaattcgtccgtggattagtggctgtccgtgcgatagattatgactactggggccaggggacccaggtcaccgtctcgtcagaacccaagacaccaaaaccacaa

SEQ ID NO: 138 - protein, 135 aa, Artificial Sequence
  The sequence has been designed and synthesized. The antibody is a camelid (VHH)
QVQLVESGGLVQAGGSLRLSCAASGRADIIYAMGWFRQAPGKEREFVAAVDWSGGSTYYADSVKGRFTISRDNAKNSVYLQMNSLKPEDTAVYYCAARRSWYRDALSPSRVYEYDYWGQGTQVTVSSEPKTPKPQ

SEQ ID NO: 139 - DNA, 405 bp, Artificial Sequence
  The sequence has been designed and synthesized. The antibody is a camelid (VHH)
caggtgcagctcgtggagtcgggaggattggtgcaggctggaggctctctgagactctcctgcgcagcctctggacgcgccgacataatctatgccatgggctggttccgccaggctccagggaaggagcgtgagtttgtagcggcagtagactggagtggtggtagcacatactatgcagactccgtgaagggccgattcaccatctccagagacaacgccaagaactcggtgtatctgcaaatgaacagcctgaaacctgaggacacggccgtttattactgtgcagcccgaaggagctggtaccgagacgcgctatccccctcccgggtgtatgaatatgactactggggccaggggacccaggtcaccgtctcctcagaacccaagacaccaaaaccacaa

SEQ ID NO: 140 - protein, 125 aa, Artificial Sequence
  The sequence has been designed and synthesized. The antibody is a camelid (VHH)
QVQLVETGGGLVQPGGSLTLSCAGSGGTLEHYAIGWFRQAPGKEHEWLVCNRGEYGSTVYVDSVKGRFTASRDNAKNTVYLQLNSLKPDDTGIYYCVSGCYSWRGPWGQGTQVTVSSAHHSEDPS

SEQ ID NO: 141 - DNA, 375 bp, Artificial Sequence
  The sequence has been designed and synthesized. The antibody is a camelid (VHH)
caggtgcagctcgtggagacgggaggaggcttggtgcagcctggggggtctctgacactctcctgtgcaggctccggtggcactttggaacattatgctataggctggttccgccaggcccctgggaaagagcatgagtggctcgtatgtaatagaggtgaatatgggagcactgtctatgtagactccgtgaagggccgattcaccgcctccagagacaacgccaagaacacggtgtatctgcaattgaacagtctgaaacctgacgacacaggcatttattactgtgtatcgggatgttactcctggcggggtccctggggccaggggacccaggtcaccgtctcctcggcgcaccacagcgaagacccctcg

SEQ ID NO: 142 - protein, 126 aa, Artificial Sequence
  The sequence has been designed and synthesized. The antibody is a camelid (VHH)
QVQLVESGGGLVQPGGSLKLSCRASGSIVSIYAVGWYRQAPGKQRELLAAITTDGSTKYSDSVKGRFTISRDNAKNTVYLQMNNLKPEDTAIYSCIGDAAGWGDQYYWGQGTQVTVSSEPKTPKPQ

SEQ ID NO: 143 - DNA, 378 bp, Artificial Sequence
  The sequence has been designed and synthesized. The antibody is a camelid (VHH)
caggtgcagctcgtggagtctgggggaggtttggtgcagcctggggggtctctgaaactctcctgtagagcctctggaagcatagtcagtatctatgccgtgggctggtaccgccaggctccagggaagcagcgcgagttgctcgcggctatcactactgatggtagcacgaagtactcagactccgtgaagggccgattcaccatctcccgagacaacgccaagaacacggtatatctgcaaatgaacaacctcaaacctgaggacacggccatctattcctgtatcggggacgcggcgggttggggcgaccaatactactggggccaggggacccaggtcaccgtctcctcagaacccaagacaccaaaaccacaa

SEQ ID NO: 144 - protein, 129 aa, Artificial Sequence
  The sequence has been designed and synthesized. The antibody is a camelid (VHH)
QVQLVESGGGLVQAGGSLRLSCAASGSIVNFETMGWYRQAPGKERELVATITNEGSSNYADSVKGRFTISGDNAKNTVSLQMNSLKPEDTAVYYCSATFGSRWPYAHSDHWGQGTQVTVSSEPKTPKPQ

SEQ ID NO: 145 - DNA, 387 bp, Artificial Sequence
  The sequence has been designed and synthesized. The antibody is a camelid (VHH)
caggtgcagctcgtggagtcaggcggaggcttggtgcaggctggggggtctctgagactctcctgtgcagcctctggaagcatcgtcaatttcgaaaccatgggctggtaccgccaggctccagggaaggagcgcgagttggtcgcaactattactaatgaaggtagttcaaactatgcagactccgtgaagggccgattcaccatctccggagacaacgccaagaacacggtgtccctgcaaatgaacagcctgaaacctgaggacacggccgtctactactgttcggcgacgttcggcagtaggtggccgtacgcccacagtgatcactggggccaggggacccaggtcaccgtctcctcagaacccaagacaccaaaaccacaa

SEQ ID NO: 146 - protein, 125 aa, Artificial Sequence
  The sequence has been designed and synthesized. The antibody is a camelid (VHH)
QVQLVETGGALVHTGGSLRLSCEVSGSTFSSYGMAWYRQAPGEQRKWVAGIMPDGTPSYVNSVKGRFTISRDNAKNSVYLHMNNLRPEDTAVYYCNQWPRTMPDANWGRGTQVTVSSEPKTPKPQ

SEQ ID NO: 147 - DNA, 375 bp, Artificial Sequence
  The sequence has been designed and synthesized. The antibody is a camelid (VHH)
caggtgcagctcgtggagacgggcggagcattggtgcacactgggggttctctgagactctcctgcgaagtctccggaagcaccttcagtagctatggcatggcctggtaccgccaagctccaggcgagcagcgtaagtgggtcgcaggtattatgccggatggtactccaagctatgtaaactccgtgaagggccgattcaccatctccagagacaacgccaagaactcggtgtatctgcacatgaacaacctgaggcctgaagacacggccgtctattattgcaaccaatggccgcgcacgatgcctgacgcgaactggggccgggggacccaggtcaccgtctcctcagaacccaagacaccaaaaccacaa

SEQ ID NO: 148 - protein, 117 aa, Artificial Sequence
  The sequence has been designed and synthesized. The antibody is a camelid (VHH)
QVQLVETGGSLRLTCVTSGSTFNNPAITWYRQPPGKQREWVASLRSGDGPVYRESVKGRFTIFRDNATDALYLRMNSLKPEDTAVYHCNTASPASWLDWGQGTQVTVSSEPKTPKPQ

SEQ ID NO: 149 - DNA, 351 bp, Artificial Sequence
  The sequence has been designed and synthesized. The antibody is a camelid (VHH)
caggtgcagctcgtggagactggggggtctctgaggctcacctgtgtaacctctggaagcaccttcaataatcctgccataacctggtaccgccagcctccagggaagcagcgtgagtgggtcgcaagtcttcgtagtggtgatggtccagtatatagggaatccgtgaagggccgattcaccatttttagagacaacgccacggacgcgctgtatctgcggatgaatagcctgaaacctgaggacacggccgtctatcactgtaacaccgcctcacctgctagttggctggactggggccaggggacccaggtcactgtctcctcagaacccaagacaccaaaaccacaa

SEQ ID NO: 150 - protein, 130 aa, Artificial Sequence
  The sequence has been designed and synthesized. The antibody is a camelid (VHH)
QVQLVETGGGLVQPGGSLRLSCATSGFPFSTERMSWVRQAPGKGLEWVSGITEGGETTLAAPSVKGRFNISRDNARNILYLQMNSLKPEDAAVYYCFRGVFFRTSFPPELARGQGTQVTVSSEPKTPKPQ

SEQ ID NO: 151 - DNA, 390 bp, Artificial Sequence
  The sequence has been designed and synthesized. The antibody is a camelid (VHH)
caggtgcagctcgtggagacgggaggaggattggtgcaacctgggggttctctgagactctcttgtgcaacctctggattccccttcagtacggagcgtatgagctgggtccgccaggctccaggaaaggggctcgagtgggtctcaggtattactgagggtggtgaaaccactctcgcggcaccctccgtgaagggccgattcaacatctccagagacaacgccaggaatatcctatatctacagatgaattccttgaaacctgaggacgcggccgtttactattgttttagaggtgttttttttagaacgagttttcctcccgaactcgcgcggggccaggggacccaggtcaccgtctcctcagaacccaagacaccaaaaccacaa

SEQ ID NO: 152 - protein, 127 aa, Artificial Sequence
  The sequence has been designed and synthesized. The antibody is a camelid (VHH)
QVQLVESGGGLVQAGGSLRLSCAASGSAVSDSFSTYAISWHRQAPGKQREWIAGISNRGATSYRDSVKGRFTISRDNAKNTVYLQMNNLKPEDTGVYYCEPWPREGLGGGQGTQVTVSSEPKTPKPQ

SEQ ID NO: 153 - DNA, 381 bp, Artificial Sequence
  The sequence has been designed and synthesized. The antibody is a camelid (VHH)
caggtgcagctcgtggagtcgggcggaggcttggtgcaggcaggggggtctttgagactctcctgtgcagcctctggaagcgccgtcagtgacagcttcagtacctatgccatctcctggcaccgccaggctccagggaagcagcgtgagtggatcgcaggtattagtaatcgtggtgcgacaagctatagagactccgtgaagggccgattcaccatctccagagacaacgccaagaacacggtatatctgcaaatgaacaacctgaaacctgaggacacgggcgtctattattgtgagccatggccacgcgaaggacttggggggggccaggggactcaggtcaccgtctcctcagaacccaagacaccaaaaccacaa

SEQ ID NO: 154 - protein, 124 aa, Artificial Sequence
  The sequence has been designed and synthesized. The antibody is a camelid (VHH)
QVQLVESGGGSVQTGGSLTLSCVVSGSTFSDYAVAWYRQVPGKSRAWVAGVSTTGSTSYTDSVRGRFTISRDNHKKTVYLSMNSLKPEDTGIYYCNLWPFTNPPSWGQGTQVTVSSAHHSEDPS

SEQ ID NO: 155 - DNA, 372 bp, Artificial Sequence
  The sequence has been designed and synthesized. The antibody is a camelid (VHH)
caggtgcagctcgtggagtcggggggaggctcggtgcanactggggggtctctgacactctcctgtgtagtctctggaagtaccttcagtgactatgcggtggcctggtaccgccaggttccaggcaaatcgcgtgcgtgggtcgcgggtgttagtactactggctcgacatcttatacagactccgtgaggggccggttcaccatctccagagacaaccacaagaagacggtgtatctttcaatgaacagcctgaaacctgaggacacgggcatctattactgcaacttatggccgttcacaaatcctccttcctggggccagggaacccaagtcaccgtttcctcggcgcaccacagcgaagacccctcg

SEQ ID NO: 156 - protein, 132 aa, Artificial Sequence
  The sequence has been designed and synthesized. The antibody is a camelid (VHH)
QVQLVESGGAVVQPGGSLRLSCATSGFTFSDDRMSWARQAPGKGLEWVSGISTASEGFATLYAPSVKGRFTISRDNAKHMLYLQMDTLKPEDTAVYYCLRGVFFRTNIPPEVLRGQGTQVTVSSAHHSEDPS

SEQ ID NO: 157 - DNA, 396 bp, Artificial Sequence
  The sequence has been designed and synthesized. The antibody is a camelid (VHH)
caggtgcagctcgtggagtctggaggagccgtggtgcaacctgggggttctctgagactctcctgtgcaacctctggattcaccttcagtgacgatcgtatgagctgggcccgccaggctccaggaaaggggctcgagtgggtctcaggtattagtactgctagtgaaggttttgctacactctacgcaccctccgtgaagggccgattcaccatctccagagacaacgccaagcatatgctgtatctgcaaatggataccttgaaacctgaggacacggccgtgtattactgtttaagaggggttttttttagaacgaacattcctcccgaggtactgcggggccaggggacccaggtcaccgtctcctcagcgcaccacagcgaagacccctcg

SEQ ID NO: 158 - protein, 126 aa, Artificial Sequence
  The sequence has been designed and synthesized. The antibody is a camelid (VHH)
QVQLVETGGDLVQPGGSLRLSCAASGSSFSRAAVGWYRQAPGKEREWVARLASGDMTDYTESVRGRFTISRDNAKHTVYLQMDNLKPEDTAVYYCKARIPPYYSIEYWGKGTRVTVSSEPKTPKPQ

SEQ ID NO: 159 - DNA, 378 bp, Artificial Sequence
  The sequence has been designed and synthesized. The antibody is a camelid (VHH)
caggtgcagctcgtggagacggggggagacttggtgcancctggggggtctctgagactctcctgtgcagcctctggaagctccttcagccgcgctgccgtgggctggtaccgtcaggctccaggaaaggagcgtgagtgggtcgcacgtctcgcgagtggtgatatgacggactataccgagtccgtgaggggccgattcactatctccagagacaacgccaagcacacggtgtatctgcaaatggacaacctgaaacctgaggacacggccgtctactattgtaaggccaggatacccccttattactctatagagtactggggcaaagggacccgggtcaccgtctcctcanaacccaagacaccaaaaccacaa

SEQ ID NO: 160 - protein, 124 aa, Artificial Sequence
  The sequence has been designed and synthesized. The antibody is a camelid (VHH)
QVQLVETGGGLVQAGGSLRLSCVVSSPLFNLYDMAWYRQAPGNQRELVAGILTDGRATYSDSVKGRFTISRNNLTNTVFLQMSSLKPEDTAVYYCNRKNSIYWDSWGQGTQVTVSSEPKTPKPQ

SEQ ID NO: 161 - DNA, 372 bp, Artificial Sequence
  The sequence has been designed and synthesized. The antibody is a camelid (VHH)
caggtgcagctcgtggagacaggtggaggcttggtgcaggctggggggtctctgagactctcctgtgtagtatctagtcccctgttcaatctttacgacatggcctggtatcgccaggctccagggaatcagcgtgagttggtcgcaggcatcttgactgatggtcgcgcaacatattcagacagcgtgaagggccgattcaccatttccagaaacaacctgacgaacacggtgtttttacaaatgagcagcctgaaacctgaggacacggccgtctattattgtaatagaaagaatagtatctactgggattcctggggccaggggacccaggtcaccgtctcctcggaacccaagacaccaaaaccacaa

SEQ ID NO: 162 - protein, 134 aa, Artificial Sequence
  The sequence has been designed and synthesized. The antibody is a camelid (VHH)
QVQLVESGGGLVQAGGSLRLSCVASGLTFSRYGMGWFRQAPGQERVVVSVISPDGGSAYYADSVKGRFTISRDNAKNTVYLQMSTLRFEDTGVYYCTAGPRNGATTVLRPGDYDYWGQGTQVTVSSEPKTPKPQ

SEQ ID NO: 163 - DNA, 402 bp, Artificial Sequence
  The sequence has been designed and synthesized. The antibody is a camelid (VHH)
caggtgcagctcgtggagtcggggggaggattggtgcaggctgggggctctctgagactctcctgcgtagcctctggactcaccttcagtcgctatggcatgggctggttccgccaggctccaggacaggagcgtgtagtcgtatcagttattagtcccgacggtggtagcgcatactacgcagactccgtgaagggccgattcaccatctccagagacaacgccaagaacacggtgtatctgcaaatgagcaccctgagatttgaggacacgggcgtttattattgtacagcagggccccggaatggagcgactacagtcctccggccaggggattatgactactggggccaggggacccaggtcactgtctcctcagaacccaagacaccaaaaccacaa

(19) United States
(12) Patent Application Publication
(10) Pub. No.: US 2014/0294826 A1
(43) Pub. Date: Oct. 2, 2014

(54) Methods and compositions with a recombinant neutralizing binding protein for treating toxin exposure

(71) Applicant: Tufts University, Boston, MA (US)

(72) Inventor: Charles B. Shoemaker, North Grafton, MA (US)

(73) Assignee: Tufts University, Boston, MA (US)

(21) Appl. No.: 14/247,628

(22) Filed: Apr. 8, 2014

Related U.S. Application Data
(63) Continuation-in-part of application No. 13/566,524, filed on Aug. 3, 2012.
Application No. 13/566,524 is a continuation-in-part of application No. 12/889,511, filed on Sep. 24, 2010, now Pat. No. 8,349,326.
Application No. 12/889,511 is a continuation-in-part of application No. 12/032,744, filed on Feb. 18, 2008.
(60) Provisional application No. 61/809,685, filed on Apr. 8, 2013.
(60) Provisional application No. 61/514,949, filed on Aug. 4, 2011.
(60) Provisional application No. 60/890,626, filed on Feb. 20, 2007.

(51) Int. Cl. C07K 16/00; C07K 16/16; C07K 16/12

(52) U.S. Cl. CPC C07K 16/005; C07K 16/1282; C07K 16/1228; C07K 16/1278; C07K 16/16; A61K 2039/505
USPC 424/134.1; 530/387.3; 536/23.1; 435/320.1; 424/130.1; 514/44 R; 530/391.3; 424/133.1; 424/136.1

(57) ABSTRACT

Methods, compositions and kits are provided for treating a subject exposed to or at risk for exposure to a disease agent using a pharmaceutical composition including at least one recombinant binding protein or a source of expression of the binding protein, wherein the binding protein neutralizes at least one or a plurality of disease agents that are toxins, for example at least one of a ricin toxin, a Shiga toxin, or an anthrax toxin.

### RELATED APPLICATIONS

This application claims the benefit of U.S. provisional application Ser. No. 61/809,685 filed Apr. 8, 2013 entitled, “Methods, compositions and kits for treating a subject using a recombinant neutralizing binding protein”, inventor Charles B. Shoemaker, and is a continuation-in-part of U.S. utility application Ser. No. 13/566,524, filed Aug. 3, 2012, which claims the benefit of U.S. provisional application Ser. No. 61/514,949 filed Aug. 4, 2011 entitled, “Methods, compositions and kits for treating a subject using a recombinant heteromultimeric neutralizing binding protein”, inventors Charles B. Shoemaker and Hanping Feng and which is a continuation-in-part of U.S. utility application Ser. No. 12/889,511 filed Sep. 24, 2010, which is a continuation-in-part application of U.S. utility application Ser. No. 12/032,744 filed Feb. 18, 2008, which claims the benefit of U.S. provisional application Ser. No. 60/890,626 filed Feb. 20, 2007, each of which is incorporated by reference herein in its entirety.

### GOVERNMENT SUPPORT

This invention was made with government support under grants AI030050 and AI057159 awarded by the National Institutes of Health. The government has certain rights in the invention.

### TECHNICAL FIELD

Compositions and methods using a recombinant neutralizing binding protein are provided for treating a subject at risk for exposure or exposed to a disease agent.

### BACKGROUND

A need exists for generating high affinity binding agents that treat both routine incidents of disease and pandemics, and efforts to discover and produce these agents are underway. The production of antibodies and their storage is a costly and lengthy process. In fact, development of a single antibody therapeutic agent often requires years of clinical study. Yet multiple, different therapeutic antibodies are necessary for the effective treatment of patients exposed to a disease agent, an infection outbreak or a bio-terrorist assault. Developing and producing multiple antibodies that can bind to different targets (e.g. microbial pathogens, viral pathogens, toxins, and cancer cells) is often a difficult task because it involves separately producing, storing and transporting multiple antibodies for each pathogen or toxin. Production and stockpiling a sufficient amount of antibodies to protect large populations is a challenge and currently has not been achieved. The shelf life of antibodies is often relatively short (e.g., weeks or months), and accordingly freshly prepared batches of antibodies have to be produced to replace the expiring antibodies.

Accordingly, there is a need for a cost effective and efficient way to provide alternatives to current therapeutic agents. Further a need exists for alternative therapeutics that are easier to develop and produce, have a longer shelf life, and bind as a single agent to multiple targets on the same disease agent, as well as to different disease agents.

### SUMMARY

An aspect of the invention provides a pharmaceutical composition for treating a subject at risk for exposure to or exposed to at least one disease agent, the pharmaceutical composition including: at least one recombinant binding protein that neutralizes the disease agent and treats the subject for exposure to the disease agent, such that the binding protein includes at least one amino acid sequence selected from the group of:

(SEQ ID NO: 96);
QVQINETGGGLVQAGDPLRLSCVASGRIVSRYDKAWFRQAPGKEREFVAG
ISWNGDTKIYADSVKGRFTISRENSRDTLDLQIDNLKPEDTAAYYCAVGI
AGVQSMARMLGVRYWGQGTQVTVSSEPKTPKPQ
(SEQ ID NO: 98);
QVQLVETGGGLVQPGGSLRLSCAASGFSLDPYVIGWFRQAPGKEREGVSC
ITSRAASRTSVDSVNERFTISRDNAKNINDLHINNLKPEDSGVYYCAAVP
PAKLPLFSLCRSLPAKYDYWGQGTQVTVSSAHHSEDPS
(SEQ ID NO: 100);
QVQLVESGGGLVQPGGSLRLSCAASGSSFSRYAMRWYRQAPGKQRELVAN
INSRGTSNYADSVKGRFTISRDNAKNTVYLQMNSLKPEDTAVYYCNAEWL
GRSEPSWGQGTQVTVSSEPKTPKPQ
(SEQ ID NO: 102);
QVQLVESGGGLVQPGGSLRLSCAASGFIFSLYTMRWFIRQAPGKERELVA
TITSATGITNYADSVKGREIISRDDAKKTGYLQMNSIKPEDTAVYYCNAV
RTTVSRDYWGQGTQVTVSSEPKTPKPQ
(SEQ ID NO: 104);
QVQLVESGGGLVQPGGSLRISCAASGIIFSTYTMGWYRQAPGKQRELVAA
IPSGPSANATDSVGGRFTITRDNAENTVYLQMNDLKPEDTAVYYCNARRG
PGIKNYWGQGTQVTVSSEPKTPKPQ
(SEQ ID NO: 106);
QVQLVESGGGLVQPGGSLSVSCAASGSIARPGAMAWYRQAPGKERELVAS
TTPGGLTNYADSVTGRFTISRDNAKRTVYLQIVINSLQPEDTAVYYCIIA
RIIPLGLGSEYRDIIWGQGTQVTVSSAHHSEDPS
(SEQ ID NO: 108);
QVQLVETGGGLVQPGGSLGLSCVVASGRSINNYGMGWYRQAPGKQRELVA
QISSGGTTNYAGSVEGRFTISRDNVKKMVYLQMNSLKPEDTAVYYCNSLL
RTFSWGQGTQVTVSSAHHSEDPS
(SEQ ID NO: 110);
QVQLVETGGLVQPGGSLRLSCAASGLTESSTAMAWFRQAPGKEREFVARI
SGAGITIYYSDSVKDRFTISRNNVENTVYLQMNSLKTEDTAVYYCAARRN
TYTSDYNIPARYPYWGQGTQVTVSSEPKTPKPQ
(SEQ ID NO: 112);
QVQLVETGGLVQPGGSLRLSCAASRSTTATIYSMNWYRQAPGKQRELVAG
MTSDGQTNYATSVKGRFTISRDNAKNTVYLLMNSLKLEDTAVYYCYVKPW
RLQGWDYWGQGTQVTVSSEPKTPKPQ
(SEQ ID NO: 114);
QVQLVESGGGLVQPGGSLRLSCAAPESIVNSRTMAWYRQAPGKQRERVAT
ITTAGSPNYADSVKGRFAISRDNAKNTVYLQMNSLKPEDTAVYYCNTLLS
TLPYGQGTQVTVSSAHHSEDPS
(SEQ ID NO: 116);
QVQLVESGGGLVQPGGSLGLSCVVASERSINNYGMGWYRQAPGKQRELVA
QISSGGTTNYADSVEGRFTISRDNVKKMVHLQVNSLKPEDTAVYYCNSUR
TFSWGQGTQVTVSSEPKTPKPQ
(SEQ ID NO: 118);
QVQLVETGGGIVQPGGSLRLSCAASGFTFSSYRMSWYRQAAGKERDVVAT
ITANGVPTGYADSVMGRFTISRDNAKNTVYLEMNSLNPEDTAVYYCNAPR
LHTSVGYWGQGTQVTVSSEPKTPKPQ
(SEQ ID NO: 120);
QVQLVESGGGLVQAGNSLRLSCTASGVIFSIYTMGWFRQAPGKEREFVAA
IGVADGTALVADSVTGRFTISRDNAKNTVYLHMNSLKPEDTAVYSCAAYL
SPRVQSPYITDSRYQLWGQGTQVTVSSEPKIPKPQ
(SEQ ID NO: 122);
TGGGLVQAGGSLRLSCAASGRYAMGWFRQAPGKEREEVATISRSGAIREY
ADSVKGRFTISRDGAENTVYLEMNSLIUDDTATYVCAEGRGATFNPEYAY
WGQGTQVTVSSAHHSEDPS
(SEQ ID NO: 124);
QVQLVESGGGLVQPGGSLRLSCAASGFTLDDYAIGWFRQVPGKEREGVAC
VKDGSTYYADSVKGRFTISRDNGAVYLQMNSLKPEDTAVYYCASRPCFLG
VPLIDEGSWGQGTQVTVSSEPKTPKPQ
(SEQ ID NO: 126);
QVQLVESGGGLVQAGGSLRLSCATSGGTFSDYGMGWFRQAPGKEREFVAA
IRRNGNGGNGIEYADSVKGRFTISRDNAKNIVHIQMNSLTPEDIAVYYCA
ASISGYAYNTIERYNYWGQGTQVTVSSEPKTPKPQ
(SEQ ID NO: 128);
QVQLVESGGGINQAGGSLSLSCAASGGDFSRNAMAWFRQAPGKEREEVAS
INWTGSGTYYLDSVKGRFTISRDNAKNALYLQMNNLKPEDTAVYYCARST
VFAEITGLAGYQSGSYDYWGQGTQVTVSSEPKTPKPQ
(SEQ ID NO: 130);
QVQLVETGGGTVQTGGSLRLSCSASGGSFSRNAMGWFRQAPGKEREFVAA
INWSASSTYYRDSVKGRFTVSRDNAKNTVYLHENSIKLEDTAAYYCAGSS
YAEMPYADSVKATSYNYWGQGTQVTVSSEPKTPKPQ
(SEQ ID NO: 132);
QVQLVETGGGLVQAGGSLRLPCSFSGFPFDNYFVGWFRQAPGKEREGVSC
ISSSDGSTYYADSVKGRFTISRDNAKNTVYLQMNSLKPEDTAVYYCGADF
LTPHRCPALYDYWGQGTQVTVSSAHHSEDPS
(SEQ ID NO: 134);
QVQLVESGGGLVQPGGSLRLHCAASGSIASIYRTCWYRQGTGKQRELVAA
ITSGGNTYYADSVKGRFTISRDNAKNTIDLQMNSLKPEDTAVYYCNADEA
GIGGINDYWGQGTQVIVSSAIIHSEDPS
(SEQ ID NO: 136);
QVQLVESGGGLVQAGGSLRLSCAASGRTFSRSSMGWERQAPGKEREFVAS
IVWADGTTLYGDSVKGRFTVSRDNVKNMVYLQMNNLKPEDTALYYCADNK
EVRGLVAVRAIDYDYWGQGTQVTVSSEPKTPKPQ
(SEQ ID NO: 138);
QVQLVESGGLVQAGGSLRLSCAASGRADIIYAMGWERQAPGKEREEVAAV
DWSGGSTYYADSVKGRETISRDNAKNSVYLQMNSLKPEDTAVYYCAARRS
WYRDALSPSRVYEYDYWGQGTQVTVSSEPKTPKPQ
(SEQ ID NO: 140);
QVQLVETGGGINQPGGSLTESCAGSGGTLEHYAIGWERQAPGKEHEWLVC
NRGEYGSTVYVDSVKGRETASRDNAKNTVYLQLNSLKPDDTGTYYCVSGC
YSWRGPWGQGTQVTVSSAHHSEDPS
(SEQ ID NO: 142);
QVQLVESGGGLVQPGGSLKLSCRASGSIVSIYAVGWYRQAPGKQRELLAA
ITTDGSTKYSDSVKGRFTISRDNAKNTVYLQMNNLKPEDTAIYSCIGDAA
GWGDQYYWGQGTQVTVSSEPKTPKPQ
(SEQ ID NO: 144);
QVQLVESGGGLVQAGGSLRLSCAASGSIVNEETMGWYRQAPGKERELVAT
ITNEGSSNYADSVKGRITISGDNAKNTVSLQMNSLKPEDTAVYYCSATEG
SRWPYAHSDHWGQGTQVTVSSEPKTPKPQ
(SEQ ID NO: 146);
QVQLVETGGALVHTGGSLRLSCEVSGSTFSSYGMAWYRQAPGEQRKWVAG
IMPDGTPSYVNSVKGRFTISRDNAKNSVYLHMNNLRPEDTAVYYCNQWPR
IMPDANWGRGTQVTVSSEPKTPKPQ
(SEQ ID NO: 148);
QVQLVETGGSLRLICVTSGSTFNNPAITWYRQPPGKQREWVASIRSGDGP
VYRESVKGRFTIFRDNATDALYERMNSIKPEDTAVYHCNTASPASWLDWG
QGTQVIVSSEPKTPKPQ
(SEQ ID NO: 150);
QVQLVETGGGIVQPGGSLRLSCATSGFPFSTERMSWVRQAPGKGLEWVSG
ITEGGETTLAAPSVKGRFNISRDNARNILYLQMNSIKPEDAAVYYCFRGV
FFRTSFPPELARGQGTQVTVSSEPKTPKPQ
(SEQ ID NO: 152);
QVQINESGGGLVQAGGSLRLSCAASGSAVSDSFSTYAISWHRQAPGKQRE
WIAGISNRGATSYRDSVKGRETISRDNAKNTVYLQMNNLKPEDTGVYYCE
PWPREGLGGGQGTQVTVSSEPKTPKPQ
(SEQ ID NO: 154);
QVQLVESGGGSVQTGGSLTLSCVVSGSTFSDYAVAWYRQVPGKSRAWVAG
VSTTGSTSYTDSVRGRFTISRDNHKKTVYLSMNSLKPEDTGIYYCNLWPF
TNPPSWGQGTQVTVSSAHHSEDPS
(SEQ ID NO: 156);
QVQLVESGGAVVQPGGSLRLSCATSGFTFSDDRMSWARQAPGKGLEWVSG
ISTASEGFATLYAPSVKGRFTISRDNAKHMLYLQMDTLKPEDTAVYYCLR
GVFFRTNIPPEVLRGQGTQVTVSSAIIHSEDPS
(SEQ ID NO: 158);
QVQLVETGGDLVQPGGSLRLSCAASGSSFSRAAVGWYRQAPGKEREWVAR
LASGDMTDYTESVRGRFTISRDNAKHTVYLQMDNLKPEDTAVYYCKARIP
PYYSIEYWGKGTRVTVSSEPKTPKPQ
(SEQ ID NO: 160); and,
QVQLVETGGGLVQAGGSLRISCVVSSPLFNLYDMAWYRQAPGNQRELVAG
ILTDGRATYSDSVKGRFTISRNNITNTVFLQMSSLKPEDTAVYYCNRKNS
IYWDSWGQGTQVTVSSEPKTPKPQ
(SEQ ID NO: 162).
QVQLVESGGGLVQAGGSLRLSCVASGLTFSRYGMGWFRQAPGQERVVVSV
ISPDGGSAYYADSVKGRFTISRDNAKNTVYLQMSTLRFEDIGVYYCTAGP
RNGATTVLRPGDYDYWGQGTQVTVSSEPKTPKPQ

In certain embodiments of the composition, the disease agent is a toxin selected from the group of: a Shiga toxin, an anthrax toxin, and a ricin toxin. For example, the ricin toxin is a ricin A chain toxin or a ricin B chain toxin. In various embodiments, the composition is formulated as a vaccine against the disease agent. In certain embodiments, the composition is for use in detecting the disease agent.

An aspect of the invention provides a pharmaceutical composition for treating a subject at risk for exposure to or exposed to at least one disease agent, the pharmaceutical composition including: a source of expression of at least one recombinant binding protein that neutralizes the disease agent to treat the subject for exposure to the disease agent, such that the source of expression includes at least one nucleotide sequence selected from the group of:

(SEQ ID NO: 97);
CAGGTGCAGCTCGTGGAGACGGGGGGAGGATTGGTGCAGGCTGGGGACCC
TCTGAGACTCTCCTGTGTAGCCTCTGGACGCACCGTCAGTCGCTATGACA
AGGCCTGGTTCCGCCAGGCTCCAGGGAAGGAGCGTGAGTTTGTAGCAGGA
ATTAGCTGGAACGGCGATACAAAAATTTATGCAGACTCCGTGAAGGGCCG
ATTCACCATCTCCAGAGAGAACTCCAGGGATACACTGGATCTGCAAATTG
ACAACCTGAAACCTGAGGACACGGCCGCGTATTACTGTGCGGTCGGAATT
GCGGGTGTTCAGAGTATGGCGCGTATGCTCGGAGTGCGCTACTGGGGCCA
GGGGACCCAGGTCACCGTCTCCTCAGAACCCAAGACACCAAAACCACAA
(SEQ ID NO: 99);
CAGGTGCAGCTCGTGGAGACGGGGGGAGGCTTGGTGCAGCCTGGGGGGTC
TCTGAGACTCTCCTGTGCAGCCTCTGGTTTCAGTTTGGACCCTTATGTGA
TAGGATGGTTCCGGCAGGCCCCAGGGAAGGAGCGTGAGGGGGTCTCATGT
ATTACGAGTAGGGCTGCTAGTCGAACGTCTGTAGACTCCGTGAACGAGCG
ATTCACCATCTCCAGAGACAACGCCAAGAATACGGTCGATCTACACATCA
ATAACCTGAAACCTGAGGACTCGGGCGTTTATTACTGTGCAGCGGTCCCC
CCTGCCAAATTACCACTTTTCAGCCTATGTCGCTCCCTGCCAGCAAAGTA
TGACTACTGGGGCCAGGGGACCCAGGTCACCGTCTCCTCAGCGCACCACA
GCGAAGACCCCTCG
(SEQ ID NO: 101);
CAGGTGCAGCTCGTGGAGTCGGGGGGAGGCTTGGTGCAGCCTGGGGGGTC
TCTGAGACTCTCCTGTGCAGCCTCTGGAAGTAGCTTCAGTAGATATGCCA
TGCGCTGGTACCGCCAGGCTCCAGGGAAGCAGCGCGAGTTGGTCGCAAAC
ATTAATAGTCGTGGTACCTCAAACTATGCAGACTCCGTGAAGGGCCGATT
CACCATCTCCAGAGACAACGCCAAGAACACGGTGTATCTGCAAATGAACA
GCCTGAAACCTGAAGACACGGCCGTCTATTATTGTAATGCAGAGTGGTTG
GGACGATCGGAGCCTTCCTGGGGCCAGGGGACCCAGGTCACCGTCTCCTC
GGAACCCAAGACACCAAAACCACAA
(SEQ ID NO: 103);
CAGGTGCAGCTCGTGGAGTCAGGAGGAGGCTTGGTGCAGCCTGGGGGGTC
TCTGAGACTCTCCTGTGCAGCCTCTGGATTCATTTTCAGTCTTTATACCA
TGAGGTGGCACCGCCAGGCTCCAGGGAAGGAGCGCGAGTTGGTCGCGACT
ATTACTAGTGCTACTGGTATTACAAACTATGCAGACTCCGTGAAGGGCCG
ATTCATCATCTCCAGAGACGATGCCAAGAAGACGGGGTATCTGCAAATGA
ACAGCCTGAAACCTGAGGACACGGCCGTGTATTACTGTAATGCAGTCCGC
ACTACCGTGTCACGAGACTACTGGGGCCAGGGGACCCAGGTCACCGTCTC
CTCAGAACCCAAGACACCAAAACCACAA
(SEQ ID NO: 105);
CAGGTGCAGCTCGTGGAGTCTGGGGGAGGCTTGGTGCAGCCTGGGGGGTC
TCTGAGACTCTCCTGTGCAGCCTCTGGAATCATCTTCAGTATCTATACCA
TGGGCTGGTACCGCCAGGCTCCAGGGAAGCAGCGCGAATTGGTCGCAGCT
ATACCTAGTGGTCCTAGCGCAAACGCTACAGACTCCGTGGGGGGCCGATT
CACCATCACCAGAGACAACGCCGAGAACACGGTGTATCTGCAAATGAACG
ACCTGAAACCTGAGGACACGGCCGTCTATTACTGTAATGCTCGGCGGGGT
CCGGGTATCAAAAACTACTGGGGCCAGGGGACCCAGGTCACCGTCTCCTC
AGAACCCAAGACACCAAAACCACAA
(SEQ ID NO: 107);
CAGGTGCAGCTCGTGGAGTCCGGGGGCGGCTTGGTGCAGCCCGGGGGGTC
TCTGAGTGTCTCCTGTGCAGCCTCTGGAAGCATCGCAAGACCAGGTGCCA
TGGCCTGGTACCGCCAGGCTCCAGGGAAGGAGCGCGAGTTGGTCGCGTCT
ATTACGCCTGGTGGTCTTACAAACTATGCGGACTCCGTGACGGGCCGATT
CACCATTTCCAGAGACAACGCCAAGAGGACGGTGTATCTGCAGATGAACA
GCCTCCAACCCGAGGACACGGCCGTCTATTACTGTCATGCACGAATAATT
CCCCTAGGACTTGGGTCCGAATACAGGGACCACTGGGGCCAGGGGACTCA
GGTCACCGTCTCCTCAGCGCACCACAGCGAAGACCCCTCG
(SEQ ID NO: 109);
CAGGTGCAGCTCGTGGAGACGGGGGGAGGCTTGGTGCAGCCTGGGGGGTC
TCTGGGACTCTCCTGTGTAGTCGCCTCTGGAAGAAGCATCAATAATTATG
GCATGGGCTGGTACCGCCAGGCTCCAGGGAAGCAGCGCGAGTTGGTCGCG
CAAATTAGTAGTGGTGGTACCACAAATTATGCAGGCTCCGTAGAGGGCCG
ATTCACCATCTCCAGAGACAACGTCAAGAAAATGGTGTATCTTCAAATGA
ACAGCCTGAAACCTGAGGACACGGCCGTCTATTACTGTAATTCACTGCTC
CGAACTTTTTCCTGGGGCCAGGGGACCCAGGTCACCGTCTCCTCGGCGCA
CCACAGCGAAGACCCCTCG
(SEQ ID NO: 111);
CAGGTGCAGCTCGTGGAGACCGGGGGGTTGGTGCAGCCTGGGGGCTCCCT
GCGACTCTCCTGTGCAGCCTCCGGACTCACCTTCAGTAGCACTGCCATGG
CCTGGTTCCGCCAGGCTCCAGGGAAGGAGCGTGAGTTTGTAGCACGTATT
AGCGGGGCTGGTATTACGATCTACTATTCGGACTCCGTGAAGGACCGATT
CACCATCTCCAGAAACAACGTCGAGAACACGGTGTATTTGCAAATGAACA
GCCTGAAAACTGAGGACACGGCCGTTTACTACTGTGCAGCAAGACGGAAT
ACTTACACTAGCGACTATAACATACCCGCCCGGTATCCCTACTGGGGCCA
GGGGACCCAGGTCACCGTCTCCTCAGAACCCAAGACACCAAAACCACAA
(SEQ ID NO: 113);
CAGGTGCAGCTCGTGGAGACGGGGGGCTTGGTGCAGCCTGGGGGGTCTCT
GAGACTCTCCTGTGCAGCCTCTAGAAGCACGACGGCCACAATTTATAGTA
TGAACTGGTACCGCCAGGCTCCAGGGAAGCAGCGCGAGTTGGTCGCGGGT
ATGACTAGTGATGGTCAGACAAACTATGCAACCTCCGTGAAGGGCCGATT
CACCATCTCCAGAGACAACGCCAAGAACACGGTATATTTGCTAATGAACA
GCCTGAAACTTGAGGACACGGCCGTCTATTATTGTTATGTAAAACCATGG
AGACTACAAGGTTGGGACTACTGGGGCCAGGGGACCCAGGTCACCGTCTC
CTCAGAACCCAAGACACCAAAACCACAA
(SEQ ID NO: 115);
CAGGTGCAGCTCGTGGAGTCGGGCGGCGGCTTGGTGCAGCCTGGGGGGTC
TCTGAGACTCTCCTGTGCAGCCCCTGAAAGCATCGTCAATAGCAGAACCA
TGGCCTGGTACCGCCAGGCTCCAGGAAAGCAGCGCGAAAGGGTCGCCACT
ATTACTACTGCTGGTAGCCCAAATTATGCAGACTCTGTGAAGGGCCGATT
CGCCATCTCCAGAGACAACGCCAAGAACACGGTATATCTGCAAATGAACA
GCCTGAAACCTGAGGACACGGCCGTCTATTACTGCAATACACTTCTCAGC
ACCCTTCCCTATGGCCAGGGGACCCAGGTCACCGTCTCCTCGGCGCACCA
CAGCGAAGACCCCTCG
(SEQ ID NO: 117);
CAGGTGCAGCTCGTGGAGTCGGGCGGAGGCTTGGTGCAGCCTGGGGGGTC
TCTGGGACTCTCCTGTGTAGTCGCCTCTGAAAGAAGCATCAATAATTATG
GCATGGGCTGGTACCGCCAGGCTCCAGGGAAGCAGCGCGAGTTGGTCGCG
CAAATTAGTAGTGGTGGTACCACAAATTATGCAGACTCCGTAGAGGGCCG
ATTCACCATCTCCAGAGACAACGTCAAGAAAATGGTGCATCTTCAAGTGA
ACAGCCTGAAACCTGAGGACACGGCCGTCTATTACTGTAATTCGCTACTC
CGAACTTTTTCCTGGGGCCAGGGGACCCAGGTCACCGTCTCCTCGGAACC
CAAGACACCAAAACCACAA
(SEQ ID NO: 119);
CAGGTGCAGCTCGTGGAGACGGGAGGAGGCTTGGTGCAGCCTGGGGGGTC
TCTGAGACTCTCCTGTGCAGCCTCTGGATTCACCTTCAGTAGTTATCGCA
TGAGCTGGTACCGGCAGGCTGCAGGGAAGGAGCGCGACGTGGTCGCAACA
ATTACTGCTAATGGTGTTCCCACAGGCTATGCAGACTCCGTGATGGGCCG
ATTCACCATTTCCAGAGACAATGCCAAGAACACGGTGTATCTGGAAATGA
ACAGCCTGAATCCTGAGGACACGGCCGTGTATTACTGTAACGCGCCCCGT
TTGCATACATCTGTAGGCTACTGGGGCCAGGGGACCCAGGTCACCGTCTC
CTCAGAACCCAAGACACCAAAACCACAA
(SEQ ID NO: 121)
CAGGTGCAGCTCGTGGAGTCGGGAGGAGGATTGGTGCAGGCTGGGAACTC
TCTGAGACTCTCCTGTACGGCCTCTGGTGTGATCTTCTCTATCTATACCA
TGGGCTGGTTCCGCCAGGCTCCAGGGAAGGAGCGTGAGTTTGTAGCAGCG
ATAGGGGTGGCTGATGGTACCGCACTTGTGGCAGACTCCGTGACGGGCCG
ATTCACCATCTCCAGAGACAACGCCAAGAACACCGTTTATCTGCATATGA
ACAGCCTGAAGCCTGAGGACACGGCCGTCTATTCCTGTGCAGCGTATCTT
AGCCCCCGTGTCCAATCCCCCTACATAACTGACTCCCGGTATCAACTCTG
GGGCCAGGGGACCCAGGTCACCGTCTCCTCAGAACCCAAGACACCAAAAC
CACAA
(SEQ ID NO: 123);
CAGGTGCAGCTCGTGGAGACTGGGGGAGGATTGGTGCAGGCTGGGGGCTC
TCTGAGGCTCTCCTGTGCAGCCTCTGGACGCTATGCCATGGGCTGGTTCC
GCCAGGCTCCAGGGAAGGAGCGTGAATTTGTAGCGACTATTAGCCGGAGT
GGTGCTATCAGAGAGTATGCAGACTCCGTGAAGGGCCGATTCACCATCTC
CAGAGACGGCGCCGAGAACACGGTGTATCTGGAAATGAACAGCCTGAAAC
CTGACGACACGGCCATTTATGTCTGTGCAGAAGGACGAGGGGCGACATTC
AACCCCGAGTATGCTTACTGGGGCCAGGGGACCCAGGTCACCGTCTCCTC
AGCGCACCACAGCGAAGACCCCTCG
(SEQ ID NO: 125);
CAGGTGCAGCTCGTGGAGTCGGGCGGAGGCTTGGTGCAGCCTGGGGGGTC
TCTGAGACTCTCCTGTGCAGCCTCTGGATTCACTTTGGATGATTATGCCA
TAGGCTGGTTCCGCCAGGTCCCAGGGAAGGAGCGTGAGGGGGTCGCATGT
GTTAAAGATGGTAGTACATACTATGCAGACTCCGTGAAGGGCCGATTCAC
CATCTCCAGAGACAACGGCGCGGTGTATCTGCAAATGAACAGCCTGAAAC
CTGAGGACACAGCCGTTTATTACTGTGCATCCAGGCCCTGCTTTTTGGGT
GTACCACTTATTGACTTTGGTTCCTGGGGCCAGGGGACCCAGGTCACCGT
CTCCTCGGAACCCAAGACACCAAAACCA
CAA
(SEQ ID NO: 127);
CAGGTGCAGCTCGTGGAGTCAGGGGGAGGATTGGTGCAGGCTGGGGGCTC
TCTGAGACTCTCCTGCGCAACCTCTGGCGGCACCTTCAGTGACTATGGAA
TGGGCTGGTTCCGCCAGGCTCCAGGGAAGGAGCGTGAGTTTGTAGCAGCT
ATTAGGCGGAATGGTAATGGCGGTAATGGCATTGAATATGCAGACTCCGT
GAAGGGCCGATTCACCATCTCCAGAGACAACGCCAAGAACACGGTGCATC
TACAAATGAACAGCCTGACACCTGAGGACACGGCCGTTTATTACTGTGCA
GCGTCAATATCGGGATACGCTTATAACACAATTGAAAGATATAACTACTG
GGGCCAGGGAACCCAGGTCACCGTCTCCTCAGGAACCCAAGACACCAAAA
CCACAA
(SEQ ID NO: 129);
CAGGTGCAGCTCGTGGAGTCCGGCGGAGGATTGGTGCAGGCGGGGGGCTC
TCTGAGTCTCTCCTGTGCAGCCTCTGGAGGTGACTTCAGTAGGAATGCCA
TGGCCTGGTTCCGTCAGGCTCCAGGGAAGGAGCGTGAATTTGTAGCATCT
ATTAACTGGACTGGTAGTGGCACATATTATCTAGACTCCGTGAAGGGCCG
ATTCACCATCTCCAGAGACAACGCCAAGAACGCCCTGTATCTGCAAATGA
ACAACCTGAAACCTGAGGACACGGCCGTTTATTACTGTGCACGCTCCACG
GTGTTTGCCGAAATTACAGGCTTAGCAGGCTACCAGTCGGGATCGTATGA
CTACTCGGGCCAGGGGACCCAGGTCACCGTCTCCTCAGAACCCAAGACAC
CAAAACCACAA
(SEQ ID NO: 131);
CAGGTGCAGCTCGTGGAGACCGGCGGAGGAACGGTGCANACTGGGGGCTC
TCTGAGACTCTCCTGTTCAGCCTCTGGCGGCTCCTTCAGTAGGAATGCCA
TGGGCTGGTTCCGCCAGGCTCCAGGGAAGGAGCGTGAATTTGTAGCAGCT
ATTAACTGGAGTGCCTCTAGTACTTATTATAGAGACTCCGTGAAGGGACG
ATTCACCGTCTCCAGAGACAACGCCAAGAACACGGTGTATCTGCATTTGA
ACAGCCTGAAACTTGAGGACACGGCCGCGTATTACTGTGCTGGAAGCTCG
GTGTATGCAGAAATGCCGTACGCCGACTCTGTCAAGGCAACTTCCTATAA
CTACTGGGGCCAGGGGACCCAGGTCACCGTCTCCTCAGAACCCAAGACAC
CAAAACCACAA
(SEQ ID NO: 133);
CAGGTGCAGCTCGTGGAGACCGGGGGAGGCTTGGTGCAGGCTGGGGGGTC
TCTGAGACTCCCCTGTTCATTCTCTGGATTCCCTTTCGATAATTATTTCG
TAGGCTGGTTCCGCCAGGCCCCAGGGAAGGAGCGTGAGGGGGTCTCATGT
ATTAGTAGTAGTGATGGTAGCACATACTATGCAGACTCCGTGAAGGGCCG
GTTCACCATCTCCAGAGACAACGCCAAGAACACGGTGTATCTGCAAATGA
ACAGTCTGAAACCTGAGGATACGGCCGTTTATTACTGTGGAGCAGATTTC
CTCACCCCACATAGGTGTCCAGCCTTATATGACTACTGGGGCCAGGGGAC
CCAGGTCACCGTCTCCTCAGCGCACCACAGCGAAGACCCCTCG
(SEQ ID NO: 135);
CAGGTGCAGCTCGTGGAGTCTGGTGGAGGCTTGGTGCAGCCTGGGGGGTC
TCTGAGACTCCACTGTGCAGCCTCTGGAAGCATCGCCAGTATCTATCGCA
CGTGCTGGTACCGCCAGGGCACAGGGAAGCAGCGCGAGTTGGTCGCAGCC
ATTACTAGTGGTGGTAACACATACTATGCGGACTCCGTTAAGGGCCGATT
CACCATCTCCAGAGACAACGCCAAAAACACAATCGATCTGCAAATGAACA
GCCTGAAACCTGAGGACACGGCCGTCTATTACTGTAATGCAGACGAGGCG
GGGATCGGGGGATAATGACTACTGGGGCCAGGGGACCCAGGTCACCGTCT
CCTCAGCGCACCACAGCGAAGACCCCTCG
(SEQ ID NO: 137);
CAGGTGCAGCTCGTGGAGTCGGGGGGAGGATTGGTGCAGGCTGGGGGCTC
TCTGAGACTCTCCTGTGCAGCCTCTGGACGCACCTTCAGTCGCAGTTCCA
TGGGCTGGTTCCGCCAGGCTCCAGGGAAGGAGCGTGAATTCGTTGCGTCC
ATTGTCTGGGCTGATGGTACGACGTTGTATGGAGACTCCGTAAAGGGCCG
ATTCACCGTCTCCAGGGACAACGTCAAGAACATGGTGTATCTACAAATGA
ACAACCTGAAACCTGAGGACACGGCCCTTTATTACTGTGCGGACAATAAA
TTCGTCCGTGGATTAGTGGCTGTCCGTGCGATAGATTATGACTACTGGGG
CCAGGGGACCCAGGTCACCGTCTCGTCAGAACCCAAGACACCAAAACCAC
AA
(SEQ ID NO: 139);
CAGGTGCAGCTCGTGGAGTCGGGAGGATTGGTGCAGGCTGGAGGCTCTCT
GAGACTCTCCTGCGCAGCCTCTGGACGCGCCGACATAATCTATGCCATGG
GCTGGTTCCGCCAGGCTCCAGGGAAGGAGCGTGAGTTTGTAGCGGCAGTA
GACTGGAGTGGTGGTAGCACATACTATGCAGACTCCGTGAAGGGCCGATT
CACCATCTCCAGAGACAACGCCAAGAACTCGGMTATCTGCAAATGAACAG
CCTGAAACCTGAGGACACGGCCGTTTATTACTGTGCAGCCCGAAGGAGCT
GGTACCGAGACGCGCTATCCCCCTCCCGGGTGTATGAATATGACTACTGG
GGCCAGGGGACCCAGGTCACCGTCTCCTCAGAACCCAAGACACCAAAACC
ACAA
(SEQ ID NO: 141);
CAGGTGCAGCTCGTGGAGACGGGAGGAGGCTTGGTGCAGCCTGGGGGGTC
TCTGACACTCTCCTGTGCAGGCTCCGGTGGCACTTTGGAACATTATGCTA
TAGGCTGGTTCCGCCAGGCCCCTGGGAAAGAGCATGAGTGGCTCGTATGT
AATAGAGGTGAATATGGGAGCACTGTCTATGTAGACTCCGTGAAGGGCCG
ATTCACCGCCTCCAGAGACAACGCCAAGAACACGGTGTATCTGCAATTGA
ACAGTCTGAAACCTGACGACACAGGCATTTATTACTGTGTATCGGGATGT
TACTCCTGGCGGGGTCCCTGGGGCCAGGGGACCCAGGTCACCGTCTCCTC
GGCGCACCACAGCGAAGACCCCTCG
(SEQ ID NO: 143);
CAGGTGCAGCTCGTGGAGTCTGGGGGAGGTTTGGTGCAGCCTGGGGGGTC
TCTGAAACTCTCCTGTAGAGCCTCTGGAAGCATAGTCAGTATCTATGCCG
TGGGCTGGTACCGCCAGGCTCCAGGGAAGCAGCGCGAGTTGCTCGCGGCT
ATCACTACTGATGGTAGCACGAAGTACTCAGACTCCGTGAAGGGCCGATT
CACCATCTCCCGAGACAACGCCAAGAACACGGTATATCTGCAAATGAACA
ACCTCAAACCTGAGGACACGGCCATCTATTCCTGTATCGGGGACGCGGCG
GGTTGGGGCGACCAATACTACTGGGGCCAGGGGACCCAGGTCACCGTCTC
CTCAGAACCCAAGACACCAAAACCACAA
(SEQ ID NO: 145);
CAGGTGCAGCTCGTGGAGTCAGGCGGAGGCTTGGTGCAGGCTGGGGGGTC
TCTGAGACTCTCCTGTGCAGCCTCTGGAAGCATCGTCAATTTCGAAACCA
TGGGCTGGTACCGCCAGGCTCCAGGGAAGGAGCGCGAGTTGGTCGCAACT
ATTACTAATGAAGGTAGTTCAAACTATGCAGACTCCGTGAAGGGCCGATT
CACCATCTCCGGAGACAACGCCAAGAACACGGTGTCCCTGCAAATGAACA
GCCTGAAACCTGAGGACACGGCCGTCTACTACTGTTCGGCGACGTTCGGC
AGTAGGTGGCCGTACGCCCACAGTGATCACTGGGGCCAGGGGACCCAGGT
CACCGTCTCCTCAGAACCCAAGACACCAAAACCACAA
(SEQ ID NO: 147);
CAGGTGCAGCTCGTGGAGACGGGCGGAGCATTGGTGCACACTGGGGGTTC
TCTGAGACTCTCCTGCGAAGTCTCCGGAAGCACCTTCAGTAGCTATGGCA
TGGCCTGGTACCGCCAAGCTCCAGGCGAGCAGCGTAAGTGGGTCGCAGGT
ATTATGCCGGATGGTACTCCAAGCTATGTAAACTCCGTGAAGGGCCGATT
CACCATCTCCAGAGACAACGCCAAGAACTCGGTGTATCTGCACATGAACA
ACCTGAGGCCTGAAGACACGGCCGTCTATTATTGCAACCAATGGCCGCGC
ACGATGCCTGACGCGAACTGGGGCCGGGGGACCCAGGTCACCGTCTCCTC
AGAACCCAAGACACCAAAACCACAA
(SEQ ID NO: 149);
CAGGTGCAGCTCGTGGAGACTGGGGGGTCTCTGAGGCTCACCTGTGTAAC
CTCTGGAAGCACCTTCAATAATCCTGCCATAACCTGGTACCGCCAGCCTC
CAGGGAAGCAGCGTGAGTGGGTCGCAAGTCTTCGTAGTGGTGATGGTCCA
GTATATAGGGAATCCGTGAAGGGCCGATTCACCATTTTTAGAGACAACGC
CACGGACGCGCTGTATCTGCGGATGAATAGCCTGAAACCTGAGGACACGG
CCGTCTATCACTGTAACACCGCCTCACCTGCTAGTTGGCTGGACTGGGGC
CAGGGGACCCAGGTCACTGTCTCCTCAGAACCCAAGACACCAAAACCACA
A
(SEQ ID NO: 151);
CAGGTGCAGCTCGTGGAGACGGGAGGAGGATTGGTGCAACCTGGGGGTTC
TCTGAGACTCTCTTGTGCAACCTCTGGATTCCCCTTCAGTACGGAGCGTA
TGAGCTGGGTCCGCCAGGCTCCAGGAAAGGGGCTCGAGTGGGTCTCAGGT
ATTACTGAGGGTGGTGAAACCACTCTCGCGGCACCCTCCGTGAAGGGCCG
ATTCAACATCTCCAGAGACAACGCCAGGAATATCCTATATCTACAGATGA
ATTCCTTGAAACCTGAGGACGCGGCCGTTTACTATTGTTTTAGAGGTGTT
TTTTTTAGAACGAGTTTTCCTCCCGAACTCGCGCGGGGCCAGGGGACCCA
GGTCACCGTCTCCTCAGAACCCAAGACACCAAAACCACAA
(SEQ ID NO: 153);
CAGGTGCAGCTCGTGGAGTCGGGCGGAGGCTTGGTGCAGGCAGGGGGGTC
TTTGAGACTCTCCTGTGCAGCCTCTGGAAGCGCCGTCAGTGACAGCTTCA
GTACCTATGCCATCTCCTGGCACCGCCAGGCTCCAGGGAAGCAGCGTGAG
TGGATCGCAGGTATTAGTAATCGTGGTGCGACAAGCTATAGAGACTCCGT
GAAGGGCCGATTCACCATCTCCAGAGACAACGCCAAGAACACGGTATATC
TGCAAATGAACAACCTGAAACCTGAGGACACGGGCGTCTATTATTGTGAG
CCATGGCCACGCGAAGGACTTGGGGGGGGCCAGGGGACTCAGGTCACCGT
CTCCTCAGAACCCAAGACACCAAAACCACAA
(SEQ ID NO: 155);
CAGGTGCAGCTCGTGGAGTCGGGGGGAGGCTCGGTGCANACTGGGGGGTC
TCTGACACTCTCCTGTGTAGTCTCTGGAAGTACCTTCAGTGACTATGCGG
TGGCCTGGTACCGCCAGGTTCCAGGCAAATCGCGTGCGTGGGTCGCGGGT
GTTAGTACTACTGGCTCGACATCTTATACAGACTCCGTGAGGGGCCGGTT
CACCATCTCCAGAGACAACCACAAGAAGACGGTGTATCTTTCAATGAACA
GCCTGAAACCTGAGGACACGGGCATCTATTACTGCAACTTATGGCCGTTC
ACAAATCCTCCTTCCTGGGGCCAGGGAACCCAAGTCACCGTTTCCTCGGC
GCACCACAGCGAAGACCCCTCG
(SEQ ID NO: 157);
CAGGTGCAGCTCGTGGAGTCTGGAGGAGCCGTGGTGCAACCTGGGGGTTC
TCTGAGACTCTCCTGTGCAACCTCTGGATTCACCTTCAGTGACGATCGTA
TGAGCTGGGCCCGCCAGGCTCCAGGAAAGGGGCTCGAGTGGGTCTCAGGT
ATTAGTACTGCTAGTGAAGGTTTTGCTACACTCTACGCACCCTCCGTGAA
GGGCCGATTCACCATCTCCAGAGACAACGCCAAGCATATGCTGTATCTGC
AAATGGATACCTTGAAACCTGAGGACACGGCCGTGTATTACTGTTTAAGA
GGGGTTTTTTTTAGAACGAACATTCCTCCCGAGGTACTGCGCTGGCCAGG
GGACCCAGGTCACCGTCTCCTCAGCGCACCACAGCGAAGACCCCTCG
(SEQ ID NO: 159);
CAGGTGCAGCTCGTGGAGACGGGGGGAGACTTGGTGCANCCTGGGGGGTC
TCTGAGACTCTCCTGTGCAGCCTCTGGAAGCTCCTTCAGCCGCGCTGCCG
TGGGCTGGTACCGTCAGGCTCCAGGAAAGGAGCGTGAGTGGGTCGCACGT
CTCGCGAGTGGTGATATGACGGACTATACCGAGTCCGTGAGGGGCCGATT
CACTATCTCCAGAGACAACGCCAAGCACACGGTGTATCTGCAAATGGACA
ACCTGAAACCTGAGGACACGGCCGTCTACTATTGTAAGGCCAGGATACCC
CCTTATTACTCTATAGAGTACTGGGGCAAAGGGACCCGGGTCACCGTCTC
CTCANAACCCAAGACACCAAAACCACAA
(SEQ ID NO: 161); and,
CAGGTGCAGCTCGTGGAGACAGGTGGAGGCTTGGTGCAGGCTGGGGGGTC
TCTGAGACTCTCCTGTGTAGTATCTAGTCCCCTGTTCAATCTTTACGACA
TGGCCTGGTATCGCCAGGCTCCAGGGAATCAGCGTGAGTTGGTCGCAGGC
ATCTTGACTGATGGTCGCGCAACATATTCAGACAGCGTGAAGGGCCGATT
CACCATTTCCAGAAACAACCTGACGAACACGGTGTTTTTACAAATGAGCA
GCCTGAAACCTGAGGACACGGCCGTCTATTATTGTAATAGAAAGAATAGT
ATCTACTGGGATTCCTGGGGCCAGGGGACCCAGGTCACCGTCTCCTCGGA
ACCCAAGACACCAAAACCACAA
(SEQ ID NO:163).
CAGGTGCAGCTCGTGGAGTCGGGGGGAGGATTGGTGCAGGCTGGGGGCTC
TCTGAGACTCTCCTGCGTAGCCTCTGGACTCACCTTCAGTCGCTATGGCA
TGGGCTGGTTCCGCCAGGCTCCAGGACAGGAGCGTGTAGTCGTATCAGTT
ATTAGTCCCGACGGTGGTAGCGCATACTACGCAGACTCCGTGAAGGGCCG
ATTCACCATCTCCAGAGACAACGCCAAGAACACGGTGTATCTGCAAATGA
GCACCCTGAGATTTGAGGACACGGGCGTTTATTATTGTACAGCAGGGCCC
CGGAATGGAGCGACTACAGTCCTCCGGCCAGGGGATTATGACTACTGGGG
CCAGGGGACCCAGGTCACTGTCTCCTCAGAACCCAAGACACCAAAACCAC
AA

In various embodiments of the composition, the binding protein is a heteromultimeric neutralizing binding protein having a plurality of binding regions that are not identical and each binding region has affinity to specifically bind a non-overlapping portion of the disease agent. In certain embodiments, the binding protein is a homodimer, a heterodimer, or a multimer. The composition in various embodiments is a multimer, e.g., a heterodimer, a heterotrimer, or a heterotetramer.

The binding protein in various embodiments of the composition further includes at least one of: a tag that is an epitope that is specifically bound by an antibody, and a linker that separates the binding regions, wherein the linker includes at least one selected from the group of: a peptide, a protein, a sugar, and a nucleic acid. For example the linker includes a plurality of amino acids that join or attach binding regions in the composition. For example the linker joins to VHHs that bind different portions of a disease agent protein.

The disease agent in various embodiments is at least one selected from a virus, a cancer cell, a fungus, a bacterium, a parasite, and a product thereof selected from a pathogenic molecule, a protein, a lipopolysaccharide, and a toxin. For example the toxin is a plant lectin such as a toxic or poisonous molecule associated with a: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein.

In various embodiments of the pharmaceutical composition, the toxin is a bacterial toxin, for example a molecule associated with a bacterial species selected from the group of: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovale; Pseudomonas for example P. aeruginosa; Neisseria for example N. gonorrhoeae; Escherichia coli for example E. coli O157:117; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

In a related embodiment of the pharmaceutical composition, at least one disease agent includes a plurality of non-identical disease agents, and the binding protein binds to the plurality of the disease agents, thereby neutralizing the disease agents. For example, the composition is multimeric and is effective for neutralizing at least two disease agents, at least three disease agents, or at least four disease agents.

In various embodiments of the pharmaceutical composition, the binding protein includes an amino acid sequence that is substantially identical to at least one of SEQ ID NO: 96, SEQ ID NO: 98, SEQ ID NO:100, SEQ ID NO:102. SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, wherein substantially identical is having at least 50% identity, 60% identity, at least 65% identity, at least 70% identity, at least 75% identity, at least 80% identity, at least 85% identity, at least 90% identity, or and at least 95% identity to the amino acid sequence of SEQ ID NOs: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162.

The source of expression of the binding protein in various embodiments of the composition is selected from of: a nucleic acid vector with a gene encoding the binding protein; a viral vector encoding the binding protein; and the binding protein expressed directly from naked nucleic acid. For example, the viral vector is derived from at least one selected from: an adenovirus, an adeno-associated virus, a herpesvirus, and a lentivirus.

In various embodiments of the pharmaceutical composition, the source of expression of the binding protein includes an nucleotide sequence that is substantially identical to at least one of SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163, wherein substantially identical is having at least 50% identity, 60% identity, at least 65% identity, at least 70% identity, at least 75% identity, at least 80% identity, at least 85% identity, at least 90% identity, or and at least 95% identity to the nucleotide sequence of SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163.

An aspect of the invention provides a method for treating a subject at risk for exposure to or exposed to at least one disease agent, the method involving: administering to the subject at least one binding protein including at least one binding region having an amino acid sequence selected from: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, such that the binding protein neutralizes the disease agent thereby treating the subject for the exposure.

The binding protein in various embodiments of the method includes at least one linker selected from the group of: a peptide, a protein, a sugar, or a nucleic acid.

In various embodiments of the method, the disease agent is at least one selected from a virus, a cancer cell, a fungus, a bacterium, a parasite and a product thereof such as a pathogenic molecule, a protein, a lipopolysaccharide, and a toxin for example the toxin is produced by or from a plant or a bacterium.

In various embodiments of the method, the toxin is a bacterial toxin, for example the toxin is produced by at least one bacterial species selected from: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovale; Pseudomonas for example P. aeruginosa; Neisseria for example N. gonorrhoeae; Escherichia coli for example E. coli O157:H7; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

The method in various embodiments further includes observing or detecting neutralization of the disease agent by the binding protein and/or survival of the subject; or identifying a reduction or remediation in at least one pathology symptom associated with the disease agent. For example, the symptom is elevated body temperature, diarrhea, vomiting, coughing, or weezing.

The disease agent in various embodiments of the method includes a plurality of disease agents, and the method involves prior to administering, engineering the binding protein to bind to a feature of each of the plurality of the disease agents. For example, the feature of the disease agents is non-identical. Alternatively, the feature of each of the disease agents is identical.

An aspect of the invention provides method for treating a subject at risk for exposure to or exposed to at least one disease agent, the method including: administering to the subject a source of expression of a binding protein, such that the source of expression of the binding protein is a nucleotide sequence encoding the binding protein, and the nucleotide sequence includes at least one selected from the group consisting of: a naked nucleic acid vector, bacterial vector, and a viral vector, such that the nucleotide sequence includes at least one selected from the group of: SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163; and, measuring neutralizing by the binding protein of the disease agent, thereby treating the subject for the exposure.

The binding protein in various embodiments of the method includes at least one linker selected from the group of: a peptide, a protein, a sugar, or a nucleic acid. In a related embodiments, the disease agent is at least one selected from a virus, a cancer cell, a fungus, a bacterium, a parasite, and a product thereof such as a pathogenic molecule, a protein, a lipopolysaccharide, and a toxin. For example, the toxin is a plant lectin such as one selected from the group of: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein.

In a related embodiment of the method, the toxin is a bacterial toxin for example the toxin is produced by at least one bacterial species selected from the group of: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovale; Pseudomonas for example P. aeruginosa; Neisseria for example N. gonorrhoeae; Escherichia coli for example E. coli O157:117; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like. For example, the method neutralizes a bacterial toxin that is a Shiga toxin or an anthrax toxin.

The method in various embodiments further includes observing or detecting neutralizing the disease agent by the binding protein and/or viability of the subject; or identifying a reduction or remediation in at least one pathology symptom associated with the disease agent. For example identifying the reduction or remediation involves observing the patient for minutes, hours, days, or weeks. In a related embodiments, identifying the reduction or remediation involves analyzing and measuring amount or activity of the disease agent from a sample from the subject.

In various embodiments of the method, the disease agent includes a plurality of disease agents, and the method involves prior to administering, engineering the binding protein to bind to a feature of each of the plurality of the disease agents. For example, the composition is a treatment agent that protects or treats subjects from multiple disease agents that the subject might be or might already have had an exposure. In various embodiments of the method, the feature is non-identical. Alternatively, the feature is identical.

In various embodiments of the method, measuring includes detecting a reduced amount of degree of at least one of: bacterial titer in a tissue or bodily fluid, fever, inflammation, pain, diarrhea, bleeding, tissue discoloration, clotting, and tachycardia.

An aspect of the invention provides a kit for treating a subject exposed to or at risk for exposure to a disease agent including: a unit dosage of a pharmaceutical composition for treating a subject at risk for exposure to or exposed to at least one disease agent, the pharmaceutical composition including: at least one recombinant binding protein that neutralizes the disease agent thereby treating the subject for exposure to the disease agent, such that the binding protein includes at least one amino acid sequence selected from the group of: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162; a container; and, instructions for use.

An aspect of the invention provides a kit for treating a subject exposed to or at risk for exposure to a disease agent, the kit including: a pharmaceutical composition for treating a subject at risk for exposure to or exposed to at least one disease agent, the pharmaceutical composition comprising: a source of expression of at least one recombinant binding protein that neutralizes the disease agent thereby treating the subject for exposure to the disease agent, such that the source of expression comprises at least one nucleotide sequence selected from the group of: SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163; a container; and, instructions for use.

In various embodiments of the kit, the disease agent is a virus, a cancer cell, a fungus, a bacterium, a parasite, and a product thereof such as a pathogenic molecule, a protein, a lipopolysaccharide, and a toxin. For example, the toxin is a plant lectin such as one selected from the group of: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein.

In a related embodiment of the kit, the toxin is a bacterial toxin for example the bacterial toxin is a Shiga toxin or an anthrax toxin.

In various embodiments of the kit, the toxin is produced by at least one bacterial species selected from the group of: Bacillus for example B. anthracis; Clostridium for example C. tetani, C. difficile, and C. perfringens; Corynebacterium for example C. diphtherias; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovale; Pseudomonas for example P. aeruginosa; Neisseria for example N. gonorrhoeae; Escherichia coli for example E. coli O157:H7; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

In various embodiments of the kit, the toxin is a fungal toxin produced by at least one fungus selected from the group consisting of: Cryptococcus for example C. Gattii and C. neoformans v. neoformans; Candida for example C. albicans; Aspergillus for example A. flavus and A. fumigatus; and the like.

The kit in various embodiments further includes an applicator, for example the applicator is a syringe, a needle, a sprayer, a sponge, a gel, a strip, a tape, a bandage, a tray, a string, or a nanostructure. In a related embodiment, the kit further includes a toxin control for example a Shiga toxin, a ricin toxin, or an anthrax toxin.

An aspect of the invention provides a kit for detecting a toxin, the kit comprising: a binding protein that binds to the toxin, such that the binding protein including an amino acid sequence selected from the group of: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, such that the is toxin selected from the group of: a Shiga toxin, a B. anthracis toxin, and a ricin toxin, such that SEQ ID NO: 96 and SEQ ID NO: 98 specifically bind the Shiga toxin, such that SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 specifically bind the anthrax toxin, such that SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 specifically bind the ricin A chain toxin, and such that SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 specifically bind the ricin B chain toxin; a container; and, instructions for use for detecting the toxin using the binding protein. In various embodiments, the kit further comprises a toxin control for example the toxin control is an attenuated or wildtype toxin selected from the group of: Shiga toxin, B. anthracis toxin, and ricin toxin. In various embodiments, the kit further includes at least one of: a microfuge tube, a test tube, a cuvette, and a multi-well plate. In various embodiments, the binding protein is attached to a substrate, for example the substrate comprises any of: a bead, a well, a column, and a tube. In various embodiments, the binding protein in multimeric, e.g., dimeric, trimeric, and tetrameric.

An aspect of the invention provides a kit for detecting a toxin comprising: a source of expression of a binding protein that specifically binds the toxin, the source comprising at least one nucleotide sequence selected from the group of: SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163, such that the toxin is selected from the group of: a Shiga toxin, a B. anthracis toxin, and a ricin toxin, such that SEQ ID NO: 96 and SEQ ID NO: 98 encode the binding protein that specifically binds the Shiga toxin, such that SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 encode the binding protein that specifically binds the anthrax toxin, such that SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 encode the binding protein that specifically binds the ricin A chain toxin, and such that SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 encode the binding protein that specifically binds the ricin B chain toxin; a container; and, instructions for use for detecting the toxin using the binding protein. In various embodiments, the kit further comprises a toxin control for example the toxin control is an attenuated or wildtype toxin selected from the group of: Shiga toxin, B. anthracis toxin, and ricin toxin.

In various embodiments, the kit further includes at least one of: a microfuge tube, a test tube, a cuvette, and a multi-well plate. The source of expression of the binding protein in various embodiments of the kit is selected from of: a nucleic acid vector with a gene encoding the binding protein; a viral vector encoding the binding protein; and the binding protein expressed directly from naked nucleic acid. For example, the viral vector is derived from at least one selected from: an adenovirus, an adeno-associated virus, a herpesvirus, and a lentivirus. In various embodiments of the kit the nucleotide sequence encodes a multimeric binding protein that binds a plurality of toxins. In various embodiments, the binding protein in multimeric, e.g., dimeric, trimeric, and tetrameric. In various embodiments, the kit further includes at least one of: a microfuge tube, a test tube, a cuvette, and a multi-well plate.

An aspect of the invention provides a method for detecting a presence of a toxin in a biological sample, the method comprising: contacting a sample and an amount of at least one binding protein that specifically bins the toxin, such that the binding protein includes a binding region having an amino acid sequence selected from: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, such that the toxin is selected from the group of: a Shiga toxin, a B. anthracis toxin, and a ricin toxin, such that SEQ ID NO: 96 and SEQ ID NO: 98 specifically bind the Shiga toxin, such that SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 specifically bind the anthrax toxin, such that SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 specifically bind the ricin A chain toxin, and such that SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 specifically bind the ricin B chain toxin.

An aspect of the invention provides a method for detecting a presence of a toxin in a biological sample, the method comprising: contacting the sample and with an amount of a source of expression of a binding protein that specifically binds the toxin, such that the source of expression of the binding protein is a nucleotide sequence encoding the binding protein, such that the nucleotide sequence includes at least one selected from the group consisting of: a naked nucleic acid vector, bacterial vector, and a viral vector, such that the nucleotide sequence contains at least one selected from the group of: SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163, such that the toxin is selected from the group of: a Shiga toxin, a B. anthracis toxin, and a ricin toxin, such that SEQ ID NO: 96 and SEQ ID NO: 98 encode the binding protein that specifically binds the Shiga toxin, such that SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 encode the binding protein that specifically binds the anthrax toxin, such that SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 encode the binding protein that specifically binds the ricin A chain toxin, and such that SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 encode the binding protein that specifically binds the ricin B chain toxin.

The source of expression of the binding protein in various embodiments of the method is selected from of: a nucleic acid vector with a gene encoding the binding protein; a viral vector encoding the binding protein; and the binding protein expressed directly from naked nucleic acid. For example, the viral vector is derived from at least one selected from: an adenovirus, an adeno-associated virus, a herpesvirus, and a lentivirus.

In various embodiments of the method, contacting involves incubating a first set of test cells with an aliquot of the sample and the amount of a source of expression of a binding protein, and measuring an indicia of toxin exposure in the first set of test cells in comparison with a second set of the test cells not so contacted and otherwise identical as a negative control, such that the decreased extent and presence of the indicia in the first set in comparison to the second set indicates the presence of the disease agent in the sample

The method in a related embodiment, further includes contacting at least a third set of cells with at least one known amount of the toxin as a positive control.

In various embodiments, the method further includes contacting at least a fourth set of cells with a different known amount of the C. difficile toxin wherein a plurality of positive controls comprises a standard curve for determining amount of the toxin present.

An aspect of the invention provides a device for detecting a toxin comprising: a composition including a binding protein having an amino acid sequence, such that the binding protein specifically binds the toxin selected from the group of: a Shiga toxin, a B. anthracis toxin, and a ricin toxin, such that the binding protein is selected from the group of: SEQ ID NO: 96 and SEQ ID NO: 98 that specifically bind the Shiga toxin; SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 that specifically bind the anthrax toxin; SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 that specifically bind the ricin A chain toxin; and SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 that specifically bind the ricin B chain toxin; and a substrate attached to the composition, sue that the toxin binds the composition for detection of the toxin.

The substrate in various embodiments of the device includes any of: a bead, a well, a column, and a tube. For example, the binding protein is attached, linked, embedded, or coated on the substrate, such that a sample (including the toxin) that contacts the substrate binds specifically to the substrate. In a related embodiment, the substrate further comprises a blocking agent that prevents non-specific binding of the toxin to the substrate. For example the blocking agent is a detergent or a protein. For example, the detergent includes Tween-20® or Triton x-100® (Sigma-Aldrick Inc.; St. Louis, Mo.). For example the protein used as a blocking agent includes bovine serum albumin, a non-fat dry milk or casein, whole normal serum, or fish gelatin.

An aspect of the invention provides a composition that specifically binds a Shiga toxin, wherein the composition comprises a binding protein having an amino acid sequence selected from SEQ ID NO:96 and (SEQ ID NO: 98).

An aspect of the invention provides a composition that specifically anthrax protective antigen, wherein the composition comprises a binding protein having an amino acid sequence selected from: SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122.

An aspect of the invention provides a composition that specifically a ricin A chain toxin, wherein the composition comprises a binding protein having an amino acid sequence selected from: SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, and SEQ ID NO: 145.

An aspect of the invention provides a composition that specifically a ricin B chain toxin, wherein the composition comprises a binding protein having an amino acid sequence selected from: SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, and SEQ ID NO: 163.

An aspect of the invention provides a composition that specifically binds a Shiga toxin, wherein the composition comprises a source of expression of a binding protein, wherein the source comprises a nucleotide sequence selected from: SEQ ID NO:97 and SEQ ID NO: 99.

An aspect of the invention provides a composition that specifically binds an anthrax protective antigen toxin, wherein the composition comprises a source of expression of a binding protein, wherein the source comprises a nucleotide sequence selected from: SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, and SEQ ID NO: 123.

An aspect of the invention provides a composition that specifically binds a ricin A chain toxin, wherein the composition comprises a source of expression of a binding protein, wherein the source comprises a nucleotide sequence selected from: (SEQ ID NO: 125), (SEQ ID NO: 127), (SEQ ID NO: 129), (SEQ ID NO: 131), (SEQ ID NO: 133), (SEQ ID NO: 135), (SEQ ID NO: 137), (SEQ ID NO: 139), (SEQ ID NO: 141), (SEQ ID NO: 143), and (SEQ ID NO: 145).

An aspect of the invention provides a composition that specifically binds a ricin B chain toxin, wherein the composition comprises a source of expression of a binding protein, wherein the source comprises a nucleotide sequence selected from: (SEQ ID NO: 147), (SEQ ID NO: 149), (SEQ ID NO: 151), (SEQ ID NO: 153), (SEQ ID NO: 155), (SEQ ID NO: 157), (SEQ ID NO: 159), (SEQ ID NO: 161), and (SEQ ID NO: 163).

An aspect of the invention provides a kit for detecting or neutralizing a toxin disease agent, the kit comprising at least one composition described herein, for example at least one selected from SEQ ID NOs:96-163. For example the toxin disease agent is at least one selected from a Shiga toxin or a ricin toxin.

An aspect of the invention provides a use of any of the compositions described herein for treating a subject exposed to the toxin. For example the composition includes at least one selected from SEQ ID NOs:96-163.

An aspect of the invention provides a use of any of the compositions described for vaccinating a subject at risk for exposure to the toxin. For example the composition includes at least one selected from SEQ ID NOs:96-163.

### BRIEF DESCRIPTION OF THE DRAWINGS

FIG. 1A-FIG. 1E are nucleotide sequences of scFv#2 (SEQ ID NO: 1), scEv43 (SEQ ID NO: 3), scFv#7 (SEQ ID NO: 5), scFv#8 (SEQ ID NO: 7), scFv#21 (SEQ ID NO: 9), scFv#E (SEQ ID NO: 11), and amino acid sequences of seFv#2 (SEQ ID NO: 2), scFv#3 (SEQ ID NO: 4), scFv#7 (SEQ ID NO: 6), scFv#8 (SEQ ID NO: 8), scFv#21 (SEQ ID NO: 10), scFv#E (SEQ ID NO: 12).

FIG. 2 is the nucleotide sequence of scFv#7-2E (SEQ ID NO: 13) and the amino acid sequence of scFv#7-2E (SEQ ID NO: 14).

FIG. 3A-FIG. 3C are the nucleotide sequences of BoNT/A holotoxin binding VHHs including JDA-D12 (SEQ ID NO: 19), JDQ-A5 (SEQ ID NO: 21), JDQ-B5 (SEQ ID NO: 23), JDQ-C2 (SEQ ID NO: 25), JDQ-F12 (SEQ ID NO: 27), JDQ-G5 (SEQ ID NO: 29), JDQ-H7 (SEQ ID NO: 31), and BoNT/B holotoxin binding VHHs including JEQ-A5 (SEQ ID NO: 33), JEQ-H11 (SEQ ID NO: 35). The Figures also show the corresponding amino acid sequences of BoNT/A holotoxin binding VHHs including JDA-D12 (SEQ ID NO: 20), JDQ-A5 (SEQ ID NO: 22), JDQ-B5 (SEQ ID NO: 24), JDQ-C2 (SEQ ID NO: 26), JDQ-F12 (SEQ ID NO: 28), JDQ-G5 (SEQ ID NO: 30), JDQ-H7 (SEQ ID NO: 32), and BoNT/B holotoxin binding VHHs including JEQ-A5 (SEQ ID NO: 34), JEQ-H11 (SEQ ID NO: 36).

FIG. 4A is a list of nucleotide sequences of VHHs identified as BoNT/A binders that were experimentally shown to bind to the same epitope. The VHH sequences are DQ-B5 (SEQ ID NO: 23), JDO-E9 (SEQ ID NO: 37), JDQ-B2 (SEQ ID NO: 39), JDQ-05 (SEQ ID NO: 41), and JDQ-F9 (SEQ ID NO: 43).

FIG. 4B is a list of corresponding VHH amino acid sequences, JDQ-B5 (SEQ ID NO: 24), JDO-E9 (SEQ ID NO: 38), JDQ-B2 (SEQ ID NO: 40), JDQ-05 (SEQ ID NO: 42), and JDQ-F9 (SEQ ID NO: 44), of the nucleic acid sequences in FIG. 4A.

FIG. 5 is a schematic drawing of a phylogenetic tree comparing the homology between BoNT/A binding VHHs within the JDQ-B5 competition group (which compete for binding, thus bind the same epitope) in comparison to control alpaca VHHs.

FIG. 6 is a schematic drawing of binding agent VHHs that are produced in different formats including formats in which the binding agents are fused to one or more E-tags or as fusion proteins.

FIG. 7 is a drawing of a single-tagged heterodimeric binding protein (exemplary VHHs) binding to the disease agent, a toxin, and leading to decoration of the toxin with two anti-tag monoclonal antibodies (mAbs).

FIG. 8 is a drawing of a double-tagged binding protein (here shown are VHHs) a heterodimeric binding to the disease agent, toxin, and leading to decoration of the toxin with four anti-tag mAbs.

FIG. 9A-FIG. 9B are a set of Meyer-Kaplan survival plots that double-tagged heterodimer E/H7/B5/E and the anti-tag mAb completely protected subjects from 1,000-fold and 1,000-fold the median lethal dose of a Botulinum neurotoxin serotype A toxin.

FIG. 9A is a Meyer-Kaplan survival plot showing percent (%) of mice surviving over a period of time (days) after receiving 1,000-fold the median lethal dose (LD₅₀) of a Botulinum neurotoxin serotype A (BoNT/A) and each of combinations of the following binding agents: H7 and B5 VHH heterodimer with a single epitopic tag (tag or E-tag) and an anti-E-tag mAb (H7/B5/E+anti-E mAb); H7 and B5 VHH monomers each with an E-tag and an anti-E-tag mAb (H7/E+B5/E+anti-E mAb); H7 and B5 VHH heterodimer with two E-tags and an anti-E-tag mAb (E/H7/B5/E+anti-E mAb) and a control (the toxin alone). The data show that administration of heterodimer E/H7/B5/E and anti-E mAb resulted in survival of subjects for seven days.

FIG. 9B is a Meyer-Kaplan survival plot showing percent (%) of subjects surviving over a period of time (days) after receiving 10,000-fold the LD₅₀ of a Botulinum neurotoxin (BoNT) and H7 and B5 VHH heterodimer with two E-tags and an anti-E-tag mAb (E/H7/B5/E+anti-E mAb) and a control (the toxin alone). Remarkably, 100% of the mice survived a 10,000 LD₅₀ challenge of BoNT/A when administered the double-tagged heterodimer and the anti-tag mAb.

FIG. 10A-FIG. 10B are nucleotide sequences and amino acid sequences of recombinant BoNT/A holotoxin binding VHHs: thioredoxin/MQ-H7(H7)/E-tag (SEQ ID NO: 45), thioredoxinaDQ-B5(B5)/E-tag (SEQ ID NO: 47), thioredoxin/H7/flexible spacer (fs)/B5/E-tag (SEQ ID NO: 49), and thioredoxin/E-tag/H7/fs/B5/E-tag (SEQ ID NO: 51). The corresponding amino acid sequences of the VHHs including amino acid sequences for thioredoxin/H7/E-tag (SEQ ID NO: 46), thioredoxin/B5/E-tag (SEQ ID NO: 48), thioredoxin/H7/fs/B5/E-tag (SEQ ID NO: 50), thioredoxin/E-tag/H7/fs/B5/E-tag (SEQ ID NO: 52), and thioredoxin (SEQ ID NO: 53) are shown.

FIG. 11A-FIG. 11B are Meyer-Kaplan survival plots showing percent survival (% survival, ordinate) of subjects as a function of time in days (abscissa) following contact with BoNT/A and later time (1.5 hours or three hours later) administered VHH binding/neutralizing agents. Subjects (five per group) were intravenously exposed to 10 LD₅₀ (ten-fold LD₅₀) of BoNT/A, and then later administered either: a mixture of 1 μg ciA-H7 monomer (SEQ ID NO: 32) and 1 μg of ciA-B5 monomer (SEQ ID NO: 24); H7/B5 heterodimeric protein (SEQ ID NO: 58); a sheep antitoxin serum; or control (no binding agent). Data show that the H7/B5 heterodimer was effective as an antitoxin neutralizing agent and protected subjects from the lethal challenge of BoNT/A.

FIG. 11A shows percent survival for subjects exposed to ten-fold LD₅₀ of BoNT/A then administered 1.5 hours later either a mixture of H7 and B5 monomers; H7/B5 heterodimer; a sheep serum antitoxin; or control toxin only (no agents).

FIG. 11B shows percent survival for subjects exposed to ten-fold LD₅₀ of BoNT/A then administered three hours later either a mixture of H7 and B5 monomers; H7/B5 heterodimer; a sheep serum antitoxin; or control toxin only (no agents).

FIG. 12A-FIG. 12C are line graphs showing that VHH monomers and VHH heterodimers neutralized C. difficile toxin b (TcdB) and protected subjects from death caused by exposure to TcdB.

FIG. 12A is a line graph showing that VHH monomers neutralized C. difficile toxin B (TcdAB) and protected cells from the toxin. The percent CT26 cells affected by TcdB affected; ordinate) is shown as a function of concentration (0.003 nM, 0.03 nM, 0.3 nM, 3 nM, 30 nM, 300 nM, or 3000 nM) of administered VHH monomers: 5D (circle), 2D (square), or E3 (light upward facing triangle). Control cells were administered toxin only (TcdB; dark downward facing triangle). Strength of neutralizing VHH activity was observed in the order 5D as strongest followed by E3 and 2D.

FIG. 12B is a line graph showing percent of cells affected by TcdB (% affected; ordinate) as a function of concentration of administered mixture of 5D and E3 monomers, 5D/E3 heterodimer (VHH; abscissa), or a toxin only control. It was observed that the 5D/E3 VHH heterodimer (squares) was about ten-fold more potent as toxin neutralizing agent than the mixture of 5D monomer and E3 monomer (triangles).

FIG. 12C is a Meyer-Kaplan survival plot of a C. difficile infection model showing percent mouse survival (ordinate) as a function of time (hours post challenge, abscissa) of subjects co-administered toxin and VHH neutralizing agents. Subjects were co-administered a lethal dose of TcdB with: a mixture of 10 μg of 5D monomers and E3 monomers (5 μg of each monomer per mouse; dashed line, blue); a mixture of 1 μg of 5D monomers and E3 monomers (500 ng of each monomer per mouse; thick solid line, blue), 5D/E3 heterodimer (250 ng per mouse; light solid line, red), or phosphate-buffered saline (PBS; thin solid line, black). Percent survival was calculated for each group of subjects.

FIG. 13A-FIG. 13C are amino acid sequences for VHH monomers and VHH heterodimers designed to specifically bind epitopes of botulism toxins serotype A (BoNT/A) and serotype B (BoNT/B). Each VHH was purified from E. coli as a thioredoxin fusion protein having a single carboxyl-terminal epitopic tag (tag or E-tag).

FIG. 13A is a set of amino acid sequences of VHH monomers that specifically recognize and bind to epitopes on BoNT/A (ciA-A5, ciA-B5, ciA-D12, ciA-F12, ciA-G5, and ciA-H7) and epitopes of BoNT/B (ciB-A11, ciB-B5, ciB-B9, and ciB-H11). The sequences are aligned to show homology. Dashed regions of the amino acid sequences are spaces inserted to align the amino acid regions.

FIG. 13B is a set of amino acid sequences of VHH monomers (ciA-D1, ciA-H5, and ciA-H11) that bind specifically to the same epitope of BoNT/B as ciA-H7.

FIG. 13C shows amino acid sequences for double-tagged VHH heterodimers, ciA-H7/ciA-B5(2E) and ciA-F12/CiA-D12(2E), that specifically bind BoNT/A.

FIG. 14A-FIG. 14B are photographs of sodium dodecyl sulfate polyacrylamide gel electrophoresis (SDS-PAGE) analyses of VHH monomers and VHH heterodimers.

FIG. 14A is a photograph of an SDS-PAGE of the tagged (E) VHH monomers ciA-D1, ciA-H4, ciA-H11, ciA-A5, ciA-C2, ciA-D12, ciA-F12, ciA-G5, and ciA-H7.

FIG. 14B is a photograph of an SDS-PAGE of single- or double-tagged VHH heterodimers including: ciA-H7/ciA-B5 singly tagged on ciA-B5 (left channel); double tagged ciA-H7/ciA-B5 having a tag on both ciA/H7 and ciA-B5 (second channel from left), ciA-F12/ciA-D12 singly tagged on ciA-B5 (third channel from the left); double tagged ciA-F12/ciA-D12 having a tag on both ciA/F12 and ciA-D12 (fourth channel from left), double tagged ciA-A11/ciA-B5 having a tag on both ciA/A11 And ciA-B5 (right channel).

FIG. 15 is a set of photographs of Western blots showing ability of VHH monomers to prevent BoNT/A from cleavaging of synaptosomal-associated protein 25 (SNAP25) in primary neurons in culture.

FIG. 16A-FIG. 16C are a set of drawings and Meyer-Kaplan survival plots showing that mouse subjects administered each of a set of mixtures of VHH monomers in combination with anti-tag clearing antibody were protected from BoNT/A.

FIG. 16A (top) is a drawing of a BoNT/A bound to two different tagged binding protein monomers that are each specifically bound by an anti-tag antibody. FIG. 16 A (bottom) is a set of graphs showing percent of survival (% survival, ordinate) as a function of time (days, abscissa) of subjects co-administered 100-fold (FIG. 16A bottom left graph) or 1,000-fold (FIG. 16A bottom right graph) the LD₅₀ of a BoNT/A and combinations of VHH monomers (ciA-D12 and ciA-F12) with or without anti-tag clearing antibody (+αE and −αE respectively). The mixture of VHH monomer B5, VHH monomer H7 and anti-tag clearing antibody protected subjects from the 100-fold LD50 of toxin.

FIG. 16B (top) is a drawing of a BoNT/A bound to three different monomeric tagged binding protein each specifically bound by an anti-tag antibody. FIG. 16 B (bottom) is a set of graphs showing percent survival on the ordinate as a function of time (days, abscissa) of subjects co-administered 1,000-fold BoNT/A LD₅₀ (FIG. 16B bottom left graph) or 10,000-fold BoNT/A LD₅₀ (FIG. 16B bottom right graph), and combinations of three VHH monomers with or without anti-tag clearing antibody (+αE and −αE respectively).

FIG. 16C (top) is a drawing of a BoNT/A bound to four different tagged binding protein monomers that are each specifically bound by an anti-tag antibody. FIG. 16 C (bottom) is a set of graphs showing percent survival, ordinate, of subjects as a function of time (days, abscissa) of subjects co-administered 1,000-fold BoNT/A LD₅₀ (FIG. 16 C bottom left graph) or 10,000-fold BoNT/A LD₅₀ (FIG. 16 C bottom right graph), and a mixture of ciA-B5, ciA-H7, ciA-D12 and ciA-F12 VHH monomers with (+αE) or without (−αE) anti-tag clearing antibody.

FIG. 17 is a set of graphs showing percent survival, ordinate, of subjects as a function of time (days, abscissa) of subjects co-administered 1,000-fold BoNT/A LD₅₀ (FIG. 17 left graph) or 10,000-fold BoNT/A LD₅₀ (FIG. 17 right graph), and mixtures of VHH monomers and anti-tag clearing antibody (αE). Control subjects received toxin only. Unless indicated otherwise, an asterisk (*) in FIGS. 17-24 indicates that the subjects administered the VHH monomer or multimer displayed no symptoms of toxin exposure.

FIG. 18A-FIG. 18B are a table showing affinity binding data for VHHs and a set of line graphs showing improved protection of subjects from very large doses of BoNT/A following administration of each of sets of mixtures of VHH monomers with strong affinity for BoNT/A and clearing antibody.

FIG. 18A is a table showing binding affinities (Kd) determined by surface plasmon resonance (SPR) analysis of each of VHH monomers ciA-H7, ciA-D1, ciA-H4, and ciA-H11. SPR analysis was used to determine the binding affinities to epitope A1 of BoNT/A for each VHH monomer. H7 has the greatest affinity and H11 the least affinity.

FIG. 18B is a set of graphs showing percent survival on the ordinate of subjects as a function of time (days, abscissa) following co-administration of BoNT/A at 100-fold (FIG. 18B left graph) or 1,000-fold (FIG. 18B right graph) the LD₅₀, and a mixture of two VHH monomers (B5+C2) or a mixture of three VHH monomers with anti-tag clearing antibody: B5+C2+H11; B5+C2+H7; B5+C2+D1; or B5+C2+H2.

FIG. 19A-FIG. 19B are drawings and graphs showing that administering heterodimers composed of neutralizing VHH components resulted in greater antitoxin efficacy than heterodimers composed of non-neutralizing VHHs, and that presence of two or more E-tags within the VHH heterodimers further increased the antitoxin efficacy.

FIG. 19A (top) is a drawing of a BoNT/A bound to two different tagged heterodimer binding proteins that are each specifically bound by an anti-tag antibody. FIG. 19 A (bottom) is a set of graphs showing percent survival on the ordinate of subjects as a function of time (days, abscissa) after co-administration of 1,000-fold (FIG. 19 A bottom left graph) or 10,000-fold (FIG. 19 A bottom right graph) the BoNT/A LD₅₀, and a VHH heterodimer composition with (+αE) or without (−αE) anti-tag clearing antibody. The tagged VHH heterodimer composition was either composed of neutralizing VHHs ciA-H7 and ciA B5 (H7/B5), or of non-neutralizing VHHs ciA-D 12 and ciA-F12 (D12/F12). Data show that subjects administered the heterodimer composition containing neutralizing VHHs ciA-B5 and ciA-H7 survived longer than subjects administered the heterodimer composition containing non-neutralizing VHHs ciA-D12 and ciA-F12. Subjects administered clearing anti-tag antibodies generally survived longer than subjects not administered clearing-tag antibodies.

FIG. 19B (top) is a drawing of a BoNT/A bound to two different double-tagged heterodimer binding proteins that are each specifically bound by two anti-tag antibodies. FIG. 19B (bottom) is a set of graphs showing percent survival, ordinate, of subjects as a function of time (days, abscissa) after co-administration of an amount of BoNT/A 1,000-fold (FIG. 19B bottom left graph) or 10,000-fold (FIG. 19B bottom right graph) the LD₅₀, and double tagged VHH heterodimers with (+αE) or without (−αE) anti-tag clearing antibody. Subjects administered neutralizing ciA-B5/ciA-H7 heterodimer survived longer than subjects administered non-neutralizing ciA-D12/ciA-F12 heterodimer. Data show that all subjects administered double-tagged ciA-B5/ciA-H7 heterodimers and anti-tag clearing antibody survived exposure to 1,000-fold (FIG. 19B bottom left graph) or 10,000-fold the LD₅₀ of BoNT/A (FIG. 19B bottom right graph).

FIG. 20 is a set of graphs showing percent survival on the ordinate of subjects as a function of time (days, abscissa) after co-administration of 100-fold (FIG. 20 left graph) or 1,000-fold (FIG. 20 right graph) BoNT/A LD₅₀, and multi-tagged VHH heterodimers with anti-tag clearing antibody. The ciA-D12/ciA-F12 heterodimer protein contained either one tag (le), two tags (2e), three tags (3e), or control no tag. Subjects (five mice per group) were administered 20 μg of the heterodimer composition or the mixture of ciA-D12 and ciA-F12 monomers (20 μg of each monomer). Control subjects were administered neither monomer nor heterodimer. Each subject received 60 picomoles of anti-E-tag clearing antibody. Data show that subjects administered ciA-D12/ciA-F12 heterodimers having either one tag or two tags survived (100% survival) the challenge of 100-fold the LD₅₀ of BoNT/A (FIG. 20 left graph). Subjects receiving 1,000-fold the LD₅₀ of BoNT/A and ciA-D12/ciA-F12 heterodimers with clearing antibody died within one day following challenge with independent of number of tags (FIG. 20 right graph).

FIG. 21 is a set of graphs showing percent survival, ordinate, of subjects treated with different amounts of anti-tag clearing antibody as a function of time (days, abscissa) after exposure to BoNT/A 100-fold (FIG. 21 left graph) or 1,000-fold (FIG. 21 right graph) the LD₅₀ and to double tagged ciA-D12/ciA-F12 heterodimer (20 picomoles). Anti-tag clearing antibody was administered at: 20 picomoles, 40 picomoles, 60 picomoles, 120 picomoles, or control (none). Control subjects received toxin only (no agents). Data show improved antitoxin efficacy in subjects co-administered amounts (40, 60 or 120 picomoles) increased anti-tag clearing antibody compared to 20 picomoles.

FIG. 22 is a graph showing percent survival, ordinate, of subjects treated with different doses of double tagged neutralizing ciA-B5/ciA-H7 heterodimers as a function of time (days, abscissa) for subjects co-administered 1,000-fold BoNT/A LD₅₀, and anti-tag clearing antibody. Heterodimer ciA-B5/ciA-H7 was administered in doses of: 1.5 picomoles, 4.4 picomoles, 13 picomoles, or 40 picomoles. Control subjects received toxin only (no agents). Data show complete survival after seven days of subjects receiving amounts of 13 picomoles or 40 picomoles double tagged neutralizing ciA-B5/ciA-H7 heterodimer, such that than 13 picomoles protected subjects fully from 1,000-fold BoNT/A LD₅₀, compared to 1.5 picomoles or 4.4 picomoles (no survival after one day).

FIG. 23A-FIG. 23B are a set of graphs showing percent survival, ordinate, after subjects were exposed to ten-fold BoNT/A LD₅₀ and were administered double-tagged heterodimer and anti-tag clearing antibody of subjects as a function of time (days, abscissa). Administration of heterodimer after toxin exposure was observed to have protected subjects from symptoms and death caused by exposure to ten-fold BoNT/A LD₅₀.

FIG. 23A is a set of graphs showing percent survival of subjects as a function administration of: double tagged ciA-D12/ciA-F12 heterodimer with anti-tag clearing antibody (+αE), double tagged ciA-D12/ciA-F12 heterodimer without anti-tag clearing antibody (−αE), a sheep serum antitoxin, or toxin only control (no agents). Prior to administration of heterodimer, subjects were exposed 1.5 hours (FIG. 23 A left graph) or three hours (FIG. 23 A right graph) to ten-fold BoNT/A LD₅₀. Data show 100% survival of subjects administered ciA-D12/ciA-F12 heterodimer and anti-tag antibody after 1.5 hours. Survival of subjects administered ciA-D12/ciA-F12 heterodimer was comparable to that in subjects administered sheep serum antitoxin.

FIG. 23B is a set of graphs showing percent survival of subjects as a function administration of: double tagged ciA-B5 and ciA-H7 heterodimer with anti-tag clearing antibody (+αE), or with double tagged ciA-B5/ciA-H7 heterodimer without anti-tag clearing antibody (−αE), or with a sheep serum antitoxin, or toxin only control (no agents). Prior to treatment with heterodimer, subjects were exposed to ten-fold BoNT/A LD₅₀ either 1.5 hours (FIG. 23 B left graph) or three hours (FIG. 23 B right graph). Data show that subjects administered ciA-B5/ciA-H7 heterodimer with or without anti-E tag antibody survived longer than subjects administered sheep serum antitoxin. Survival of subjects administered ciA-B5/ciA-H7 heterodimer was greater than subjects administered sheep serum antitoxin.

FIG. 24A-FIG. 24B are line graphs showing that subjects administered ciA-A11/ciA-B5 heterodimers with anti-tag clearing antibody were protected from BoNT/B exposure.

FIG. 24A is a graph showing survival on the ordinate as a function of time (days, abscissa) co-administration of 1,000-fold (FIG. 24 A left graph) or 10,000-fold (FIG. 24 A right graph) BoNT/B LD₅₀ and a combination of ciB-A11 And ciB-B5 heterodimer with (+αE) or without (−αE) anti-tag clearing antibody, or toxin only control (no agents). Data show that subjects administered ciA-A11/ciA-B5 heterodimer and anti-E-tag clearing antibody survived and were protected longer from BoNT/A than control subjects administered no agents and no anti-E tag antibody.

FIG. 24B is a set of graphs showing subject survival (ordinate) as a function of time, abscissa, after administration of: double tagged ciB-A11 And ciB-B5 heterodimer and anti-tag clearing antibody (+αE), or double tagged ciB-A11 And ciB-B5 heterodimer without anti-tag clearing antibody (−αE), a sheep serum antitoxin, or toxin only control. Following 1.5 hours (FIG. 24 B left graph) or three hours (FIG. 24 B right graph) exposure to ten-fold BoNT/B LD₅₀, the subjects were administered the heterodimer. A greater percentage of subjects administered ciB-A11 And ciB-B5 heterodimer survived exposure to BoNT/B than subjects administered sheep serum antitoxin.

FIG. 25 is a line graph of percent of cells affected by C. difficile toxin A (TcdA) and protection of cells from the toxin by VHH monomers. The percent CT26 cells affected by TcdA (% affected; ordinate) is shown as a function of concentration (0.1 nM, 0.48 nM, 2.4 nM, 12 nM, 60 nM, or 300 nM) of each administered VHH monomer: A3H (circle), AUG (light square); AC1 (upward dark empty triangle), AE1 (upward light triangle), AH3 (downward triangle), or AA6 (dark empty square). Control cells were administered toxin only (TcdA; dark downward triangle). Strength of neutralizing VHH activity was observed in the following order: AA6 as strongest, then AH3, AC1, AE1, AUG, and A3H as weakest.

FIG. 26 is a line graph showing percent CT26 cells affected after 24 hours of TcdA exposure (ordinate) as a function of concentration administered (abscissa: 0.03 ng/mL, 0.1 ng/mL, 1 ng/mL, 3 ng/mL, 10 ng/mL, 30 ng/mL, 100 ng/mL, 300 ng/mL, or 1000 ng/mL), or toxin only control (TcdA; vertical line). Agents administered were: VHH monomer AH3 (AH3, diamond), VHH monomer AA6 (AA6, square), a mixture of VHH monomers AH3 and AA6 (AH3+AA6, triangle), VHH heterodimer of AH3 and AA6 (AH3/AA6, -x-); or a homodimer of heterodimer (tetramer) containing AH3 and AA6 using a dimerizer sequence oAgBc (AH3/AA6/oAgBc, stars; SEQ ID NO: 95). Control cells were treated with medium only. Percent cell rounding was analyzed using a phase contrast microscope. It was observed that the homodimer of the heterodimer containing AH3 and AA6 resulted in the strongest TcdA neutralization.

FIG. 27 is a set of line graphs showing percent affected CT26 cells exposed to toxin (ordinate) and then contacted with VHH heterodimer of 5D and AA6 (FIG. 27 left graph) or with heterodimer of 5D and AH3 (FIG. 27 right graph) as a function of concentration of VHH (abscissa: 0.01 nM, 0.03 nM, 0.1 nM, 0.3 nM, 1 nM, 3 nM, 10 nM, or 30 nM). CT26 cells were exposed overnight to TcdA (2 ng/mL; diamond) or TcdB (0.1 ng/mL; square), and then treated with either heterodimer 5D/AA6 (FIG. 27 left graph) or heterodimer 5D/AH3 (FIG. 27 right graph). Each heterodimer included a VHH monomer (5D) that neutralized TcdB, and a VHH monomer (AA6 or AH3) that neutralized TcdA. Data show that the treatment was effective to protect cells from both toxins.

FIG. 28A-FIG. 28C are a drawing, a line graph and a bar graph showing that a VHH heterodimer of 5D and AA6 protected mouse subjects from TcdA and TcdB in an oral C. difficile spore challenge model.

FIG. 28A is a protocol for a clinically relevant murine C. difficile infection model. Administration of VHH is given after a spore challenge.

FIG. 28B shows percent survival (ordinate) as a function of time following spore challenge (abscissa) for subjects administered 5D/AA6 heterodimer as described in FIG. 28 A. Data show that after the spore challenge, 90% of 5D/AA6 heterodimer contacted-subjects survived, and all control subjects not administered 5D/AA6 heterodimer or other agent died within two days.

FIG. 28C showing percent diarrhea (ordinate) as a function of time following spore challenge (abscissa) for subjects administered 5D/AA6 heterodimer (5D/AA6 TrxA; left bar), or control PBS (right bar) as described in FIG. 28 A. Data show that 5D/AA6 heterodimer administered-subjects were five-fold less likely to display symptoms of diarrhea than control untreated subjects.

FIG. 29A-FIG. 29B are amino acid sequences and nucleotide sequences for VHHs that specifically bind either Shiga toxin, anthrax protective antigen, ricin A chain (RTA) antigen, or ricin B chain (RTB) antigen. The nucleotide SEQ ID NOs: 131, 155 and 159 shown in FIG. 29 B include the letter “N” in the nucleotide sequences in FIG. 29 B which indicates a position in the nucleotide sequence for which an adenine (A) residue or a guanine (G) residue may be inserted to encode the corresponding amino acid in FIG. 29 A.

FIG. 29A is a list of amino acid sequences of VHHs identified that bind each target as indicated:

Shiga toxin: JET-H12 (SEQ ID NO:96) and JFG-H6 (SEQ ID NO: 98);

anthrax protective antigen: JHD-B6 (SEQ ID NO: 100), JHE-D9 (SEQ ID NO: 102), JIJ-A12 (SEQ ID NO: 104), JIJ-B8 (SEQ ID NO: 106), JIJ-C11 (SEQ ID NO: 108), JU-D3 (SEQ ID NO: 110), JIJ-E9 (SEQ ID NO: 112), JD-F11 (SEQ ID NO: 114), JIK-B8 (SEQ ID NO: 116), MK-B10 (SEQ ID NO: 118), JIK-B12 (SEQ ID NO: 120), and JIK-F4 (SEQ ID NO: 122);

RTA: JIV-F5 (SEQ ID NO: 124), JIV-F6 (SEQ ID NO: 126), JIV-G12 (SEQ ID NO: 128), JIY-A7 (SEQ ID NO: 130), JIY-D9 (SEQ ID NO: 132), JIY-D10 (SEQ ID NO: 134), JIY-E1 (SEQ ID NO: 136), JIY-E3 (SEQ ID NO: 138), JIY-E5 (SEQ ID NO: 140), JIY-F10 (SEQ ID NO: 142), and JIY-G11(SEQ ID NO: 144); and,

RTB: JIW-B1 (SEQ ID NO: 146), JIW-C12 (SEQ ID NO: 148), JIW-D12 (SEQ ID NO: 150), JIW-G5 (SEQ ID NO: 152), JIW-G10 (SEQ ID NO: 154), JIZ-B7 (SEQ ID NO: 156), JIZ-B9 (SEQ ID NO: 158), JIZ-D8 (SEQ ID NO: 160), and JIZ-G4 (SEQ ID NO: 162).

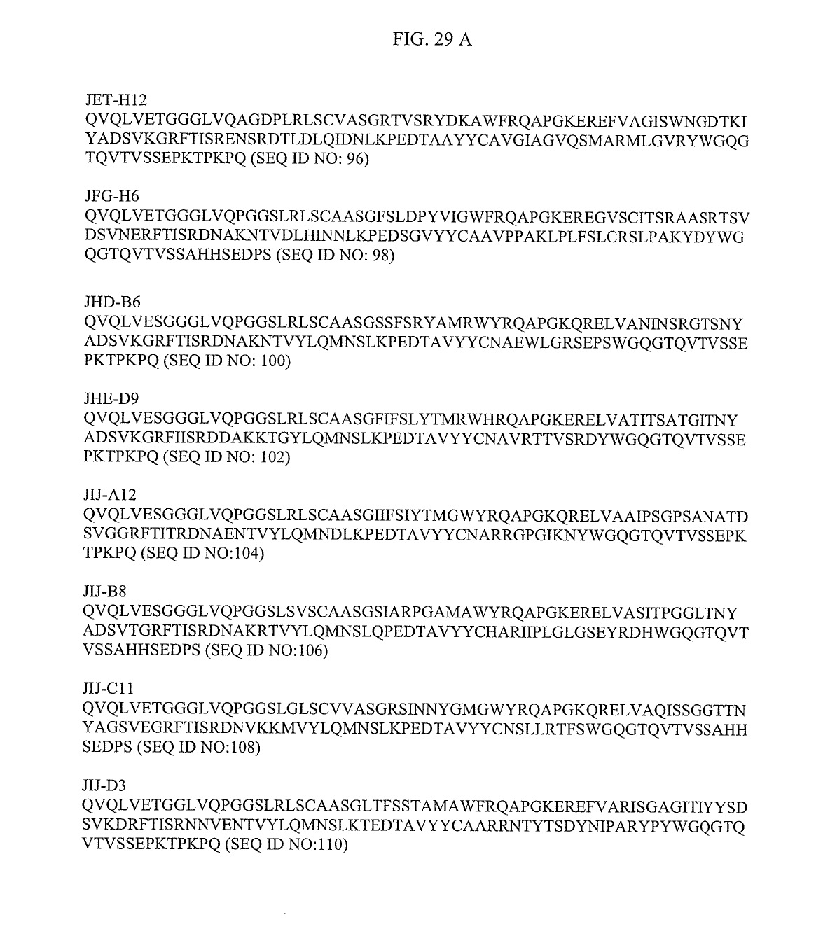

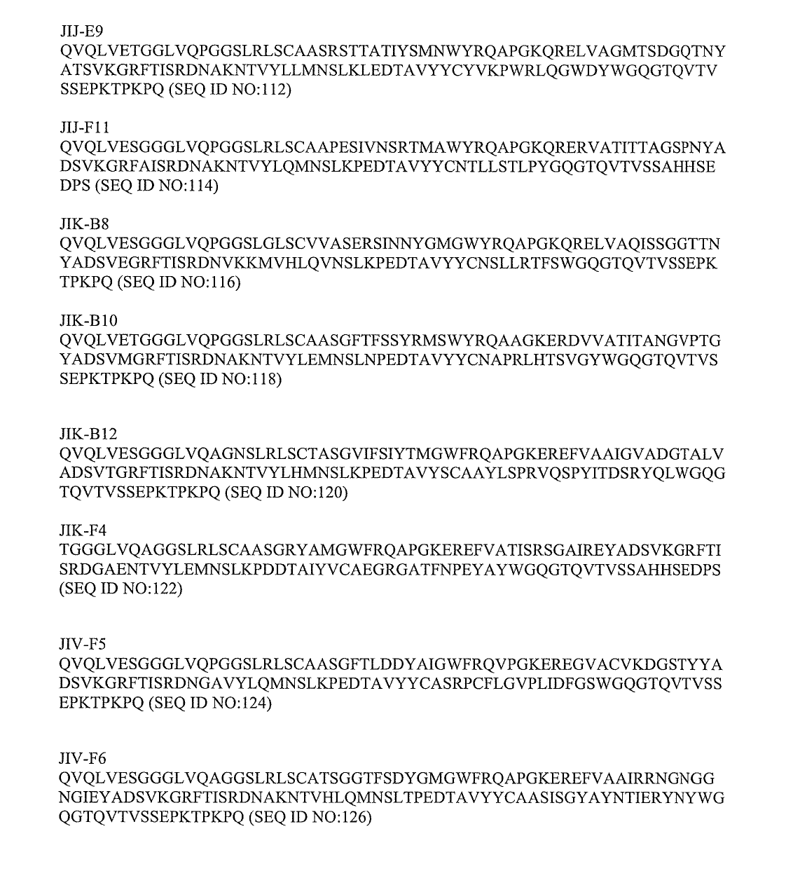

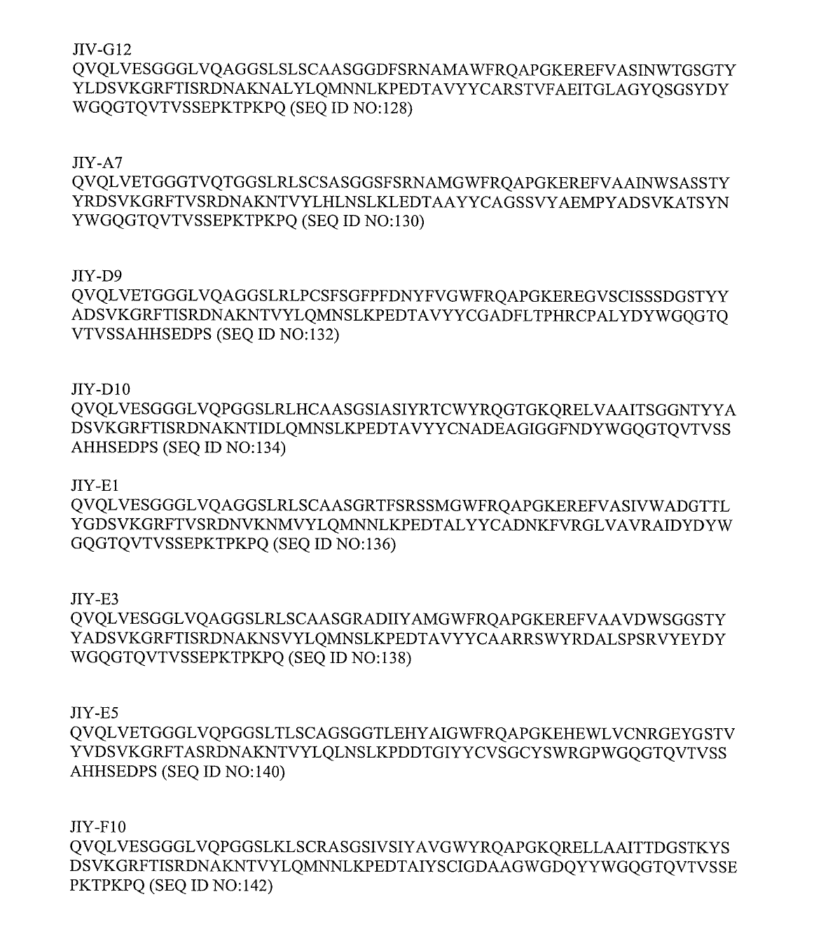

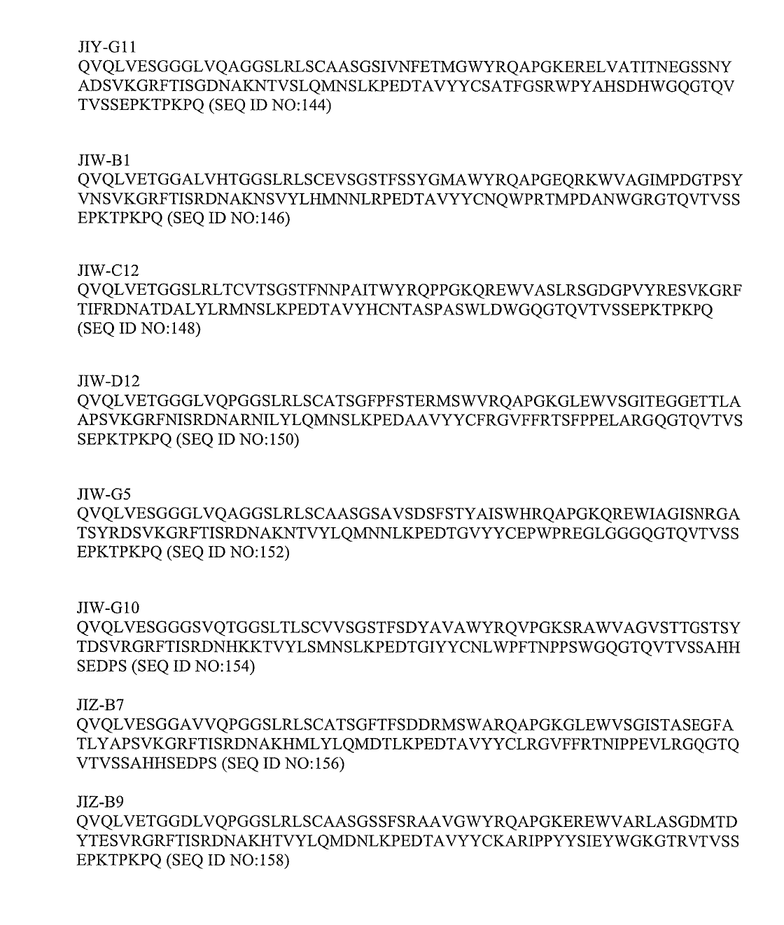

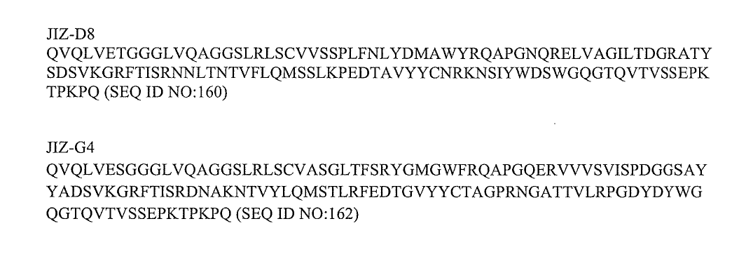

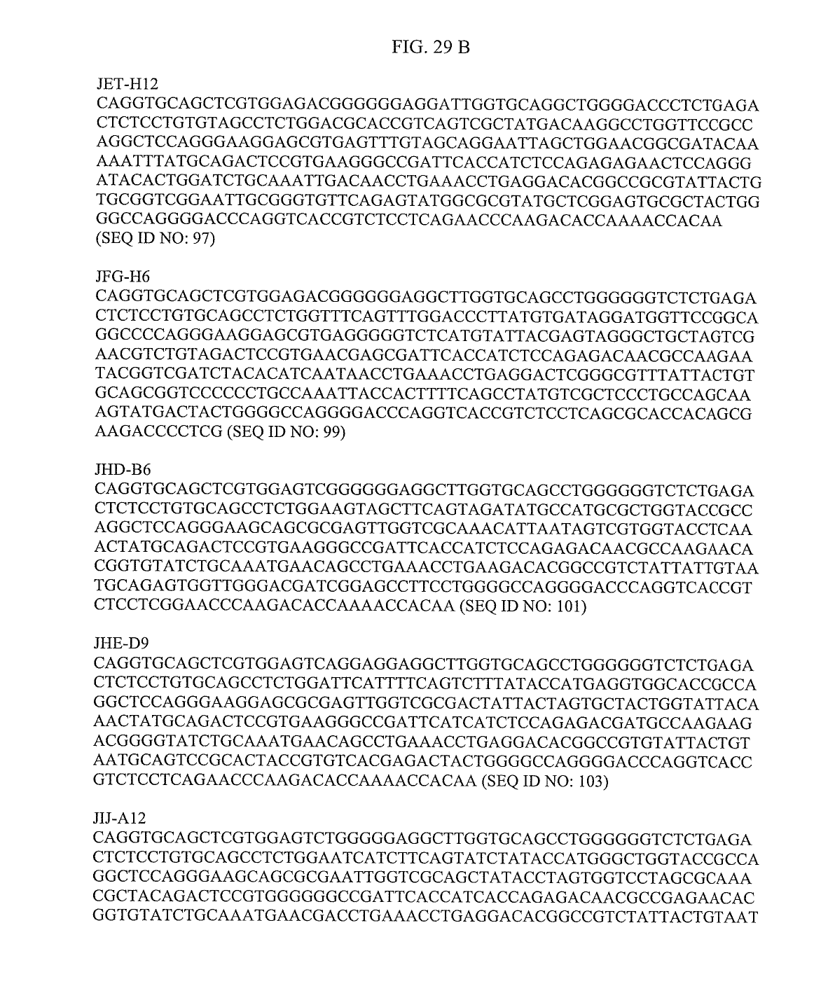

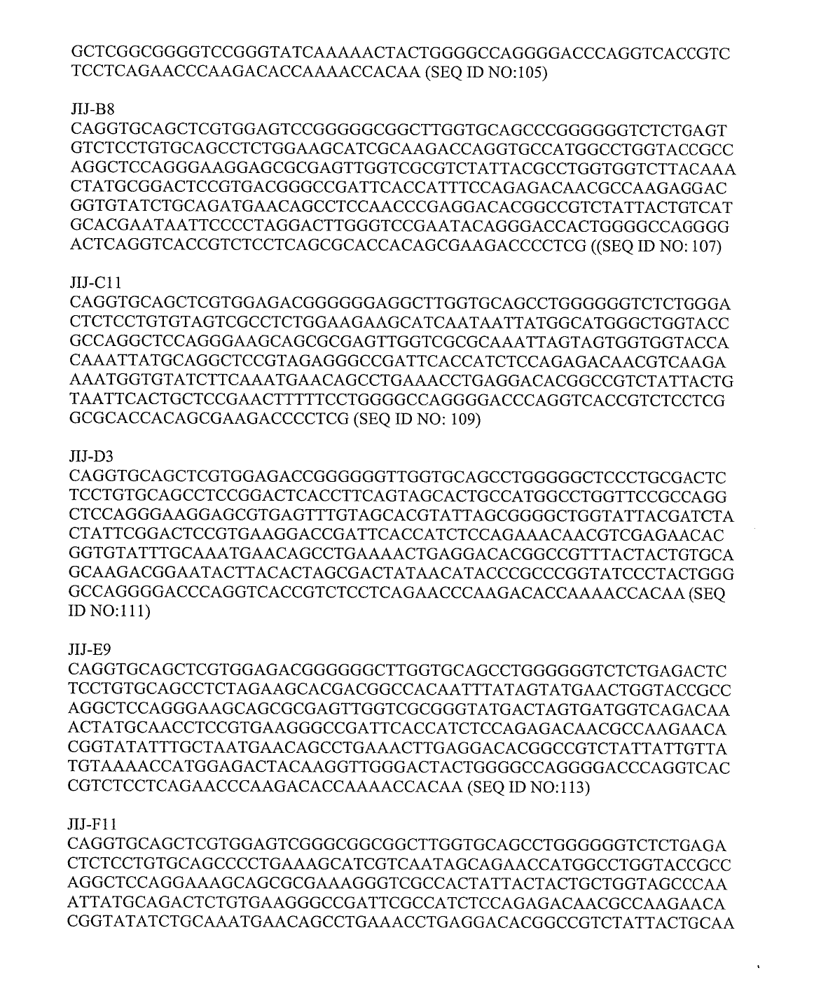

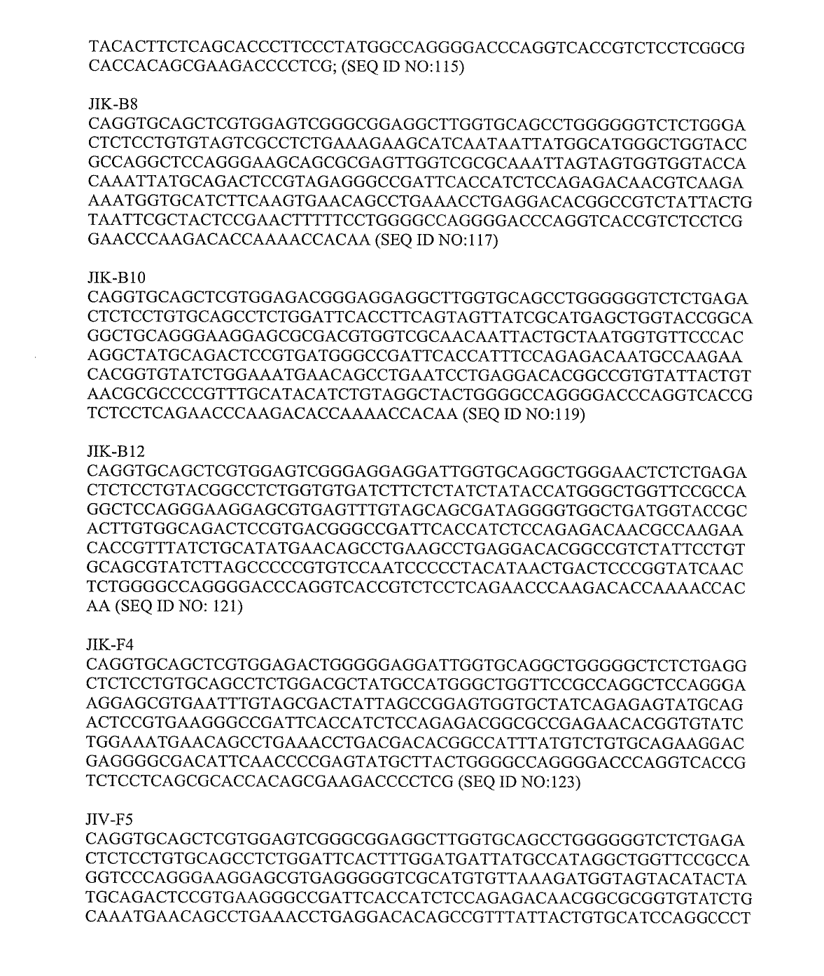

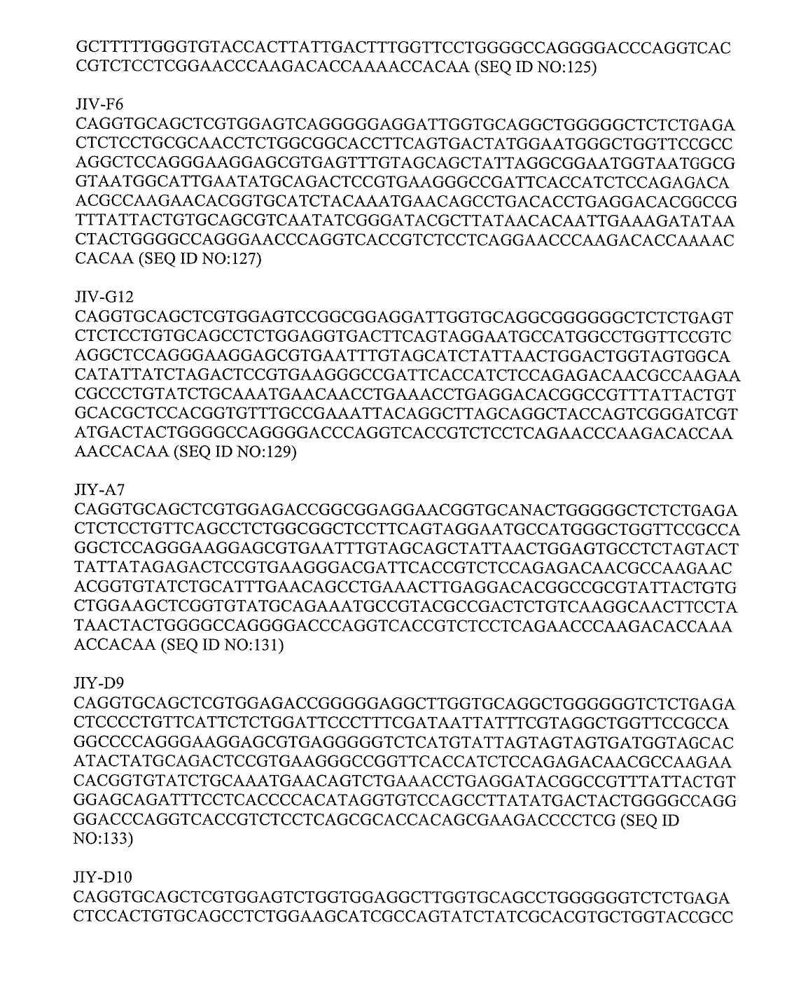

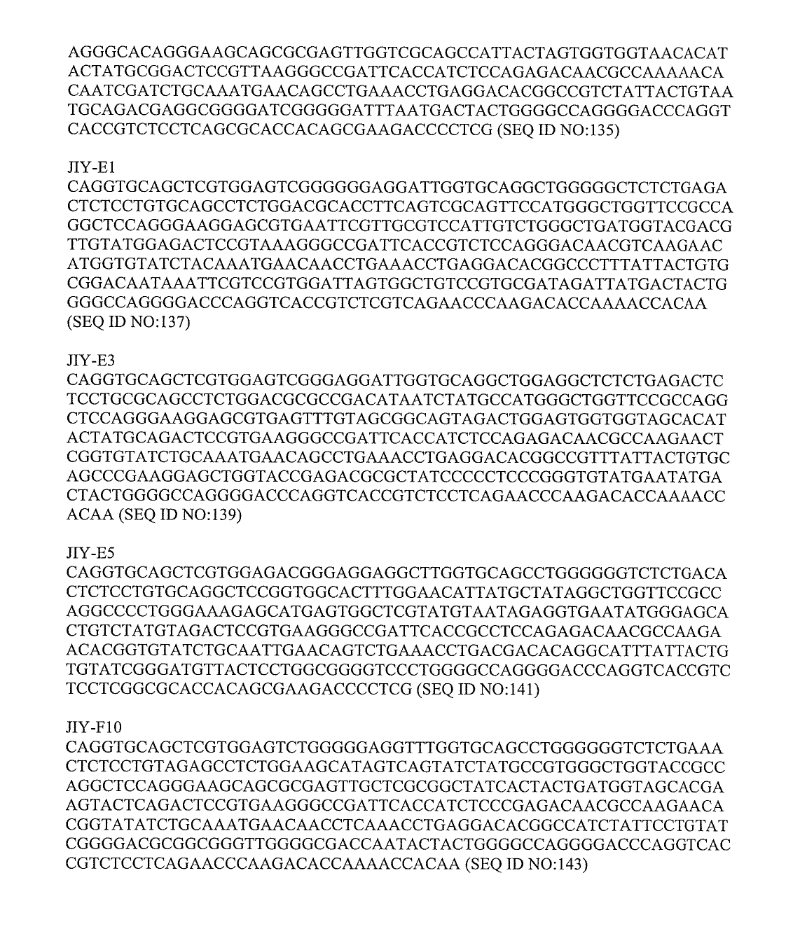

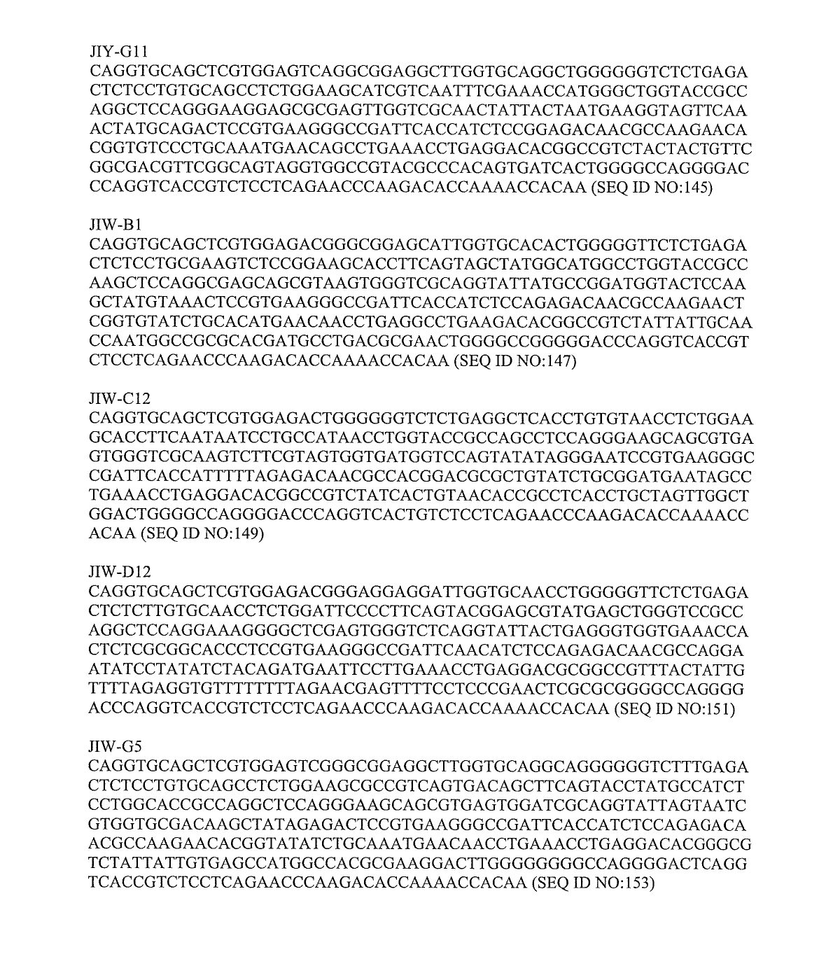

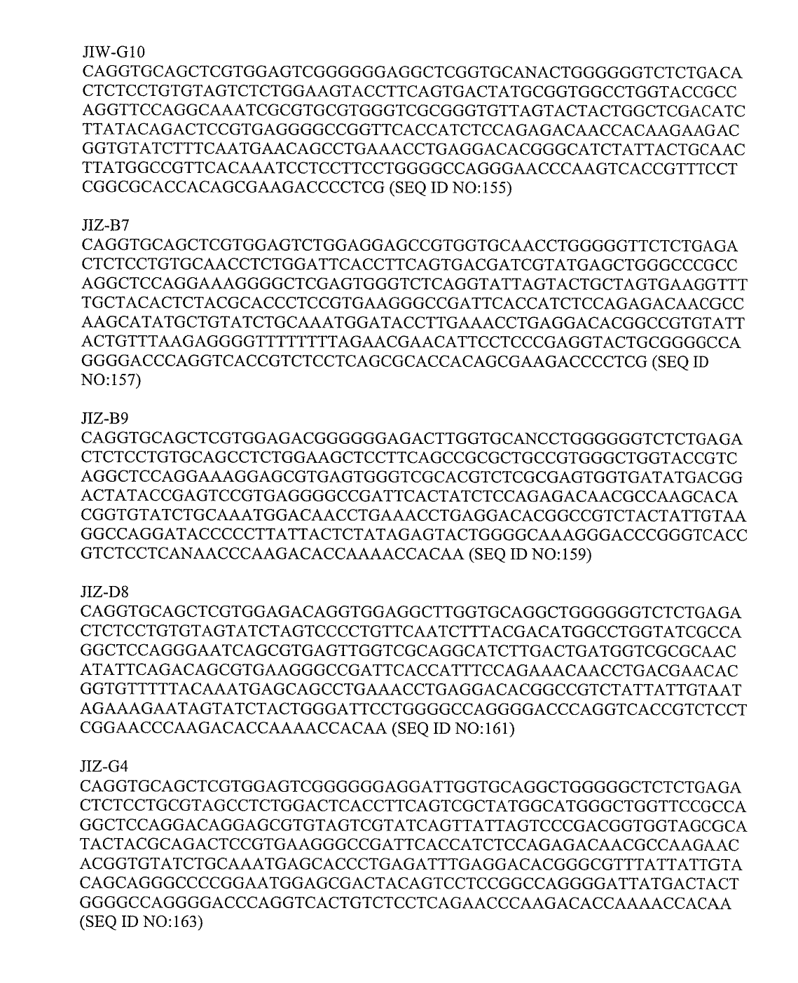

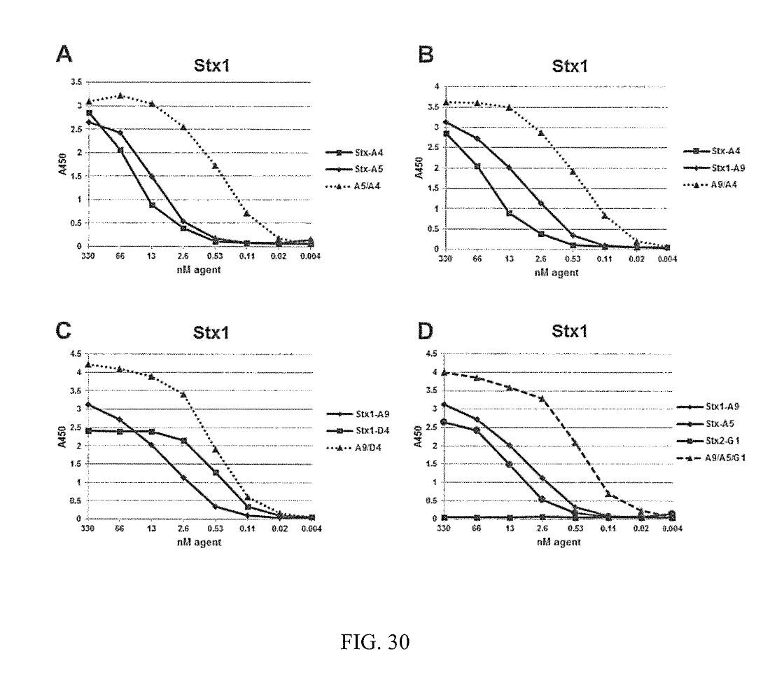

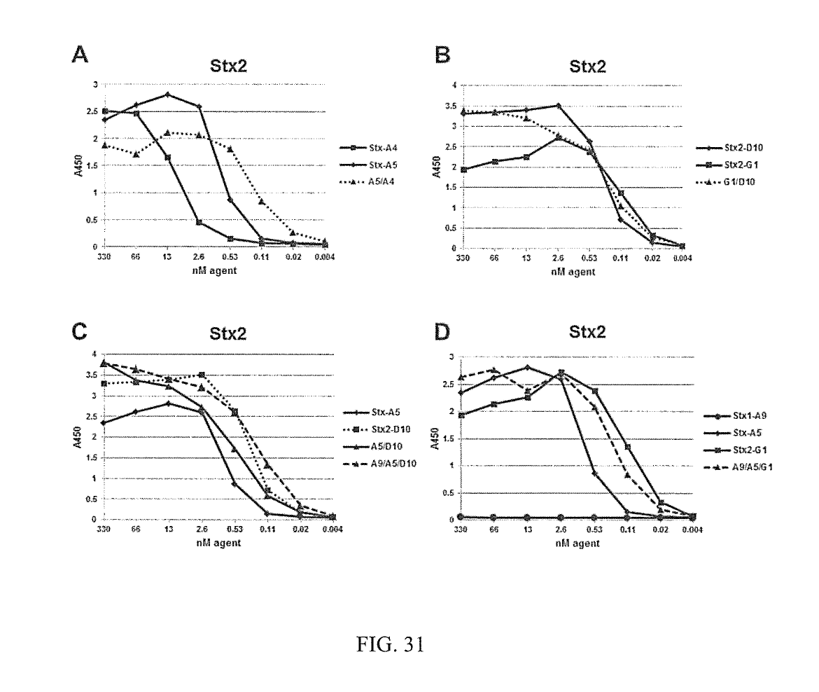

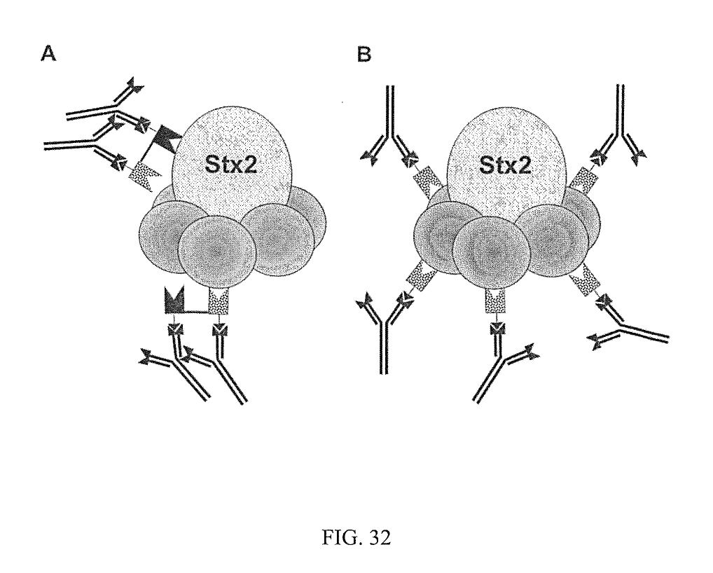

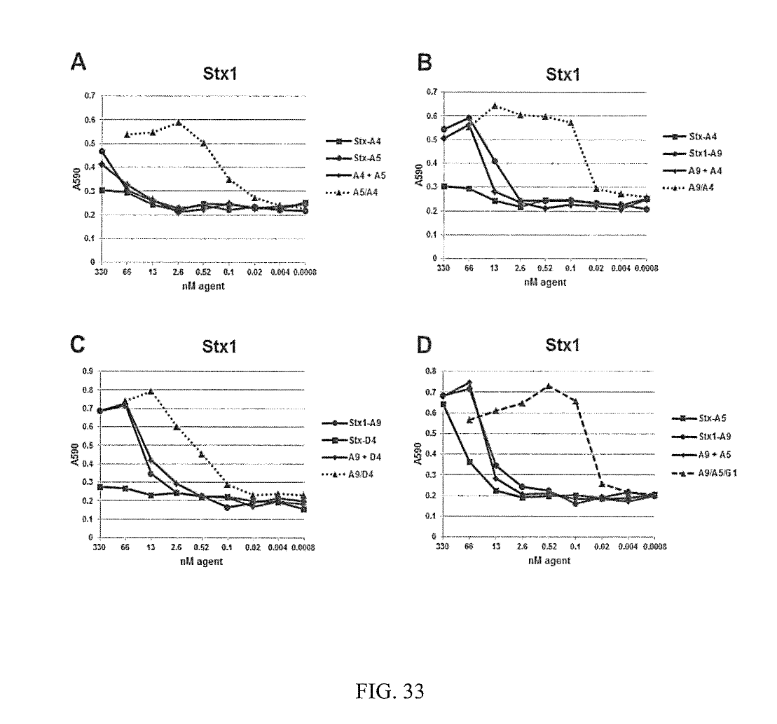

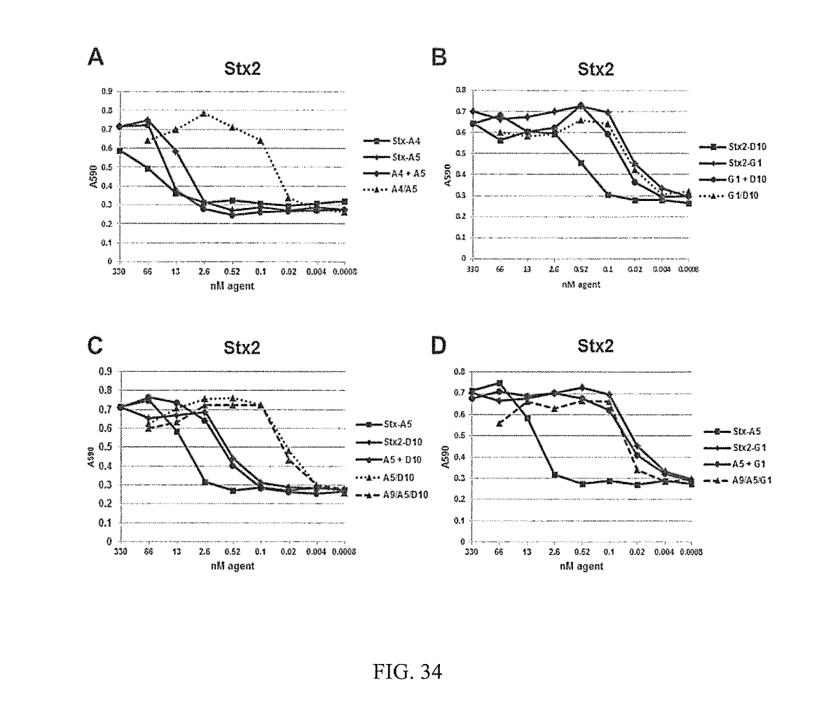

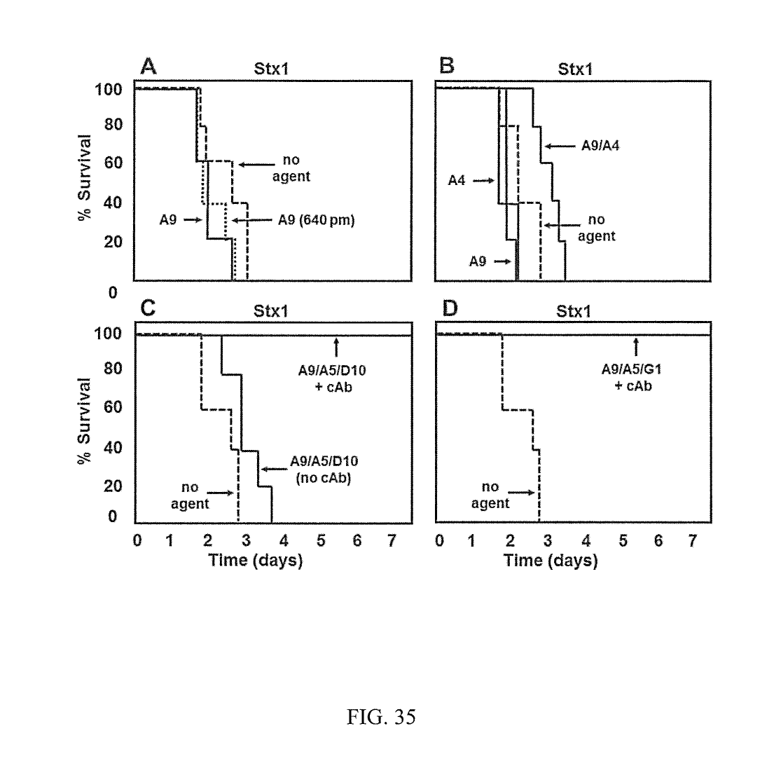

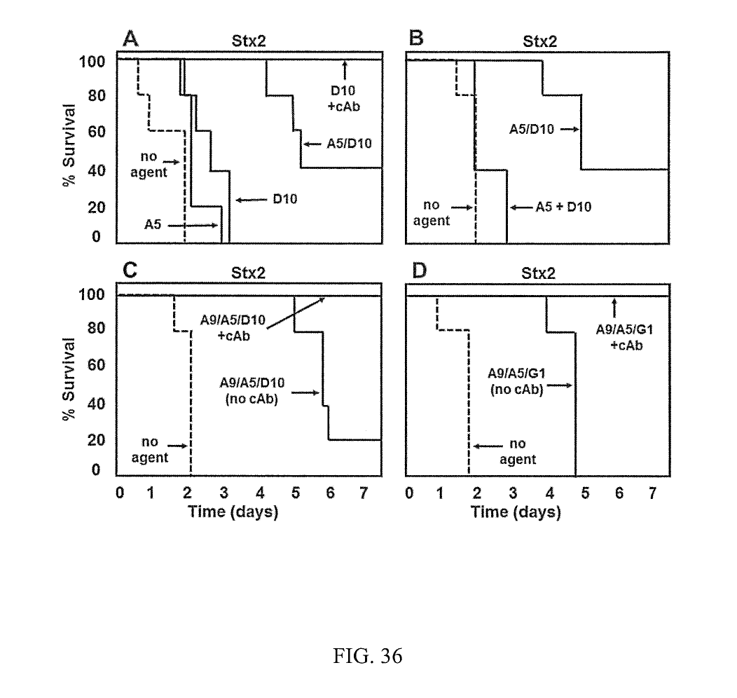

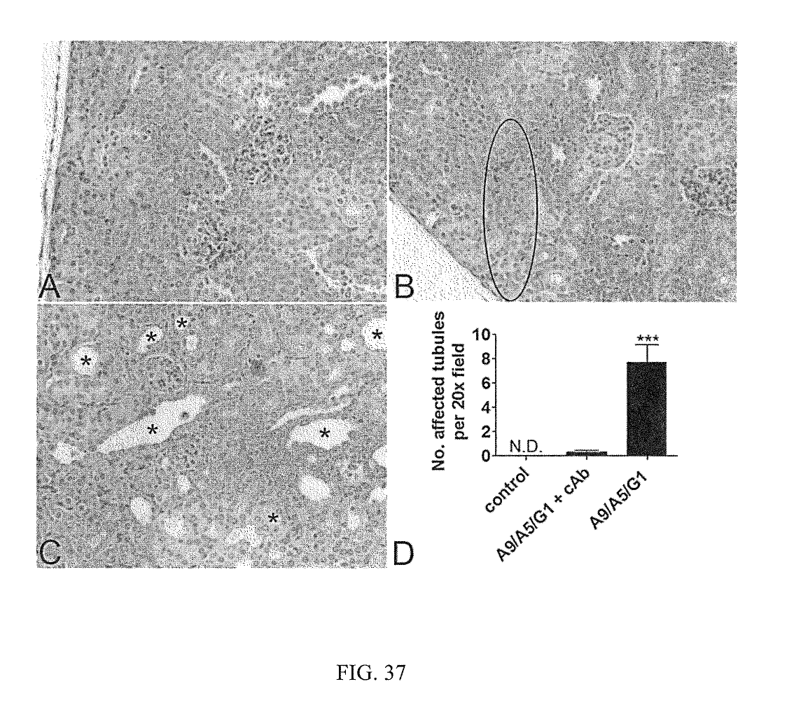

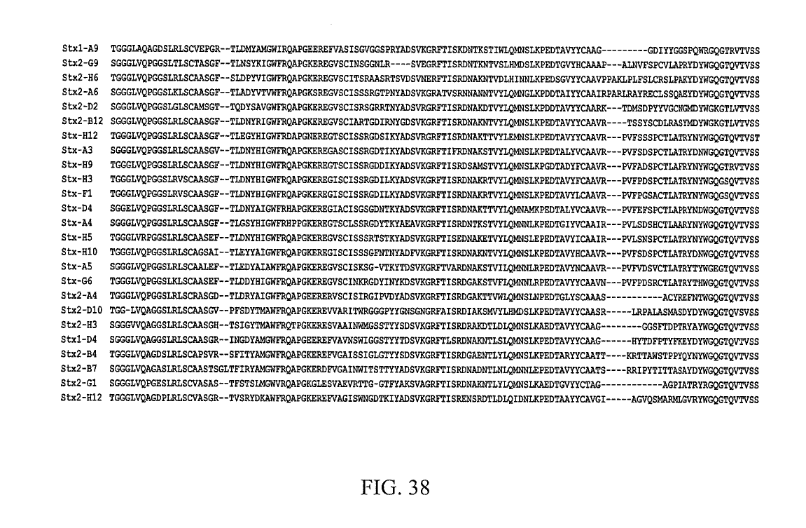

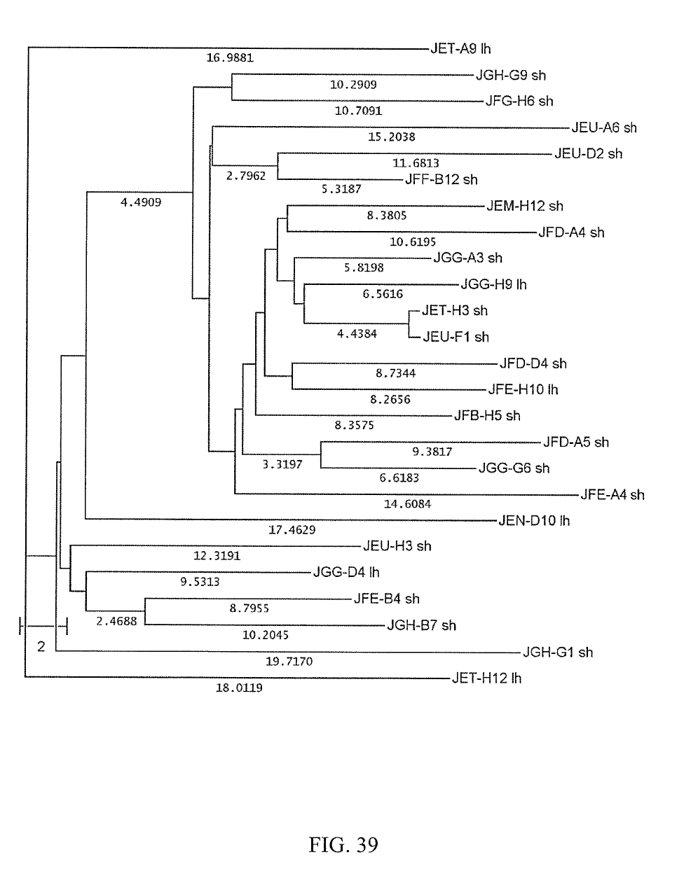

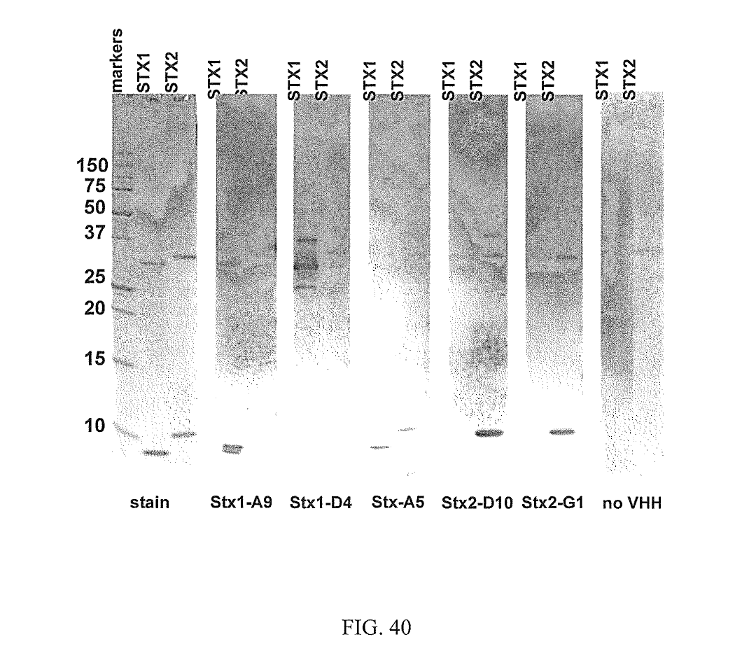

FIG. 29B is a list of nucleotide sequences that encode the VHH amino acid sequences listed in FIG. 29 A. The nucleotide sequences encode VHHs that bind each target as indicated:

Shiga toxin: JET-H12 (SEQ ID NO:97) and JFG-H6 (SEQ ID NO: 99);

anthrax protective antigen: JHD-B6 (SEQ ID NO: 101), JHE-D9 (SEQ ID NO: 103), JIJ-A12 (SEQ ID NO: 105), JU-B8 (SEQ ID NO: 107), JU-C11 (SEQ ID NO: 109), JIJ-D3 (SEQ ID NO: 111), JIJ-E9 (SEQ ID NO: 113), JIJ-F11 (SEQ ID NO: 115), JIK-B8 (SEQ ID NO: 117), JIK-B10 (SEQ ID NO: 119), JIK-B12 (SEQ ID NO: 121), and JIK-F4 (SEQ ID NO: 123);

RTA: JIV-F5 (SEQ ID NO: 125), JIV-F6 (SEQ ID NO: 127), JIV-012 (SEQ ID NO: 129), JIY-A7 (SEQ ID NO: 131), JIY-D9 (SEQ ID NO: 133), JIY-D10 (SEQ ID NO: 135), JIY-E1 (SEQ ID NO: 137), JIY-E3 (SEQ ID NO: 139), JIY-E5 (SEQ ID NO: 141), JIY-F10 (SEQ ID NO: 143), and JIY-G11 (SEQ ID NO: 145); and,

RTB: JIW-B1 (SEQ ID NO: 147), JIW-C12 (SEQ ID NO: 149), JIW-D12 (SEQ ID NO: 151), JIW-G5 (SEQ ID NO: 153), JIW-G10 (SEQ ID NO: 155), JIZ-B7 (SEQ ID NO: 157), JIZ-B9 (SEQ ID NO: 159), JIZ-D8 (SEQ ID NO: 161), and JIZ-G4 (SEQ ID NO: 163).

FIG. 30A-FIG. 30D are a set of line graphs showing VHH binding to Stx1 toxin as a function of input VHH concentration. Dilution ELISAs were performed by coating plates with 0.5 μg/ml of 4D3 mAb Stx1. The plates were blocked and then incubated with 0.3 μg/ml of Stx1. For standard ELISAs, plates were coated with 1.5 μg of Stx1. VHH agents to be tested were serially diluted, incubated for 1 hour at room temperature, washed and the bound VHH agent were detected with HRP-anti-E-tag. The bound VHH-HRP tagged agents were detected using the TMB kit by Sigma and values were plotted as a function of the input VHH concentration.

FIG. 30A is a line graph of Stx-A4 VHH, Stx-A5 VHH and heterodimer StxA4-A5 VHH binding to Stx1 toxin as a function of input VHH concentration. VHH heterodimer Stx A4-A5 is displayed by dotted line.

FIG. 30B is a line graph of Stx-A4 VHH, Stx1-A9 VHH and heterodimer StxA4-A9 VHH binding to Stx1 toxin as a function of input VHH concentration. VHH heterodimer Stx A4-A9 is displayed by dotted line.

FIG. 30C is a line graph of Stx1-A9 VHH, Stx1-D4 VHH and heterodimer StxA9-D4 VHH binding to Stx1 toxin as a function of input VHH concentration. VHH heterodimer StxA9-D4 is displayed by dotted line.

FIG. 30D is a line graph of Stx1-A9 VHH, Stx-A5 VHH, Stx2-G1 VHH and heterotrimer StxA9-A5-G1 VHH binding to Stx1 toxin as a function of input VHH concentration. VHH heterotrimer StxA9-A5-G1 is displayed by dashed line.

FIG. 31A-FIG. 31D are a set of line graphs showing VHH binding to Stx1 toxin as a function of input VHH concentration. Dilution ELISAs were performed by coating plates with 0.5 μg/ml of 3D1 mAb Stx2. The plates were blocked and then incubated with 0.3 μg/ml of Stx1. For standard ELISAs, plates were coated with 1.5 μg of Stx2. VHH agents to be tested were serially diluted, incubated for 1 hour at room temperature, washed and the bound VHH agent were detected with HRP-anti-E-tag. The bound tagged agents were detected using the TMB kit by Sigma and values were plotted as a function of the input VHH concentration.

FIG. 31A is a line graph of Stx-A4 VHH, Stx-A5 VHH and heterodimer StxA4-A5 VHH binding to Stx2 toxin as a function of input VHH concentration. VHH heterodimer Stx A4-A5 is displayed by dotted line.

FIG. 31B is a line graph of Stx2-D10 VHH, Stx2-G1 VHH and heterodimer Stx G1-D10 VHH binding to Stx2 toxin as a function of input VHH concentration. VHH heterodimer Stx G1-D10 is displayed by dotted line.

FIG. 31 C is a line graph of Stx-A5 VHH, Stx2-D10 VHH, heterodimer Stx-A5-D10 VHH and heterotrimer Stx A9-A5-D10 VHH binding to Stx2 toxin as a function of input VHH concentration. VHH heterotrimer Stx A9-A5-D10 is displayed by dashed line.

FIG. 31 D is a line graph of Stx1-A9 VHH, Stx-A5 VHH, Stx2-G1 VHH and heterotrimer StxA9-A5-G1 VHH binding to Stx2 toxin as a function of input VHH concentration. VHH heterotrimer StxA9-A5-G1 is displayed by dashed line.

FIG. 32A-FIG. 32B are schematic drawings showing binding of multiple efAb molecules to Shiga toxin directed by a double-tagged VHH heterodimer targeting two epitopes (called a VNA), or to a single-tagged VHH-monomer which binds the pentameric B subunit.

FIG. 32A is a schematic drawing of a VHH-heterodimer VNA binding to a toxin, such as Shiga toxin (Stx), at two separate, non-overlapping epitopes. If the heterodimer contains two copies of an epitopic ‘tag’, then two molecules of the anti-tag efAb bind each bound heterodimer molecule leading to decoration of each toxin molecule by four efAb molecules.

FIG. 32B is a schematic drawing of a VHH-monomer binding to an epitope that is present at multiple sites on the toxin, such as the pentameric B-subunit of Stx, thereby binding at multiple sites on the toxin. If the VHH contains an epitopic tag, the efAb decorates each toxin molecule at five sites.

FIG. 33A-FIG. 33D are a set of line graphs of Stx1 toxin neutralization in a cell based assay as a function of VHH agent concentration. A Stx1 dose (about 15 pmoles) that induced about 100% Vero cell and killed them after 48 hours was selected. A VHH monomer, VHH monomer pool or VHH heterodimer, as labeled, were pre-mixed with Stx1 in culture medium and applied to Vero cells. Toxin neutralization was assessed after 48 hours by cell staining at A590 as described in examples herein. The extent of cell staining was plotted as a function of the VHH-agent concentration employed.

FIG. 33A is a line graph of Stx1 toxin neutralization in a cell based assay by Stx-A4 VHH, Stx-A5 VHH, a monomer pool of Stx-A4 and Stx-A5 VHHs and heterodimer Stx-A4-A5 VHH as a function of VHH concentration. VHH heterodimer Stx-A4-A5 is displayed by dotted line.

FIG. 33B is a line graph of Stx1 toxin neutralization in a cell based assay by Stx-A4 VHH, Stx1-A9 VHH, a monomer pool of Stx-A4 and Stx1-A9 VHHs and heterodimer Stx1-A4-A9 VHH as a function of VHH concentration. VHH heterodimer Stx-A4-A9 is displayed by dotted line.

FIG. 33C is a line graph of Stx1 toxin neutralization in a cell based assay by Stx1-A9 VHH, Stx-D4 VHH, a monomer pool of Stx1-A9 and Stx-D4 VHHs and heterodimer Stx1-A9-D4 VHH as a function of VHH concentration. VHH heterodimer Stx1-A9-D4 is displayed by dotted line.

FIG. 33D is a line graph of Stx1 toxin neutralization in a cell based assay by Stx-A5 VHH, Stx1-A9 VHH, a monomer pool of Stx-A5 and Stx1-A5 VHHs and heterotrimer Stx1-A9-A5-G1 VHH as a function of VHH concentration. VHH heterotrimer Stx1-A9-A5-G1 is displayed by dashed line.

FIG. 34A-FIG. 34D are a set of line graphs of Stx2 toxin neutralization in a cell based assay as a function of VHH agent concentration. A Stx2 dose (about 35 pmoles) that induced about 100% Vero cell and killed them after 48 hours was selected. A VHH monomer, VHH monomer pool or VHH heterodimer, as labeled, were pre-mixed with Stx2 in culture medium and applied to Vero cells. Toxin neutralization was assessed after 48 hours by cell staining at A590 as described in examples herein. The extent of cell staining was plotted as a function of the VHH-agent concentration employed.

FIG. 34A is a line graph of Stx2 toxin neutralization in a cell based assay by Stx-A4 VHH, Stx-A5 VHH, a monomer pool of Stx-A4 and Stx-A5 VHHs and heterodimer Stx-A4-A5 VHH as a function of VHH concentration. VHH heterodimer Stx-A4-A5 is displayed by dotted line.

FIG. 34B is a line graph of Stx2 toxin neutralization in a cell based assay by Stx2-D10 VHH, Stx2-G1 VHH, a monomer pool of Stx2-D10 and Stx2-G1 VHHs and heterodimer Stx2-G1-D10 VHH as a function of VHH concentration. VHH heterodimer Stx2-G1-D10 is displayed by dotted line.

FIG. 34C is a line graph of Stx2 toxin neutralization in a cell based assay by Stx-A5 VHH, Stx2-D10 VHH, a monomer pool of Stx-A5 and Stx2-D10 VHHs, heterodimer Stx-A5-D10 VHH and heterotrimer Stx-A9-A5-D10 as a function of VHH concentration. VHH heterodimer Stx1-A5-D10 is displayed by dotted line and VHH heterotrimer Stx-A9-A5-D10 is displayed by dashed line.

FIG. 34D is a line graph of Stx2 toxin neutralization in a cell based assay by Stx-A5 VHH, Stx2-G1 VHH, a monomer pool of Stx-A5 and Stx2-G1 VHHs and heterotrimer Stx-A9-A5-G1 VHH as a function of VHH concentration. VHH heterotrimer Stx-A9-A5-G1 is displayed by dashed line.

FIG. 35A-FIG. 35D are a set of Meyer-Kaplan survival plots for percent survival of subjects as a function of time in days following contact with Stx1 toxin and later time administered VHH binding/neutralizing agents. Subjects, groups of five mice were injected with 20 pmoles of Stx1 premixed with 40 pmoles of the labeled VHH-based antitoxin agent (or 640 pmoles of VHH-A9 where indicated) and monitored for illness and death for one week. The percent survival is plotted as a function of time. In some subjects, an 80 pmole dose of efAb was included in the treatment.

FIG. 35A is a Meyer-Kaplan survival plot for percent survival of subjects exposed to Stx1 toxin and then administered either Stx-A9 VHH, Stx-A9 (640 pm) VHH or no VHH agent.

FIG. is a Meyer-Kaplan survival plot for percent survival of subjects exposed to Stx1 toxin and then administered either Stx-A9 VHH, Stx-A4 VHH Stx-A9-A4 heterodimer VHH or no VHH agent.

FIG. 35C is a Meyer-Kaplan survival plot for percent survival of subjects exposed to Stx1 toxin and then administered either heterotrimer Stx-A9-A5-D10 VHH without efAb, heterotrimer Stx-A9-A5-D10 VHH with efAb or no VHH agent.

FIG. 35D is a Meyer-Kaplan survival plot for percent survival of subjects exposed to Stx1 toxin and then administered either heterotrimer Stx-A9-A5-G1 VHH with efAb or no VHH agent.

FIG. 36A-FIG. 36D are a set of Meyer-Kaplan survival plots for percent survival of subjects as a function of time in days following contact with Stx2 toxin and later time administered VHH binding/neutralizing agents. Subjects, groups of five mice were injected with 1 pmoles of Stx2 premixed with 40 pmoles of the labeled VHH-based antitoxin agent and monitored for illness and death for one week. The percent survival is plotted as a function of time. In some subjects, an 80 pmole dose of efAb was included in the treatment.

FIG. 36A is a Meyer-Kaplan survival plot for percent survival of subjects exposed to Stx2 toxin and then administered either Stx-A5 VHH, Stx-D10 VHH, Stx-D10 with efAb, heterodimer Stx-A5-D10 VHH, or no VHH agent.

FIG. is a Meyer-Kaplan survival plot for percent survival of subjects exposed to Stx2 toxin and then administered either a mixture of Stx-A5 VHH and Stx-D10. Stx-A5-D10 heterodimer VHH or no VHH agent.

FIG. 36C is a Meyer-Kaplan survival plot for percent survival of subjects exposed to Stx2 toxin and then administered either heterotrimer Stx-A9-A5-D10 VHH without efAb, heterotrimer Stx-A9-A5-D10 VHH with efAb or no VHH agent.

FIG. 36D is a Meyer-Kaplan survival plot for percent survival of subjects exposed to Stx2 toxin and then administered either heterotrimer Stx-A9-A5-G1 VHH with efAb, heterotrimer Stx-A9-A5-G1 VHH without efAb or no VHH agent.

FIG. 37A-FIG. 37D are a set of mircographs and a bar graph showing that VNA plus efAb protect subjects from Stx2 induced renal damage. Formalin-fixed, paraffin embedded and hematoxylin and eosin stained 3 μm sections were examined by light microscopy from untreated age- and sex-matched controls (FIG. 37A), mice receiving the A9/A5/G1 VNA+efAb (FIG. 37B), and mice receiving only A9/A5/G1 VNA (FIG. 36C). The numbers of tubules with lesions such as epithelial apoptosis/necrosis, attenuation and restitution, hypertrophy, hyperplasia, luminal dilation, tubular atrophy/collapse, interstitial cell proliferation and early interstitial fibrosis were quantified in 6 random 20× fields per mouse totaling 114 measurements and plotted in a bar graph (FIG. 37D). Examples of lesions are highlighted by black oval in FIG. 37B and the asterisks in FIG. 37C. (N.D.=None Detected)

FIG. 38 is a listing of amino acid sequences of VHHs selected for binding to Stx1 or Stx2. Sequences shown begin within framework 1 At the site of the primer binding employed in coding sequence DNA amplification from the immune alpaca cDNA and continue through the end of framework 4. The parentheses at the end indicate whether the VHH contains a long hinge (lh) or a short hinge (sh). The three-complementarity determining regions (CDRs) are indicated at the top.

FIG. 39 is a dendrogram of VHHs selected for binding to Stx1 or Stx2. The VHH sequences shown in FIG. 38 were analyzed for homology to create a dendrogram. Longer branch lengths indicate less sequence homology. The central node labeled as the ‘cross-specific homology group’ indicate VHHs that recognize both Stx1 And Stx2 and possess significant homology in CDR3 (see FIG. 38).

FIG. 40 is a photograph of Western blot for VHH binding to Stx1 And Stx2. Purified Stx1 and Stx2 were resolved by SDS-PAGE and the gel stained for protein (stain). Molecular weight markers are shown to the left. Similar lanes containing Stx1 And Stx2 were transferred to filters for Western blot. The blots were incubated with 10 μg/ml of the indicated VHHs or control. Bound VHH was visualized with HRP/anti-E-tag.

### DETAILED DESCRIPTION

The presence of toxins in the circulation is the cause of a wide variety of human and animal illnesses. Antitoxins are therapeutic agents that prevent toxin infection or reduce further development of negative symptoms in patients that have been exposed to a toxin (a process referred to as “intoxication”). Typically, antitoxins are antisera obtained from large animals (e.g., sheep, horse, and pig) that were immunized with inactivated or non-functional toxin. More recently, antitoxin therapies have been developed using combinations of antitoxin monoclonal antibodies including yeast-displayed single-chain variable fragment antibodies generated from vaccinated humans or mice. See Nowakowski et al. 2002. Proc Natl Acad Sci USA 99: 11346-11350; Mukherjee et al. 2002. Infect Immun 70: 612-619; Mohamed et al. 2005 Infect Immun 73: 795-802; Walker, K. 2010 Interscience Conference on Antimicrobial Agents and Chemotherapy—50th Annual Meeting—Research on Promising New Agents: Part 1. IDrugs 13: 743-745. Antisera and monoclonal antibodies can be difficult to produce economically at scale, usually requiring long development times and resulting in problematic quality control, shelf-life and safety issues. New therapeutic strategies to develop and prepare antitoxins are needed.

Antitoxins function through two key mechanisms neutralization of toxin function and clearance of the toxin from the body. Toxin neutralization occurs through biochemical processes including inhibition of enzymatic activity and prevention of binding to cellular receptors. Antibody mediated serum clearance occurs subsequent to the binding of multiple antibodies to the target antigen (Dacron M. 1997 Annu Rev Immunol 15: 203-234; Davies et al. 2002 Arthritis Rheum 46: 1028-1038; Johansson et al. 1996 Hepatology 24: 169-175; and Lovdal et al. 2000 J Cell Sci 113 (Pt 18): 3255-3266). Multimeric antibody decoration of the target is necessary to permit binding to low affinity Fc receptors (Davies et al. 2002 Arthritis Rheum 46: 1028-1038 and Lovdal et al. 2000 J Cell Sci 113 (Pt 18): 3255-3266). Without being limited by any particular theory or mechanism of action, it is here envisioned that an ideal antitoxin therapeutic would both promote toxin neutralization to immediately block further toxin activity and also accelerate toxin clearance to eliminate future pathology if neutralization becomes reversed.

Effective clearance of botulinum neurotoxin (BoNT), a National Institute of Allergy and Infectious Diseases (NIAID) Category A priority pathogen, is believed by some researchers to require three or more antibodies bound to the toxin. Nowakowski et al. 2002. (Proc Natl Acad Sci USA 99: 11346-11350) determined that effective protection of mice against high dose challenge of BoNT serotype A (BoNT/A) required co-administration of three antitoxin monoclonal antibodies, and that all three antibodies presumably promoted clearance. Data have shown that administration of a pool of three or more small binding agents, each produced with a common epitopic tag, reduced serum levels of a toxin when co-administered with an anti-tag monoclonal antibody (Shoemaker et al. US. published application 2010/0278830 A1 published Nov. 4, 2010 and Sepulveda et al. 2009 Infect Immun 78: 756-763, each of which is incorporated herein in its entirety). The tagged binding agents directed the binding of anti-tag monoclonal antibody to multiple sites on the toxin, thus indirectly decorating the toxin with antibody Fc domains and leading to its clearance through the liver.

Pools of scFv domain binding agents with specificity for BoNT/A and each containing a common epitopic tag (E-tag), had been shown to be effective for decorating the botulinum toxin with multiple anti-tag antibodies (Shoemaker et al. US. utility patent publication number 2010/0278830 published Nov. 4, 2010 and US. continuation-in-part patent publication number 2011/0129474 published Jun. 2, 2011, each of which is incorporated herein by reference in its entirety). Data showed that the administration of binding agents and clearance antibodies to subjects resulted in clearance via the liver with an efficacy in mouse assays equivalent to conventional polyclonal antitoxin sera. Ibid. and Sepulveda et al. 2009 Infect Immun 78: 756-763. The tagged scFvs toxin targeting agents and the anti-tag monoclonal antibodies were effective for treating subjects at risk for or having been contacted with a disease agent.

The use of small binding agents to direct the decoration of toxin with antibody permits new strategies for the development of agents with improved therapeutic and commercial properties. Examples herein show that a single recombinant heterodimeric binding protein/agent including two or more high-affinity BoNT binding agents (camelid heavy-chain-only Ab VH (VHH) domains) and two epitopic tags, co-administered with an anti-tag mAb, protected subjects from botulism caused negative symptoms and lethality. Further the binding protein resulted in antitoxin efficacy equivalent to and greater than conventional BoNT antitoxin serum in two different in vivo assays. Examples herein compare neutralizing or non-neutralizing binding agents administered with or without clearing antibody, and show the relative contributions of toxin neutralization and toxin clearance to antitoxin efficacy. Examples herein show that both toxin neutralization and toxin clearance contribute significantly to antitoxin efficacy in subjects. Toxin neutralization or toxin clearance using heterodimer binding protein antitoxins sufficiently protected subjects from BoNT lethality in a therapeutically relevant, post-intoxication assay. Methods in Examples herein optionally further include a clearing antibody for example a monoclonal anti-E-tag antibody.

It was observed in Examples herein that VHH binding agents that neutralized toxin function significantly improved the antitoxin efficacy and even obviated the need for clearing antibody in a clinically relevant post-intoxication BoNT/A assay. The methods, compositions and kits using the multimeric binding proteins described herein have widespread application in antitoxin development and other therapies in which neutralization and/or accelerated clearance of a target molecule benefits a patient. For example, the target molecule is an exogenous disease agent that infects or is at risk to infect a patient. Exogenous disease agent for example is a virus, a cancer cell, a fungus, a bacterium, a parasite and a product thereof such as a pathogenic molecule, a protein, a lipopolysaccharide, or a toxin. Alternatively, the molecule is an endogenous (body produced) molecule that is produced in the patient and that causes or produces harmful effects on the patient. For example, the molecule is a hormone or a protein that is associated with a disease or condition, e.g., inflammation, cancer, transplant rejection, kidney failure, or a defect in blood clotting such as hemophilia and thrombophilia. In various embodiments, the disease agent is a toxin of C. difficile.

C. difficile is a gram-positive, spore forming, anaerobic bacterium that is the leading cause of antibiotic-associated diarrhea, the severity of which ranges from mild diarrhea to life threatening pseudomembranous colitis (Bartlett J G. 2002 N Engl J Med 346:334-9 and Feng et al. PCT/US10/58701 filed Dec. 2, 2010, each of which is incorporated by reference in its entirety). Pathogenic C. difficile strains excrete exotoxins A (TcdA) and B (TcdB) that have been intimately linked to its pathogenicity. Both TcdA and TcdB are enterotoxic, capable of inducing intestinal epithelial damage and increasing mucosal permeability, and hence are thought to be responsible for the pathogenesis of C. difficile-associated colitis (Kelly C P et al. 1998 Annu Rev Med 49:375-90). C. difficile has emerged as a leading cause of hospital-acquired enteric infections with rapidly escalating annual health care costs in the United States (Kyne L et al. 2002 Clin Infect Dis 34:346-353). The severity of C. difficile-associated infections ranges from mild diarrhea to life threatening pseudomembranous colitis (Bartlett J G et al. 2002 N Engl J Med 346:334-339; Borriello S P 1998 Antimicrob Chemother 41 Suppl C:13-19). Several hospital outbreaks of C. difficile-associated diarrhea (CDAD), with high morbidity and mortality in the past few years in North America, have been attributed to the widespread use of broad-spectrum antibiotics.

The emergence of more virulent C. difficile strains contributes also to the increased incidence and severity of the disease (Loo V G et al. 2005 N Engl J Med 353:2442-2449; McDonald L C et al. 2005 N Engl J Med 353:2433-2441). Antibiotic usage results in a reduction of commensal microflora in the gut, which permits C. difficile to proliferate more extensively, leading to the further production of toxins (Owens J R et al. 2008 Clinical Infectious Diseases 46(s1):S19-S31). C. difficile infection (CDI) includes a range of symptoms varying from mild diarrhea to severe fulminate lethal disease (Kuijper E J et al. 2007 Curr Opin Infect Dis 20(4):376-383). Recent outbreaks of highly virulent C. difficile strains (McDonald L C et al. 2005 N Engl J Med 353(23):2433-2441; Loo V G et al. 2005 N Engl J Med 353(23):2442-2449) have increased the urgency to devote greater resources towards the understanding of the molecular, genetic, and biochemical basis for the pathogenesis, with a view to use such information to develop novel preventive and treatment modalities.

A cell-based immunocytotoxicity assay for detecting C. difficile toxins described in Feng et al. (PCT/US2009/003055 published Nov. 19, 2009 as WO 2009/139919) uses an anti-C. difficile toxin A (TcdA) monoclonal antibody, named A1113, which substantially enhanced the activity of TcdA on Fc gamma receptor I (FcγRI)-expressing cells (He X, Sun X, Wang J, et al. Antibody-enhanced, Fc{gamma}R-mediated endocytosis of C. difficile toxin A. Infect Immun 2009). Feng et al. shows use of A1H3 enhancing antibody, in combination with an electronic sensing system to develop a real-time and ultrasensitive assay for the detection of biological activity of C. difficile toxins.

Toxin A (TcdA) and toxin B (TcdB) are the major virulence factors contributing to pathogenic C. difficile strains. These strains are enterotoxic, inducing intestinal epithelial cell damage, disrupting epithelium tight junctions leading to increased mucosal permeability (Pothoulakis C et al. 2001 Am J Physiol Gastrointest Liver Physiol 280:G178-183; Riegler M et al. 1995 J Clin Invest 95:2004-2011; Savidge T C et al. 2003 Gastroenterology 125:413-420). Moreover, these toxins induce production of immune mediators, leading to subsequent neutrophil infiltration and severe colitis (Kelly C P et al. 1994 J Clin Invest 93:1257-1265; Kelly C P et al. 1998 Annu Rev Med 49:375-390). TcdA and TcdB are structurally homologous, and contain a putative N-terminal glucosyltransferase and a cysteine proteinase domain, a transmembrane domain, and a C-terminal receptor binding domain (von Eichel-Streiber C et all 996 Trends Microbiol 4:375-382) (Jank T et al. 2008 Trends in microbiology 16:222-229; Voth D E et al. 2005 Clin Microbiol Rev 18:247-263).

Interaction between the toxin C-terminus and the host cell receptors initiates a receptor-mediated endocytosis (Florin I et al. 1983 Biochim Biophys Acta 763:383-392; Karlsson K A 1995 Curr Opin Struct Biol 5:622-635; Tucker K D et al. 1991 Infect Immun 59:73-78). Although the intracellular mode of action remains unclear, it has been proposed that the toxins undergo conformational change at low pH in the endosomal compartment, leading to membrane insertion and channel formation (Florin I et al. 1986 Microb Pathog 1:373-385; Giesemann. T et al. 2006 J Biol Chem 281:10808-10815; Henriques B et. al.. 1987 Microb Pathog 2:455-463; Qa'Dan M et al. 2000 Infect Immun 68:2470-2474). A host cofactor is then required to trigger a second structural change which is accompanied by an immediate autocatalytic cleavage and release of the glucosyltransferase domain into cytosol (Pfeifer G et al. 2003 J Biol Chem 278:44535-44541; Reineke J e al. 2007 Nature 446:415-419; Rupnik M et al. 2005 Microbiology 151:199-208). Once the glucosyltransferase domain reaches the cytosol, it inactivates proteins of the Rho/Rac family, leading to alterations of cytoskeleton and ultimately cell death (Just I et al. 1995 Nature 375:500-503; Sehr P et al. 1998 Biochemistry 37:5296-5304).

The clinical manifestation of CDI is highly variable, from asymptomatic carriage, to mild self-limiting diarrhea, to the more severe pseudomembranous colitis. The prevalence of systemic complication and death in CDI has become increasingly common (Siemann M et al. 2000 Intensive care medicine 26:416-421). In life-threatening cases of CDI, systemic complications are observed, including cardiopulmonary arrest (Johnson S et al. 2001 Annals of internal medicine 135:434-438), acute respiratory distress syndrome (Jacob S S et al. 2004 Heart Lung 33:265-268), multiple organ failure (Dobson G et al. 2003 Intensive care medicine 29:1030), renal failure (Cunney R J et al. 1998 Nephrol Dial Transplant 13:2842-2846), and liver damage (Sakurai T et al. 2001 J Infect Dis 33:69-70). The exact reason for these negative complications is unclear, and may be caused by entry of the toxin into the circulation and systemic dissemination (Hamm E E et al. 2006 Proc Natl Acad Sci USA 103:14176-14181).

Standard therapy depends on treatment with vancomycin or metronidazole, neither of which is fully effective (Zar et al. 2007 Clinical Infectious Diseases 45:302-307). Moreover, an estimated 15% to 35% of those infected with C. difficile relapse following treatment (Barbut et al. 2000 J Clin Microbiol 38: 2386-2388; Tonna et al: Postgrad Med J 81: 367). Unfortunately, the primary treatment option for recurrent CDI is still metronidazole or vancomycin. Other options, such as probiotics, toxin-absorbing polymer and anion-exchange resins, have limited efficacy (Gerding, D. N., Muto, C. A. & Owens, R. C., Jr. 2008 Clin Infect Dis 46 Suppl 1: S32-42). Therefore, immune-based therapies are the probably the most promising approaches to control the disease. Antibodies specific for both of these toxins, and not against TcdA or TcdB alone, protect against toxigenic C. difficile infection in a hamster model (Libby et al, 1982 Infect Immun 36: 822-829; Fernie et al, 1983 Dev Biol Stand 53: 325; and Kim et al, 2006 Infection and immunity 74: 6339). Human serum antibodies specific for both TcdA and TcdB are associated also with protection against symptomatic disease and recurrence. Recent phase II clinical trial led by Merck demonstrated that the systemically administered human IgG monoclonal antibodies against TcdA and TcdB prevents disease relapse in CDI patients (Lowy et al, 2010 The New England journal of medicine 362: 197). However, the treatment involved the injection of a large quantity of two individual antibodies against each toxin.

Examples herein show a new approach to the development of antitoxins that employs a single recombinant protein to promote toxin decoration with multiple copies of a single monoclonal antibody leading to its neutralization and clearance from the body. The methods, compositions, and kits herein are useful for treating a great number of the most common pathogenic biological targets by accelerating neutralization and clearance from the subject or patient.

Examples herein show that camelid VHH binding domains, which have multiple commercial advantages over scFvs due in part to the case and reduced cost of producing VHHs, were effective as toxin targeting agents both with and without being administered with clearing antibody. An important advantage of VHHs is the ability of medical professionals and scientists to express these binding agents as heterodimers in which each component VHH remains fully functional. The multimeric fusion proteins containing at least two VHH binding regions resulted in the component VHHs binding to different epitopes on the same toxin target. Without being limited by any particular theory or mechanism of action, it is believed that incorporation of two epitope tags on the heterodimers resulted in decoration of the toxin with two clearing antibodies at each epitope, and resulted in a total of four monoclonal clearing antibodies binding to the heterodimers on the toxin. In addition, with certain heterodimers the decoration promoted efficient toxin clearance. Either neutralization or clearance or both are important mechanisms of remediating toxin exposure. As each double-tagged heterodimeric binding agent was bound only to only two monoclonal antibodies, the heterodimeric agent itself may not be effectively cleared by low affinity Fc receptors unless actually bound to the toxin.

The ability of antitoxin antibodies to protect mammalian subjects from the symptoms of toxin exposure is influenced by several factors that are described herein. Examples herein used intoxication models and varied the dose of antitoxin agent and the timing of antitoxin administration relative to exposure to toxin in order to determine whether both the dose and the timing of the antitoxin are factors that influence antitoxin efficacy. In addition, examples herein analyzed the role that affinity of the antibody for the toxin has on the ability of the antibody to bind (Kₒₙ) and remain bound (K_{off}) to the toxin and exert its effect. Data show that the ability of the antibody monomer/heterodimer to inhibit the enzymatic activity of the toxin and/or prevent its entry into target cells (i.e. neutralization) is a major factor in effective antitoxin treatment of subjects. Specifically data show that the greater the binding affinity of the binding protein to the target molecule, the greater the potential neutralization and clearance of the binding protein. Examples herein show also that the multimeric binding proteins promoted the clearance of the toxin from the serum and minimized further negative symptoms or lethality by the target molecule or disease agent. A portion of this work was published Jan. 6, 2012 in the Public Library of Science One and was entitled, “A Novel Strategy for Development of Recombinant Antitoxin Therapeutics Tested in a Mouse Botulism”, authored by Jean Mukherjee, Jacqueline M. Tremblay, Clinton E. Leysath, Kwasi Ofori, Karen Baldwin, Xiaochuan Feng, Daniela Bedenice, Robert P. Webb, Patrick M. Wright. Leonard A. Smith, Saul Tzipori, and Charles B. Shoemaker (Mukherjee J. et al. 2012 PLoS One. 7(1):e29941), which is incorporated by reference herein in its entirety.

Methods for engineering and selecting proteins for binding to disease agents are shown for example in U.S. utility application Ser. No. 13/566,524 filed Aug. 3, 2012; US. publication number 2011/0129474 published Jun. 2, 2011 (U.S. application Ser. No. 12/889,511 filed Sep. 24, 2010), which is a continuation-in-part application of US. publication number 2010/0278830 published Nov. 4, 2010 (U.S. utility application Ser. No. 12/032,744 filed Feb. 18, 2008), each of which is incorporated by reference herein in its entirety.

An aspect of the invention provides a method for treating a subject at risk for exposure to or exposed to a disease agent, the method including: contacting the subject with at least one recombinant heteromultimeric neutralizing binding protein including two or multiple binding regions, such that the binding regions are not identical, and each binding region specifically binds a non-overlapping portion of the disease agent, such that the binding protein neutralizes the disease agent, thereby treating the subject for exposure to the disease agent.

In various embodiments of the method, the binding protein includes at least one tag. For example the tag is a molecule or epitope that is attached or genetically fused to the binding protein and/or binding regions. The tag in various embodiments of the method induces endogeneous clearance of the disease agent from the body in vivo. For example the tag includes SEQ ID NO: 15. In a related embodiment, the tag includes an antibody epitope.

In certain embodiments of the method, the binding protein is selected from: a single-chain antibody (scFv); a recombinant camelid heavy-chain-only antibody (VHH); a shark heavy-chain-only antibody (VNAR); a microprotein; a darpin; an anticalin; an adnectin; an aptamer; a Fv; a Fab; a Fab′; and a F(ab′)₂. In an embodiment, the binding protein is heterodimeric, for example the binding protein has greater potency than each individual monomer. In alternative embodiments, the heteromultimeric neutralizing binding protein is multimeric and the multimeric components are associated non-covalently or covalently.

The binding protein in certain embodiments of the method includes a linker that separates multimeric components of the binding regions. In various embodiments, the linker includes at least one selected from: a peptide, a protein, a sugar, or a nucleotide. For example, the linker includes amino acid sequence GGGGS (SEQ ID NO: 54), or includes amino acid sequence GGGGSGGGGSGGGGS (SEQ ID NO: 55) or a portion thereof. In a related embodiment, the linker is a flexible linker located within subunits/domains of the binding protein, such that the linker does not negatively affect the function of the binding protein to the disease agent. For example the linker includes amino acid sequences/residues including serine and glycine, and in various embodiments is at least about three to five amino acids long, or about five to eight amino acids long, or about eight to fifteen amino acids long.

In certain embodiments, the disease agent is a biological target or biological molecule. For example, the biological target or the biological molecule is naturally occurring within the subject, for example a molecule or compound synthesized by the subject. An example of a biological molecule synthesized by the subject is an IgE that is associated with an allergy or an auto antibody or an MHC protein (e.g., HLA class I antigens A and B and HLA class II antigen DR) associated with an autoimmune disease. For example the autoimmune disease is selected from: lupus erythematosus, Graves' disease, rheumatoid arthritis, Sjögren's syndrome, myasthenia gravis, and Hashimoto's thyroiditis.

The disease agent in various embodiments of the method includes a plurality of non-identical disease agents, for example two or more bacterial toxins, or a viral toxin and a fungal species. In various embodiments, the binding regions of the binding protein are specific to each non-identical disease agent and bind to and neutralize the plurality of disease agents.

In various embodiments of the method, the disease agent is at least one selected from: a virus, a cancer cell, a fungus, a bacterium, a parasite and a product thereof such as a pathogenic molecule, a protein, a lipopolysaccharide, and a toxin. In certain embodiments, the toxin includes a protein, a lipid, a lipopolysaccharide, and a small molecule toxin such as an aflatoxin or a dinoflagellate toxin. The toxin for example is a Botulinum neurotoxin comprising a serotype selected from: A, B, C, D, E, F, and G. In certain embodiments of the method, the toxin is a Clostridium exotoxin comprising toxin A (TcdA) and toxin B (TcdB).

In various embodiments of the method, the toxin is at least one selected from: staphylococcal α-hemolysin, staphylococcal leukocidin, aerolysin cytotoxic enterotoxin, a cholera toxin, Bacillus cereus hemolysis II toxin, a Helicobacter pylori vacuolating toxin, a Bacillus anthracis toxin, a cholera toxin, a Escherichia coli serotype O157:H7 toxin, a Escherichia coli serotype O104:H7 toxin, a lipopolysaccharide endotoxin, a Shiga toxin, a pertussis toxin, a Clostridium perfringens iota toxin, a Clostridium spiroforme toxin, a Clostridium difficile toxin A, a Clostridium difficile toxin B, a Clostridium septicum a toxin, and a Clostridium botulinum C2 toxin. In a related embodiment of the method, the disease agent is an infectious strain, for example a bacterial strain or a viral strain. In a related embodiment, the disease agent is a Gram-negative strain or a Gram positive strain.

The bacterium in various embodiments of the method is selected from the group consisting of: B. anthracis, B. cereus, C. botulinum, C. difficile, C. perfringens, C. spiroforme, and V cholerae.

In certain embodiments, the binding regions bind to different disease agents, such that the binding protein is specific for a plurality of disease agents, e.g., a Clostridium toxin and an Escherichia toxin. For example, the binding protein includes a chimeric fusion protein specific to at least two different disease agents described herein. In certain embodiments of the method, the binding protein is a humanized antibody derived from a non-human species for example a mouse, a rabbit, an alpaca, a llama, or horse.

In a related embodiment, the method further includes observing neutralizing of the disease agent by the binding protein and/or survival of the subject. In certain embodiments of the method, observing further includes measuring an amount of the disease agent or a disease agent product in a sample from the subject. In various embodiments, the sample is selected from: a cell, a fluid, and a tissue. For example, the fluid is at least one selected from: blood, serum, plasma, mucosal fluid, saliva, cerebrospinal fluid, semen, tears, and urine. In certain embodiments of the method, the cell or the tissue is at least one selected from: fecal; vascular; epithelial; endothelial; dermal; dental; connective; muscular; neuronal; facial; cranial; soft tissue including cartilage and collagen; brain; bone; bone marrow; joint tissue; and articular joints. For example, the method includes collecting the fluid, the cell, or the tissue from a biopsy. In certain embodiments, the method includes collecting the fluid, the cell, or the tissue from an ex vivo sample or aliquot. Alternatively, the method includes collecting from fluid, cell, or tissue that is in vivo or in situ.

The method further includes in a related embodiment observing a reduction or a remediation in at least one pathology symptom associated with the disease agent. In various embodiments, the method further includes prior to contacting the subject with the binding protein, observing and/or detecting in the subject an indicium of the exposure to the disease agent selected from: diarrhea, vomiting, breathing difficulty, fever, inflammation, bleeding, pain, numbness, loss of consciousness, tissue necrosis, or organ failure. For example, the subject is a transplant recipient or an immunosuppressed patient.

In a related embodiment, the method further includes contacting the subject with the binding protein at a period of time such as seconds, minutes, or hours after observing the indicium. Alternatively, the method further includes contacting the subject with the binding protein seconds, minutes, hours, or days prior to an event that is associated with the risk for the exposure. For example, the method includes contacting the subject prior to or after the subject's entering a potentially hazardous or dangerous environment such as biohazard facility, a combat zone, or a hazardous waste site.

The method in related embodiments includes contacting the subject with the binding protein by injecting a solution including the binding protein into the subject. In various embodiments, injecting involves at least one selected from: subcutaneous, intravenous, intramuscular, intraperitoneal, intradermal, intramedullary, transcutaneous, and intravitreal. In various embodiments of the method, contacting the subject with binding protein includes at least one technique selected from: topically, ocularly, nasally, bucally, orally, rectally, parenterally, intracisternally, intravaginally, or intraperitoneally. In a related embodiment, contacting the subject involves using an applicator, for example the applicator is a syringe, a needle, a sprayer, a sponge, a gel, a strip, a tape, a bandage, a tray, a string, or a device used to apply a solution to a cell or a tissue.

In a related embodiment of the method, contacting the subject with the binding protein includes administering to the subject a source of expression of the binding protein. In various embodiments of the method, the source of expression of the binding protein is a nucleotide sequence encoding the binding protein, such that the source of the expression includes at least one selected from the group consisting of: a naked nucleic acid vector, bacterial vector, and a viral vector. For example, the bacterial vector is derived from at least one selected from the group consisting of: E. coli, Bacillus spp, Clostridium spp, Lactobacillus spp, and Lactococcus spp.

In a related embodiment of the method, contacting further includes administering the vector, for example the naked nucleic acid vector, the bacterial vector, or the viral vector.

In a related embodiment, the nucleotide acid sequence further includes an operably linked signal for promoting expression of the binding protein. For example, the signal includes a mammalian promoter or a non-viral promoter. In a related embodiment, the method involves engineering the binding protein or the source of expression of the binding protein (e.g., viral vector or bacterial vector) using a dimerizer sequence for example having an amino acid sequence including SEQ ID NO: 94 or a portion or homolog thereof. For example, the dimerizer sequences forms a covalent bond or disulfide linkage between at least two amino acid sequences to form a homodimer, a heterodimer, or a multimer. The method in various embodiments includes, prior to contacting, engineering the binding protein using an agent that multimerizes at least one binding region or a multimer, e.g., a heterodimer, a heterotrimer, and a heterotetramer, to form the binding protein.

In a related embodiment of the method, the viral vector is derived from at least one selected from: an adenovirus, an adeno-associated virus, a herpesvirus, and a lentivirus. The method in various embodiments further includes contacting the subject with a gene delivery vehicle selected from at least one of: a liposome, a lipid/polycation (LPD), a peptide, a nanoparticle, a gold particle, and a polymer. For example, the gene delivery vehicle specifically targets a cell or tissue in the body by contacting or binding a receptor located on the cell or tissue.

An aspect of the invention provides a pharmaceutical composition for treating a subject at risk for exposure to or exposed to a disease agent, the pharmaceutical composition including: at least one recombinant heteromultimeric neutralizing binding protein including two or more binding regions, such that the binding regions are not identical, and each binding region specifically binds a non-overlapping portion of the disease agent, such that the binding protein neutralizes the disease agent, thereby treating the subject for exposure to the disease agent.

In a related embodiment, the composition is compounded with a pharmaceutically acceptable buffer or diluent. For example the composition is compounded for parenteral administration such as intravenous, mucosal administration, topical administration, or oral administration.

In various embodiments, the subject is at least one selected from: a human, a dog, a cat, a goat, a cow, a pig, and a horse. For example, the human subject is a: sick child or adult, health-care profession (e.g., doctor and nurse), aid worker, member of the military, or an immunosuppressed patient such as a transplant recipient. In certain embodiments, the pharmaceutical composition is formulated to protect the subject against the exposure, for example that exposure includes a picogram amount, nanogram amount, microgram amount, or gram amount of the disease agent or a plurality of disease agents.

The binding protein or binding regions in various embodiments of the composition is selected from the group of: a single-chain antibody (scFv); a recombinant camelid heavy-chain-only antibody (VHH); a shark heavy-chain-only antibody (VNAR); a microprotein; a darpin; an anticalin; an adnectin; an aptamer; a Fv; a Fab; a Fab; and a F(ab′)₂. In various embodiments, the binding regions are of a different type, for example at least one binding region is a VHH and at least other binding region is a scFv, an Fab or any of the types described herein.

The composition in various embodiments further includes at least one agent selected from the group of: an antitoxin, an anti-inflammatory, an anti-tumor, an antiviral, an antibacterial, an anti-mycobacterial, an anti-fungal, an anti-proliferative, an anti-apoptotic, an anti-allergy, and an anti-immune suppressant.

In an embodiment, the composition further includes a labeled detectable marker selected from the group consisting of: detectable, fluorescent, colorimetric, enzymatic, radioactive, and the like. For example, the marker is detectable in a sample taken from the subject, the sample exemplified by a cell, a fluid or a tissue. In a related embodiment, the marker includes a peptide, a protein, a carbohydrate, and a polymer.

In an embodiment of the composition, the binding protein includes a linker that separates the binding regions. The linker in a related embodiment separates the binding regions and/or subunits of the multimeric protein. In certain embodiments, the binding protein includes a linker that covalently joins each binding region of the heterodimeric or the multimeric protein. In various embodiments, the linker includes at least one selected from the group of: a peptide, a protein, a sugar, or a nucleic acid. In a related embodiment, the linker includes amino acid sequence GGGGS (SEQ ID NO: 54) or a portion thereof. In a related embodiment, the linker includes amino acid sequence GGGGSGGGGSGGGGS (SEQ ID NO: 55) or a portion thereof or multiples thereof. The linker in various embodiments stabilizes the binding protein and does not prevent the respective binding of the binding regions to the disease agent or to a plurality of disease agents.

In various embodiments of the pharmaceutical composition, the binding protein and/or binding regions include at least one tag that is attached or genetically fused to the binding protein and/or binding regions. The tag for example is a peptide, sugar, or DNA molecule that does not inhibit or prevent binding of the binding protein and/or binding regions to the disease agent. In various embodiments, the tag is at least about: three to five amino acids long, five to eight amino acids long, eight to twelve amino acids long, twelve to fifteen amino acids long, or fifteen to twenty amino acids long. For example, the tag includes SEQ ID NO: 15.

In various embodiments, the disease agent for which the binding protein is specific is at least one selected from: a virus, a cancer cell, a fungus, a bacterium, a parasite and a product thereof such as a pathogenic molecule, a protein, a lipopolysaccharide, or a toxin. In related embodiments of the composition, the toxin includes a protein, a lipid, a lipopolysaccharide, and a small molecule toxin such as an aflatoxin or a dinoflagellate toxin. For example, the toxin is a Botulinum neurotoxin comprising a serotype selected from: A, B, C, D, E, F, and G. In various embodiments of the composition, the toxin is at least one selected from: staphylococcal α-hemolysin, staphylococcal leukocidin, aerolysin cytotoxic enterotoxin, a cholera toxin, Bacillus cereus hemolysis II toxin, a Helicobacter pylori vacuolating toxin, a Bacillus anthracis toxin, a cholera toxin, a Escherichia coli serotype O157:117 toxin, a Escherichia coli serotype O104:H7 toxin, a lipopolysaccharide endotoxin, a Shiga toxin, a pertussis toxin, a Clostridium perfringens iota toxin, a Clostridium spiroforme toxin, a Clostridium difficile toxin A, a Clostridium difficile toxin B, a Clostridium septicum a toxin, and a Clostridium botulinum C2 toxin. In certain embodiments, the disease agent includes a plurality of non-identical disease agents such that the binding regions of the binding protein bind to and neutralize the plurality of disease agents.

In various embodiments of the composition, the bacterium for which the binding protein is specific is selected from: B. anthracis, B. cereus, C. botulinum, C. difficile, C. perfringens, V. cholerae, and C. spiroforme. In a related embodiment, the bacterium is a virulent bacterium or apathogenic bacterium.

The composition in various embodiments is compounded or formulated for a route of delivery selected from the group of topical, ocular, nasal, bucal, oral, rectal, parenteral, intracisternal, invaginal, and intraperitoneal.

In various embodiments of the composition, the binding protein is specific for a toxin which is a C. botulinum toxin, and the binding regions of the binding protein includes a recombinant camelid heavy-chain-only antibody, and the composition includes an amino acid sequence selected from the group:

(VHH H7, SEQ ID NO: 56)
LVQVGGSLRLSCVVSGSDISGIAMGWYRQAPGKRREMVADIFSGGSTDYAGSVKGRFTISR
DNAKKTSYLQMNNVKPEDTGVYYCRLYGSGDYWGQGTQVTVSSAHHSEDP;
(VHH B5, SEQ ID NO: 57)
LVHPGGSLRLSCAPSASLPSTPFNPFNNMVGWYRQAPGKQREMVASIGLRINYADSVKGRF
TISRDNAKNTVDLQMDSLRPEDSATYYCHIEYTHYWGKGTLVTVSSEPKTPKPQ;
and
(H7/B5 heterodimer, SEQ ID NO: 58)
QVQLVESGGGLVQVGGSLRLSCVVSGSDISGIAMGWYRQAPGKRREMVADIFSGGSTDYA
GSVKGRFTISRDNAKKTSYLQMNNVKPEDTGVYYCRLYGSGDYWGQGTQVTVSSAHHSE
DPTSAIAGGGGSGGGGSGGGGSLQGQLQLVESGGGLVHPGGSLRLSCAPSASLPSTPFNPFN
NMVGWYRQAPGKQREMVASIGLRINYADSVKGRFTISRDNAKNTVDLQMDSLRPEDSAT
YYCHIEYTHYWGKGTLVTVSSEPKTPKPQ.

In a related embodiment of the composition, the binding protein is specific for a toxin which is a C. difficile toxin A, and the binding region of the binding protein includes a recombinant camelid heavy-chain-only antibody having an amino acid sequence selected from the group of:

(AH3, SEQ ID NO: 59)
QVQLVETGGLVQPGGSLRLSCAASGFTLDYSSIGWFRQAPGKEREGVSCISSSGDSTKYAD
SVKGRFTTSRDNAKNTVYLQMNSLKPDDTAVYYCAAFRATMCGVFPLSPYGKDDWGKG
TLVTVSSEPKTPKPQP;
(AA6, SEQ ID NO: 60)
QLQLVETGGGLVQPGGSLRLSCAASGFTFSDYVMTWVRQAPGKGPEWIATINTDGSTMRD
DSTKGRFTISRDNAKNTLYLQMTSLKPEDTALYYCARGRVISASAIRGAVRGPGTQVTVSS
EPKTPKPQP;
(A3H, SEQ ID NO: 61)
QVQLVESGGGLVQPGGSLRLSCAASGFTLDYYAIGWFRQAPGKEREGVSGISSVDGSTYYA
DSVRGRFTISRDNAKNTVYLQMNSLKPEDTAVYYCAADQSPIPIHYSRTYSGPYGMDYWG
KGTLVTVSSAHHSEDP;
(AC1, SEQ ID NO: 62)
QLQLVESGGGLVQPGGSLRLSCAASGFTLDYYAIGWFRQAPGKEREGVSGISFVDGSTYYA
DSVKGRFAISRGNAKNTVYLQMNSLKPEDTAVYYCAADQSSIPMHYSSTYSGPSGMDYW
GKGTLVTVSSEPKTPKPQP;
(A11G, SEQ ID NO: 63)
QLQLVETGGGLVQAGGSLRLSCAASGRTLSNYPMGWFRQAPGKEREFVAAIRRIADGTYY
ADSVKGRFTISRDNAWNTLYLQMNGLKPEDTAVYFCATGPGAFPGMVVTNPSAYPYWGQ
GTQVTVSSEPKTPKPQP;
(AE1, SEQ ID NO: 64)
QLQLVESGGGLVQPGGSLRLSCAASGFTLDYYAIGWFRQAPGKEREGVSGISSSDGSTYYA
DSVKGRFTISRDNATNTVYLQMNSLKPEDTAVYYCAADQAAIPMHYSASYSGPRGMDYW
GKGTLVTVSSEPKTPKPQP;
(SEQ ID NO: 87)
MSDKIIHLTDDSFDTDVLKADGAILVDFWAEWCGPCKMIAPILDEIADEYQGKLTVAKLNI
DQNPGTAPKYGIRGIPTLLLFKNGEVAATKVGALSKGQLKEFLDANLAGSGSGHMHHHHH
HSSGLVPRGSGMKETAAAKFERQHMDSPDLGTDDDDKAMAISDPNSQVQLVESGGGLVQ
PGGSLRLSCEASGFTLDYYGIGWFRQPPGKEREAVSYISASARTILYADSVKGRFTISRDNA
KNAVYLQMNSLKREDTAVYYCARRRFSASSVNRWLADDYDVWGRGTQVAVSSEPKTPK
PQTSAIAGGGGSGGGGSGGGGSLQAMAAASQVQLVESGGGLVQTGGSLRLSCASSGSIAG
FETVTWSRQAPGKSLQWVASMTKTNNEIYSDSVKGRFIISRDNAKNTVYLQMNSLKPEDT
GVYFCKGPELRGQGIQVTVSSEPKTPKPQPARR;
and,
(SEQ ID NO: 95)
MSDKIIHLTDDSFDTDVLKADGAILVDFWAEWCGPCKMIAPILDEIADEYQGKLTVAKLNI
DQNPGTAPKYGIRGIPTLLLFKNGEVAATKVGALSKGQLKEFLDANLAGSGSGHMHHHHH
HSSGLVPRGSGMKETAAAKFERQHMDSPDLGTDDDDKAMAISDPNSQVQLVETGGLVQP
GGSLRLSCAASGFTLDYSSIGWFRQAPGKEREGVSCISSSGDSTKYADSVKGRFTTSRDNAK
NTVYLQMNSLKPDDTAVYYCAAFRATMCGVFPLSPYGKDDWGKGTLVTVSSEPKTPKPQ
PTSAIAGGGGSGGGGSGGGGSLQAMAAAQLQLVETGGGLVQPGGSLRLSCAASGFTFSDY
VMTWVRQAPGKGPEWIATINTDGSTMRDDSTKGRFTISRDNAKNTLYLQMTSLKPEDTAL
YYCARGRVISASAIRGAVRGPGTQVTVSSEPKTPKPQPARQTSPSTVRLESRVRELEDRLEE
LRDELERAERRANEMSIQLDEC.

In certain embodiments of the composition, the binding protein is specific for a toxin which is a C. difficile toxin B, and the binding region of the binding protein includes a recombinant camelid heavy-chain-only antibody having an amino acid sequence selected from the group consisting of:

(2D, SEQ ID NO: 65)
QVQLVESGGGLVQPGGSLRLSCAASGFSLDYYGIGWFRQAPGKERQEVSYISASAKTKLYS
DSVKGRFTISRDNAKNAVYLEMNSLKREDTAVYYCARRRFDASASNRWLAADYDYWGQ
GTQVTVSSEPKTPKPQ;
(2Ds, SEQ ID NO: 66)
QVQLVESGGGLVQAGGSLRLSCVSSERNPGINAMGWYRQAPGSQRELVAIWQTGGSLNY
ADSVKGRFTISRDNLKNTVYLQMNSLKPEDTAVYYCYLKKWRDQYWGQGTQVTVSSEPK
TPKPQ;
(5D, SEQ ID NO: 67)
QVQLVESGGGLVQPGGSLRLSCEASGFTLDYYGIGWFRQPPGKEREAVSYISASARTILYA
DSVKGRFTISRDNAKNAVYLQMNSLKREDTAVYYCARRRFSASSVNRWLADDYDVWGR
GTQVAVSSEPKTPKPQ;
(E3, SEQ ID NO: 68)
QVQLVESGGGLVQTGGSLRLSCASSGSIAGFETVTWSRQAPGKSLQWVASMTKTNNEIYS
DSVKGRFIISRDNAKNTVYLQMNSLKPEDTGVYFCKGPELRGQGIQVTVSSEPKTPKPQ;
(7F, SEQ ID NO: 69)
QVQLVESGGGLVEAGGSLRLSCVVTGSSFSTSTMAWYRQPPGKQREWVASFTSGGAIKYT
DSVKGRFTMSRDNAKKMTYLQMENLKPEDTAVYYCALHNAVSGSSWGRGTQVTVSSEP
KTPKPQ;
(5E, SEQ ID NO: 70)
VQLVESGGGLVQAGGSLRLSCAASGLMFGAMTMGWYRQAPGKEREMVAYITAGGTESY
SESVKGRFTISRINANNMVYLQMTNLKVEDTAVYYCNAHNFWRTSRNWGQGTQVTVSSE
PKTPKP;
(B12, SEQ ID NO: 71)
VQLVESGGGLVQAGDSLTLSCAASESTFNTFSMAWFRQAPGKEREYVAAFSRSGGTTNYA
DSVKGRATISTDNAKNTVYLHMNSLKPEDTAVYFCAADRPAGRAYFQSRSYNYWGQGTQ
VTVSSAHHSEDP;
(A11, SEQ ID NO: 72)
VQLVESGGGSVQIGGSLRLSCVASGFTFSKNIMSWARQAPGKGLEWVSTISIGGAATSYAD
SVKGRFTISRDNANDTLYLQMNNLKPEDTAVYYCSRGPRTYINTASRGQGTQVTVSSEPKT
PKP;
(AB8, SEQ ID NO: 73)
VQLVESGGGLVQAGGSLRLSCVGSGRNPGINAMGWYRQAPGSQRELVAVWQTGGSTNY
ADSVKGRFTISRDNLKNTVYLQMNSLKPEDTAVYYCYLKKWRDEYWGQGTQVTVSSAH
HSEDP;
(C6, SEQ ID NO: 74)
VQLVESGGGLVQAGESLRLSCVVSESIFRINTMGWYRQTPGKQREVVARITLRNSTTYADS
VKGRFTISRDDAKNTLYLKMDSLKPEDTAVYYCHRYPLIERNSPYWGQGTQVIVSSEPKTP
KP;
(C12, SEQ ID NO: 75)
VQLVESGGGLVQAGESERLSCVVSESIFRINTMGWYRQTPGKQREVVARITLRNSTTYADS
VKGRFTISRDDAKNTLYLKMDSLKPEDTAVYYCHRYPLIFRNSPYWGQGTQVTVSSEPKTP;
(A1, SEQ ID NO: 76)
VQLVESGGGLVQAGGSLRLSCAAPGLTFTSYRMGWFRQAPGKEREYVAAITGAGATNYA
DSAKGRFTISKNNTASTVHLQMNSLKPEDTAVYYCAASNRAGGYWRASQYDYWGQGTQ
VTVSSAHHSEDP; SEQ ID NO: 87; and SEQ ID NO: 95.

In related embodiments of the composition, the binding protein is specific for a toxin which is a Shiga toxin, and the binding region of the binding protein includes a recombinant camelid heavy-chain-only antibody having an amino acid sequence selected from the group:

(JET-A9, SEQ ID NO: 77)
QVQLVETGGGLAQAGDSLRLSCVEPGRTLDMYAMGWIRQAPGEEREFVASISGVGGSPRY
ADSVKGRFTISKDNTKSTIWLQMNSLKPEDTAVYYCAAGGDIYYGGSPQWRGQGTRVTVS
SEPKTPKPQ;
(JGG-D4, SEQ ID NO: 78)
QVQLVESGGGLVQAGGSLRLSCAASGRINGDYAMGWFRQAPGEEREFVAVNSWIGGSTY
YTDSVKGRFTLSRDNAKNTLSLQMNSLKPEDTAVYYCAAGHYTDFPTYFKEYDYWGQGT
QVTVSSEPKTPKPQ;
(JEN-D10, SEQ ID NO: 79)
QVQLVETGGLVQAGGSLRLSCAASGVPFSDYTMAWFRQAPGKEREVVARITWRGGGPYY
GNSGNGRFAISRDIAKSMVYLHMDSLKPEDTAVYYCAASRLRPALASMASDYDYWGQGT
QVSVSSEPKTPKPQ;
(JGH-G1, SEQ ID NO: 80)
QVQLVESGGGLVQPGESLRLSCVASASTFSTSLMGWVRQAPGKGLESVAEVRTTGGTFYA
KSVAGRFTISRDNAKNTLYLQMNSLKAEDTGVYYCTAGAGPIATRYRGQGTQVTVSSAHH
SEDP;
(JEU-A6, SEQ ID NO: 81)
QVQLVESGGGLVQPGGSLKLSCAASGFTLADYVTVWFRQAPGKSREGVSCISSSRGTPNYA
DSVKGRATVSRNNANNTVYLQMNGLKPDDTAIYYCAAIRPARLRAYRECLSSQAEYDYW
GQGTQVTVSSAHHSEDP;
(JEU-D2, SEQ ID NO: 82)
QVQLVESGGGLVQPGGSLGLSCAMSGTTQDYSAVGWFRQAPGKEREGVSCISRSGRRTNY
ADSVRGRFTISRDNAKDTVYLQMNSLKPDDTAVYYCAARKTDMSDPYYVGCNGMDYWG
KGTLVTVSSAHHSEDP;
(JGH-G9, SEQ ID NO: 83)
QVQLVESGGGLVQPGGSLTLSCTASGFTLNSYKIGWFRQAPGKEREGVSCINSGGNLRSVE
GRFTISRDNTKNTVSLHMDSLKPEDTGVYHCAAAPALNVFSPCVLAPRYDYWGQGTQVTV
SSAHHSEDP;
(JFD-A4, SEQ ID NO: 84)
QVQLVESGGGLVQPGGSLRLSCAASGFTLGSYHIGWFRHPPGKEREGTSCLSSRGDYTKYA
EAVKGRFTISRDNTKSTVYLQMNNLKPEDTGIYVCAAIRPVLSDSHCTLAARYNYWGQGT
QVTVSSAHHSEDP;
(JFD-A5, SEQ ID NO: 85)
QVQLVESGGGLVQPGGSLRISCAALEFTLEDYAIAWFRQAPGKEREGVSCISKSGVTKYTD
SVKGRFTVARDNAKSTVILQMNNLRPEDTAVYNCAAVRPVFVDSVCTLATRYTYWGEGT
QVTVSSAHHSEDP;
and
(JGG-G6, SEQ ID NO: 86)
QVQLVETGGGLVQPGGSLKLSCAASEFTEDDYHIGWFRQAPGKEREGVSCINKRGDYINY
KDSVKGRFTISRDGAKSTVFLQMNNERPEDTAVYYCAAVNPVFPDSRCTLATRYTHWGQG
TQVTVSSAHHSEDP.

In certain embodiments amino acid sequence SEQ ID NO: 77, amino acid QVQLVETGGGLAQAGDSLRLSCVEPGRTLDMYAMGWIRQAPGEEREFVASISGVGGSPRY ADSVKGRFTISKDNTKSTIWLQMNSLKPEDTAVYYCAAGGDIYYGGSPQWRGQGTRVTVS SEPKTPKPQ (JET-A9) binds to Stx1 or a portion or homolog thereof.

In certain embodiments amino acid sequence SEQ ID NO: 78, amino acid QVQLVESGGGLVQAGGSLRLSCAASGRINGDYAMGWFRQAPGEEREFVAVNSWIGGSTY YTDSVKGRFTLSRDNAKNTLSLQMNSLKPEDTAVYYCAAGHYTDFPTYFKEYDYWGQGT QVTVSSEPKTPKPQ (JGG-D4) binds to Stx1 or a portion or homolog thereof.

In certain embodiments amino acid sequence SEQ ID NO: 79, amino acid QVQLVETGGLVQAGGSLRLSCAASGVPFSDYTMAWFRQAPGKEREVVARITWRGGGPYY GNSGNGRFAISRDIAKSMVYLHMDSLKPEDTAVYYCAASRLRPALASMASDYDYWGQGT QVSVSSEPKTPKPQ (JEN-D10) binds to Stx2 or a portion or homolog thereof.

In certain embodiments amino acid sequence SEQ ID NO: 80, amino acid QVQLVESGGGILQPGESLRLSCVASASTFSTSLMGWVRQAPGKGLESVAEVRTTGGTFYA KSVAGRFTISRDNAKNTLYLQMNSLKAEDTGVYYCTAGAGPIATRYRGQGTQVTVSSAHH SEDP (JGH-G1) binds to Stx2 or a portion or homolog thereof.

In certain embodiments amino acid sequence SEQ ID NO: 81, amino acid QVQLVESGGGLVQPGGSLKLSCAASGFTLADYVTVWFRQAPGKSREGVSCISSSRGTPNYA DSVKGRATVSRNNANNTVYLQMNGLKPDDTAIYYCAAIRPARLRAYRECLSSQAEYDYW GQGTQVTVSSAHHSEDP (JEU-A6) binds to Stx2 or a portion or homolog thereof.

In certain embodiments amino acid sequence SEQ ID NO: 82, amino acid QVQLVESGGGLVQPGGSLGLSCAMSGTTQDYSAVGWFRQAPGKEREGVSCISRSGRRTNY ADS VRGRFTISRDNAKDTVYLQMNSLKPDDTAVYYCAARKTDMSDPYYVGCNGMDYWG KGTLVTVSSAHHSEDP (JEU-D2) binds to Stx2 or a portion or homolog thereof.

In certain embodiments amino acid sequence SEQ ID NO: 83, amino acid QVQLVESGGGLVQPGGSLTLSCTASGFTLNSYKIGWFRQAPGKEREGVSCINSGGNLRSVE GRFTISRDNTKNTVSLHMDSLKPEDTGVYIICAAAPALNVFSPCVLAPRYDYWGQGTQVTV SSAHHSEDP (JGH-G9) binds to Stx2 or a portion or homolog thereof.

In certain embodiments amino acid sequence SEQ ID NO: 84, amino acid QVQLVESGGGLVQPGGSLRLSCAASGFTLGSYHIGWFRHPPGKEREGTSCLSSRGDYTKYA EAVKGRFTISRDNTKSTVYLQMNNLKPEDTGIYVCAAIRPVLSDSHCTLAARYNYWGQGT QVTVSSAHHSEDP (JFD-A4) binds to Stx1, Stx2, or both Stx1 And Stx2. In various embodiments SEQ ID NO: 84 binds to at least one of Stx1, Stx2, or a portion or homolog thereof.

In certain embodiments amino acid sequence SEQ ID NO: 85, amino acid QVQLVESGGGLVQPGGSLRLSCAALEFTLEDYAIAWFRQAPGKEREGVSCISKSGVTKYTD SVKGRFTVARDNAKSTVILQMNNLRPEDTAVYNCAAVRPVFVDSVCTLATRYTYWGEGT QVTVSSAHHSEDP (JFD-A5) binds to Stx1, Stx2, or both Stx1 And Stx2. In various embodiments SEQ ID NO: 85 binds to at least one of Stx1, Stx2, or a portion or homolog thereof.

In certain embodiments amino acid sequence SEQ ID NO: 86, amino acid QVQLVETGGGLVQPGGSLKLSCAASEFTLDDYHIGWFRQAPGKEREGVSCINKRGDYINY KDSVKGRFTISRDGAKSTVFLQMNNLRPEDTAVYYCAAVNPVFPDSRCTLATRYTHWGQG TQVTVSSAHHSEDP (JGG-G6) binds to Stx1, Stx2, or both Stx1 And Stx2. In various embodiments SEQ ID NO: 86 binds to at least one of Stx1, Stx2, or a portion or homolog thereof.

In various embodiments, the amino acid sequence of the composition further includes an amino acid analog, an amino acid derivative, or a conservative substitution of an amino acid residue. The binding protein in various embodiments includes an amino acid sequence that is substantially identical to the amino acid sequence of SEQ ID NOs: 56-87 and 95. In related embodiments, substantially identical means that the amino acid sequence or the binding protein has at least about 50% identity, at least about 60% identity, at least about 65% identity, at least about 70% identity, at least about 75% identity, at least about 80% identity, at least about 85% identity, at least about 90% identity, at least about 95% identity, at least about 97% identity, at least about 98% identity, or at least about 99% identity to the amino acid sequence of SEQ ID NOs: 56-87 and 95. Alternatively, the binding protein is encoded by at least one nucleotide sequence or the protein includes amino acid sequence selected from the group of SEQ ID NOs: 1-87 and 95, and substantially identical to any of these sequences.

The composition in various embodiments further includes the binding protein or a source of expression of the binding protein selected from the group of a purified binding protein preparation; a nucleic acid vector with a gene encoding the binding protein; a viral vector encoding the binding protein; and a naked nucleic acid encoding the binding protein which is expressed from the DNA. In related embodiments, the viral vector is derived from a genetically engineered genome of at least one virus selected from: an adenovirus, an adeno-associated virus, a herpes virus, and a lentivirus.

In a related embodiment of the composition, the binding protein is heterodimeric. In various embodiments, the heterodimeric binding protein includes a first binding region and a second binding region. For example the first binding region and the second binding region include VHHs, and the first binding region binds specifically to a C. difficile TcdA and the second binding region binds specifically to a C. difficile TcdB.

An aspect of the invention provides a kit for treating a subject exposed to or at risk for exposure to a disease agent including: a pharmaceutical composition for treating a subject at risk for exposure to or exposed to a disease agent, the pharmaceutical composition including: at least one recombinant heteromultimeric neutralizing binding protein comprising a plurality binding regions, such that the binding regions are not identical, and each binding region specifically binds a non-overlapping portion of the disease agent, such that the binding protein neutralizes the disease agent, thereby treating the subject for exposure to the disease agent; a container; and, instructions for use. In various embodiments, the instructions for use include instructions for a method for treating a subject at risk for exposure to or exposed to a disease agent using the pharmaceutical composition.

In various embodiments of the kit, the binding protein is selected from the group of: a single-chain antibody (scFv); a recombinant camelid heavy-chain-only antibody (VHH); a shark heavy-chain-only antibody (VNAR); a microprotein; a darpin; an anticalin; an adnectin; an aptamer; a Fv; a Fab; a Fab′; and a F(ab′)₂.

In a related embodiment of the kit, the binding protein includes a linker. In various embodiments, the linker includes at least one selected from: a peptide, a protein, a sugar, or a nucleic acid. For example, the linker includes amino acid sequence GGGGS (SEQ ID NO: 54), or GGGGSGGGGSGGGGS (SEQ ID NO: 55), or a portion thereof. Alternatively, the linker includes a single amino acid or a plurality of amino acids.

In related embodiments of the kit, the disease agent for which the binding protein and binding regions are specific is selected from: a virus, a cancer cell, a fungus, a bacterium, a parasite, and a product of one of those such as a pathogenic molecule, a protein, a lipopolysaccharide, or a toxin. In related embodiments, the toxin for which the binding protein is specific is a Botulinum neurotoxin including a serotype selected from: A, B, C, D, E, F, and G. In various embodiments of the kit, the toxin for which the binding protein is specific is at least one selected from the group of: staphylococcal α-hemolysin, staphylococcal leukocidin, aerolysin cytotoxic enterotoxin, a cholera toxin, a Bacillus cereus hemolysis II toxin, a Helicobacter pylori vacuolating toxin, a Bacillus anthracisi toxin, a cholera toxin, an Escherichia coli serotype O157:H7 toxin, an Escherichia coli serotype O104:H7 toxin, a lipopolysaccharide endotoxin, a Shiga toxin, a pertussis toxin, a Clostridium perfringens iota toxin, a Clostridium spiroforme toxin, a Clostridium difficile toxin A, a Clostridium difficile toxin B, a Clostridium septicum a toxin, and a Clostridium botulinum C2 toxin. In certain embodiments, the binding regions of the binding protein are specific to different classes of disease agents, e.g., each of the plurality of binding regions is different and is specific for an agent from bacteria, virus, fungus, cancer, and a pathogenic molecule. For example a binding region is specific for a virus and another binding region is specific for a bacterium.

In a related embodiment of the kit, the binding protein is specific for a toxin which is a C. botulinum toxin, and the binding region includes a recombinant camelid heavy-chain-only antibody, such that the pharmaceutical composition includes the binding protein that has an amino acid sequence selected from the group consisting of: SEQ ID NO: 56, SEQ ID NO: 57, SEQ ID NO: 58, or a portion thereof.

In a related embodiment of the kit, the binding region of the binding protein is specific for a toxin which is a C. botulinum toxin A, such that the binding region of the binding protein includes a recombinant camelid heavy-chain-only antibody having an amino acid sequence selected from the group of: SEQ ID NO: 59, SEQ ID NO: 60, SEQ ID NO: 61, SEQ ID NO: 62, SEQ ID NO: 63, SEQ ID NO: 64, SEQ ID NO: 87, SEQ ID NO: 95, and a portion thereof.

In a related embodiment of the kit, the toxin for which the binding protein is specific is a C. difficile toxin B, and the binding region of the binding protein includes a recombinant camelid heavy-chain-only antibody having an amino acid sequence selected from: SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 87, SEQ ID NO: 95, and a portion thereof. In certain embodiments, the binding protein and/or binding regions are encoded by a nucleotide sequence or the binding protein and/or regions include an amino acid sequence selected from the group of SEQ ID NOs: 1-87 and 95, or are substantially identical to these sequences.

In a related embodiment, the binding protein is specific for a Shiga toxin, and the binding region of the binding protein includes a recombinant camelid heavy-chain-only antibody having an amino acid sequence selected from: SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and SEQ ID NO: 86.

An aspect of the invention provides a composition including at least one amino acid sequence selected from the group of: SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94, SEQ ID NO: 95 or a portion thereof. The composition in various embodiments includes an amino acid sequence that is substantially identical to the amino acid sequence of SEQ ID NOs: 59-86. In related embodiments, substantially identical means an amino acid sequence that has at least 60% identity, at least 65% identity, at least 70% identity, at least 75% identity, at least 80% identity, at least 85% identity, at least 90% identity, at least 95% identity, at least about 97% identity, at least about 98% identity, or at least 99% identity to an amino acid sequence of any of SEQ ID NOs: 56-87 and 95.

An aspect of the invention provides a method for treating a subject at risk for exposure to or exposed to a plurality of disease agents, the method including: contacting the subject with at least one recombinant heteromultimeric neutralizing binding protein including two or more binding regions, such that the binding protein neutralizes at least two (plurality) of disease agents, thereby treating the subject for exposure to the plurality of disease agents.

In a related embodiment of the method, the at least two of the binding regions are identical. Alternatively, the at least two binding regions include at least two non-identical binding regions. In related embodiments of the method, the binding protein is at least one selected from the group of: a heterodimer, a trimer, a tetramer, a pentamer, and a hexamer. In various embodiments, the tetramer includes a homodimer of a heterodimer, for example a heterodimer of AH3 and AA6 as is shown in SEQ ID NO: 95.

In various embodiments, the plurality from which the exemplary disease agents are selected from a virus, a cancer cell, a fungus, a bacterium, a parasite and a product thereof such as a pathogenic molecule, a protein, a lipopolysaccharide, or a toxin. For example the disease agents include toxins such as TcdA and TcdB.

In related embodiments of the method, the binding protein includes at least one selected from the group of SEQ ID NOs: 56-87 and 95 or a portion or a homologue.

In related embodiments of the method, the binding protein is selected from the group of: a single-chain antibody (scFv); a recombinant camelid heavy-chain-only antibody (VHH); a shark heavy-chain-only antibody (VNAR); a microprotein; a darpin; an anticalin; an adnectin; an aptamer; a Fv; a Fab; a Fab′; and a F(ab′)₂. In a related embodiment of the method, the binding protein includes a linker located between each of the multimeric components of the binding regions. In various embodiments, the linker is at least one selected from the group of: a peptide, a protein, a sugar, or a nucleic acid. For example, the linker comprises amino acid sequence GGGGS (SEQ ID NO: 54) or amino acid sequence GGGGSGGGGSGGGGS (SEQ ID NO: 55).

In a related embodiment, the method further includes prior to contacting, engineering the binding protein using a dimerization agent. In a related embodiment, the dimerization agent includes amino acid sequence TSPSTVRLESRVRELEDRLEELRDELERAERRANEMSIQLDEC (SEQ ID NO:94), or a portion thereof.

In various embodiments of the method, the plurality of disease agents is at least two selected from the group of: Staphylococcal α-hemolysin, Staphylococcal leukocidin, aerolysin cytotoxic enterotoxin, a cholera toxin, Bacillus cereus hemolysis II, and Helicobacter pylori vacuolating toxin, Bacillus anthracis, cholera toxin, Escherichia coli serotype O157:H7, Escherichia coli serotype O104:H7, lipopolysaccharide endotoxin, Shiga toxin, pertussis toxin, Clostridium perfringens iota toxin, Clostridium spiroforme toxin, Clostridium difficile toxin A, Clostridium difficile toxin B, Clostridium septicum a toxin, and Clostridium botulinum C2 toxin. In related embodiments of the method, the binding protein includes at least one selected from the group of: SEQ ID NOs: 56-87 and 95.

### Binding Agent

The binding agent or binding protein is in one embodiment, a molecule that binds to a portion of a target molecule, disease agent, or disease agent target. The binding protein treats the subject by any or all of several mechanisms, including promoting clearance, phagocytosis, neutralization, inhibition, and activation of the immune response. The term “binding agent” or “binding protein”, includes in addition to full-length antibodies, molecules such as antibody fragments (e.g., single chain antibodies, and VHHs), microproteins (also referred to as cysteine knot proteins or knottins), darpins, anticalins, adnectins, peptide mimetic molecules, aptamers, synthetic molecules, and refers to any composition that binds to a target and/or disease agent and elicits an immune effector activity against the molecule target and/or disease agent. In certain embodiments, the binding protein is a recombinant multimeric neutralizing binding protein including two or more binding regions, such that the binding regions are not identical, and each and/or disease agent. Alternatively, the binding protein includes binding regions that bind specifically to different types of disease agents such as different types of pathogenic molecules such as bacteria, viruses, fungi, allergens, and toxins. For example at least one binding region of the binding protein bind to a virus surface protein, and at least one different binding regions binds to a bacterial toxin.

The multimeric neutralizing binding protein herein in certain embodiments includes one or a plurality of epitopic tags. In certain embodiments, the binding protein includes a linker that covalently connects each binding region of the heterodimer. For example, the linker is a single amino acid or a sequence of a plurality of amino acids that does not affect or reduce the stability, orientation, binding, neutralization, and/or clearance characteristics of the binding regions and binding protein. In certain embodiments, each binding region is specific to a non-identical disease agent. For example the binding protein in certain embodiments includes a binding region specific to a bacterium or bacterial toxin, and at least one other binding region is specific to a virus, fungus, allergen, or to a non-identical bacterium or bacterial toxin. For example, a multimeric binding protein in certain embodiments has binding regions specific to a TcdA and to a TcdA or to a Shiga toxin, or the respective binding regions are specific to each of a Botulinum toxin and a virus.

In certain embodiments, the binding protein neutralizes or inhibits the molecule target and/or disease agent for example by preventing the disease agent entry into cells. In certain embodiments, the binding protein upon being administered to the subject neutralizes the toxin and/or triggers an antibody mediated effector activity in the subject.

The binding protein is in certain embodiments a monomer (e.g., a single unit), or includes a covalently bound protein including a plurality of monomers such as for example a dimer, a trimer, a tetramer, a pentamer, an octamer, a 10-mer, a 15-mer, a 20-mer, or any multimer. In certain embodiments, the binding protein is a monomer and the binding protein has one binding region that binds to an epitope of the molecule target and/or disease agent. Alternatively, the binding protein in certain embodiments has two or more connected or joined monomers each with a binding region and each binding to an epitope of a disease agent or to a plurality of epitopes of disease agents. The multimeric binding protein in certain embodiments includes the same monomer. Alternatively the multimeric binding protein includes monomers or binding regions or a combination thereof (i.e., heteromulteric). Accordingly, the multimers can be homogeneous such that each includes two or more monomers having a binding region that binds to the same site of a disease agent. Alternatively the multimers are heterogeneous and include two or more monomers having a binding region that binds to two or more different sites of one or more disease agents. The heterogeneous multimers (heteromultimers) bind non-overlapping portions of the molecule target and/or disease agent. In various embodiments, the binding protein is a homodimer of a heterodimer or a heterotrimer. In a related embodiment, the heteromultimers bind a plurality of non-identical epitopes on a plurality of disease agents.

In certain embodiments the binding protein includes a single tag, multiple tags, for example each multimeric binding protein includes two or more tags on each component binding region (i.e., monomer). Alternatively, the heterodimer comprises no tag attached to the monomers and/or linker. In certain embodiments, presence of the tag on or operably fused to the binding protein and/or binding region synergistically induces clearance of the disease agent from the body. For example the tag attached to the binding protein induces an immune response from a patient or subject contacted with a pharmaceutical composition containing the tagged-binding protein. In certain embodiments the tag includes a portion (e.g., conserved, unique, in-activated, and non-functional) of a pathogenic molecule. In certain embodiments, the tag is an adjuvant. See Gerber et al. U.S. Pat. No. 7,879,333 issued Feb. 1, 2011 which is incorporated by reference herein in its entirety. For example, the tag is a peptide, carbohydrate, polymer, or nucleic acid that is effective for enhancing neutralization and/or clearance of the disease agent or plurality of disease agents.

The multimeric binding protein in certain embodiments is a heterodimer having two tags, one tag attached to each monomer, or alternatively the heterodimer includes one tag on each monomer or one tag total on one of the two monomers. The term “heterodimer” includes a single protein having two different monomers are joined by a linker. Data herein shown that a heterodimers having two E-tags effectively protected animals exposed to hundreds-fold and/or thousands-fold the lethal dose of a single disease agent such as a C. difficile toxin A. Examples herein show that recombinant multimeric binding proteins, having two or more non-identical binding regions, administered to subjects either before or after contact with a disease agent resulted in comparable and better antitoxin efficacy than serum-based polyclonal antitoxins.

The binding agents/proteins described herein include binding agent/protein portions, regions, and fragments. For example, the binding protein is an antibody and, in certain embodiments the binding protein includes antibody fragments. The term “antibody fragment” refers to portion of an immunoglobulin having specificity to an molecule target and/or disease agent, or a molecule involved in the interaction or binding of the molecule target and/or disease agent. The term “antibody fragment” encompasses fragments from binding protein, for example both polyclonal and monoclonal antibodies including transgenically produced antibodies, single-chain antibodies (scFvs), recombinant Fabs, and recombinant heavy-chain-only antibodies (VHHs), e.g., from any organism producing VHH antibody such as a camelid, a shark, or a designed VHH.

VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3). The cloned and isolated VHH domain is a stable polypeptide harboring the antigen-binding capacity of the original heavy-chain antibody. See Castorman et al. U.S. Pat. No. 5,840,526 issued Nov. 24, 1998; and Castorman et al. U.S. Pat. No. 6,015,695 issued Jan. 18, 2000, each of which is incorporated by reference herein in its entirety. VHHs are commercially available from Ablynx Inc. (Ghent, Belgium) under the trademark of Nanobodies™.

Suitable methods of producing or isolating antibody fragments having the requisite binding specificity and affinity are described herein and include for example, methods which select recombinant antibody from a library, by PCR (See Ladner U.S. Pat. No. 5,455,030 issued Oct. 3, 1995 and Devy et al. U.S. Pat. No. 7,745,587 issued Jun. 29, 2010, each of which is incorporated by reference herein in its entirety).

Functional fragments of antibodies, including fragments of chimeric, humanized, primatized, veneered or single chain antibodies, can also be produced. Functional fragments or portions of the foregoing antibodies include those which are reactive with the disease agent. For example, antibody fragments capable of binding to the disease agent or portion thereof, including, but not limited to scFvs, Fabs, VHHs, Fv, Fab, Fab′ and F(ab′)₂ are encompassed by the invention. Such fragments can be produced by enzymatic cleavage or by recombinant techniques. For instance, papain or pepsin cleavage are used generate Fab or F(ab′)₂ fragments, respectively. Antibody fragments are produced in a variety of truncated forms using antibody genes in which one or more stop codons has been introduced upstream of the natural stop site. For example, a chimeric gene encoding a F(ab′)₂ heavy chain peptide portion can be designed to include DNA sequences encoding the CH₁ peptide domain and hinge region of the heavy chain. Accordingly, the present invention encompasses a polynucleic acid that encodes the binding protein described herein (e.g., a binding fragment with a tag). Binding proteins in certain embodiments are made as part of a multimeric protein, the monomer or single binding region (e.g., antibody fragments, microproteins, darpins, anticalins, adnectins, peptide mimetic molecules, aptamers, synthetic molecules, etc) can be linked. Any combination of binding protein or binding region types can be linked. In an embodiment, the monomer or binding region of a multimeric binding protein can be linked covalently. In another embodiment, a monomer binding protein can be modified, for example, by attachment (directly or indirectly (e.g., via a linker or spacer)) to another monomer binding protein. A monomer in various embodiments is attached or genetically fused to another monomer e.g., by recombinant protein that is engineered to contain extra amino acid sequences that constitute the monomers. Thus, the DNA encoding one monomer is joined (in reading frame) with the DNA encoding the second monomer, and so on. Additional amino acids in certain embodiments are encoded between the monomers that produce an unstructured region separating the different monomers to better promote the independent folding of each monomer into its active conformation or shape. Commercially available techniques for fusing proteins are used in various embodiments to join the monomers into a multimeric binding protein of the present invention.

The term “antagonist” as used herein includes proteins or polypeptides that bind to the disease agent, inhibit function of the disease agent, and are included in certain embodiments to the binding region of the binding protein.

A binding protein includes any amino acid sequence that binds to the disease agent or target including molecules that have scaffolds. Examples of binding proteins having scaffolds are DARPins, Anticalins, and AdNectins. DARPins are derived from natural ankyrin repeat proteins and bind to proteins including e.g., human receptors, cytokines, kinases, human proteases, viruses and membrane proteins (Molecular Partners AG Zurich Switzerland). Anticalins are derived from lipocalins, and comprise a hypervariable loops supported by a conserved β-sheet framework, which acts as a binding protein. (Pieris AG, Germany). The scaffold for anticalins are lipocalins. AdNectins are derived from human fibronectin (e.g., the scaffold), and bind to targets of various medical conditions and are commercially available from Adnexus (Waltham, Mass.). See also Alexandru et al. U.S. Pat. No. 7,867,724 issued Jan. 11, 2011, which is incorporated by reference herein in its entirety. In certain embodiments, the binding protein having the scaffold is encoded by a nucleotide sequence or the binding protein includes an amino acid sequence that is substantially identical or homologous to the sequences described herein, for example SEQ ID NO: 1-87 and 95. Recombinant multimeric binding proteins herein include amino acid sequences from a binding protein sequence having conservative sequence modifications. As used herein, the term “conservative sequence modifications” refers to amino acid modifications that do not significantly affect or alter the characteristics (e.g., neutralization, clearance, binding, stability, and orientation) of the binding protein, i.e., amino acid sequences of binding protein that present these side chains at the same relative positions will function in a manner similar to the binding protein. Such conservative modifications include amino acid substitutions, additions and deletions. Modification of the amino acid sequence of recombinant multimeric binding protein is achieved using any known technique in the art e.g., site-directed mutagenesis or PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. Conservative amino acid substitutions are modifications in which the amino acid residue is replaced with an amino acid residue having a similar side chain such as replacing a small amino acid with a different small amino acid, a hydrophilic amino acid with a different hydrophilic amino acid, etc. Examples herein show that a molecule target and/or disease agent is bound by a binding protein, the molecule target and/or disease agent exemplified by a bacterial toxin released by the pathogen, for example a botulinum toxin. Botulinum toxin serotypes A to G are synthesized by organisms including Clostridium botulinum, Clostridium baratii, and Clostridium butyricum. Simpson, L. L 2004 Annu. Rev. Pharmacol. Toxicol. 44: 167-193. C. botulinum produces serotypes A to G, C. baratii produces serotype F, and C. butyricum produces serotype E only. The structures and substrates for each of the botulism toxin serotypes as well as the serotype specific cleavage sites have been determined, and the mechanism of toxin killing has been elucidated. The botulinum toxin acts preferentially on peripheral cholinergic nerve endings to block acetylcholine release, and causes disease (i.e., botulism) and can be used to treat disease (e.g., dystonia). Ibid., Abstract. The toxigenicity of botulinum toxin depends on penetration of the toxin through cellular and intracellular membranes. Thus, toxin that is ingested or inhaled binds to epithelial cells and is transported to the general vascular circulation. Toxin that reaches peripheral nerve endings binds to the cell surface then penetrates the plasma membrane by receptor-mediated endocytosis and the endosome membrane by pH-induced translocation. Ibid., Abstract. Internalized toxin acts in the cytosol as a metalloendoprotease to cleave polypeptides that are essential for exocytosis.

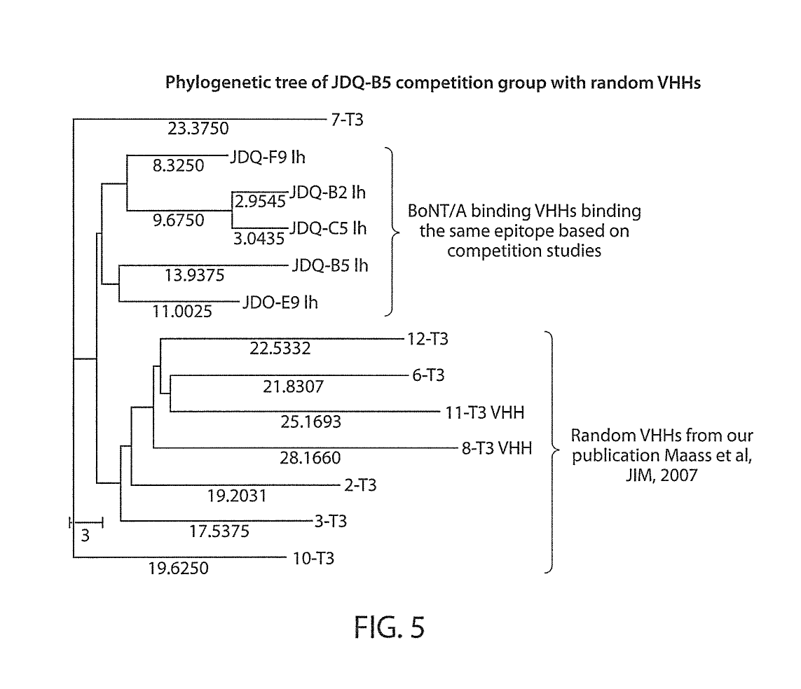

Examples herein show binding proteins/agents that specifically bind each of a variety of distinct serotypes of a microbial neurotoxin that causes botulism, BoNT/A and BoNT/B. The amino acid sequence of the binding agents include scFvs and VHHs for example SEQ ID NOs: 2, 4, 6, 8, 10, 12, 14, 20, 22, 24, 26, 28, 30, 32, 34, 36, 38, 40, 42, 44, 46, 48, 50, 52 or combinations or portions thereof. The corresponding nucleic acid sequences of binding agents are shown in SEQ ID NOs: 1, 3, 5, 7, 9, 11, 13, 19, 21, 23, 25, 27, 29, 31, 33, 35, 37, 39, 41, 43, 45, 47, 49, 51 or a combination thereof. In various embodiments the amino acid sequence of the binding agents includes VHHs for example SEQ ID NO: 56-87 or 95 or combinations or portions thereof. In certain embodiments, the binding agent includes a tag that was engineered as a portion of the binding agent, for example the tag has amino acid sequence of SEQ ID NO: 15, and is genetically fused to the carboxyl end of the binding agents. In certain embodiments, the tag enhances ability of the binding protein to neutralize and/or clear the disease agent from the subject. FIG. 5 shows a phylogenetic tree of JDQ-B5 (SEQ ID NO: 24), a VHH binding agent that specifically binds to BoNT/A and other VHHs that compete with JDQ-B5 for binding to BoNT/A. The length of the branches in the tree represents the relatedness of the sequences with the shorter branches indicating greater relatedness (i.e., homology) and the longer branches indicating less homology of the amino acid sequences.

The present invention provides a number of different binding proteins, each having binding regions with specificity and affinity to target different areas of one or more disease agents. In an embodiment, two or three binding proteins specific to different epitopes of a disease agent are used. In a disease having a number of disease agents involved in causing the disease or condition, such as botulism, multiple disease agents are targeted by the compositions and methods herein. In the case of botulism, since any one of at least seven neurotoxin serotypes are involved, a pool/mixture of binding proteins is prepared containing binding proteins for a plurality of known serotypes that cause the disease in humans. Botulism is often caused by exposure to a single BoNT serotype, and it is generally difficult to quickly determine which serotype is the cause. Thus, the standard of care in treating botulism includes administration of a number of antibodies to protect against most if not all of the serotypes that cause the disease in human. Hence, it is appropriate to protect subjects from botulism, to stockpile binding proteins that bind to several or preferably all known serotypes that cause botulism.

The present invention in various embodiments further encompasses compositions that are multimeric binding proteins having two or more monomers in which a monomer is exemplified by a VHH amino sequence herein. In various embodiments, the composition includes at least one selected from the group of SEQ ID NOs: 56-87 and 95. Compositions further include nucleic acid sequences that encode the amino acids sequences herein, for example SEQ ID NO: 56-87 and 95. In certain embodiments, the monomer or binding region includes at least one sequence described herein, for example SEQ ID NOs: 1-87 and 95. An embodiment of a multimeric binding protein includes two or more of the VHH sequences herein expressed as a single protein. Any combination of two or more of the VHH sequences forms a multimeric binding protein of the present invention. In a particular embodiment, the present invention relates to a heterodimer, i.e., protein, in which any two different VHH sequences herein are expressed as a single protein, i.e., linked and expressed as a genetic fusion.

The binding protein in certain embodiments is a multimeric fusion protein engineered and produced using a multimerization agent to form a complex that effectively binds to and neutralizes a disease agent or plurality of disease agents (Shoemaker et al. US. publication number 20130058962 published Mar. 7, 2013, which is incorporated by reference herein in its entirety). In certain embodiments, the multimerization agent includes a dimerization sequence for example including an amino acid sequence shown in SEQ ID NO: 94. For example the dimerization agent complexes peptide fragments each containing at least: about five to 25 amino acids, about 25 to 50 amino acids, about 50 to 100 amino acids, about 100 to 150 amino acids, and about 150 amino acids to about 200 amino acids. Multimerization agents and methods of using the agents for forming multimeric binding proteins are shown herein in Example 21. See also Moore et al. U.S. Pat. No. 7,763,445 issued Jul. 24, 2012 and Carter et al. U.S. Pat. No. 8,216,865 issued Jul. 10, 2012, each of which is incorporated by reference herein in its entirety.

The disease agent target is any from different classes of pathogens, infectious agents or other unwanted material. A multi-target approach is within the scope of the methods and compositions herein, exemplified by a binding protein that binds to a viral disease agent, a bacterial disease agent, a parasite disease agent, a cancer cell, and a protein produced therefrom and any combination thereof. In various embodiments, a binding protein neutralizes a plurality of pathogens or unwanted material. Examples herein show a VHH heterodimer that binds to and neutralizes both TcdA and TcdB.

The disease agent, pathogen or infectious agent that is neutralized by the binding agent is any molecule, virus or bacterium that infects a mammal (e.g., human, horse, dog, goat, and cow) or a mammalian cell. In certain embodiments, the disease agent is a bacterium selected from Actinobacillus, Bacillus, Borrelia, Brucella, Campylobacter, Chlamydia, Clostridium, Coxiella, Enterococcus, Escherichia, Francisella, Hemophilus, Legionella, Mycobacterium, Neisseria, Pasteurella, Pneumophila, Pseudomonas, Rickettsia, Salmonella, Shigella, Staphylococcus, Streptococcus, Treponema, and Yersinia. Alternatively, the disease agent is a virus including for example human immunodeficiency virus, foot-and-mouth disease virus, avian influenza virus, and sheep pox virus.

The binding agent in various embodiments binds to and neutralizes an infectious agent and/or a disease agent associated with a pathology resulting from overexpression of a self protein in the subject such as an immunoglobulin, a leukocyte, a cytokine, and a growth factor. For example the overexpression is of an inflammatory agent such as a tumor necrosis factor (e.g., TnFa) or an interleukin (IL) such as IL-1Beta, or IL-6. Alternatively, an infectious agent and/or a disease agent is associated with expression of a mutated or modified molecule such as a protein, a sugar, a glycoprotein, or expression of a cell carrying a nucleotide sequence encoding the disease agent.

The binding agent in various embodiments binds to a cancer cell and/or cancer marker. For example the cancer cell includes a melanoma; a carcinoma (e.g., colon carcinoma); a pancreatic cancer; a sarcoma; a lymphoma; a leukemia; a brain tumor such as glioma; a lung cancer; an esophageal cancer; a mammary (breast) cancer; a bladder cancer; a prostate cancer; a head and neck cancer; an ovarian cancer; a kidney cancer; or a liver cancer.

The binding agents described herein are used in certain embodiments to treat symptoms of an autoimmune disease, a class of disorder which includes Hashimoto's thyroiditis; idiopathic myxedema, a severe hypothyroidism; multiple sclerosis, a demyelinating disease marked by patches or hardened tissue in the brain or the spinal cord; myasthenia gravis which is a disease having progressive weakness of muscles caused by autoimmune attack on acetylcholine receptors at neuromuscular junctions; Guillain-Barre syndrome, a polyneuritis; systemic lupus erythematosis; uveitis; autoimmune oophoritis; chronic immune thrombocytopenic purpura; colitis; diabetes; Grave's disease, which is a form of hypothyroidism; psoriasis; pemphigus vulgaris; and rheumatoid arthritis (RA).

It will be appreciated that in certain embodiments, the binding agent (e.g., peptide, protein, or portion or homolog thereof) of this invention can be obtained from a peptide synthesizer or any commercial supplier of custom peptides produced synthetically, e.g., by solid phase procedures. For example, peptide synthesis can be performed using various solid-phase techniques (Roberge et al. 995 Science 269:202) and automated synthesis may be achieved, for example, using the 431A peptide synthesizer (available from Applied Biosystems of Foster City, Calif.) in accordance with the instructions provided by the manufacturer. See also Horowitz et al. U.S. Pat. No. 8,131,480 issued Mar. 6, 2012.

### Molecule Target and Disease Agent Target

A molecule target and/or disease agent target is any target which is biological (e.g., protein, sugar, carbohydrate, DNA, RNA) or chemical to which the binding protein binds, and is any target associated with a disease, defect or negative condition. The molecule target or disease agent target is any molecule capable of being bound, or whose activity is altered (e.g., neutralized, reduced or ceased), or that can be recognized by immune effectors and leads for example to clearance, opsonization, killing, and phagocytosis. For example, the disease agent target in certain embodiments is a portion of a pathogen or a molecule released or secreted by the pathogen (e.g. toxin). A pathogen is an agent that causes a disease or condition, and includes a virus, cancer cell, bacterium, parasite or pathogenic protein. The disease agent target includes a pathogenic protein that is derived from normal cells, such as prions. The pathogenic protein or other molecule that is disease agent target is either independent of the pathogen or is associated with or produced by the pathogen.

In certain embodiments, the disease agent is a molecule (e.g, peptide) that is naturally produced by a plant or bacterium that inactivates or disrupts normal function of cellular membranes, cellular compartments, or cellular organelles. For example the disease agent disrupts function of ribosomes.

A virus is a microscopic particle that infects the cells of a biological organism and replicates in the host cell. In various embodiments, viral antigens including viral proteins, are targeted by the binding protein. Binding proteins bind to molecules or receptors on the virus, and are neutralized and/or cleared using the methods described herein. Examples of viruses that are neutralized and/or cleared by the binding protein herein include Influenza, Rhinovirus, Rubeola, Rubella, Herpes, Smallpox, Chickenpox, Human Papilloma, Rabies, and Human Immunodeficiency viruses.

A parasite is an organism that lives on or in a different organism. Parasites have or express molecules that are used as a target by the binding agent. Types of parasites include endoparasites (e.g., parasites that live inside the body of the host) and ectoparasites (e.g., parasites that live on the outside of the host's body). Examples of parasites that are treated by the methods, compositions, and kits herein are shown in Horvitz et al. US. patent publication 20110010782 published Jan. 13, 2011. Exemplary parasites include a protozoan (e.g., a plasmodium, a cryptosporidium, a microsporidium, and isospora), a tick, a louse and a parasitic worm.

Molecules on cancer cells also are targets of the binding agent. In related embodiments, the target is a protein on the cancer cell such as a cancer marker. Examples of proteins or receptors associated with cancer cells include CD33, HER2/neu, CA 125 (MUC16), prostate-specific antigen (PSA), and CD44.

The disease agent target in certain embodiments includes bacteria including Gram negative and Gram positive bacteria. Examples of pathogenic bacteria bound by the binding protein include Clostridium, Staphylococcus, Neisseria, Streptococcus, Moraxella, Listeria, any of the Enterobacteriaceae, Escherichia coli, Corynebacterium, Klebsiella, Salmonella, Shigella, Proteus, Pseudomonas, Haemophilus, Bordetella, Legionella, Campylobacter, Helicobacter, and Bacteroides. For example, the disease agent target is Bacillus anthracis (Decker, J. 2003 Deadly Diseases and Epidemics, Anthrax. Chelesa House Publishers. pages 1-112).

Enterohemorrhagic Escherichia coli (EHEC) is an emerging food- and water-borne pathogen that colonizes the distal ileum and colon and produces potent cytotoxins (Donnenberg, “Infections due to Escherichia coli and other enteric gram-negative bacilli,” in ACP Medicine, WebMD Professional Publishing, Danbury Conn., Chapter 7, pp. 8-1 to 8-18, 2005). After ingestion of contaminated food, humans develop symptoms ranging from mild diarrhea to the severe, and at times life-threatening, hemolytic uremic syndrome (HUS). Currently, EHEC is the most common cause of pediatric renal failure in the United States (Mead et al, Emerg Infect Dis, 5:607-625, 1999). Several EHEC serotypes cause disease, but the 0157 serotype is by far the most common cause of EHEC-related disease in North America, Europe and Japan (Feng, “Escherichia coli” in Garcia (ed.) Guide to Foodborne Pathogens. John Wiley and Sons, Inc., pp. 143-162, 2001). See also Waldor et al., US. patent publication number 2010/0092511A1 published Apr. 15, 2012, which is incorporated by reference herein in its entirety.

Shiga toxins are a family of related toxins with two major groups, Stx1 And Stx2 (Friedman et al., 2001 Curr Opin Microbiol 4 (2): 201-7). The toxins are named for Kiyoshi Shiga, who first described the bacterial origin of dysentery caused by Shigella dysenteriae. The most common sources for Shiga toxin are the bacteria S. dysenteriae and the Shigatoxigenic group of Escherichia coli (STEC), which includesserotypes 0157:147, 0104:H4, and other enterohemorrhagic E. coli, EHEC (Spears et al. 2006 HMS Microbiology Letter 187-202; Sandvig et al. 2000 EMBO J 19 (22): 5943-5950; and Krautz-Peterson et al. 2008 Infection and Immunity 76(5) 1931-1939; and Vermeij U.S. Pat. No. 7,807,184 issued Oct. 5, 2010, each of which is incorporated by reference herein in its entirety. Symptoms associated with Shiga toxin-exposure caused infection by EHEC include watery stool followed by severe abdominal pain and bloody stool. Exposed persons develop complications leading to HUS, encephalopathy, and even death (Masuda et al., U.S. Pat. No. 7,345,161 issued Mar. 18, 2008).

Methods for ascertaining the target molecule or disease agent are described herein and depend on the type of molecule being inhibited. For example, in a case in which a class or group of bacteria are to be inhibited, conserved regions of bacteria are targeted, and binding agents that bind to these targets are constructed. Methods for targeting a conserved region or polymorphic region of a nucleotide sequence that encodes the target molecule, or the target molecule having an amino acid sequence are shown in Cicciarelli et al., US. patent publication number 2005/0287129 A1 published Dec. 29, 2005 which is incorporated by reference herein in its entirety. In other embodiments, if a specific disease agent such as a bacterium is to be inhibited, a non-conserved region of the disease agent is targeted with the binding agents. The binding of the agents are determined and/or measured for example using standard assays, for example an enzyme-linked immunosorbent assay (ELISA), western blot and radioimmunoassay.

A molecule target or a disease agent target includes pathogenic molecules including polypeptides or toxins to which the binding protein described herein binds, neutralizes and/or clears. The term “pathogenic protein” refers to a protein that can cause, directly or indirectly, a disease, or condition in an individual. A pathogenic protein is for example a protein or a toxin produced by a bacterium, a virus, or a cancer cell. A recombinant multimeric binding protein described herein binds non-overlapping areas of the disease agent target (e.g., a toxin produced by a bacterium) and protects the subject from the pathology of the disease agent target by neutralizing and/or clearing the target. The binding protein protects subjects from negative symptoms caused by exposure to the disease agent target, and the risk of negative symptoms caused by a potential exposure to the target.

Anti-tag antibody described herein is used in various embodiments to effect or facilitate effector functions. The anti-tag antibody includes for example an immunoglobulin such as IgA, IgD, IgE, IgG, and IgM, and subtypes thereof. In addition to monoclonal antibodies, polyclonal antibodies specific to the tag are used in the methods, compositions and kits described herein. Effector functions are performed for example immune molecules interaction with the Fc portion of the immunoglobulin. Depending on the type of immunoglobulin chosen, the effector functions results in clearance of the disease agent (e.g., excretion, degradation, lysis or phagocytosis).

Mammalian antibody types IgA, IgD, IgE, IgG, and IgM, and antibody subtypes are classified according to differences in their heavy chain constant domains. Each immunoglobulin class differs in its biological properties and characteristics. IgA is found for example in areas containing mucus (e.g. in the gut, respiratory tract, and urogenital tract) and prevents the colonization of mucosal areas by pathogens. IgD functions as a disease agent receptor on B cells. IgE binds to allergens and triggers histamine release from mast cells and also provides protection against helminths (worms). IgG, in four forms, provides the majority of antibody-based immunity against invading pathogens. IgM has a very high affinity for eliminating pathogens in the early stages of B cell mediated immunity, and is expressed on the surface of B cells and also in a secreted form.

Leukocytes such as mast cells and phagocytes have specific receptors on the cell surface for binding antibodies. These Fc receptors interact with the Fc region of classes of antibodies (e.g. IgA, IgG, IgE). The engagement of a particular antibody with the Fc receptor on a particular cell triggers the effector function of that cell. For example, phagocytes function to perform phagocytosis, and mast cells function to degranulate. Effector functions generally result in destruction of an invading microbe. In various embodiments, the type of immunoglobulin is chosen specifically for a type of desired effector function.

The present invention includes methods of administering one or more recombinant multimeric binding proteins to a subject (e.g., human, cow, horse, pig, mouse, dog, and cat). The binding protein is administered in certain embodiments as a monomer, or as a multimeric binding protein comprising a plurality of monomers having different binding regions. The methods and compositions herein involve administration of one or more multimeric binding agents that include monomers that each has a binding region that is specific to the disease agent. The binding agent for example includes one or more tags. The binding agent/protein binds to the target region on the disease protein. Administration of two or more binding proteins (e.g., monomer binding proteins or multimeric binding proteins), in various embodiments, increased the effectiveness of the antibody therapy, and reduced the severity of one or more negative symptoms of exposure of the disease protein target. The binding protein is administered in various embodiments as a single monomer, a mixture of multiple (e.g., two or more) monomers, a multimeric binding protein including a plurality of monomers that are same or different, a mixture of multiple (e.g., two or more) multimeric binding proteins comprising more than one monomer, or any combination thereof. Examples herein show that administration of a binding protein containing more than one copy of the tag resulted in increased protection against a disease agent target, e.g., botulinum toxin serotype A. A single anti-tag antibody type in certain embodiments binds to all binding proteins having a tag. In certain embodiments in which the binding proteins have multiple copies (e.g., two or more) of the same tag, the anti-tag antibody binds to each copy of the tag on the binding protein. The phrase, “antibody therapeutic proteins” or “antibody therapeutic preparation” refers to one or more compositions that include at least one binding protein and optionally at least one anti-tag antibody. The multimeric binding protein preparation in certain embodiments contains additional elements including carriers as described herein.

The administration of the one or more binding proteins and/or anti-tag antibody is performed in related embodiments simultaneously or sequentially in time. The binding protein in certain embodiments is administered before, after or at the same time as another binding protein or the anti-tag antibody, providing that the binding proteins and/or the anti-tag antibodies are administered close enough in time to have the desired effect (e.g., before the binding proteins have been cleared by the body). Thus, the term “co-administration” is used herein to mean that the binding proteins and another binding protein or the anti-tag antibody are administered at time points to achieve effective treatment of the disease, and reduction in the level of the pathogen (e.g., virus, bacteria, cancer cell, proteins associated therewith, or combination thereof) and symptoms associated with it. The methods of the present invention are not limited by the amount of time in between which the binding proteins and/or anti-tag antibody are administered; providing that the compositions are administered close enough in time to produce the desired effect. In certain embodiment, the binding proteins is administered only, alternatively the binding protein and/or anti-tag antibody are premixed and administered together. The binding proteins and/or anti-tag antibody are in certain embodiments co-administered with other medications or compositions suitable to treating the disease agent.

The binding protein in certain embodiments is administered prior to the potential risk of exposure to the disease target agent to protect the subjects from symptoms of the disease agent target. For example, the binding protein and/or clearing antibody is administered minutes, hours or days prior to the risk of exposure. Alternatively, the binding protein is administered contemporaneously to the risk of exposure to the disease agent target, or slightly after the risk of exposure. For example, the binding protein is administered to a subject at the moment the subjects contacts, enters or passes through an environment (e.g., room, hallway, building, and field) containing the risk of exposure to the disease agent.

The methods of the present invention include treating a bacterial disease, a parasitic infection, a viral disease, a cancer, small unwanted molecule, a protein or a toxin associated therewith. This is accomplished by administering the binding proteins and anti-tag antibodies described herein to the affected individual or individual at risk. Administration ameliorates or reduces the severity of one or more the symptoms of the disease or condition. The presence, absence or severity of symptoms is measured for example using tests and diagnostic procedures known in the art. Presence, absence and/or level of the disease agent are measured in certain embodiments using methods known in the art. Symptoms or levels of the disease agent can be measured at one or more time points (e.g., before, during and after treatment, or any combination thereof) during the course of treatment to determine if the treatment is effective. A decrease or no change in the level of the disease agent, or severity of symptoms associated therewith indicates that treatment is working, and an increase in the level of the disease agent, or severity of symptoms indicates that treatment is not working. Symptoms and levels of disease agents are measured in various embodiments using methods known in the art. Symptoms that are monitored in certain embodiments include fever, plain including headache, joint pain, muscular pain, difficulty breathing, lethargy, and impaired mobility, appetite and unresponsiveness. Toxin protection is assessed as increased survival and reduction or prevention of symptoms. Methods, compositions and kits using the binding protein decrease and alleviate the symptoms of the disease target agent and also improve survival from exposure to the agent.

The antibody therapeutic agents including one or more binding proteins or agents, and/or an anti-tag antibody are administered in various embodiments with one or more pharmaceutical carriers. The terms “pharmaceutically acceptable carrier” and a “carrier” refer to any generally acceptable excipient or drug delivery device that is relatively inert and non-toxic. The binding agents and anti-tag antibody are administered with or without a carrier. Exemplary carriers include calcium carbonate, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.), the teachings of which are incorporated herein by reference in their entirety. The binding agents and anti-tag antibody are administered systemically or locally (e.g., by injection or diffusion).

Suitable carriers (e.g., pharmaceutical carriers) include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. The binding protein preparations are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. The binding protein preparations in certain embodiments are combined where desired with other active substances, e.g., enzyme inhibitors, to reduce metabolic degradation. A carrier (e.g., a pharmaceutically acceptable carrier) is used optionally in certain embodiments to administer one or more binding agents and an anti-tag antibody.

The binding agents and anti-tag antibodies in certain embodiments are administered topically (as by powders, ointments, or drops), orally, rectally, mucosally, sublingually, parenterally, intracisternally, intravaginally, intraperitoneally, bucally, ocularly, or intranasally, depending on preventive or therapeutic objectives and the severity and nature of a exposure or risk of exposure to the disease agent target. The composition in various embodiments is administered in a single dose or in more than one dose over a period of time to confer the desired effect.

An effective amount of compositions of the present invention varies according to choice of the binding agent, the particular composition formulated, the mode of administration and the age, weight and condition of the patient, for example. As used herein, an effective amount of the binding agents and/or anti-tag antibody is an amount which is capable of reducing one or more symptoms of the disease or conditions caused by the molecule target or disease agent target. Dosages for a particular patient are determined by one of ordinary skill in the art using conventional considerations, (e.g. by means of an appropriate, conventional pharmacological protocol).

A composition in certain embodiments includes one or more nucleotide sequences described herein that encode the binding protein. In various embodiments, a nucleotide sequence is either present as a mixture or in the form of a DNA molecule a multimer. A various embodiments, the composition includes a plurality of nucleotide sequences each encoding the binding protein including a monomer or polypeptide, or any combination of molecules described herein, such that the binding protein is generated in situ. In such compositions, a nucleotide sequence is administered using any of a variety of delivery systems known to those of ordinary skill in the art, including nucleic acid expression systems, bacterial and viral expression systems. Appropriate nucleic acid expression systems contain appropriate nucleotide sequences operably linked for expression in the patient (such as a suitable promoter and terminating signal). Bacterial delivery systems involve administration of a bacterium (such as Bacillus-Calmette-Guerrin) that expresses the polypeptide on its cell surface. In an embodiment, the DNA can be introduced using a viral expression system (e.g., vaccinia or other pox virus, retrovirus, or adenovirus), which uses a non-pathogenic (defective), replication competent virus. Techniques for incorporating DNA into such expression systems are well known to those of ordinary skill in the art. The DNA can also be “naked,” as described, for example, in Ulmer et al., Science 259:1745-1749, 1993 and reviewed by Cohen, Science 259:1691-1692, 1993. The uptake of naked DNA can be increased by coating the DNA onto biodegradable beads, which are efficiently transported into recipient cells.

Systems or kits of the present invention include in various embodiments one or more binding agents having a binding region and one or more tags, and an anti-tag antibody having an anti-tag region (e.g., an anti-tag antibody), as described herein.

The methods, compositions and kits described herein in certain embodiments include isolated polypeptide molecules that have been engineered or isolated to act as binding agents or binding proteins. A binding protein composition includes for example an amino acid sequence selected from SEQ ID NOs: 2, 4, 6, 8, 10, 12, 14, 20, 22, 24, 26, 28, 30, 32, 34, 36, 38, 40, 42, 44, 46, 48, 50, 52 or combinations thereof. In various embodiments, a binding protein composition includes a nucleotide sequence that encodes an amino acid sequence, for example the nucleotide sequence is selected from SEQ ID NOs: 1, 3, 5, 7, 9, 11, 13, 19, 21, 23, 25, 27, 29, 31, 33, 35, 37, 39, 41, 43, 45, 47, 49, 51 or combinations thereof. The bindings protein composition includes for example a tag, for example a tag having an amino acid sequence of SEQ ID NO:15.

As used herein, the term “polypeptide” encompasses amino acid chains of any length, including full length proteins (i.e., disease agents), in which the amino acid residues are linked by covalent peptide bonds. A polypeptide comprises a portion of the binding agent, the entire binding agent, or contains additional sequences. The polypeptides of the binding agents of the present invention referred to herein as “isolated” are polypeptides that are separated away and purified from other proteins and cellular material of their source of origin. The compositions and methods of the present invention also encompass variants of the above polypeptides and DNA molecules. A polypeptide “variant,” as used herein, is a polypeptide that differs from the recited polypeptide by having one or more conservative substitutions and/or modifications, such that the functional ability of the binding agent to bind to the disease agent target is retained.

The present invention also encompasses proteins and polypeptides, variants thereof, or those having amino acid sequences analogous to the amino acid sequences of binding agents described herein. Such polypeptides are defined herein as analogs (e.g., homologues), or mutants or derivatives. “Analogous” or “homologous” amino acid sequences refer to amino acid sequences with sufficient identity of any one of the amino acid sequences of the present invention so as to possess the biological activity (e.g., the ability to bind to the disease agent target). For example, an analog polypeptide can be produced with “silent” changes in the amino acid sequence wherein one, or more, amino acid residues differ from the amino acid residues of any one of the sequence, yet still possesses the function or biological activity of the polypeptide. The binding protein includes for example an amino acid having at least about 60% (e.g., 65%, 70%, 75%, 80%, 85%, 90% or 95%) identity or similarity with SEQ ID NOs: 2, 4, 6, 8, 10, 12, 14, 20, 22, 24, 26, 28, 30, 32, 34, 36, 38, 40, 42, 44, 46, 48, 50, 52, 56-87, 95 or combination thereof. Percent “identity” refers to the amount of identical nucleotides or amino acids between two nucleotides or amino acid sequences, respectfully. As used herein, “percent similarity” refers to the amount of similar amino acids between two amino acid sequences, i.e., having conservative amino acid changes compared to the original sequences, or to the amount of similar nucleotides between two nucleotide sequences.

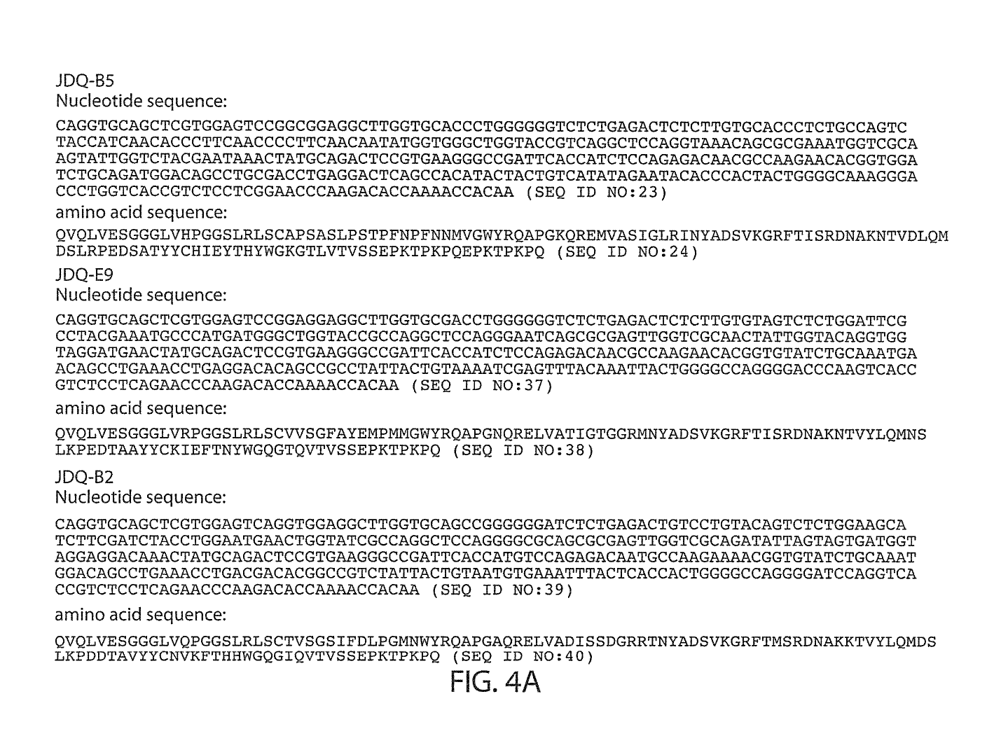

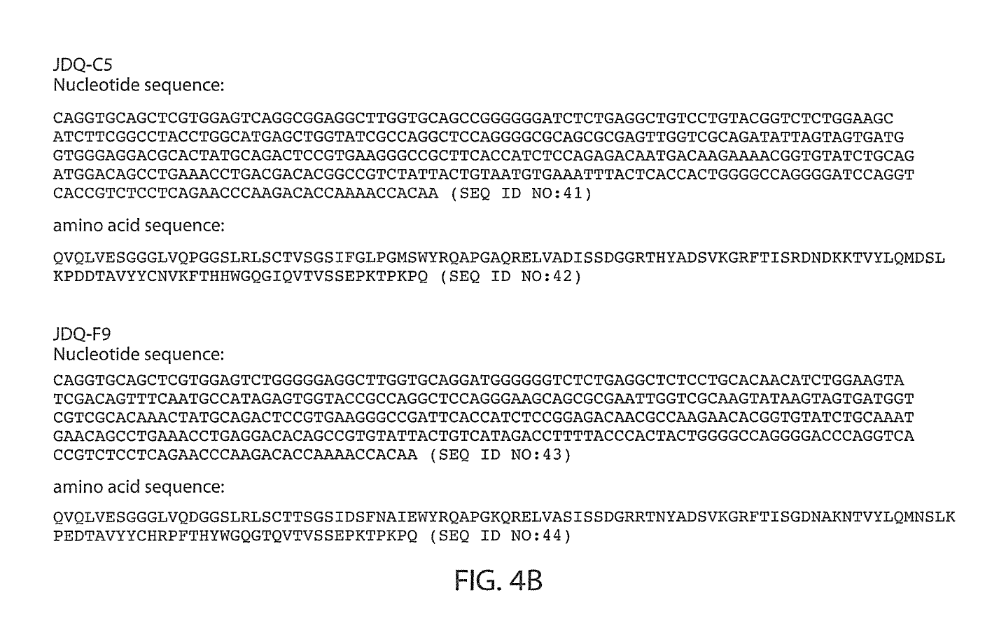

Referring to FIGS. 4 and 5, by comparing the B5 (SEQ ID NO: 24) polypeptide sequence to the other polypeptide sequences in the chart, the polypeptide sequence similarity is determined as follows: E-9 (SEQ ID NO: 38) is 74% similar, C5 (SEQ ID NO: 42) is 67% similar, B2 (SEQ ID NO: 40) is 68% similar, and F9 (SEQ ID NO: 44) is 73% similar. The BLAST was done using default parameters on the NCBI website. Since these VHHs have been shown to compete with B5, i.e., for binding to the target, the present invention includes those sequences having a sequence similarity of at least about 65%. In like manner, by comparing the B5 (SEQ ID NO: 23) nucleic acid sequence to the other nucleic acid sequences in the chart, the polypeptide sequence similarity is determined as follows: E-9 (SEQ ID NO: 37) is 81% identical, C5 (SEQ ID NO: 41) is 75% identical, B2 (SEQ ID NO: 39) is 86% identical, and F9 (SEQ ID NO: 43) is 80% identical. The present invention includes those nucleic acid sequences having a sequence identity of at least about 75%.

Homologous polypeptides are determined using methods known to those of skill in the art. Initial homology searches are performed at NCBI by comparison to sequences found in the GenBank, EMBL and SwissProt databases using, for example, the BLAST network service. Altschuler, S. F., et al., J. Mol. Biol., 215:403 (1990), Altschuler, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 1998 73:237-244, e.g., using default parameters). In certain embodiments, the recombinant multimeric binding protein acid sequence is an amino acid sequence that is substantially identical to sequences described herein, for example any of SEQ ID NOs: 56-87 and 95. The term “substantially identical” is used herein to refer to a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60% identity, or at least 75%, 85%, 95%, 96%, 98%, or 99% identity.

Calculations of sequence identity between sequences are performed as follows. To determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences are accomplished using a mathematical algorithm. Percent identity between two amino acid sequences is determined using an alignment software program using the default parameters. Suitable programs include, for example, CLUSTAL W by Thompson et al., Nuc. Acids Research 22:4673, 1994 (www.ebi.ac.uk/clustalw), BL2SEQ by Tatusova and Madden, FEMS Microbial. Lett. 174:247, 1999 (www.ncbi.nlm.nih.gov/blast/bl2seq/bl2.html), SAGA by Notredame and Higgins, Nuc. Acids Research 24:1515, 1996 (igs-server.cnrs-mrs.fr/˜enotred), and DIALIGN by Morgenstern et al., Bioinformatics 14:290, 1998 (bibiserv.techfak.uni-bielefeld.de/dialign).

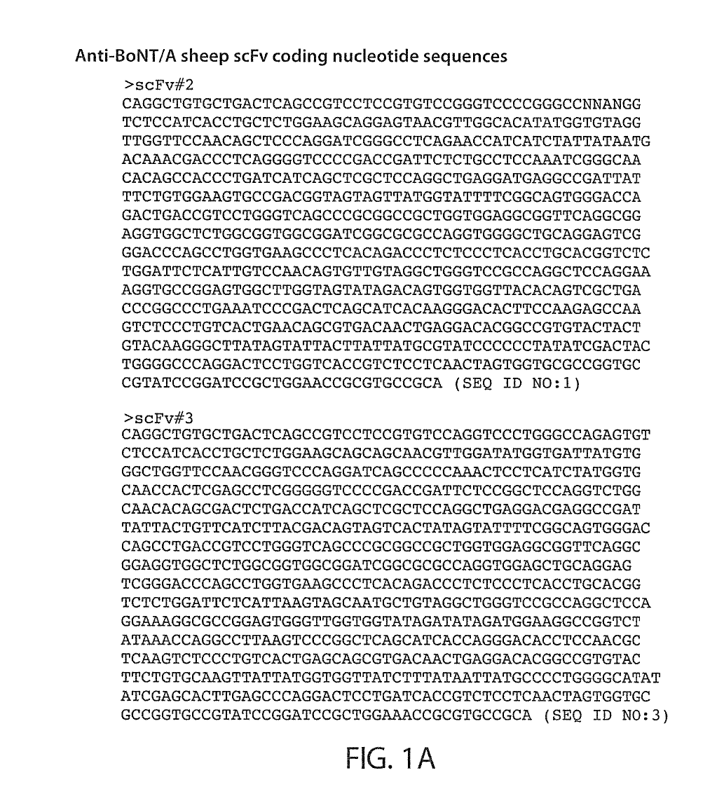

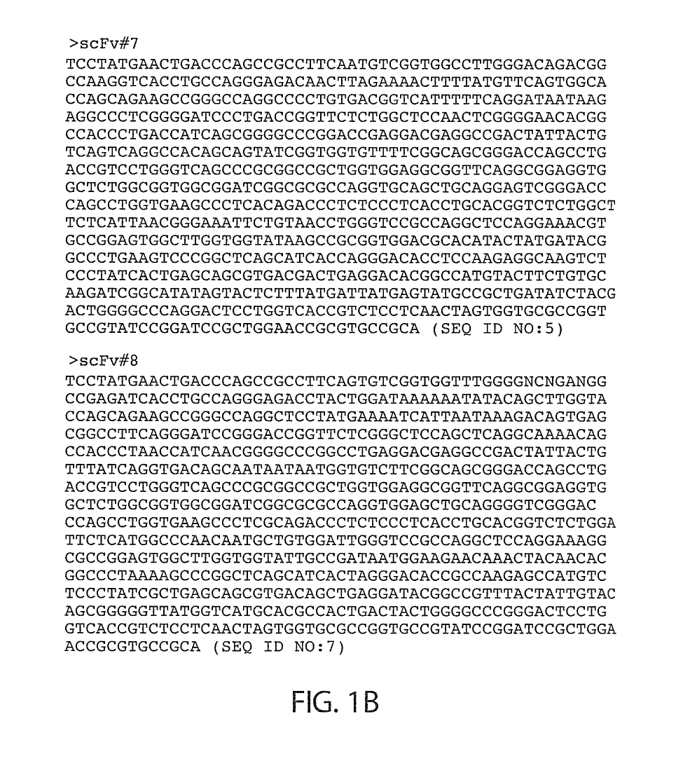

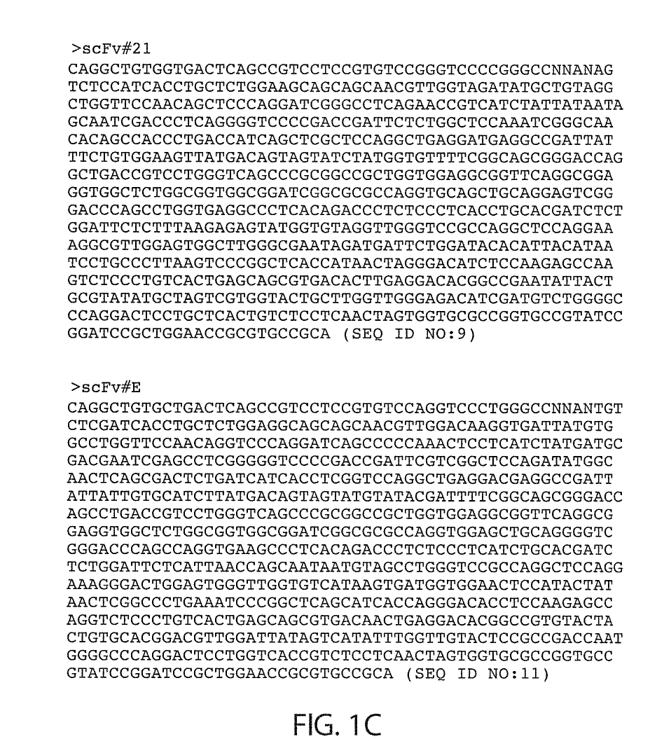

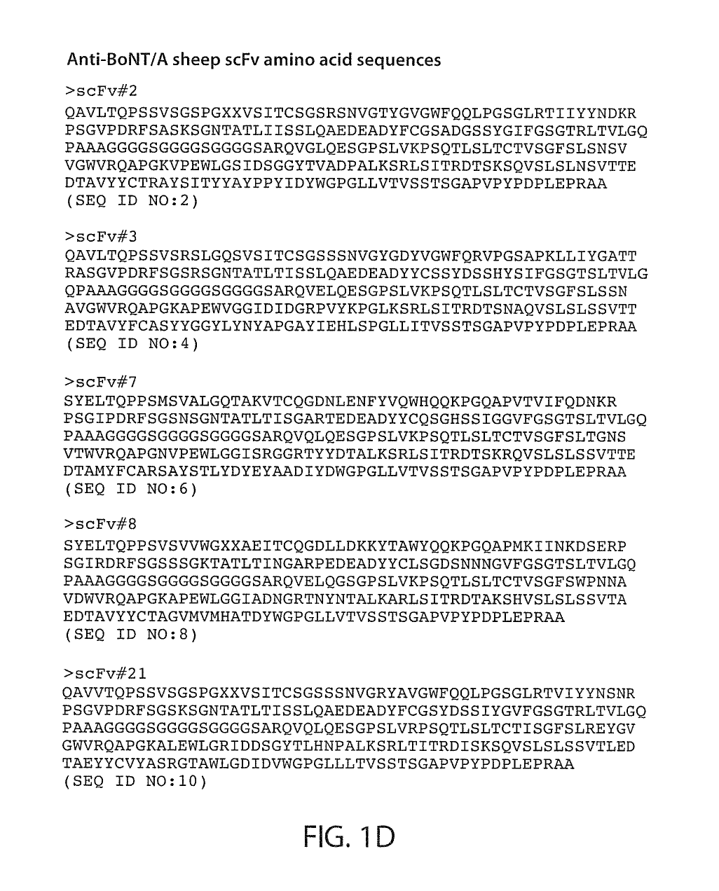

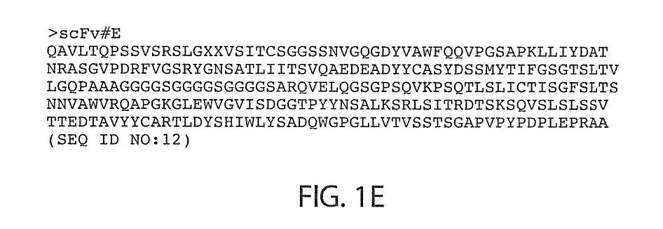

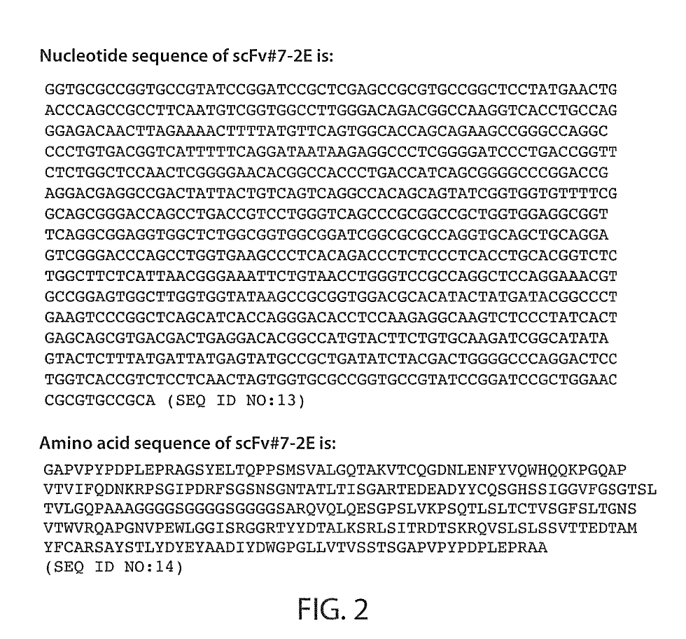

The methods, compositions and kits described herein in various embodiments include nucleotide sequence or an isolated nucleic acid molecule (encoding the binding protein) having a nucleotide sequence of SEQ ID NOs: 1, 3, 5, 7, 9, 11, 13, 19, 21, 23, 25, 27, 29, 31, 33, 35, 37, 39, 41, 43, 45, 47, 49, 51 or combinations thereof. See FIGS. 1, 3 and 4. As used herein, the terms “DNA molecule” or “nucleic acid molecule” include both sense and anti-sense strands, cDNA, genomic DNA, recombinant DNA, RNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as taught, for example, by Adelman et al. (DNA 2:183, 1983). Nucleotide variants are naturally occurring allelic variants, or non-naturally occurring variants. Variant nucleotide sequences in various embodiments exhibit at least about 70%, more preferably at least about 80% and most preferably at least about 90% homology to the recited sequence. Such variant nucleotide sequences hybridize to the recited nucleotide sequence under stringent conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses isolated nucleic acid sequences that encode the binding agents and in particular, those which encode a polypeptide molecule having an amino acid sequence of SEQ ID NOs: 2, 4, 6, 8, 10, 12, 14, 20, 22, 24, 26, 28, 30, 32, 34, 36, 38, 40, 42, 44, 46, 48, 50, 52, 56-87, 95 or combinations thereof.

As used herein, an “isolated” nucleotide sequence is a sequence that is not flanked by nucleotide sequences which in nature flank the gene or nucleotide sequence (e.g., as in genomic sequences) and/or has been completely or partially purified from other transcribed sequences (e.g., as in a cDNA or RNA library). Thus, an isolated gene or nucleotide sequence can include a gene or nucleotide sequence which is synthesized chemically or by recombinant means. Nucleic acid constructs contained in a vector are included in the definition of “isolated” as used herein. Also, isolated nucleotide sequences include recombinant nucleic acid molecules and heterologous host cells, as well as partially or substantially or purified nucleic acid molecules in solution. The nucleic acid sequences of the binding agents of the present invention include homologous nucleic acid sequences. “Analogous” or “homologous” nucleic acid sequences refer to nucleic acid sequences with sufficient identity of any one of the nucleic acid sequences described herein, such that once encoded into polypeptides, they possess the biological activity of any one of the binding agents described herein. In particular, the present invention is directed to nucleic acid molecules having at least about 70% (e.g., 75%, 80%, 85%, 90% or 95%) identity with SEQ ID NOs: 1, 3, 5, 7, 9, 11, 13, 19, 21, 23, 25, 27, 29, 31, 33, 35, 37, 39, 41, 43, 45, 47, 49, 51 or combinations thereof.

Also encompassed by the present invention are nucleic acid sequences, DNA or RNA, which are substantially complementary to the DNA sequences encoding the polypeptides of the present invention, and which specifically hybridize with their DNA sequences under conditions of stringency known to those of skill in the art. As defined herein, substantially complementary means that the nucleotide sequence of the nucleic acid need not reflect the exact sequence of the encoding original sequences, but must be sufficiently similar in sequence to permit hybridization with nucleic acid sequence under high stringency conditions. For example, non-complementary bases can be interspersed in a nucleotide sequence, or the sequences can be longer or shorter than the nucleic acid sequence, provided that the sequence has a sufficient number of bases complementary to the sequence to allow hybridization therewith. Conditions for stringency are described in e.g., Ausubel, F. M., et al., Current Protocols in Molecular Biology, (Current Protocol, 1994), and Brown, et al., Nature, 366:575 (1993); and further defined in conjunction with certain assays.

The invention also provides vectors, plasmids or viruses containing one or more of the nucleic acid molecules having the sequence of SEQ ID NO:1, 3, 5, 7, 9, 11, 13, 19, 21, 23, 25, 27, 29, 31, 33, 35, 37, 39, 41, 43, 45, 47, 49, 51 or combinations thereof). Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

Any of a variety of expression vectors known to those of ordinary skill in the art can be employed to express recombinant polypeptides of this invention. Expression can be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast, insect cells, or a mammalian cell line such as COS or CHO. The DNA sequences expressed in this manner can encode any of the polypeptides described herein including variants thereof.

Uses of plasmids, vectors or viruses containing the nucleic acids of the present invention include generation of mRNA or protein in vitro or in vivo. In related embodiments, the methods, compositions and kits encompass host cells transformed with the plasmids, vectors or viruses described above. Nucleic acid molecules can be inserted into a construct which can, optionally, replicate and/or integrate into a recombinant host cell, by known methods. The host cell can be a eukaryote or prokaryote and includes, for example, yeast (such as Pichia pastoris or Saccharomyces cerevisiae), bacteria (such as E. coli, or Bacillus subtilis), animal cells or tissue, insect Sf9 cells (such as baculoviruses infected SF9 cells) or mammalian cells (somatic or embryonic cells, Human Embryonic Kidney (HEK) cells, Chinese hamster ovary cells, HeLa cells, human 293 cells and monkey COS-7 cells). Host cells suitable in the present invention also include a mammalian cell, a bacterial cell, a yeast cell, an insect cell, and a plant cell.

The nucleic acid molecule can be incorporated or inserted into the host cell by known methods. Examples of suitable methods of transfecting or transforming cells include calcium phosphate precipitation, electroporation, microinjection, infection, lipofection and direct uptake. “Transformation” or “transfection” as used herein refers to the acquisition of new or altered genetic features by incorporation of additional nucleic acids, e.g., DNA. “Expression” of the genetic information of a host cell is a term of art which refers to the directed transcription of DNA to generate RNA which is translated into a polypeptide. Methods for preparing such recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), for example.

The host cell is maintained under suitable conditions for expression and recovery of the polypeptides of the present invention. In certain embodiments, the cells are maintained in a suitable buffer and/or growth medium or nutrient source for growth of the cells and expression of the gene product(s). The growth media are not critical to the invention, are generally known in the art and include sources of carbon, nitrogen and sulfur. Examples include Luria-Bertani broth, Superbroth, Dulbecco's Modified Eagles Media (DMEM), RPMI-1640, M199 and Grace's insect media. The growth media can contain a buffer, the selection of which is not critical to the invention. The pH of the buffered Media can be selected and is generally one tolerated by or optimal for growth for the host cell.

The host cell is maintained under a suitable temperature and atmosphere. Alternatively, the host cell is aerobic and the host cell is maintained under atmospheric conditions or other suitable conditions for growth. The temperature is selected so that the host cell tolerates the process and is for example, between about 13-40° Celsius.

The invention having now been fully described, it is further illustrated by the following claims and by the examples, which are found in a paper published in the Public Library of Science (PLoS) One and entitled, “A Novel Strategy for Development of Recombinant Antitoxin Therapeutics Tested in a Mouse Botulism Model”, co-authored by Jean Mukherjee, Jacqueline M. Tremblay, Clinton E. Leysath, Kwasi Ofori, Karen Baldwin, Xiaochuan Feng, Daniela Bedenice, Robert P. Webb, Patrick M. Wright, Leonard A. Smith, Saul Tzipori, and Charles B. Shoemaker (12 pages; Mukherjee J et al. 2012 PLoS ONE 7(1): e29941. doi:10.1371/journal.pone.0029941). This published paper is hereby incorporated by reference herein in its entirety.

Numerous embodiments of the invention are provided herein including Appendix B (11 pages) which is attached hereto. Appendix B is a document entitled “Sequences of new toxin-binding VHH proteins and coding DNAs” prepared by Prof. Charles Shoemaker.

Plants species have evolved chemical defenses against other organisms (Linskens, Hans F.; Jackson, John F. (Eds.) Plant Toxin Analysis 1992, XXVI, 389 p. 33 illus). Plants contain and secrete a variety of toxic compounds sometimes referred to as called “secondary compounds” that affect the behavior and productivity of wild and domestic animals. Classes of toxic compounds include soluble phenolics, alkaloids, and terpenoids. Soluble phenolics include flavonoids, isoflavonoids, and hydrolysable and condensed tannins.

Exemplary plants toxin molecules that in certain embodiments are treated using the compositions, methods and kits described herein are: Akar saga (Abrus precatorius), Deathcamas, Amianthium Angel's Trumpet (Brugmansia), Angel Wings (Caladium), Anticlea, Autumn crocus (Colchicum autumnale), Azalea (Rhododendron), Bittersweet nightshade (Solanum dulcumara), Black hellebore (Helleborus niger), Black locust (Robinia pseudoacacia), Black nightshade (Solanum nigrum), Bleeding heart (Dicentra cucullaria), Blind-your-eye mangrove (Excoecaria agallocha), Blister Bush (Peucedanum galbanum), Bloodroot (Sanguinaria canadensis), Blue-green algae (Cyanobacteria), Bobbins (Arum maculatum). Bracken (Pteridium aquilinum), Broom (Cytisus scoparius), calabar bean (Physostigma venenosum), castor bean, Christmas rose (Helleborus niger), Columbine (Aquilegia), Corn cockle (Agrostemma githago), corn lily (veratrum), cowbane (Cicuta), cows and bulls (Arum maculatum), crab's eye (Abrus precatorius), cuckoo-pint (Arum maculatum), daffodil (Narcissus), Darnel (Latium temulentum), Deadly nightshade (Atropa belladonna), Devils and angels (Arum maculatum), False acacia (Robinia pseudoacacia), False hellebore (Veratrum), Foxglove (Digitalis purpurea), Frangipani (Plumeria), Doll's eyes (Actaea pachypoda), Dumbcane (Dieffenbachia), Dutchman's breeches (Dicentra cucullaria), Elder/Elderberry (Sambucus), Giant hogweed (Heracleum mantegazzianum), Giddee giddee, Gifblaar (Dichapetalum cymosum), Greater celandine (Chelidonium majus), Gympie gympie (Dendrocnide moroides), Heart of Jesus (Caladium), hemlock (Conium maculatum), hemlock water-dropwort (Oenanthe crocata), henbane (Hyoscyamus niger), Horse chestnut (Aesculus hippocastanum), Holly (Ilex aquifolium), Hyacinth (Hyacinthus orientalis), Indian licorice, Jack in the pulpit, Jamestown weed, jequirity, Jerusalem cherry, Jimson weed, John Crow bead, Jumbie bead, Lily of the Valley, Lords and Ladies, Madiera winter cherry, Mayapple, Meadow saffron, Milky mangrove, Monkshood, Moonseed, Passion flower, Plumeria, Poison hemlock, Poison ivy, Poison oak, Poison parsnip, Poison sumac, Poison ryegrass, Pokeweed, Precatory bean, Privet, ragwort, redoul, River poison tree, Robinia pseudoacacia (also known as black locust and false acacia), Rosary pea, Sosnowsky's Hogweed, Spindle tree, Starch-root, Stenanthium, Stinging tree, Stinkweed, Strychnine tree, Suicide tree (Cerbera odollam), thorn apple, Toxicoscordion, Wake robin, Water hemlock, White baneberry, White snakeroot, Wild arum, Winter cherry, Wolfsbane, Yellow Jessamine, Yew, and Zigadenus

Abrus precatorius is known commonly as jequirity, crab's eye, rosary pea, ‘John Crow’ bead, precatory bean, Indian licorice, akar saga, giddee giddee, jumbie bead, ruti, and weather plant. The attractive seeds (usually about the size of a ladybug, glossy red with one black dot) contain abrin, which is related to ricin, a very potent toxic substance to humans as a single seed can kill an adult human. Symptoms of poisoning include nausea, vomiting, convulsions, liver failure, and death, usually after several days. The seeds have been used as beads in jewelry, which is dangerous; inhaled dust is toxic and pinpricks can be fatal. The seeds are unfortunately attractive to children.

Aconitum species, commonly called aconite, wolfsbane and monkshood are poisonous even by casual skin contact should be avoided; symptoms include numbness, tingling, and cardiac irregularity. The toxin is an alkaloid called aconitine, which disables nerves, lowers blood pressure, and can stop the heart. It has been used as poison for bullets (by Germany in WWII), as a bait and arrow poison (ancient Greece), and to poison water supplies (reports from ancient Asia). If ingested, it usually causes burning, tingling, and numbness in the mouth, followed by vomiting and nervous excitement.

Actaea pachypoda known as doll's eyes or white baneberry are poisonous berries, and other parts of the plant are toxic. Consumption of the berries has a sedative effect on cardiac muscle tissue and can cause cardiac arrest.

Adam and Eve (Arum maculatum) is a common woodland plant species of the Araceae family. It is widespread across temperate northern Europe and is known by an abundance of common names including Wild arum, Lords and Ladies, Devils and Angels, Cows and Bulls, Cuckoo-Pint, Adam and Eve, Bobbins, Naked Boys, Starch-Root and Wake Robin, Adenium obesum (also known as sabi star, kudu or desert-rose). The plant exudes a highly toxic sap which is used by the Meridian High and Hadza in Tanzania to coat arrow-tips for hunting.

Aesculus hippocastanum (horse-chestnut) produces a toxin causing nausea, muscle twitches, and sometimes paralysis. Ageratina altissima (white snakeroot) produces a toxin, causing nausea and vomiting. Milk from cattle that have eaten white snakeroot can sicken or kill humans. Aquilegia (columbine) seeds and roots that contain cardiogenic toxins causing both severe gastroenteritis and heart palpitations if consumed, and poisoning by this plant is often fatal. Areca catechu (betel nut palm and pinyang) a nut contains an alkaloid related to nicotine which is addictive, mildly intoxicating, and if swallowed causes nausea. Use is correlated with mouth cancer, asthma and heart disease. Arum maculatum (jack in the pulpit) bright red berries contain oxalates of saponins and causes skin, mouth and throat irritation, resulting in swelling, burning pain, breathing difficulties and stomach upset. Atropa belladonna (deadly nightshade, and belladonna) is one of the most toxic plants found in the Western hemisphere, producing tropane alkaloids including atropine, hyoscine (scopolamine), and hyoscyamine, which have anticholinergic properties. The consumption of two to five berries by children and ten to twenty berries by adults can be lethal. The symptoms of poisoning include dilated pupils, sensitivity to light, blurred vision, tachycardia, loss of balance, staggering, headache, rash, flushing, dry mouth and throat, slurred speech, urinary retention, constipation, confusion, hallucinations, delirium, and convulsions. Ingestion of a single leaf of the plant can be fatal to an adult, and casual contact with the leaves causes skin pustules. The berries pose the greatest danger to children because they look attractive and have a somewhat sweet taste. In 2009 a case of A. belladonna mistaken for blueberries, with six berries ingested by an adult woman, resulted in severe anticholinergic syndrome. A. belladonna is toxic also to many domestic animals, causing narcosis and paralysis. Brugmansia (angel's trumpet) contains the tropane alkaloids scopolamine and atropine, and can be fatal.

Caladium (commonly known as angel wings, elephant ear and heart of Jesus) produces symptoms such as generally irritation, pain, and swelling of tissues in subjects. If the mouth or tongue swell, breathing may be fatally blocked. Cerbera odollam (suicide tree) produces seeds that contain cerberin, a potent alkaloid toxin related to digoxin. The poison blocks the calcium ion channels in heart muscle, causing disruption of the heart beat which is typically fatal. Chelidonium majus also known as greater celandine is toxic in moderate doses as it contains a range of isoquinoline alkaloids. The main alkaloid present in the herb and root is coptisine, with berberine, chelidonine, sanguinarine and chelerythrine also present. Sanguinarine is particularly toxic with an LD₅₀ of only 18 mg per kg body weight. Cicuta (water hemlock, cowbane, wild carrot, snakeweed, poison parsnip, false parsley, children's bane and death-of-man) is extremely poisonous and contains the toxin cicutoxin, a central nervous system stimulant, resulting in seizures. Colchicum autumnale (autumn crocus and meadow saffron) bulbs contain colchicine, having poisoning symptoms that include burning in the mouth and throat, fever, vomiting, diarrhea, abdominal pain and kidney failure. There is no specific antidote for colchicine, although various treatments do exist. Conium maculatum (commonly known as hemlock, poison hemlock, spotted parsley, spotted cowbane, bad-man's oatmeal, poison snakeweed and beaver poison) contains the alkaloid coniine which causes stomach pains, vomiting, and progressive paralysis of the central nervous system. Consolida commonly known as larkspur is a poisonous plant that causes nausea, muscle twitches, paralysis and is often fatal.

Convallaria majalis (lily of the valley) is a poisonous woodland flowering plant that contains cardiac glycosides fatal in humans. Coriaria myrtifblia (redoul) contains the toxin coriamyrtin. Ingestion of this plant produces digestive, neurological and respiratory problems. The poisonous fruit resemble blackberries and are often mistakenly eaten by children and adults. Cyanobacteria, a phylum of bacteria, is commonly known as blue-green algae. Many different species, including Anacystis cynea and Anabaena circinalis, produce several different toxins known collectively as cyanotoxins. Cyanotoxins include neurotoxins, hepatotoxins, endotoxins and cytotoxins. Cytisus scoparius (commonly known as broom or common broom) contains toxic alkaloids that depress the heart and nervous system. The alkaloid sparteine is a class 1a antiarrhythmic agent and a sodium channel blocker. The berries of Daphne are either red or yellow and are poisonous, causing burns to mouth and digestive tract, followed by coma. Datura contains the alkaloids scopolamine and atropine. Datura has been used as a hallucinogenic drug by the native peoples of the Americas and others. Incorrect consumption of this plant can lead to death. Datura stramonium (jimson weed, thorn apple, stinkweed and Jamestown weed) causes abnormal thirst, vision distortions, delirium, incoherence, and coma. Deathcamas, including Amianthium, Anticlea, Stenanthium, Toxicoscordion and Zigadenus, are poisonous in many cases due to the presence of alkaloids in the plants. Ingestion of the plant by grazing animals, such as sheep and cattle, often results in death.

Delphinium (also known as larkspur) contains the alkaloid delsoline. Young plants and seeds of Delphinium are poisonous, causing nausea, muscle twitches, and paralysis. Dendrocnide moroides (also known as stinging tree and gympie gympie) causes a painful sting when touched and in some cases of widespread contact may be fatal. The stinging may last for several days and is exacerbated by touching, rubbing, and cold. Dicentra cucullaria (also known as bleeding heart and Dutchman's breeches) has leaves and roots that are poisonous and cause convulsions and other nervous symptoms. Dichapetalum cymosum (also known as gifblaar) is a well known as a livestock poison in South Africa. The plant contains the metabolic poison fluoroacetic acid. Dieffenbachia (a houseplant dumbcane) causes intense burning, irritation, and immobility of the tongue, mouth, and throat. Swelling can be severe enough to block breathing, leading to death. Digitalis purpurea (foxglove) leaves, seeds, and flowers are poisonous, containing cardiac or other steroid glycosides. These cause irregular heartbeat, general digestive upset, and confusion. Euonymus europaeus (commonly known as spindle, European spindle or spindle tree). produces a poisonous fruit that contains amongst other substances, the alkaloids theobromine and caffeine, as well as an extremely bitter terpene. Poisoning by this plant is more common in young children, who are enticed by the brightly-coloured fruit of the plant. Ingestion of the fruit results in liver and kidney damage and even death.

Excoecaria agallocha (milky mangrove) has a milky sap that causes skin irritation and blistering. Eye contact with the sap can even cause temporary blindness. Gelsemium sempervirens commonly known as yellow jessamine is poisonous, causing nausea, vomiting and even death. Hedera helix (English ivy) contains leaves and berries that can be poisonous, causing stomach pains, labored breathing, possible coma. Helleborus niger (Christmas rose) contains protoanemonin or ranunculin, which has an acrid taste and can cause burning of the eyes, mouth and throat, oral ulceration, gastroenteritis and hematemesis. Heracleum mantegazzianum (giant hogweed) produces a sap that is phototoxic, causing phytophotodermatitis (severe skin inflammations) when affected skin is exposed to sunlight or to UV-rays. Presence of minute amounts of sap in the eyes can lead to temporary or even permanent blindness. Hippomane mancinella (manehineel) contains toxic phorbol esters typical of the Euphorbiaceae plant family. Contact with the milky white sap of the plant produces strong allergic dermatitis. The fruit is fatal if eaten. Hyacinthus orientalis (hyacinth) bulbs are poisonous, causing nausea, vomiting, gasping, convulsions, and possibly death. Even handling the bulbs can cause skin irritation.

Hyoscyamus niger (henbane) has seeds and foliage contain hyoscyamine, scopolamine and other tropane alkaloids that produces dilated pupils, hallucinations, increased heart rate, convulsions, vomiting, hypertension and ataxia. Ilex aquifolium (European holly) berries cause gastroenteritis, resulting in nausea, vomiting and diarrhea. Jacobaea vulgaris (ragwort) contains alkaloids, including jacobine, jaconine, jacozine, otosenine, retrorsine, seneciphylline, senecionine, and senkirkine. Kalanchoe delagoensis (mother of millions) contains bufadienolide cardiac glycosides which cause cardiac poisoning, particularly in grazing animals. Kalmia latifolia (mountain laurel) contains andromedotoxin and arbutin. The green parts of the plant, flowers, twigs, and pollen are all toxic, and symptoms of toxicity begin to appear about six hours following ingestion. Poisoning produces anorexia, repeated swallowing, profuse salivation, depression, uncoordination, vomiting, frequent defecation, watering of the eyes, irregular or difficulty breathing, weakness, cardiac distress, convulsions, coma, and eventually death. Laburnum produces seeds that are poisonous and are lethal if consumed in excess. The main toxin in the seeds is cytisine, a nicotinic receptor agonist. Symptoms of poisoning may include intense sleepiness, vomiting, excitement, staggering, convulsive movements, slight frothing at the mouth, unequally dilated pupils, coma and death. Ligustrum (privet) berries and leaves that are poisonous. The berries contain syringin, which causes digestive disturbances, nervous symptoms. Privet is one of several plants which are poisonous to horses. Lolium temulentum (poison ryegrass) produces seeds that contain the alkaloids temuline and loliine. The fungus ergot grow on the seed heads of rye grasses, as an additional source of toxicity.

Mango peel and sap contains urushiol, the chemical in poison ivy and poison sumac that can cause urushiol-induced contact dermatitis in susceptible people. Cross-reactions between mango contact allergens and urushiol have been observed. Those with a history of poison ivy or poison oak contact dermatitis may be most at risk for such an allergic reaction. Narcissus bulbs and stems are poisonous, and cause nausea, vomiting, diarrhea, headaches, vomiting, and blurred vision.

Oenanthe crocata (hemlock water dropwort) contains oenanthotoxin in the stems and especially the carbohydrate-rich roots that are poisonous. Peucedanum galbanum (commonly known as blister bush) is poisonous and contact to the body causes painful blistering that is intensified with exposure to sunlight. Physostigma venenosum (calabar bean) contains parasympathomimetic alkaloid physostigmine toxin, a reversible cholinesterase inhibitor. Symptoms of poisoning include copious saliva, nausea, vomiting, diarrhea, anorexia, dizziness, headache, stomach pain, sweating, dyspepsia and seizures. Phytolacca (pokeweed) leaves, berries and roots contain phytolaccatoxin and phytolaccigenin. Ingestion of poisonous parts of the plant cause severe stomach cramping, persistent diarrhoea, nausea, vomiting (sometimes bloody vomiting), slow and difficult breathing, weakness, spasms, hypertension, severe convulsions, and even death. Podophyllum peltatum (mayapple) contains the non-alkaloid toxin podophyllotoxin, which causes diarrhea, severe digestive upset. Pteridium aquilinum (commonly known as bracken) if ingested is carcinogenic to humans and animals such as mice, rats, horses and cattle. The carcinogenic compound in the pant is ptaquiloside (PTQ), which can leach from the plant into the water supply. Pteridium aquilinum (African sumac) is closely related to poison ivy. The tree contains low levels of a highly irritating oil with urushiol. Skin reactions to contacting the plan include blisters and rashes that be further spread by contacting clothing of an exposed subjects. The smoke of burning Rhus lancia can cause reactions in the lungs, and can be fatal. Ricinus communis (castor oil plant) seeds contain ricin, an extremely toxic water-soluble protein, ricinine, an alkaloid, and an irritant oil.

Sambucus (commonly known as elder or elderberry) roots are poisonous and cause nausea and digestive upset. Sanguinaria canadensis (bloodroot) rhizome or stem contains morphine-like benzylisoquinoline alkaloids, and the toxin sanguinarine. Sanguinarine kills animal cells by blocking the action of Na+/K+-ATPase transmembrane proteins. Solanum dulcamara (bittersweet nightshade) contains solanine which causes fatigue, paralysis, convulsions, and diarrhea in subjects exposed to the plant. Solanum nigrum (black nightshade) contains the toxic glycoalkaloid solanine. Solanine poisoning is primarily displayed by gastrointestinal and neurological disorders. Symptoms include nausea, diarrhea, vomiting, stomach cramps, burning of the throat, cardiac dysrhythmia, headache and dizziness. Taxus baccata (yew) contains toxic taxanes. The plant seeds themselves are particularly toxic if chewed. Toxicodendron contain a highly irritating oil with urushiol. Species of toxicodendrons include Toxicodendron radicans (commonly known as poison ivy), Toxicodendron diversilobum (commonly known as poison-oak), and Toxicodendron vernix (commonly known as poison sumac. These plants cause skin reactions such as blisters and rashes. Urtica ferox (ongaonga) cause a painful sting that lasts several days. Veratrum (false hellebore and corn lily) contain a highly toxic steroidal alkaloids (e.g. veratridine) that activate sodium ion channels and cause rapid cardiac failure and death if ingested. Symptoms typically occur between 30 minutes and four hours after ingestion and include nausea and vomiting, abdominal pain, numbness, headache, sweating, muscle weakness, bradycardia, hypotension, cardiac arrhythmia, and seizures. Xanthium (commonly known as cocklebur) includes X. strumarium, a native of North America, that is poisonous to livestock, including horses, cattle, and sheep. The seedlings and seeds are the most toxic parts of the plants and produce unsteadiness and weakness, depression, nausea and vomiting, twisting of the neck muscles, rapid and weak pulse, difficulty breathing, and eventually death. Zantedeschia (Calla lily) contain calcium oxalate and other toxins that producing irritation and swelling of the mouth and throat, acute vomiting and diarrhea.

Endosperm of castor seeds contain two highly toxic proteins (Ghetie et al. U.S. Pat. No. 5,578,706 issued Nov. 26, 1996; and Lord et al., 1994 The FASEB Journal 8, 201-208, each of which is incorporated by reference herein in its entirety). R. communis agglutinin (RCA), a 120 kDa hemagglutinin lectin, and ricin, a 65 kDa cytotoxic lectin, are lethal to eukaryotic cells. Ricin has two polypeptide chains, A and B, which together are highly lethal to mammalian cells. Agglutinin protein has four polypeptides, linked by disulfide bonds (Butterworth et al., 1983 Eur. J. Biochem. 137, 57-65), two of which are similar to the A chain (ricin A chain; RTA) and two similar to the B chain of ricin (ricin B chain, RTB). The A and B chains of ricin, together. Ricin E is a variant of the ricin toxin, having an A chain similar to ricin and a B chain, which is a hybrid of the ricin and RCA and B chains (Ladin et al. 1987 Plant Molecular Biology 9: 287-295).

Ricin A chain (32 kDa) has a ribosome-inactivating activity (Lord et al., 1994 The FASEB Journal 8, 201-208), irreversibly altering the ribosomal RNA subunits involved in translation. The A chain specifically binds 28S ribosomal subunits; the A chain requires the B chain to enter the cells as a heterodimeric toxin.

Ricin's B chain is a lectin which specifically binds glycoproteins and glycolipids on the cell surface terminating in galactose or N-acetylgalactosamine (Lord et al., 1994 The FASEB Journal 8, 201-208). The B chain binds more strongly to complex galatosides than to simple sugars. The B chain has four disulfide bonds and has a galactose/N-acetylgalactosamine binding activity. The N-terminal and C-terminal halves of the B chain contain 41 homologous pairs of amino acids when the two disulfide bonds in each half are aligned, yielding a bilobal structure with two galactose binding sites. Subdomains formed by the four disulfide bonds represent a conserved peptide which is repeated four times (Roberts et al., 1985 Journal of Biological Chemistry 260, 15682-8). Up to 108 ricin B chains bind to an individual cell by hydrogen bonds (Lord et al., 1994 The FASEB Journal 8, 201-208). The B chain attaches to the eukaryotic cell and the intact toxin enters the cell by receptor mediated endocytosis (Bilge et al., 1995 Journal of Biological Chemistry 1995; 270(40):23720-23725). The B chain protects the A chain from proteolytic activities of lysosomes and cathepsins. Mannose residues attached to ricin are bound by cellular mannose receptors and initiate endocytosis (Montfort et al., 1987 Journal of Biological Chemistry 262, 5398-403).

Numerous embodiments of the invention are provided in examples herein and published in the journal Infection and Immunity entitled, “A single VHH-based toxin neutralizing agent and an effector antibody protects mice against challenge with Shiga toxins 1 And 2” by Jacqueline M. Tremblay, Jean Mukherjee, Clinton E. Leysath, Michelle Debatis, Kwasi Ofori, Karen Baldwin, Courtney Boucher, Rachel Peters, Gillian Beamer, Daniela Bedenice, Saul Tzipori, and Charles B. Shoemaker, Infect. Immun. IAI.01033-13; September 2013 the contents of which are incorporated herein in its entirety.

Shiga toxin-producing E. coli (STEC) bacteria cause both sporadic and major outbreaks of diarrheal disease through consumption of contaminated food or water. For example, in 2011, an outbreak of STEC in Germany was due to contaminated sprouts (Buchholz U, et al. 2011 N Engl J Med 365: 1763-1770, Frank C, et al. 2011 N Engl J Med 365: 1771-1780). STEC (which include enterohemorrhagic E. coli or EHEC) infection generally causes acute bloody diarrhea and abdominal cramping. In about 2-10% of patients, mostly children and elderly, hemolytic-uremic syndrome (HUS) develops as a complication, which is characterized by acute renal failure, hemolytic anemia, and thrombocytopenia. HUS is a severe complication and requires blood transfusion, kidney dialysis, and in some cases kidney transplantation. The major virulence determinents of STEC are attributed to Shiga toxins Stx1 And Stx2 (Karmali M A, et al. 1985 J Infect Dis 151: 775-782). Both toxins contribute to disease in animal models (Melton-Celsa A, et al. 2011 Current topics in microbiology and immunology), but in humans Stx2 is more often linked to HUS (Boerlin P, et al. 1999 Journal of clinical microbiology 37: 497-503; Friedrich A W, et al. 2002 J Infect Dis 185: 74-84; Hedican E B, et al. 2009 Clinical infectious diseases: an official publication of the Infectious Diseases Society of America 49: 358-364; Kawano K, et al. 2008 European journal of clinical microbiology & infectious diseases: official publication of the European Society of Clinical Microbiology 27: 227-232)

Stx1 And Stx2 each consist of an A subunit N-glycosidase and five B subunits that bind to the Gb3 receptor leading to cell internalization (Lingwood C A, et al. 1987 J Biol Chem 262: 8834-8839; Cohen A, et al. 1987 J Biol Chem 262: 17088-17091) and inhibition of protein synthesis which triggers apoptosis (Melton-Celsa A, et al. 2011. Nat Rev Microbiol 8: 105-116, Cherla R P, et al. 2003 FEMS microbiology letters 228: 159-166). The toxins primarily affect the glomerular endothelial endothelium in humans (Obrig T G, et al. 1993 J Biol Chem 268: 15484-15488) and renal tubular epithelium in mice (Psotka M A, et al. 2009 Infect Immun 77: 959-969), which express the Gb3 receptor. The systemic consequences of intoxication are vascular dysfunction, leukocyte recruitment, and thrombus formation that can lead to HUS (reviewed in Zoja C, et al. 2010 Pediatric nephrology 25: 2231-2240).

Antibiotic treatment is not recommended for STEC infection (Wong C S, et al. 2012 Clinical infectious diseases: an official publication of the Infectious Diseases Society of America 55: 33-41) so treatment is limited to fluid replacement and supportive care (Hunt J M 2010 Clinics in laboratory medicine 30: 21-45; Melton-Celsa A, et al. 2011 Current topics in microbiology and immunology). Thus, there is a need for new treatment options. Currently anti-Stx monoclonal Abs (mAbs) show promise in animal models (Yamagami S, et al. 2001 J Infect Dis 184: 738-742; Mukherjee J, et al. 2002 Infect Immun 70: 612-619; Mukherjee J, et al. 2002 Infect Immun 70: 5896-5899; Tzipori S, et al. 2004 Clin Microbiol Rev 17: 926-941, table of contents; Dowling T C, et al. 2005 Antimicrobial agents and chemotherapy 49: 1808-1812; Sauter K A, et al. 2008 Infect Immun 76: 4469-4478) and clinical trials are ongoing (Thallion Pharmaceuticals). It remains unknown whether antitoxin antibodies administered after the onset of diarrheal symptoms will prevent or modify the outcome of HUS (Tzipori 5, et al. 2004 Clin Microbiol Rev 17: 926-941, table of contents; Sauter K A, et al. 2008 Infect Immun 76: 4469-4478). Even if effective, the use of mAb-based antitoxins may be too costly to stockpile as a therapeutic option since different mAbs are likely required to neutralize the two Shiga toxins and multiple different mAbs targeting each toxin may be needed to decorate the toxins and promote their clearance via low affinity Fc receptors (FcRs) (Davies K A, et al., Arthritis Rheum 46: 1028-1038, 2002; Lovdal T, et al. 2000 J Cell Sci 113 (Pt 18): 3255-3266).

An alternative antitoxin platform (Mukherjee J, et al. 2012 PLoS ONE 7: e29941) that has advantages over current strategies has been developed herein. The antitoxins contain two proteins; a ‘VHH-based neutralizing agent’ (VNA) and an ‘effector Ab’ (efAb) (Mukherjee J, et al. 2012 PLoS ONE 7: e29941). The VNAs consist of linked VHHs, produced as heteromultimers that bind and neutralize their toxin targets. The VHH components of VNAs are 14 kDa camelid heavy-chain only Ab V_{H} domains. VHHs are robustly expressed by recombinant E. coli and thus economical to produce (Mukherjee J. et al. 2012 PLoS ONE 7: e29941; Gibbs W W Nanobodies. Sci Am 293: 78-83, 2005). To promote toxin clearance, the VNA are co-administered with a single anti-tag mAb, called the ‘effector Ab’ or efAb that binds to multiple epitopic tags engineered into each VNA molecule. VNAs are bound at separate sites on the toxin, and each VNA is bound to two or more efAbs through the tags, the toxin then becomes decorated by sufficient efAbs to promote liver clearance (Sepulveda J, et. al. 2010 Infect Immun 78: 756-763), presumably by low affinity FcRs.

In examples herein the Stx-binding VHHs that neutralize each of the Shiga toxins, Stx1 And Stx2, and some VHHs that neutralize both toxins are identified. VHH heterotrimer VNAs are described in which a single VNA protein potently neutralizes both Stxs through binding at two separate sites on each toxin. The heterotrimeric VNAs have much greater antitoxin efficacy when the VNA is co-administered with the efAb. These simple antitoxin agents effective against both Shiga toxins should offer new therapeutic options for treating STEC infections to prevent HUS sequelae.

An antitoxin strategy that employs a VHH-based neutralizing agent (VNA), consisting of two antitoxin VHHs flanked by two copies of an epitopic tag, to direct the binding of up to four anti-tag effector Ab (efAb) molecules to the toxin and promote both toxin neutralization and toxin clearance from serum has been reported. See Mukherjee J, et al. 2012 PLoS ONE 7: e29941. VNAs in which a single protein agent neutralizes both of the Shiga toxins produced by Shiga-like toxin-producing E. coli (STEC) infection are described in examples herein. STEC disease can lead to serious, sometimes fatal, complications such as HUS and encephalopathy for which no specific therapy currently exists. VNA administered together with the efAb to mice mitigated induced by Shiga toxin and minimized renal damage.

To develop an antitoxin agent effective against both Shiga toxins, VHHs capable of binding Stx1 And/or Stx2 was identified and expressed. The VHHs were characterized for their subunit specificity, and their toxin binding and neutralization properties. Most Shiga toxin-binding VHHs recognized the B subunit and these VHHs neutralized the their targets in cell assays. One class of B subunit-binding VHHs recognized both Stx1 And Stx2. Donohue-Rolfe et al 1989 Infect Immun 57: 3888-3893 described a mAb (4D1) with similar binding characteristics. Only one Shiga toxin-binding VHH, a Stx1-specific VHH (Stx1-D4), recognized the A subunit, and this proved incapable of neutralizing either toxin. In total, 9/10 of the unique VHHs tested (Table 6) proved capable of neutralizing their targets, a much higher proportion than previously observed with toxin-binding mAbs. See Chow S K, et al. 2012 Toxins 4: 430-454. This high proportion is related to the reported ability of VHHs to bind preferentially to active site grooves on their targets. See Wesolowski J, et al. 2009 Med Microbiol Immunol 198: 157-174.

The antitoxin strategy uses VNAs consisting of two or more linked, toxin-neutralizing, VHHs recognizing non-overlapping epitopes on the toxin. VHH heteromultimers were initially developed to facilitate the decoration of toxins at multiple sites so as to promote clearance of the toxin from serum when the VNA is co-administered with efAb. See Mukherjee J, 2012 PLoS ONE 7: e29941. The examples described herein highlight another frequent advantage of linking VHHs together; increased toxin binding affinity and potency of neutralization. In every instance tested, VHH heterodimer VNAs functioned more effectively as antitoxins in cell and animal assays than did equimolar pools of the component VHHs. In some cases, linking VHHs into VNAs improved the antitoxin potency as much as 100 fold (FIGS. 33 and 34, Table 7) and substantially improved in vivo efficacy (FIGS. 35 and 36).

The identification of cross-specific VHHs that recognized Stx1 And Stx2 facilitated the development of a VHH heterotrimer VNA capable of binding to two separate epitopes on each of the two Shiga toxins. Although these cross-specific VHHs were relatively poor at toxin neutralization on their own, when these VHHs were linked to an Stx1- or Stx2-specific VHH, the resulting heterodimers proved to be extremely potent, displaying sub-nanomolar in vitro IC50 values. Double-tagged VHH heterotrimer VNAs were prepared consisting of a cross-specific VHH linked to a Stx1-specific VHH and a Stx2-specific VHH. These agents retained high toxin neutralizing potency and were effective in protecting mice from exposure to both Shiga toxins, especially when co-administered with the efAb (FIGS. 35 and 36).

The contribution of serum clearance to improved efficacy was observed more with Stx1 than Stx2, probably because Stx1 toxin is less potent in mice. Since a 20-fold higher dose of Stx1 was required for a MLD than with Stx2, the molar excess of VNA to toxin was 20-fold less with Stx1 And this may have contributed to the poor efficacy of the antitoxin VNAs in protecting mice from toxemia and death. By including the efAb to promote serum clearance, Stx1 Becomes decorated with up to four efAbs and is thus rapidly cleared through the liver, (Sepulveda J, et. al. 2010 Infect Immun 78: 756-763) and this treatment resulted in the complete asymptomatic survival of all mice. The important role of serum clearance was less dramatically demonstrated with Stx2. In this model, mice often survived 1.25 MLD of toxin when given the VNA alone, but developed demonstrable kidney damage. Co-administration of efAb fully protected the mice receiving Stx2 from death and kidney pathology.

Since Shiga toxins, which inactivate ribosomes, should be toxic to virtually all mammalian cells in which they enter, a concern existed that clearance of Shiga toxins using VNAs co-administered with efAb might lead to selective killing of cells responsible for the clearance. Previous publications (Sepulveda J, et al. 2010 Infect Immun 78: 756-763) have demonstrated that agent clearance occurs in the liver, presumably by low affinity Fe-receptor-mediated endocytosis primarily in Kupffer cells. See Lovdal T, et. al. 2000 J Cell Sci 113 (Pt 18): 3255-3266. Selective killing of these important cells could be a consequence of promoting Shiga toxin clearance. Mice treated with VNAs together with efAb did not display clinical signs or microscopic evidence of liver damage perhaps because toxin neutralization by VNAs continued after cell uptake.

The examples described herein employ VNAs to treat the disease associated with STEC infection. Shiga toxins, especially Stx2, cause neurological signs and kidney damage in rodents and cause STEC-associated HUS in humans. Several groups generated and tested anti-Stx mAb-based treatments for STEC infection and their use has shown promise in animal models. See Yamagami S, et al. 2001 J Infect Dis 184: 738-742; Mukherjee J, et al. 2002 Infect Immun 70: 612-619; Mukherjee J, et al. 2002 Infect Immun 70: 5896-5899; Tzipori S, et al. 2004 Clin Microbial Rev 17: 926-941, table of contents; Dowling T C, et al. 2005 Antimicrobial agents and chemotherapy 49: 1808-1812; Sauter K A, et al. 2008 Infect Immun 76: 4469-4478.

However, to ensure protection against both Shiga toxins, such treatments likely requires at least two mAbs that potently neutralize each toxin, and further mAbs may be required to promote serum clearance. Therapeutic agents to prevent HUS consisting of multiple mAbs are complicated and expensive to develop, manufacture and test in clinical trials. Therefore, VNA antitoxins could lead to more practical and effective therapies for STEC infection.

A major consideration in development of treatments that prevent HUS must be the timing of the kidney injury in relation to the onset of gastrointestinal symptoms. If kidney injury occurs early in infection and prior or simultaneous to the onset of bloody diarrhea, then inactivation of toxins is unlikely to improve the outcome unless it is administered prior to these symptoms. See Tarr P I, et al. 2005 Lancet 365: 1073-1086. This might be possible, for example, by treating patients who display early signs of gastrointestinal upset, or patients suspected to have ingested food contaminated by STEC. Treatment of large populations only considered to be at potential risk of STEC infection would be impractical unless the treatment was extremely safe and inexpensive.

A single VNA that neutralize both Shiga toxins makes possible new, more practical approaches to preventing STEC sequelae. One option is to engineer gene therapy vehicles, such as adenoviruses, that promote transient secretion of the VNA (and efAb if enhanced potency was needed) into the circulation. Alternatively, strategies for oral delivery of a VNA may be possible that are sufficiently safe and economical to permit prophylactic use in at-risk populations. For example, a VNA could be expressed and secreted in the GI tract by genetically-engineered commensal bacteria, similar to an approach employed to treat inflammatory bowel disease in an animal model. See Vandenbroucke K, et al. 2009 Mucosal Immunol. Alternatively, a VNA could be delivered to the GI tract in capsules or other vehicles that protect the agent through the stomach.

The examples described herein show a single VNA that is capable of neutralizing both Shiga toxins and if co-administered with efAb to promote toxin clearancecan effectively protect mice from lethal doses of Stx1 And Stx2. Since the single agent neutralizes both Shiga toxins, it is capable of protecting patients from STEC sequelae such as HUS. The simplicity of the agent and its ease of production make possible a variety of alternative treatment strategies including genetic and oral delivery routes.

A skilled person will recognize that many suitable variations of the methods may be substituted for or used in addition to those described above and in the claims. It should be understood that the implementation of other variations and modifications of the embodiments of the invention and its various aspects will be apparent to one skilled in the art, and that the invention is not limited by the specific embodiments described herein and in the claims Therefore, it is contemplated to cover the present embodiments of the invention and any and all modifications, variations, or equivalents that fall within the true spirit and scope of the basic underlying principles disclosed and claimed herein.

The following examples and claims are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references including issued patents and published patent applications cited in this application are hereby incorporated by reference.

### EXAMPLES

### Example 1

### Toxins and Reagents

Botulinum neurotoxin serotype A1 (BoNT/A) and serotype B (BoNT/B) were obtained from Metabiologics Inc. Each batch of toxin was calibrated to establish the LD₅₀ dose in mice and stored in aliquots at −80° C. until use. Purified recombinant BoNT serotype A1 And B holotoxins containing mutations rendering them catalytically inactive (ciBoNTA, ciBoNTB) obtained. Sheep anti-BoNT/A1 Antiserum was produced by immunization of sheep with BoNT/A 1 toxoid followed by BoNT/A1 holotoxin. Less than 1 μl of this sheep antitoxin serum protects mice from lethality when co-administered with 10,000-fold the LD₅₀ of BoNT/A1. Reagents for Western blotting were purchased from KPL (Gaithersburg, Md.).

C. difficile holotoxins TcdA and TcdB were generated by transformation of shuttle vectors pHis1522 (pHis-TcdA and pHis-TcdB respectively) into B. megaterium described in Yang et al. 2008 BMC Microbiology 8:192. Point mutations were introduced into conserved amino acids that are responsible for binding to the substrate, uridine diphosphoglucose (UDP-Glucose), in order to generate GT-deficient holotoxins. To generate GT-mutant holotoxin A, a unique restriction enzyme (BamHI) site was designed and constructed between sequences encoding GT and CPD domains using overlapping PCR. The primer sets used were:

| SEQ ID NO: 90 | |
|---|---|
| pHis-F | (5′- TTTGTTTATCCACCGAACTAAG-3′;), |
| SEQ ID NO: 91 | |
| Bam-R | (5′- TCTTCAGAAAGGGATCCACCAG-3′;), |
| SEQ ID NO: 92 | |
| Bam-F | (5′- TGGTGGATCCCTTTCTGAAGAC-3′;), |
| and | |
| SEQ ID NO: 93 | |
| Bpu-R | (5′- ACTGCTCCAGTTTCCCAC-3′;. |

The final PCR product was digested with BsrGI and Bpu10I, and was used to replace the corresponding sequence in pHis-TcdA. The resulting plasmid was designated pH-TxA-b. Sequences encoding triple mutations (W101A, D287N, and W519A) in the GT were synthesized by Geneart (Regensburg, Germany) and cloned into pH-TxA-b through BsrGI/BamHI digestion. To generate the mutant holotoxin B construct, the sequence between BsrGI and NheI containing two point mutations (W102A and D288N) was synthesized and inserted into pHis-TcdB at the same restriction enzyme sites, leading to a new plasmid pH-aTcdB. The mutant aTcdA and aTcdB were expressed and purified identical to the wild types in B. megaterium as described by Yang et al. 2008 BMC Microbiology 8:192. The purified aTcdA and aTcdB were used to immunize alpacas.

### Example 2

### Alpaca Immunization and VHH-Display Library Preparation

Purified, catalytically inactive mutant forms of full-length recombinant BoNT/A (ciBoNTA) and BoNT/B (ciBoNTB) proteins were obtained as described in Webb et al. 2009 Vaccine 27: 4490-4497. Alpacas (two animals per immunization type) were immunized with either ciBoNTA or with ciBoNTB. Additional alpacas were immunized with aTcdA or aTcdB. The immunization regimen employed 100 μg of protein in the primary immunization and 50 μg in three subsequent boosting immunizations at three weekly intervals in aluminum hydroxide gel adjuvant in combination with oligodeoxynucleotides containing unmethylated CpG dinucleotides (alum/CpG; Superfos Biosector; Copenhagen, Denmark) adjuvant. Five days following the final boost immunization, blood from each animal was obtained for lymphocyte preparation and VHH-display phage libraries were prepared from the immunized alpacas as previously described (Maass et al. 2007 Int J Parasitol 37: 953-962 and Tremblay et al. 2010 Toxicon. 56(6): 990-998). Independent clones (greater than 10⁶ total) were prepared from B cells of alpacas successfully immunized with each of the BoNT immunogens.

### Example 3

### Anti-BoNT VHH Identification and Preparation

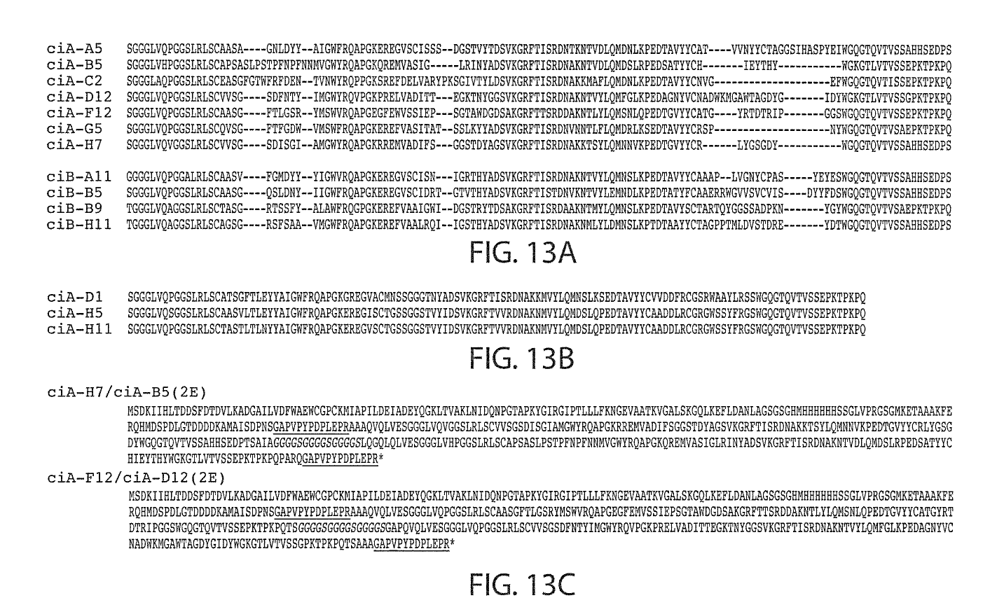

The VHH-display phage libraries were panned for binding to ciBoNTA or ciBoNTB targets that were coated onto each well of a 12-well plate. Coating was performed by overnight incubation at 4° C. with one ml of a 5 μg/ml target solution in PBS, followed by washing with PBS and two hours incubation at 37° C. with blocking agent (4% non-fat dried milk powder in PBS). Panning, phage recovery and clone fingerprinting were performed as previously described (Ibid.). Based on phage ELISA signals, a total of 192 VHH clones were identified as strong candidate clones for binding to BoNT/A, and 142 VHH clones were identified as strong positives for binding to BoNT/B respectively. Of the strong positives, 62 unique DNA fingerprints were identified among the VI-Ms selected for binding to BoNT/A and 32 unique DNA fingerprints were identified for VHHs selected for binding to BoNT/B. DNA sequences of the VHH coding regions were obtained for each phage clone and compared for identifying homologies. Based on these data, twelve of the anti-BoNT/A VHHs and eleven anti-BoNT/B VHHs were identified as unlikely to have common B cell clonal origins and were selected for protein expression. Expression and purification of VHHs in E. coli as recombinant thioredoxin (Trx) fusion proteins containing hexahistidine was performed as previously described in Tremblay et al. 2010 Toxicon. 56(6): 990-998. For heterodimers, DNA encoding two different were joined in frame downstream of Trx and separated by DNA encoding a fifteen amino acid flexible spacer having the amino acid sequence (GGGGS)₃. VHHs were expressed with a carboxyl terminal E-tag epitope. Furthermore, a number of VHH expression constructions were engineered to contain a second copy of the E-tag by introducing the coding DNA in frame between the Trx and VHH domains. An example of a Trx fusion to a VHH heterodimer with two E-tags is ciA-H7/ciA-B5(2E) shown in FIG. 13C.

### Example 4

### VHH Target Binding Competition Analysis

Phage displaying individual VHHs were prepared and titered by phage dilution ELISA for recognition of ciBoNTA or ciBoNTB using HRP/anti-M13 Ab for detection (Maass et al. 2007 Int J Parasitol 37: 953-962). A dilution was selected for each phage preparation that produced a signal near the top of the linear range of the ELISA signal. The selected phage dilution (100 μl) for each VHH-displayed phage preparation were added to 96 well plate that has been coated with ciBoNTA or ciBoNTB and then pre-incubated for 30 minutes with 100 μl of a 10 μg/ml solution containing a purified Trx/VHH fusion protein test agent or control in PBS. After an hour, the wells were washed and phage binding was detected. Test VHHs that reduced target binding of phage-displayed VHHs by less than two-fold compared to controls were considered to recognize distinct epitopes. Positive controls were prepared in which the Trx/VHH competitor contained the same VHH as displayed on phage and typically reduced the ELISA signal detected by greater than 95%.

### Example 5

### Characterization of VHH Binding Properties

VHHs were tested for binding to native or atoxic mutant BoNT holotoxins by standard ELISA using plates coated with 100 μl of 1 μg/ml protein. VHHs were also tested for recognition of BoNT subunits by ELISA using plates coated with 5 μg/ml purified recombinant BoNT light chain or 1 μg/ml BoNT heavy chain. See Tremblay et al. 2010 Toxicon. 56(6): 990-998. VHHs were also characterized for recognition of subunits by Western blotting on BoNT holotoxin following SDS-PAGE electrophoresis under reducing conditions. VHHs were detected with HRP-anti-E-tag mAb (GE Healthcare) by standard procedures.

### Example 6

### Kinetic Analysis by Surface Plasmon Resonance

Assays to assess the kinetic parameters of the VHHs were performed using a ProteOn XPR36 Protein Interaction Array System (Bio-Rad, Hercules, Calif.) after immobilization of ciBoNT/A by amine coupling chemistry using the manufacturer recommended protocol. Briefly, after activation of a GLH chip surface with a mixture of 0.4 M ethyl (dimethylaminopropyl) carbodiimide (EDC) and 0.1 M N-hydroxysulfosuccinimide (sulfo-NHS) injected for 300 s at 30 μL/min, ciBoNT/A was immobilized by passing a 60 μg/mL solution of the protein at pH 5 over the surface for 180 s at 25 μL/min. The surface was deactivated with a 30 μL/min injection of 1 M ethanolamine for 300 s. A concentration series for each VHH (between 2.5 nM and 1000 nM, optimized for each antibody fragment) was passed over the surface at 100 μL/min for 60 s, then dissociation was recorded for 600 s or 1200 s. The surface was then regenerated with a 36 s injection of 10 mM glycine, pH 2.0 at 50 μL/min. The running buffer used for these assays was 10 mM Hepes, pH 7.4, 150 mM NaCl, 0.005% Tween-20. Data was evaluated with ProteOn Manager software (version 2.1.2) using the Langmuir interaction model.

### Example 7

### BoNT Neutralization Assay Using Primary Neurons

Neuronal granule cells from the pooled cerebella of either 7-8 day old Sprague-Dawley rats or 5-7 day old CD-1 mice were harvested (Skaper et al 1979 Dev Neurosci 2: 233-237) and cultured in 24 well plates as described by Eubanks et al 2010 ACS Med Chem Lett 1: 268-272. After at least a week of culture the well volumes were adjusted to 0.5 ml containing various VHH dilutions or buffer controls followed immediately by addition of BoNT/A in 0.5 ml to a final 10 pM. After overnight at 37° C., cells were harvested and the extent of SNAP25 cleavage assessed by Western blot as previously described (Eubanks, L. M. et al. 2007 Proc. Natl. Acad. Sci. USA 104: 2602-2607).

### Example 8

### Mouse Toxin Lethality Assay

Female CD1 mice (Charles River) about 15-17 g each were received from three to five days prior to use. On the day each assay was initiated, mice were weighed and placed into groups in an effort to minimize inter-group weight variation. Appropriate dilutions of the VHH agents were prepared in PBS. BoNT holotoxins were separately prepared in PBS at the desired doses. Amounts (600 μl) of VHH agent and (600 μl) of the toxin were combined and incubated at room temperature for 30-60 minutes. An amount (200 μl) of each mixture was administered by intravenous injection at time point zero to groups of mice (five mice per group). Mice were monitored at least four times per day and assessed for symptoms of toxin exposure and lethality/survival. Moribund mice were euthanized. Time to onset of symptoms and time to death were established for each mouse.

### Example 9

### Mouse Toxin Lethality Assay with Agents Administered Post-Intoxication

Groups of mice were prepared as described in the description of the mouse toxin lethality assay. Subjects were administered 10 LD₅₀ of BoNT/A by intraperitoneal injection. At indicated times post-intoxication, mice were administered 200 ul of material (e.g., VHH monomer or VHH heterodimer) in PBS by intravenous injection. Mice were monitored for symptoms of intoxication and death as described herein.

### Example 10

### Single-Chain Fvs (scFv) that Recognize and Bind BoNT/A

To improve therapies that involve multiple monoclonal antibodies (mAbs) by using small recombinant peptide, protein or polynucleotide agents that have the same binding specificity as the mAbs, each of the recombinant binding agents is produced containing the same epitopic tag. A single mAb that recognizes the epitopic tag is co-administered to patients with the binding agents. The different agents bind to the same targets as the multiple mAbs and the anti-tag mAb binds to these agents through the epitopic tag. This permits delivery of the same therapeutic effect that is achieved with multiple mAb therapy, but requires only a single mAb. If desired, mAbs of different isotypes, or polyclonal anti-tag antibodies, could be used therapeutically to deliver different immune effector activities.

A number of small recombinant protein agents were generated. They were called single-chain Fvs (scFvs) and were observed to recognize botulinum neurotoxin serotype A (BoNT/A). These scFvs are recombinant proteins that represent the antigen combining region of an immunoglobulin. Several anti-BoNT/A scFvs were produced and were purified. Each scFv contains the amino acid sequence (GAPVPYPDPLEPR; SEQ ID NO: 15) near the carboxyl terminus which is an epitopic tag referred to herein as “E-tag.” An scFvs (scFv#2) was shown to neutralize BoNT/A in a cell-based toxin assay (IC50˜7 nM). A second scFv (scFv#7) had little or no neutralization activity in the assay, and was found to bind to BoNT/A with high affinity (Kd˜1 nM).

The scFvs were tested for their ability to protect mice from the botulinum toxin BoNT/A by intravenous administration of the agents and toxin. The two scFvs were administered individually or together, and were given to mouse subjects with and without anti-E-tag mAb by intravenous administration. Each subject was administered a dose of 10 LD₅₀ of BoNT/A (i.e., an amount of BoNT/A ten-fold the LD₅₀), five mice per group. The results are shown in Table 1.

TABLE 1
scFv administration with and without anti-tag antibody
alleviates toxin morbidity

| Agents Administered (dose) | Survival | Observations |
|---|---|---|
| none | 0% | Death within a day |
| scFv#2 (20 μg) | 0% | Death after about a |
| | | day |
| scFv#7 (20 μg) | 0% | Death after less than |
| | | a day |
| scFv#2 (20 μg) + | 100% | Symptoms severe |
| anti-E-tag mAb (25 μg) | | |
| scFv#7 (20 μg) + | 0% | Death after several |
| anti-E-tag mAb (25 μg) | | days |
| scFv#2 (10 μg) + | 100% | No symptoms |
| scFv#7 (10 μg) + | | |
| anti-E-tag mAb (25 μg) | | |

The results shown in Table 1 clearly show that a BoNT/A neutralizing scFv (scFv#2) alone did not significantly protect mice from the toxin. Subjects survived (100%) following co-administration scFV#2 and mAb that recognizes an epitopic tag (E-tag) on the scFv. More importantly, co-administering two scFvs, each with E-tag, and anti-tag mAb dramatically improved the protective effect.

Subjects were administered 10 LD₅₀ and lower doses of the scFvs and the anti-E-tag mAb, and were analyzed for percent survival. Further, two additional non-neutralizing anti-BoNT/A scFvs (scFv#3 and scFv#21) were tested in combination with the neutralizing scFv#2. Whether the anti-E-tag mAb would function upon administration at a different site and time than the toxin was also tested.

The results in Table 2 confirm those data herein and further show that the mAb specific for the epitopic tag does not have to be pre-mixed with the scFv containing the epitopic tag to be effective. In fact, doses were administered at different sites and times. Combinations of two scFvs (each with E-tags) and the single anti-E-tag mAb, provided greater protection than with one scFv alone. This synergistic protective effect occurred using different scFvs and was observed at significantly lower doses of the scFvs or mAb than used in the data observed in Table 1.

TABLE 2
Anti-E-tag mAbs administered separately protected subjects from toxin

| Agents Administered (dose) | Survival | Observations |
|---|---|---|
| none | 0% | Death within a day |
| scFv#2 (10 μg) | 0% | Death after about |
| | | 2 days |
| scFv#2 (10 μg) + anti-E-tag mAb (10 μg) | 100% | Symptoms moderate |
| (mAb administered intraperitoneally) | | |
| scFv#2 (10 μg) + anti-E-tag mAb (10 μg) | 100% | Symptoms mild |
| scFv#2 (10 μg) + anti-E-tag mAb (2 μg) | 100% | Symptoms mild |
| scFv#2 (2 μg) + anti-E-tag mAb (2 μg) | 100% | Symptoms moderate |
| scFv#2 (5 μg) + scFv#7 (3 μg) + | 100% | No symptoms |
| anti-E-tag mAb (10 μg) | | |
| scFv#2 (1 μg) + scFv#7 (1 μg) + | 100% | No symptoms |
| anti-E-tag mAb (10 μg) | | |
| scFv#2 (5 μg) + scFv#3 (4 μg) + | 100% | No symptoms |
| anti-E-tag mAb (10 μg) | | |
| scFv#2 (5 μg) + scFv#21 (3 μg) + | 100% | No symptoms |
| anti-E-tag mAb (10 μg) | | |

Examples herein tested whether combinations of three and four scFvs with anti-tag mAb protect subjects from an amount of BoNT/A 100-fold, 1000-fold, or 10,000-fold the LD₅₀, i.e., 100 LD₅₀ BoNT/A, 1000 LD₅₀ BoNT/A or 10,000 LD₅₀ BoNT/A.

The data shown in Table 3 demonstrate the excellent potency of a tagged binding agent as an antitoxin. Specifically, completely protection of subjects from even mild symptoms of intoxication by 1,000 LD₅₀ was observed using combinations of three or four scFvs with anti-E-tag mAb. Subjects were protected from lethality from a 10,000 LD₅₀ dose with a combination of four scFvs, although moderate symptoms were observed. The ability to protect mice receiving up to 10,000 LD₅₀ of BoNT/A is equivalent to the highest level of protection reported with pools of different anti-BoNT/A mAbs (Nowakowski et al, Proc Natl Acad Sci USA, 99:11346-50).

TABLE 3
Combinations of scFv protect from 100, 1000, and 10,000 fold LD₅₀ BoNT/A doses in
presence of 10 μg of anti-E-tag mAb

| BoNT/A | Additional agents administered (dose) | Survival | Observations |
|---|---|---|---|
| 100 LD₅₀ | None | 0% | Death in less than a day |
| 100 LD₅₀ | scFv#2 (2 μg) + scFv#3 (2 μg) + scFv#21 (2 μg) | 100% | No symptoms |
| 1,000 LD₅₀ | None | 0% | Death in less than a day |
| 1,000 LD₅₀ | scFv#2 (2 μg) + scFv#3 (2 μg) + scFv#21 (2 μg) | 100% | No symptoms |
| 1,000 LD₅₀ | scFv#2 (2 μg) + scFv#3 (2 μg) + scFv#7 (2 μg) + | 100% | No symptoms |
| | scFv#21 (2 μg) | | |
| 10,000 LD₅₀ | None | 0% | Death in a few hours |
| 10,000 LD₅₀ | scFv#2 (2 μg) + scFv#3 (2 μg) + scFv#21 (2 μg) | 0% | Death delayed one day |
| 10,000 LD₅₀ | scFv#2 (2 μg) + scFv#3 (2 μg) + scFv#7 (2 μg) + | 100% | Moderate symptoms |
| | scFv#21 (2 μg) | | |

The next example tested efficacy of a binding agent containing two copies of the epitopic tag. The anti-BoNT/A binding agent, scFv#7, was engineered to contain another copy of the E-tag peptide. The E-tag peptide was present on the carboxyl terminus of each scFv. An altered version of seFv#7 (called scFv#7-2E) was engineered to be identical to scFv#7 and to have an additional copy of the E-tag peptide fused to the amino terminus.

TABLE 4
Protection from BoNT/A using scFvs having multiple tag sequences
in presence of 10 μg of anti-E-tag mAb

| BoNT/A | | | |
|---|---|---|---|
| LD₅₀ | Additional agents administered (1 μg each) | Survival | Observations |
| 100 | None | 0% | Death within 6 hours |
| 100 | scFv#2 + scFv#3 + scFv#7 | 100% | No symptoms |
| 100 | scFv#2 + scFv#3 + scFv#7-2E | 100% | No symptoms |
| 1,000 | None | 0% | Death within 2 hours |
| 1,000 | scFv#2 + scFv#3 + scFv#7 | 0% | Death after 2 days |
| 1,000 | scFv#2 + scFv#3 + scFv#7-2E | 100% | No symptoms |
| 10,000 | None | 0% | Death within 2 hours |
| 10,000 | scFv#2 + scFv#3 + scFv#7 | 0% | Death after less than a |
| | | | day |
| 10,000 | scFv#2 + scFv#3 + scFv#7-2E | 20% | Death after many days |
| 10,000 | scFv#2 + scFv#3 + scFv #21 + scFv#7 | 0% | Death after 2 days |
| 10,000 | scFv#2 + scFv#3 + scFv #21 + scFv#7-2E | 100% | Moderate symptoms |

The results in Table 4 demonstrate that the binding agent with two epitope tags dramatically improved the in vivo antitoxin efficacy of the tagged binding agent. With a combination of three scFvs, including scFvs#2, scFvs#3 and scFvs#7 or scFvs#7-2E, clearly the use of scFvs#7-2E was substantially superior in protection of mice to the use of scFvs#7 with only one E-tag. The improvement by presence of two copies of tag was particularly evident in the groups of mice challenged with 1,000 LD₅₀. In these groups, the triple combination of scFv#2+scFv#3+scFv#7 was insufficient to allow survival of the mice. When scFv#7 was replaced with scFv#7-2E, all the mice survived without symptoms. Furthermore, use of a pool of scFv#2+scFv#3+scFv#7-2E permitted the survival of one of five mice challenged with 10,000 LD₅₀ and delayed the death of the other mice by several days. The equivalent pool with scFv#7 having only one E-tag only delayed death for one day in mice challenged with 10,000 LD₅₀. Finally, an identical combination of four scFvs (#2, #3, #21 And #7) in which the efficacy using scFv#7 was compared with scFv#7-2E. Administering only one μg of each scFv, the presence of scFv#7 did not result in survival of mice challenged with 10,000 LD₅₀ and the same combination the scFv#7-2E was protective. These data show that mice were effectively protected from high doses of toxin by administering a smaller number high affinity binding agents, each containing two or more epitope tags together with an anti-tag mAb.

The method herein improves therapeutic agent flexibility, provides highly stable binding agents with long shelf life, substantially reduces the cost of production, and permits commercially feasible therapeutic applications that involve multiple target agents. Furthermore, the strategy herein will permit much more rapid development of new antitoxins. The binding agents are much more quickly developed to commercialization than mAbs. The single anti-tag mAb needed for co-administration is the same for therapies requiring different tagged binding agents and thus can be pre-selected for its commercial scale up properties and stockpiled in advance of the development of the binding agents.

An immediate application is in anti-toxin therapy, an area of high interest because of bioterrorist threats. For example, it is now thought that effective prevention of botulinum intoxication using toxin neutralizing mAbs will require administration of three different mAbs each targeting the same toxin. Since there are at least seven different botulinum toxins, this suggests that 21 different mAbs (or more) may need to be stockpiled for use in the event of a major botulism outbreak as might occur through bioterror. Monoclonal antibodies are very expensive to produce and have relatively short shelf lives. Methods and compositions herein would make it possible to produce 21 different recombinant binding agents, each having longer shelf-life and lower production costs, and then stockpile only a single mAb. It is possible that this approach could open up many other mAb therapeutic strategies that involve multiple binding targets, but which have not been pursued because of prohibitive development and production costs and poor product shelf life. Methods and compositions herein permit the use of mAbs of different antibody isotypes to be used with the same binding agents to provide greater therapeutic flexibility.

### Example 11

### BoNT/A VHHs Binding Agents

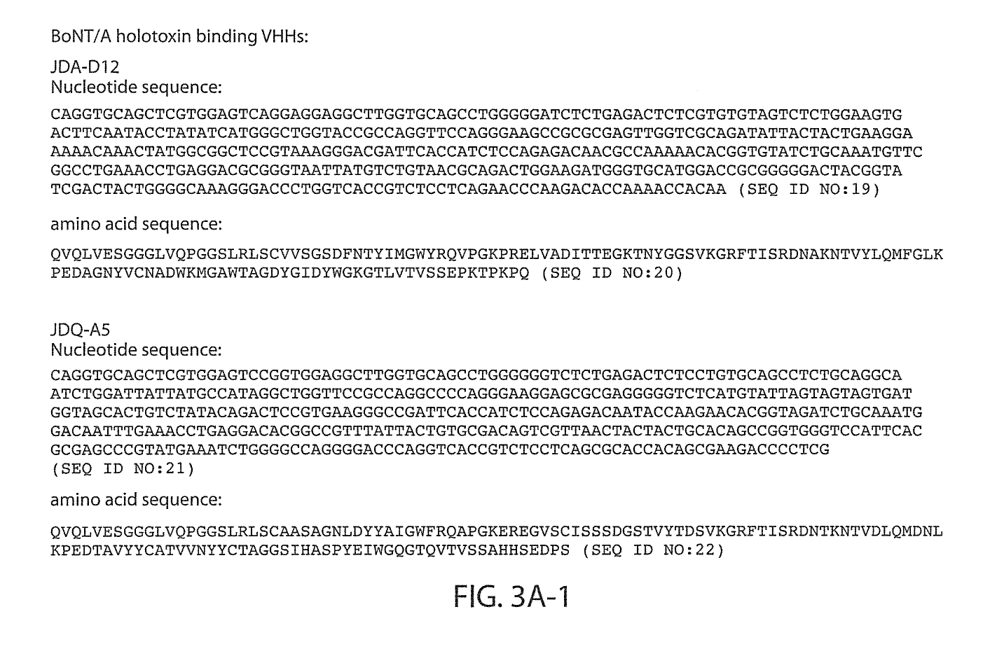

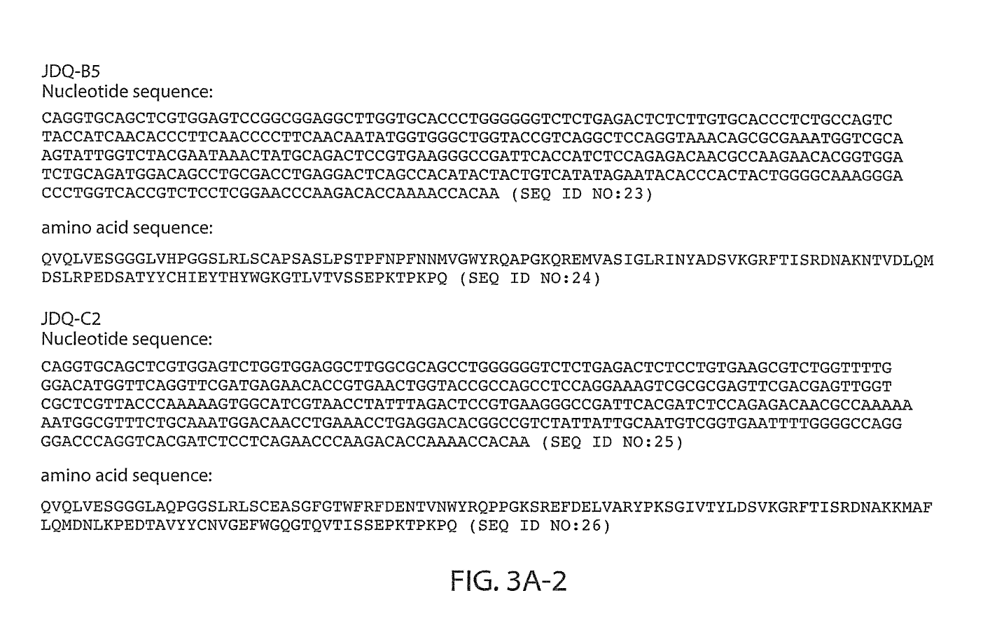

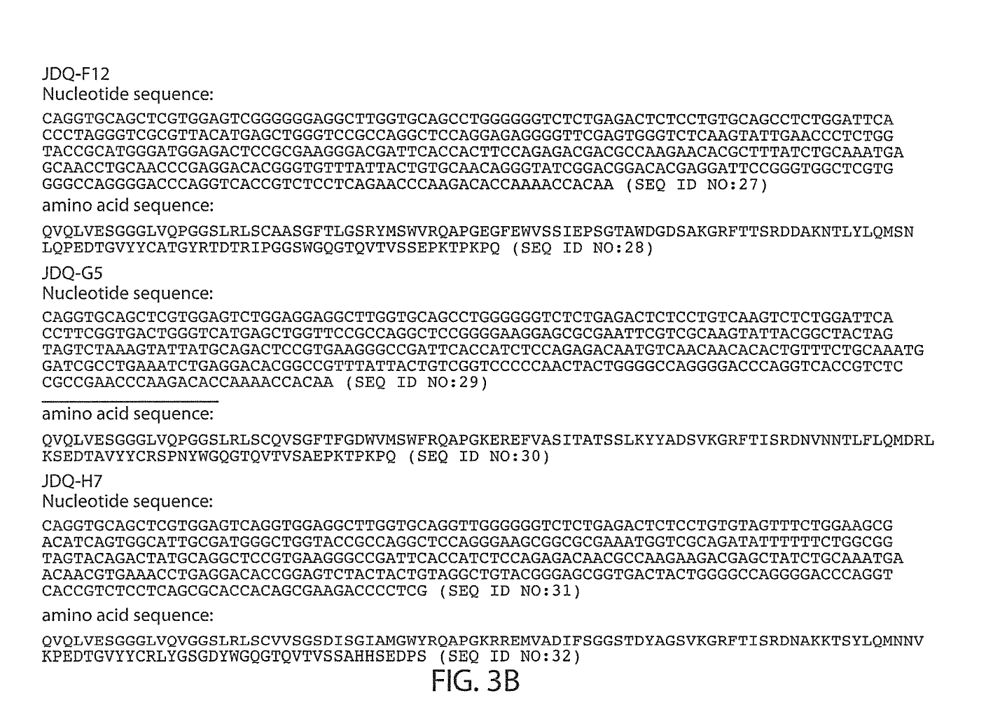

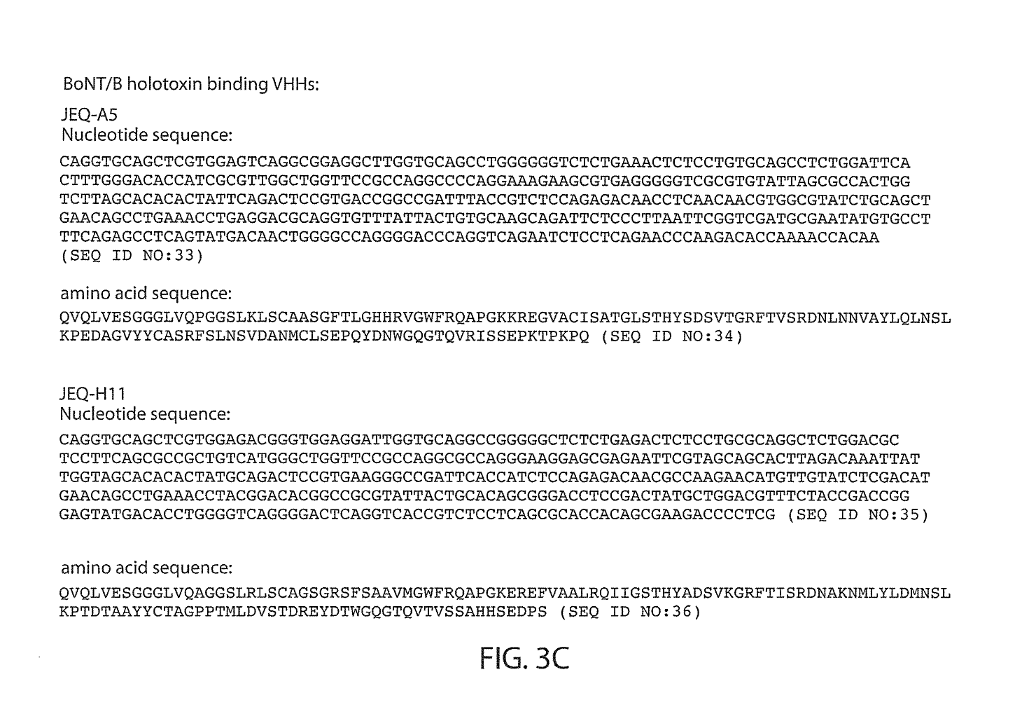

VHH binding agents were identified, produced and purified that were specific to each of botulinum neurotoxin serotype A (BoNT/A) and serotype B (BoNT/B). The VHHs made herein included nine amino acids at the amino coding end and which are associated with the forward PCR primer sequence. See FIG. 3 A-C for the sequences. These sequences derive from ‘framework 1’ and include minor variants of the original coding sequence. The most common amino acid sequence is QVQLVESGG (SEQ ID NO: 16) and which is the amino acid sequence used in assays shown in FIG. 3A-FIG. 3C.

At the carboxyl coding end of the VHHs either amino sequence, AHHSEDPS (SEQ ID NO: 17), or the amino sequence, EPKTPKPQ (SEQ ID NO: 18) is located, present in the VHHs sequence as shown in FIG. 3A-FIG. 3C, and these were observed to be interchangeable without loss of function. Identical clones were identified from alpacas that vary only in the hinge sequence and retain virtually the same target binding function. See also D. R. Maass et al. 2007 Journal of Immunological Methods 324:13-25.

As a result of the altered splicing, the amino acid sequence that joins the VH domain to the CH2 domain in heavy chain IgGs is called the “hinge” region, and is unique to this class of camelid antibodies (See D. R. Maass et al. 2007 Journal of Immunological Methods 324:13-25 which is incorporated by reference in its entirety). The two distinct hinge sequence types found in camels and llamas are referred to as the “short” hinge and the “long” hinge respectively. SEQ ID NO: 17 is a short hinge amino acid sequence derived from a camel, and SEQ ID NO: 18 is a long hinge amino acid sequence derived from a llama.

During screening for VHH binding agents, different coding sequences are identified that display significant homology among randomly identified clones. VHH sequences that are homologous are predicted to be related and thus to recognize the same epitope on the target to which they have been shown to bind. Examples herein experimentally demonstrate epitope recognition by methods for binding competition. These findings demonstrate that significant variation is permitted in VHH amino acid sequences without loss of target binding. An example of the extent of variation permitted is shown in FIG. 4A-FIG. 4B. Each VHH identified in FIG. 4A-FIG. 4B as a BoNT/A binder was experimentally observed to bind to the same epitope as JDQ-B5 based on binding competition assays.

FIG. 5 is a drawing of a phylogenetic tree that compares the homology among BoNT/A binding VHHs within the JDQ-B5 competition group to random alpaca VHHs. The homology comparison uses the unique amino acids that are present between the forward PCR primer sequences and the hinge region (above). The distance of the lines is a measure of homology; the shorter the distance separating two VHHs, the more homologous. Four VHHs that bind to the same epitope as JDQ-B5 cluster within a group that is distinct from the random VHHs as shown, strong evidence of relatedness of these clones. The results show that substantial variation in the VHH sequence is tolerated without loss of the target binding capability.

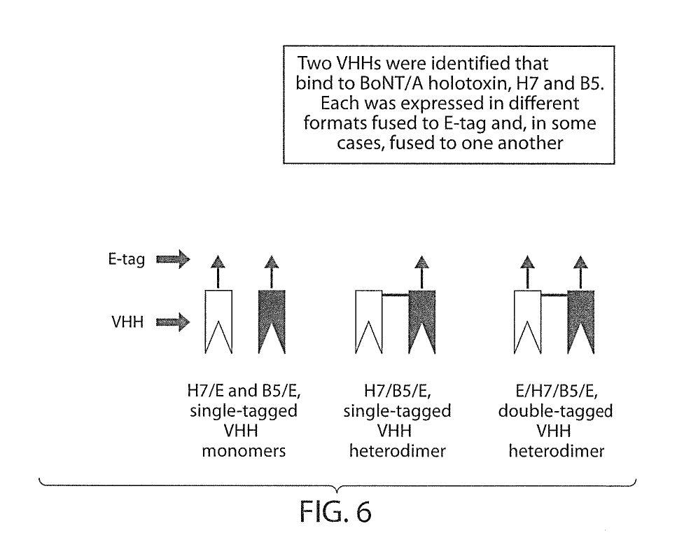

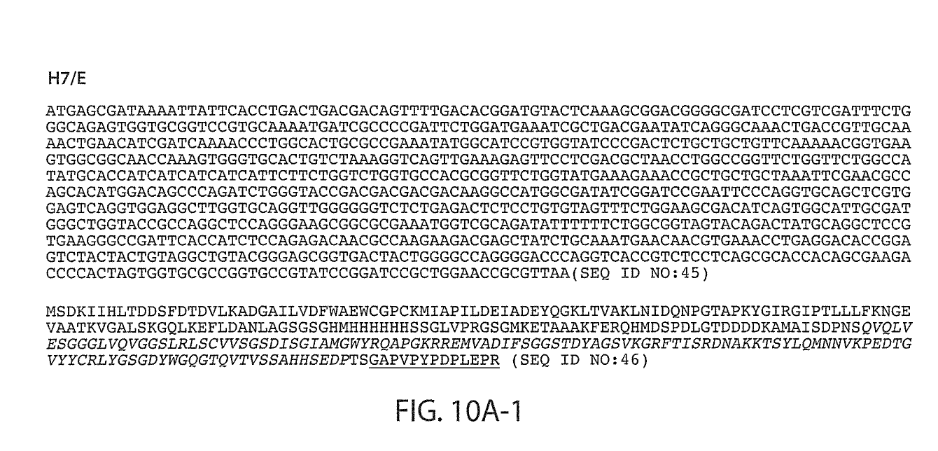

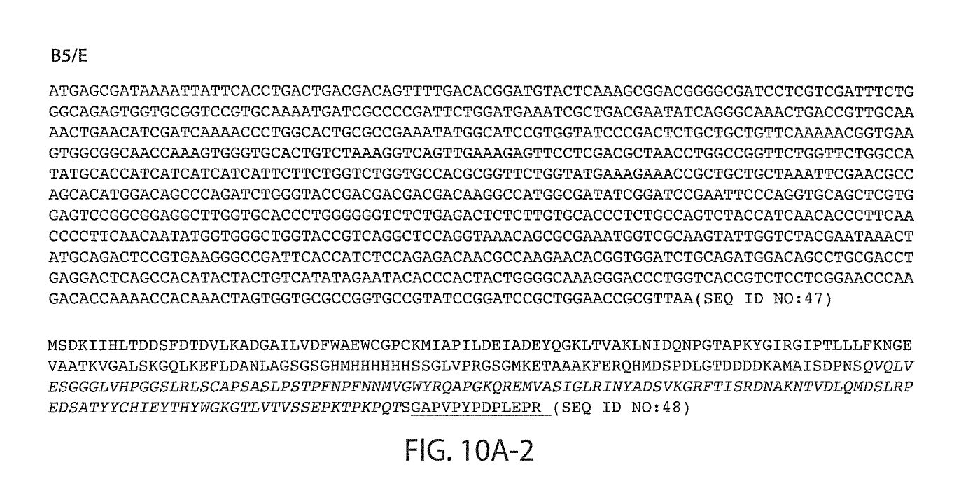

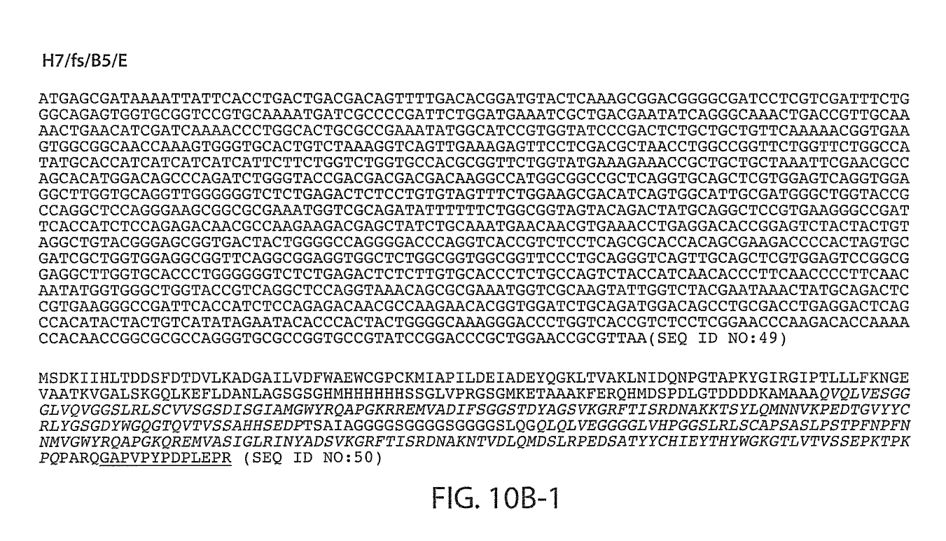

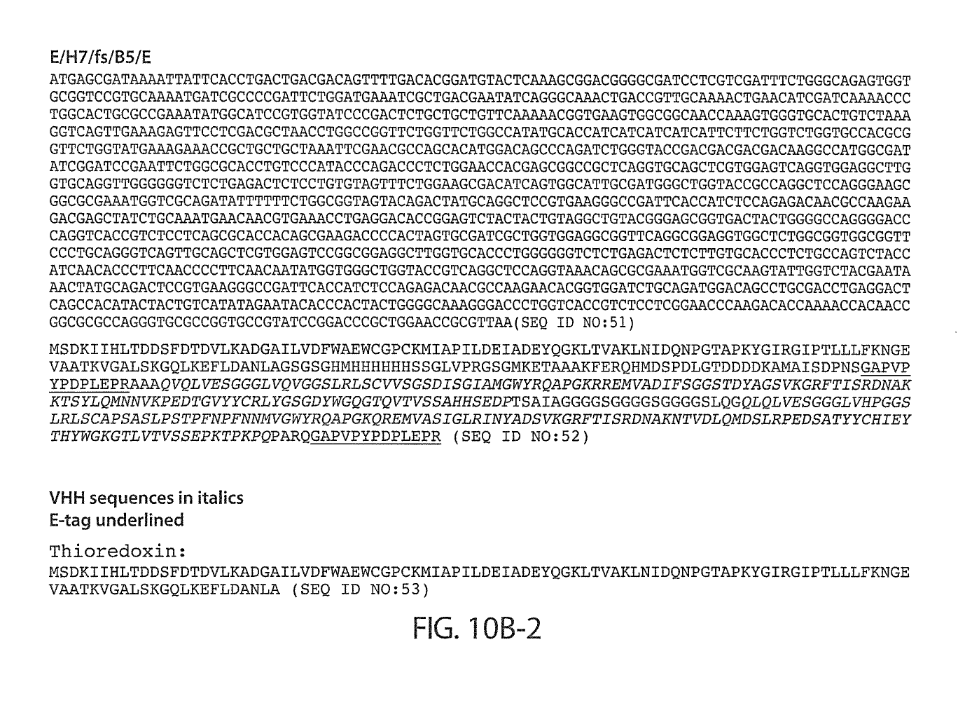

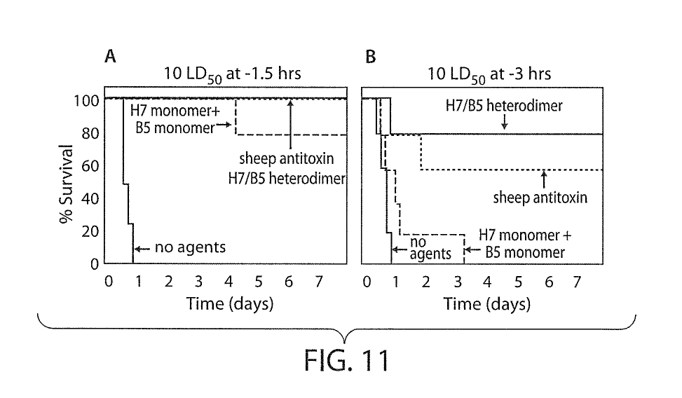

The coding DNAs for two different VHH monomers were cloned in an E. coli expression vector in several different ways to produce different recombinant proteins. To produce single VHH monomers, the VHH coding DNA was inserted into the plasmid pET32b to fuse the VHH in frame with an amino terminal bacterial thioredoxin and a carboxyl terminal epitopic tag (E-tag GAPVPYPDPLEPR; SEQ ID NO: 15). Additional coding DNA deriving from the pET32b expression vector DNA was also present between the thioredoxin and VHH coding sequences, the DNA encoding six histidines (to facilitate purification) and an enterokinase cleavage site. DDDDK to permit enzymatic separation of thioredoxin from the VHH. The resulting expression vectors were used for expression of VHH monomers. VHH monomers JDQ-H7 (SEQ ID NO: 32, referred to as “H7) and JDQ-B5 (SEQ ID NO: 24, referred to as “B5”) were expressed using this system (FIG. 6). The nucleotide sequences and the amino acid sequences of the two monomer VHH proteins produced by these expression vectors, labeled H7/E and B5/E, are shown in FIG. 10A.

Expression vectors were prepared in pET32b in which DNA encoding two iterations of the VHH monomer (e.g., SEQ ID NOs: 46 and 48) was present, and the monomers joined in frame to yield heterodimers. For these constructions, the two nucleic acid sequences encoding the VHHs were separated by a nucleotide sequence encoding a 15 amino acid linker, SEQ ID NO: 55, that functions as a flexible spacer (fs) between the expressed VHH proteins to separate the domains and facilitate independent folding. The E-tag coding DNA followed the second VHH coding DNA (SEQ ID NO: 49) in frame to obtain a single-tagged VHH heterodimer H7B5/E (SEQ ID NO: 50), the amino acid sequence of which is shown in FIG. 10B. A second copy of the F-tag coding DNA (e.g., SEQ ID NO: 51) was included upstream of the first VHH (at the amino coding end) for construction of a double-tagged VHH heterodimer E/H7/B5/E (SEQ ID NO: 52) shown in FIG. 10B.

The thioredoxin fusion partner was included to improve expression and folding of the VHHs, and was observed as not necessary for VHH function. The activity of the VHH agents to protect mice from BoNT/A intoxication in mouse lethality assays was tested using VHH agents in which thioredoxin was cleaved (by enterokinase) from the VHH. It was observed that absence of thioredoxin caused no significant reduction in activity.

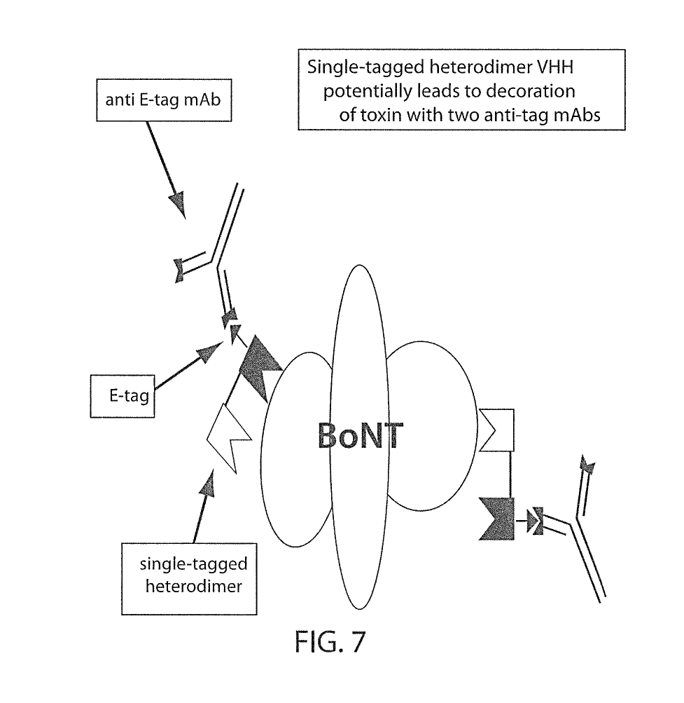

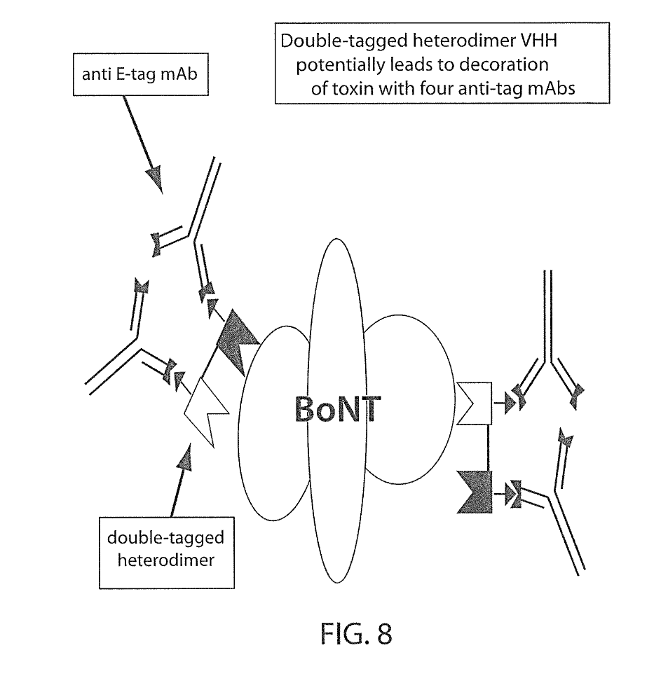

A single-tagged heterodimer VHH was predicted to lead to decoration of the BoNT toxin by the anti E-Tag mAb in a ratio of 1:1. Accordingly, a single-tagged heterodimer was expected to bind at two sites on the toxin and lead to decoration of the toxin with two anti E-tag antibodies (see FIG. 7). A double-tagged heterodimer provides for binding of the anti E-tag antibody in a ratio of 2:1 And thus should bind at two sites on the toxin and lead to decoration of the toxin with four anti-tag antibodies (see FIG. 8). These agents were tested for their ability to protect mice from BoNT/A toxin.

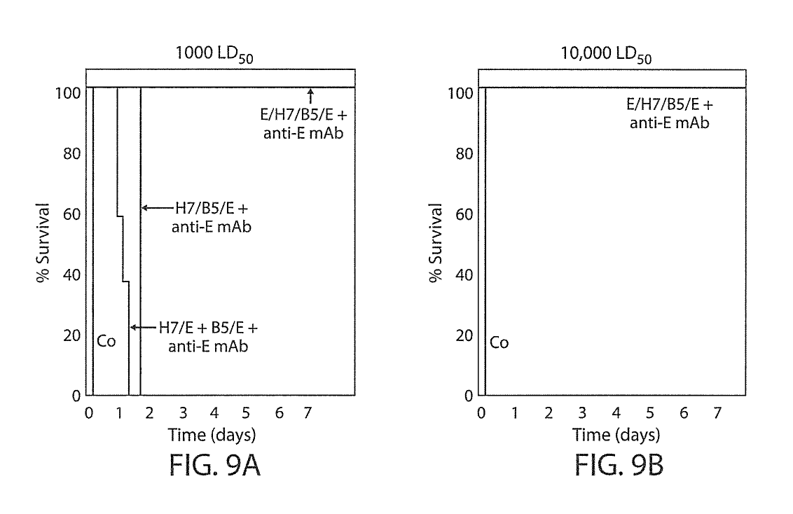

For these examples, the VHH agents and the toxin were pre-mixed and then intravenously administered to groups of five subjects (mice) per group. The subjects were monitored and the time to death was noted for those that succumbed to the toxin. In the results shown in FIG. 9A, a pool of two VHH monomers, H7/E and B5/E (1 μg of each monomer per subject), in the presence of anti-E-tag mAb (Phadia, Sweden) (5 μg/mouse) delayed death only about one day in mice exposed to 1,000 LD₅₀ of BoNT/A. The single-tagged VHH heterodimer, H7/B5/E (2 μg/mouse) in the presence of anti-E-tag mAb (5 μg/mouse) delayed death by about a day and a half in mice exposed to 1,000 LD₅₀ of BoNT/A.

In contrast, it was observed that the double-tagged heterodimer, E/H7/B5/E (2 μg/mouse) administered with anti-E-tag mAb resulted in full survival of mice exposed to 1,000 LD₅₀ and even 10,000 LD₅₀ of BoNT/A (FIG. 9B). Mice given the double-tagged VHH heterodimer, E/H7/B5/E, in the absence of co-administered anti-E-tag mAb, did not survive a 1,000 LD₅₀ amount of BoNT/E, showing that the anti-tag mAb was necessary for full efficacy. The ability of the double-tagged VHH heterodimer, E/H7/B5/E, administered with anti-E-tag mAb to protect mice against 10,000 LD₅₀ demonstrates that this treatment achieved a level of efficacy similar to that obtained with a commercial polyclonal antitoxin sera.

In other examples, the BoNT/A-binding VHH heterodimer agents were tested for their ability to prevent death in subjects previously exposed to BoNT/A. In these examples, groups of five subjects were first exposed to 10 LD₅₀ BoNT/A. Then after 1.5 or three hours from exposure mice were treated either with the E/H7/B5/E heterodimer agent (2 μg/subject) administered with anti-E-tag mAb (5 μg/subject), or with a dose of potent polyclonal anti-BoNT/A sera that had been prepared in sheep. This sera had been previously shown to protect subjects against 10,000 LD₅₀ of BoNT/A when it was co-administered with the toxin (assays performed as in previous paragraph). All subjects were monitored and the time to death was determined for non-survivors. Control subjects (2 groups of five each) died within about a day. All subjects treated with polyclonal antisera 1.5 hour post-intoxication (five) survived, and four of five subjects treated three hours post-exposure both 1.5 hours and three hours post-intoxication survived. Five out of five subjects treated with the VHH heterodimer and anti-E-tag mAb both 1.5 hours and three hours post-exposure survived. Thus the VHH heterodimer and anti-E-tag treatment was at least as effective as conventional polyclonal antitoxins at protecting subjects from BoNT exposure in the more clinically relevant post-exposure challenge model.

### Example 12

### Neutralization of Botulinum Neurotoxin Using VHH Binding Proteins

Examples herein show that scFv antitoxin compositions prevent development of disease symptoms in subjects exposed to a botulinum toxin. These antitoxin agents were antibodies that bound the toxin and neutralized the activity of the toxin and/or promoted rapid clearance from the body. Data show that effective neutralization was achieved using a mixture of two high-affinity toxin VHH binding agents, each of which strongly neutralized toxin in cell-based assays. Administration of a multimeric composition was much more effective at protecting subjects from toxin than a pool of two neutralizing monomer binding agents only.

Camelid heavy chain only Vh domain (VHH) binding agents with high affinity for Botulinum neurotoxin serotype A (BoNT/A) were produced including H7 (SEQ ID NO: 56), B5 (SEQ ID NO: 57). Methods of generating VHH binding agents are shown in Shoemaker et al. U.S. application Ser. No. 12/032,744 which is application 2010/0278830 A1 published Nov. 4, 2010, and Shoemaker et al. U.S. application Ser. No. 12/899,511 which is application 2011/0129474 A1 published Jun. 2, 2011, each of which is incorporated herein by reference in its entirety.

VHHs (H7, B5 and C2) displayed potent BoNT/A neutralization activity in assays of exposure or intoxication of primary neurons in culture. The 117 VHH and B5 VHH monomers were expressed in E. coli and a single heterodimeric polypeptide (H7/B5) was constructed and expressed with the H7 and B5 VHH domains/subunits separated by a fifteen amino acid flexible spacer having three repeats of amino acid sequence GGGGS (SEQ ID NO: 55). A combination of the H7 monomer binding agent and B5 monomer binding, and a H7/B5 single chain heterodimer binding agent were tested to determine ability to protect mouse subjects from death caused by BoNT/A. The subjects received ten-fold the lethal dose of BoNT/A that causes death in 50% of mice (10 LD₅₀), and either 1.5 hours or three hours later were administered either: 1 micrograms (μg) of H7 binding agent; a sheep antitoxin serum produced against BoNT/A; 1 μg of B5 monomer binding agent; or 2 μg of H7/B5 single chain heterodimer binding agent (FIG. 11A-FIG. 11 B). The amount of sheep antitoxin serum administered was equivalent to the amount of commercial antitoxin serum generally administered.

Data show that subjects administered a combination of monomeric H7 and B5 binding agents died within three days. Control subjects administered no therapeutic agent died within one day (FIG. 11A-FIG. 11B). Subjects administered the sheep antitoxin serum survived at 80%. Most important, subjects administered H7/B5 single chain heterodimer binding agent survived additional days compared to the control subjects, with 80% of subjects administered H7/B5 heterodimer binding agent surviving for seven days.

### Example 13

### Neutralization of C. difficile Toxins Using Heteromultimer Binding Agents

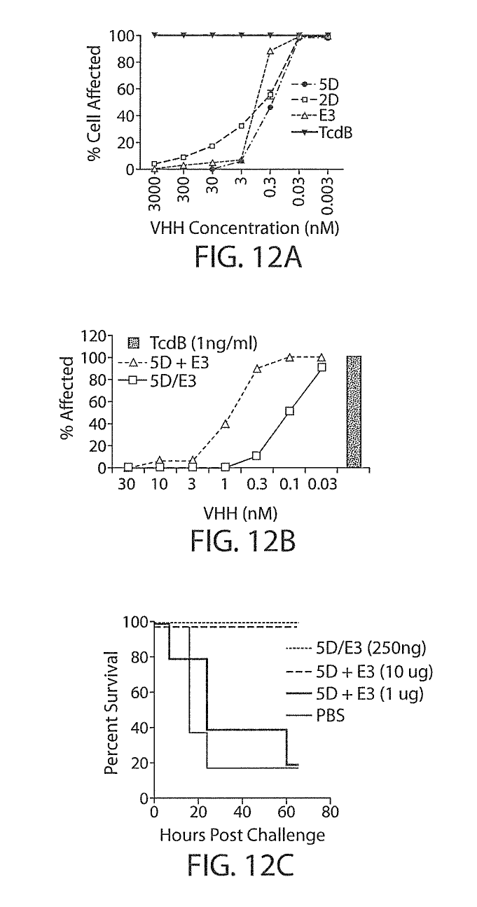

A set of VHH binding agents that bind Clostridium difficile toxin B (TcdB) were obtained and shown in Examples herein to inhibit the ability of the toxin to intoxicate or infect cells (FIG. 12A). Potent anti-TcdB neutralizing VHHs were selected, identified by codes names 5D and E3, and were expressed as separate monomers or as a heterodimer. A pool/mixture of VHH monomers, 5D and E3, was compared in for ability to prevent TcdB lethality to cells to the 5D/E3 heterodimer.

CT26 cells were exposed to TcdB (100 picograms/ml) in the presence of different concentrations (0.03 nM, 0.1 nM, 0.3 nM, 1 nM, 3 nM, 10 nM, or 30 nM) of: a mixture of 5D VHH monomer (SEQ ID NO: 67) and E3 VHH monomer (SEQ ID NO: 68), or a 5D/E3 heterodimer (SEQ ID NO: 87). Control cells were not administered neutralizing agents. Cell rounding caused by TcdB was monitored using a phase-contrast microscope.

Culture media from expressing cells were administered with either the mixture of 5D and E3 VHH monomers, or the 5D/E3 VHH heterodimer were found to be effective in protecting the cells from TcdB associated cell rounding. Control cells (100%) showed cell rounding and negative indicia of TcdB following toxin exposure.

It was observed that administering 0.1 nM 5D/E3 heterodimer to subjects prior to TcdB exposure resulted in 50% cell rounding (i.e., 50% TcdB infection; FIG. 12B). The same level cell rounding protection (50%), was achieved with 1 nM of the mixture of 5D and E3 monomers. Thus, the 5D/E3 VHH heterodimer was observed to be about ten-fold more potent as a toxin neutralizing agent than a pool containing the same two VHHs as monomers (FIG. 12B).

The improved antitoxin and protective potency 5D/E3 heterodimer was further analyzed using an in vivo toxin challenge mouse model. Subjects were co-administered a lethal dose of TcdB (1 ng/mL) with either a mixture of 500 nanograms (ng) of 5D monomer and 500 ng E3 VHH monomer; or with 250 ng of 5D/E3 VHH heterodimer; or with phosphate buffered saline, PBS. See FIG. 12C. See Data show that each of the VHH binding agents was a more effective TcdB neutralizing agent for subjects than the PBS control. Survival was observed at 100% for subjects administered 5D/E3 VHH heterodimer (250 ng) and at about 40% for subjects administered a mixture of 5D and E3 VHH monomers. Control subjects receiving PBS survived at a rate of 20%.

Data show that subjects administered a mixture of 5D and E3 monomers survived for fewer days and were less protected from a lethal TcdB challenge than subjects administered the 5D/E3 heterodimer (FIG. 12C). Most important the improved protection and neutralizing ability of the 5D/E3 heterodimers was observed even if the amount of heterodimer administered was 75% less than the amount of the mixture of 5D and E3 monomers. Further analysis was performed in Examples below to determine the relative factors for VHH monomers and heterodimers to effectively neutralize and clear disease agent targets from the body (FIG. 12A-FIG. 12C).

### Example 14

### Identification and Characterization of Anti-BoNT VHHs

Serum clearance of Botulinum neurotoxin serotype A (BoNT/A) was dramatically accelerated by administering a pool of different epitopically-tagged single-chain Ig variable fragment (scFv) domain binding agents with an anti-tag monoclonal antibody (Shoemaker et al. U.S. Ser. No. 12/032,744 application 2010/0278830 A1 published Nov. 4, 2010; Shoemaker et al. U.S. Ser. No. 12/899,511 Application 2011/0129474 A1 published Jun. 2, 2011; Sepulveda et al. 2009 Infect Immun 78: 756-763, and Tremblay et al. 2010 Toxicon. 56(6): 990-998, each of which is incorporated herein in its entirety).

To determine whether a more commercially and clinically acceptable binding agent than scFvs could be identified, a of camelid heavy-chain-only Vh (VHH) binding agents having high affinity for epitopes of BoNT/A holotoxin was produced. VHHs were obtained that bound to an epitope of a distinct BoNT serotype, BoNT/B holotoxin, and these VHHs were tested for antitoxin efficacy. Competition ELISAs were performed to identify the VHHs with the highest affinity for unique epitopes on BoNT/A and BoNT/B. VHHs specific for each of BoNT/A (FIG. 13A) and for BoNT/B (FIG. 13B) were identified.

The VHHs in FIG. 13 A-B include amino acid sequence QLQLVE (SEQ ID NO: 88) and QVQLVE (SEQ ID NO: 89) at the amino terminus region. The sequence was encoded by the PCR primer used to generate the VHH-display library (Maass et al. 2007 Int J Parasitol 37: 953-962). The eight amino acids shown at the carboxy-terminus end were encoded by the short hinge or long hinge PCR primers that were used to generate the VHH library.

The amino acid sequences for double-tagged VHH heterodimer antitoxins that specifically bind BoNT/A: ciA-H7/ciA-B5(2E) and ciA-F12/ciA-D12(2E) are shown in FIG. 13 C. Each heterodimer included two VHH monomers and two epitopic tags. The amino acid sequences of the tags within the amino acid sequences of the heterodimers are underlined (FIG. 13 C). The amino acid sequence preceding the first E-tag in each VHH protein contained the thioredoxin fusion partner and hexahistidine encoded by the pET32b expression vector. The VHH sequences were flanked by the two E-tag peptides and were separated by the unstructured spacer having amino acid sequence (GGGGS)₃. SEQ ID NO: 55.

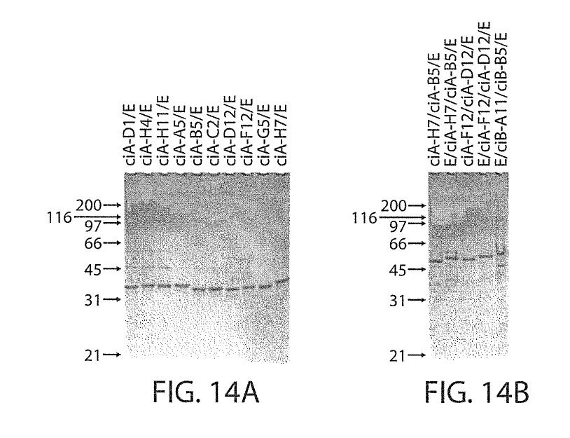

Each VHH was purified from E. coli as a thioredoxin fusion protein containing a single carboxyl-terminal epitopic tag (E-tag). Sodium dodecyl sulfate polyacrylamide gel electrophoresis (SDS-PAGE) analyses of VHH monomers and VHH heterodimers was performed (FIG. 14A-FIG. 14B). The channels were loaded with one microgram (μg) of each VHH monomer or heterodimer. Molecular weight markers (12, 31, 45, 66, 97, 116 and 200 kilodaltons) are shown on the border of each gel. FIG. 14 A shows SDS-PAGE analysis of the tagged (E) VHH monomers: ciA-D1, ciA-H4, ciA-H11, ciA-A5, ciA-C2, ciA-D12, ciA-F12, ciA-G5, and ciA-H7. Dark bands were observed at approximately 35-38 kilodalton molecular weight for all single tagged VHH monomers. Channels loaded with ciA-D1, ciA-H4, ciA-H11, and ciA-B5 showed light bands at about 45-46 kilodaltons (kDa), and at about 59 kDa to about 62 kDa molecular weight. SDS-PAGE analysis was performed also on single- or double-tagged VHH heterodimers: ciA-H7/ciA-B5 singly tagged on ciA-B5; double tagged ciA-H7/ciA-B5 having a tag on both ciA/H7 and ciA-B5, ciA-F12/ciA-D12 singly tagged on ciA-B5; double tagged ciA-F12/ciA-D12 having a tag on both ciA/F12 and ciA-D12, double tagged ciA-A11/ciA-B5 having a tag on both ciA/A11 And ciA-B5 (FIG. 14 B). Strong dark bands at about 48 kDa to about 58 kDa molecular weight were observed for each heterodimer (FIG. 14B).

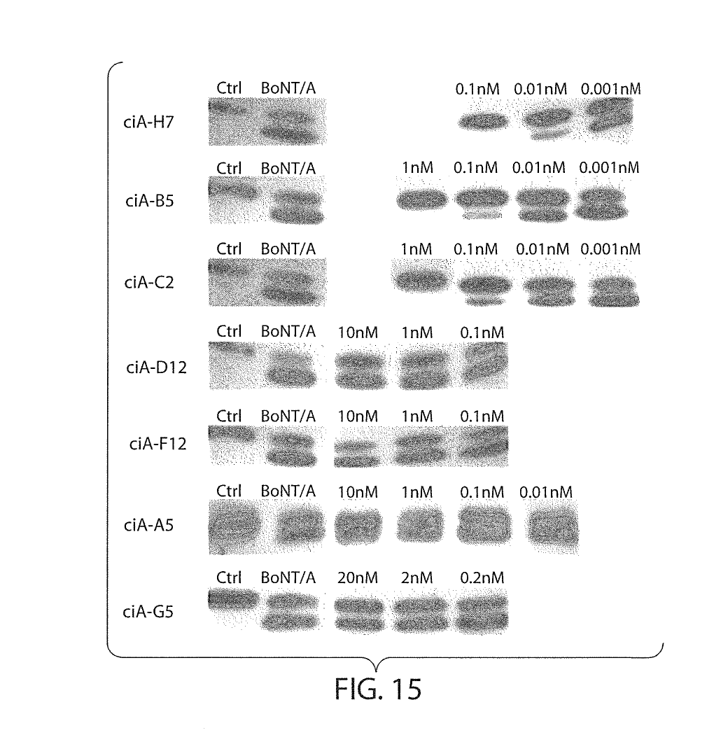

The unique BoNT/A binding VHHs were further characterized and analyzed for ability to affinity target BoNT/A using surface plasmon resonance (SPR) in which a lower Kd indicates stronger binding/affinity between the VHH and the toxin target. Analysis was performed also to determine the ability of the BoNT/A binding VHHs to prevent intoxication of primary neurons in culture (FIG. 15 and Table 5).

Neuronal granule cells from pooled cerebella of seven day old to eight day old Sprague-Dawley rats or five day old to seven day old CD-1 mice were harvested as described by Skaper et al 1979 Dev Neurosci 2:233-237. The cells were then cultured in 24-well plates as described by Eubanks et al 2010 ACS Med Chem Lett 1: 268-272. After a week or more of culture, each culture well was adjusted to a volume of 0.5 ml with dilutions of VHHs (ciA-H7, ciA-B5, ciA-C2, ciA-D12, ciA-F12, ciA-A5 or ciA-G5) or a buffer control, and BoNT/A (ten picomoles) was added. After overnight incubation at 37° C., cells were harvested and the extent of synaptosomal-associated protein 25 (SNAP25) cleavage was determined by Western blot using commercially available rabbit anti-SNAP25 (Sigma-Aldrich Inc.). See FIG. 15. SNAP-25 is a membrane bound protein anchored to the cytosolic face of membranes by palmitoyl side chains within the molecule that is involved in the regulation of neurotransmitter release. Botulinum toxin serotypes including serotypes A, C and E function to cleave SNAP-25, resulting in paralysis and clinically developed botulism.

The upper band shown in the Western blot photographs is uncleaved SNAP25, and the lower band indicates cleaved SNAP25 (FIG. 15). SNAP25 cleavage (i.e., presence of a lower band) resulting from exposure to botulinum toxin was observed. VHHs were identified by the criterion that at concentrations of less than 0.1 nanomoles (nM) were observed to inhibit BoNT/A cleavage of SNAP25 (i.e., no lower band), are strong neutralizing agents. Weak neutralizing VHHs were identified as VHHs that required greater than 1 nM to inhibit BoNT/A cleavage of SNAP25. VHHs that required greater than 10 nM to prevent SNAP25 cleavage were identified as having no toxin neutralizing ability (FIG. 15).

It was observed that about equimolar amounts of ciA-B5, ciA-C2 and ciA-H7 VHHs prevented intoxication of neurons with 10 picomoles of BoNT/A. Two VHHs (ciA-D12 and ciA-F12) were observed to have no or negligible toxin neutralizing activity even at 1,000-fold excess VHH to toxin. Two VHHs (ciA-A5 and G5) displayed intermediate neutralizing activity compared to ciA-B5, ciA-C2 and ciA-H7, the strongly neutralizing VHHs, and ciA-D12 and ciA-F12, the non-neutralizing VHHs (FIG. 15 and Table 5).

Thus, ciA-B5, ciA-C2 and ciA-H7 were determined to be strong neutralizing \THIN. Other isolates including ciA-D12 and ciA-F12 were observed to be non-neutralizing VHHs that produced no detectable toxin neutralization.

### Example 15

### Protection from BoNT/A Lethality Using Monomeric Anti-BoNT/A VHHs

Epitopically tagged anti-BoNT/A VHH compositions were shown in the Example herein to prevent toxin induced lethality in the presence or absence of the clearing anti-tag mAb. Methods of testing VHHs are shown in Sepulveda et al. 2009 Infect Immun 78:756-763, and Tremblay et al. 2010 Toxicon. 56(6): 990-998. Pools/mixtures of two, three, four or six different anti-BoNT/A VHH monomers with or without anti-E-tag clearing antibody were co-administered to subjects with an amount (1000 LD₅₀ or 10,000 LD₅₀) of BoNT/A holotoxin. Subjects were then monitored for symptoms of toxin lethality and were observed for time to death.

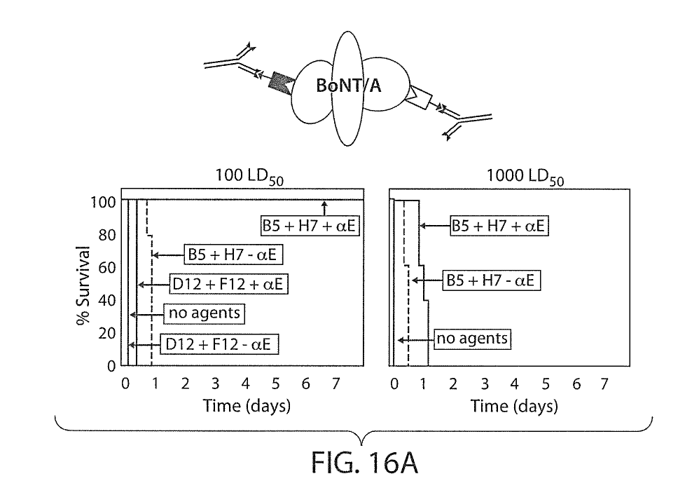

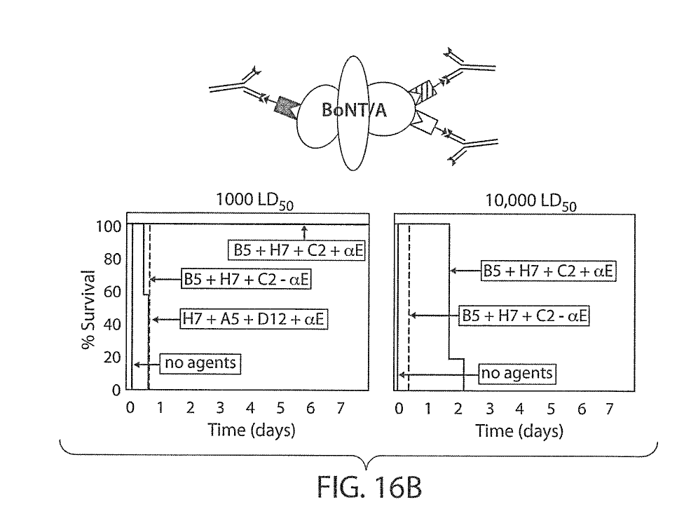

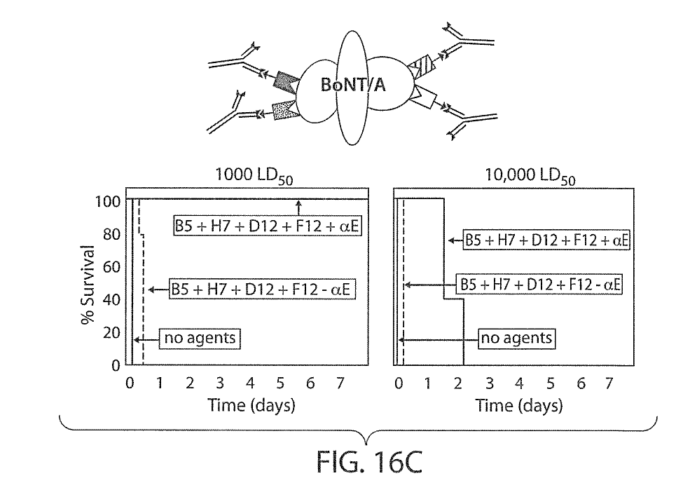

The subjects were co-administered BoNT/A with either a mixture of ciA-H7 and ciA-B5 monomers, or a mixture of ciA-D12 and ciA-F12 monomers (FIG. 16 A bottom graphs). Each mixture was administered with (+αE) or without (−αE) anti-E-tag clearing antibody that specifically bound the epitopic tags located on the VHHs. Control subjects were administered toxin only. Unless indicated otherwise, a dashed line in FIGS. 16-24 indicates that no anti-E-tag antibody was administered to the subjects. Each monomeric VHH was used at a total dose of two micrograms (μg) per mouse to ensure that the only the complexity and/or identity of the VHH mixtures was varied among groups and was the cause of observed antitoxin efficacy.

Results obtained show that subjects administered ciA-D12 and ciA-F12, two anti-BoNT/A VHH monomers previously determined not to neutralize BoNT/A in cell assays, did not survive toxin challenge for any greater time than did control subjects administered toxin only (FIG. 16A bottom graphs). Administration of 5 μg amounts of anti-E-tag clearing antibody (αE) to subjects only slightly prolonged time before death. Data show that subjects administered neutralizing VHH monomers ciA-H7 and ciA-B5 with anti-E-tag clearing antibody were slightly protected against BoNT/A compared to subjects administered ciA-D12 and ciA-F12, and anti-E-tag clearing antibodies. Thus, the decoration of BoNT/A with two clearing antibodies provided little or no therapeutic benefit to the subjects.

Administration to subjects of a mixture of ciA-B5, and ciA-H7 monomers absent clearing antibody only delayed time to death. Data show that subjects challenged with 100-fold the LD₅₀ of BoNT/A (approximately 5 nanograms total) survived longer following administration of a mixture of neutralizing ciA-B5 and ciA-H7 compared to control subjects administered no VHHs. Most important, it was observed that co-administration of clearing antibody and the neutralizing VHHs resulted in 100% survival of subjects challenged with 100-fold the LD₅₀ of BoNT/A (FIG. 16A bottom left graph). At a challenge of 1,000-fold the LD₅₀ of BoNT/A, death was delayed about one additional day for subjects co-administered a mixture of ciA-B5 and ciA-H7 and anti-E-tag clearing antibody compared to subjects administered VHHs alone or control subjects (FIG. 16A bottom right graph). Thus, it was observed that administering a mixture of toxin neutralization VHH monomers with clearing antibody provided greater therapeutic benefit and protection against BoNT/A than administering VHHs absent the clearing antibody. Relative affinity of each VHH influences the therapeutic effect of the VHH, likewise for VHHs having similar sub-nanomolar affinities (See Table 5).

Whether mixtures of VHH monomers containing both neutralizing VHHs and non-neutralizing VHHs were effective antitoxin agents was further tested. Subjects were co-administered 1,000-fold or 10,000-fold BoNT/A LD₅₀ and one VHH monomer mixture of either a mixture of ciA-B5, ciA-117, and ciA-C2; or a mixture of ciA-H7, ciA-A5 and ciA-D12 with (+αE) or without (−αE) an anti-E-tag clearing antibody preparation that specifically binds the epitopic tags located on the VHHs (FIG. 16B bottom graphs). Control subjects were administered toxin only.

Administration of a mixture of ciA-B5, ciA-H7, ciA-C2 monomers, each capable of potent toxin neutralization, delayed death less than a day in mice exposed to 1000-fold the LD₅₀ of BoNT/A (FIG. 16B bottom left graph). Subjects were completely protected (100% survival) at 1000-fold the LD₅₀ of BoNT/A following administration mixture of ciA-B5, ciA-H7, and ciA-C2 monomers and clearing antibody. Co-administration of 10,000-fold the LD₅₀ of BoNT/A (a total amount of 0.5 μg), a mixture of ciA-B5, ciA-H7, ciA-C2 monomers and clearing antibody delayed death more than two days in subjects (See FIG. 16B bottom right graph) compared to control subjects.

It was observed that administration of a mixture of ciA-H7, ciA-A5, and ciA-D12 in which two VHH monomers (ciA-A5 and ciA-D12) in the mixture of monomers were weak toxin neutralizers, resulted in subjects surviving much less after exposure to an amount of BoNT/A 1,000-fold BoNT/A LD₅₀ (FIG. 16B bottom left graph).

Thus, administration of the mixture of ciA-B5, ciA-H7, and ciA-C2 tagged monomers, each of which are strong neutralizing VHHs, to subjects provided greater protection against BoNT/A than the mixture of ciA-H7, ciA-A5 and ciA-D12, in which two of the three VHH monomers do not neutralize BoNT/A. Data show that 100% of subjects administered the mixture of ciA-B5, ciA-H7, and ciA-C2 with the anti-tag clearing antibody survived a dose of BoNT/A that was 1,000-fold the LD₅₀ of a BoNT/A (FIG. 16B bottom left graph), and survived additional days following administration of 10,000-fold the LD₅₀ of a BoNT/A (FIG. 16B bottom left graph).

TABLE 5
SPR binding data for VHH monomers and heterodimers

| clone | protein | epitope^{#} | neutralization* | SPR Kd (nM) | subunit\hat{ } | Genbank |
|---|---|---|---|---|---|---|
| JDY-33 | ciA-H7 | A1 | strong | 0.06 ± 0.07 | Lc | HQ700708 |
| JDT-2 | ciA-D1 | A1 | strong | 0.71 ± 0.004 | Lc | |
| JEC-3 | ciA-H4 | A1 | not done | 1.54 ± 0.06 | Lc | |
| JEC-11 | ciA-H11 | A1 | not done | 4.3 ± 0.09 | Lc | |
| JDY-46 | ciA-C2 | A2 | strong | 2.7 ± 3.1 | Lc | HQ700705 |
| JDY-9 | ciA-B5 | A3 | strong | 0.17 ± 0.06 | Hc | HQ700704 |
| JED-27 | ciA-F12 | A4 | none | 0.24 ± 0.03 | Lc | HQ700706 |
| JDU-26 | ciA-D12 | A5 | none | 0.21 ± 0.1 | Lc | HQ700702 |
| JDY-2 | ciA-A5 | A6 | weak | 1.05 ± 0.05 | none | HQ700703 |
| JDY-59 | ciA-G5 | A7 | weak | 0.32 ± 0.03 | none | HQ700707 |
| JFA-10 | ciB-H11 | B1 | not done | 0.26 ± 0.01 | none | |
| JFX-30 | ciB-A11 | B2 | not done | 0.84 ± 0.68 | none | |
| JFV-48 | ciB-B5 | B3 | not done | 0.97 ± 0.04 | none | |
| JFV-40 | ciB-B9 | B4 | not done | 23 ± 5.8 | none | |
| JEZ-2 | ciA-H7/B5 | A1/A3 | strong | 0.014 ± 0.007 | not done | |
| JFK-21 | ciA-F12/D12 | A4/A5 | not done | 0.097 ± 0.038 | not done | |
| JGA-3 | ciB-A11/B5 | B2/B3 | not done | 5.3 ± 1.5 | not done | |

Complete survival (100%) was observed for subjects administered a mixture of ciA-B5, ciA-H7, ciA-D12 and ciA-F12 tagged monomers and anti-tag clearing antibodies of the challenge with an amount of BoNT/A that was 1,000-fold the LD₅₀ (FIG. 16 C bottom left graph). Administering a pool of anti-BoNT/A VHHs (ciA-B5, ciA-H7, ciA-D12 and ciA-F12) in which only two VHHs (ciA-B5, ciA-H7) were strong toxin neutralizers only slightly delayed death in subjects exposed to 1000-fold the LD₅₀ of BoNT/A (FIG. 16C bottom left graph). At 10,000-fold the LD₅₀ of a BoNT/A, subjects co-administered the mixture of four VHH tagged monomers and anti-tag clearing antibody survived additional days compared to control subjects (FIG. 16C bottom left graph).

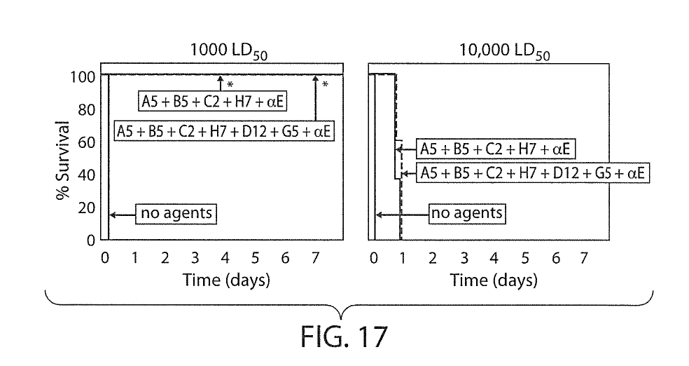

The antitoxin efficacy of a pool of four anti-BoNT/A VHHs tagged monomers (ciA-A5, ciA-B5, ciA-C2 and ciA-H7) was compared to a pool of six different VHH tagged monomers (ciA-A5, ciA-B5, ciA-C2, ciA-H7, ciA-D12, and ciA-G5). The pool of six VHH monomers contained the same VHHs as the pool of four VHHs and further included two VHHs (ciA-D12, and ciA-G5) that were weak neutralizers of BoNT/A (FIG. 17 and Table 5). The different pools of VHH monomers were each administered in the presence of clearing anti-tag antibody. It was observed that 100% of subjects administered either the pool of four VHH tagged monomers or the pool of six VHHs tagged monomers with anti-tag clearing antibody survived challenge with 1,000-fold the LD₅₀ of BoNT/A (FIG. 17 left graph). Subjects challenged with 10,000-fold the LD₅₀ of BoNT/A survived one day, following co-administration of either the pool of four VHH monomers or the pool of six VHH monomers with clearing anti-tag antibody in comparison to control subjects administered only toxin that died immediately (FIG. 17 right graph). These results show that decoration of BoNT/A with a greater number of VHH antibodies, four or more VHHs, greatly improved antitoxin efficacy. Administering a pool of four VHH monomers or a pool of six VHH monomers to the subjects provided additional antitoxin efficacy compared to administering three or fewer VHH monomers.

These data show that toxin clearance was rendered much more effective under conditions in which BoNT is decorated by at least three VHH antibodies and at least about three clearing antibodies. It was observed also that mixtures of monomers having greater number or percentage of toxin neutralization VHHs greatly contributed to percent survival of subjects co-administered a vast excess of the lethal dose of BoNT/A.

### Example 16

### VHH Affinity and Antitoxin Efficacy

Toxin neutralization and clearance mechanisms were observed to depend on affinity of antitoxin binding to the toxin. Without being limited by a particular theory or mechanism of action, the kinetics of toxin binding (Kₒₙ) and release (K_{off}) by the antitoxin binding agents were observed to have contributed to the antitoxin efficacy.

To determine the relationship of toxin affinity to antitoxin efficacy and the role of each, assays were performed to identify multiple VHHs recognizing the same epitope. In the course of anti-BoNT/A VHH screening and based on competition ELISA analysis, several VHHs (ciA-D1, ciA-H4 and ciA-H11) were identified that recognized the same epitope as ciA-H7. SPR analysis showed that each VHH monomer recognized and bound the ciA-H7 epitope with a different affinity. The dissociation constant (Kd) identifies the strength of binding or affinity between a ligand and a receptor, between the VHH antibody and the toxin.

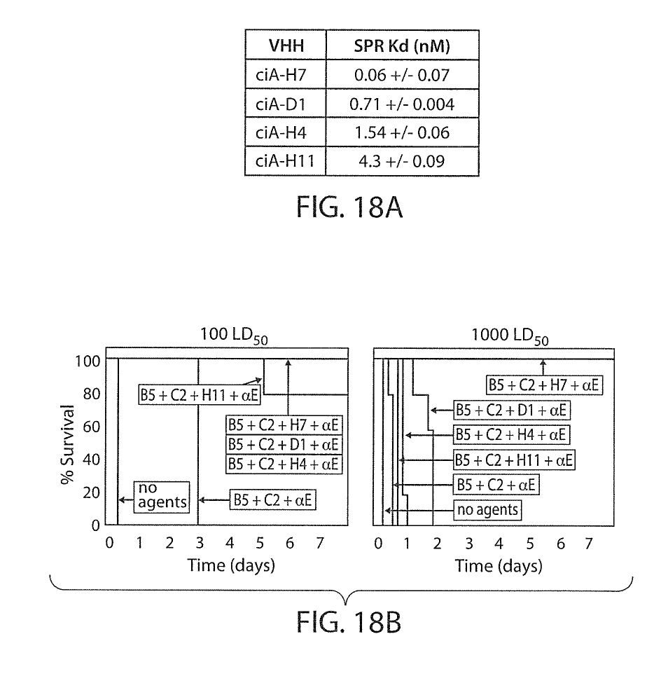

The VHH Kd values for the VHHs having the stronger binding to BoNT/A were determined to be 0.06±0.07 nM for ciA-H7, 0.71±0.004 for ciA-D1, and the VHH Kd values for the VHHs having the weakest binding to BoNT/A were determined to be the 1.54±0.06 for ciA-H4, and 4.3±0.09 for ciA-H11 respectively (FIG. 18A). These four VHHs were tested with anti-tag clearing antibody for their efficacy as antitoxin VHHs in combination with the two VHHs (ciA-B5, ciA-C2) that recognize distinct, non-overlapping epitopes of BoNT/A (FIG. 18B left and right graphs).

Subjects (five mice per group) were co-administered BoNT/A and one of four mixtures containing three VHH monomers: ciA-H7, ciA-B5 and ciA-C2; ciA-D1, ciA-B5 and ciA-C2; ciA-H4, ciA-B5 and ciA-C2; or ciA-H11, ciA-B5 and ciA-C2. Each mixture included two strong neutralizing VHH monomers (ciA-B5 and ciA-C2), and one VHH of ciA-H7, ciA-D1, ciA-H4, or ciA-H11. Control subjects received toxin only.

Data show that 100% of subjects survived following co-administration of 100 BoNT/A LD₅₀ and VHH mixtures containing ciA-B5 and ciA-C2 and either ciA-H7, ciA-D1 or ciA-H4. Subjects administered the VHH mixture of ciA-B5, ciA-C2 and ciA-H11 survived the 100 LD₅₀ of BoNT/A at 80% (FIG. 18 B left graph). Among subjects challenged with 1,000-fold the LD₅₀ of a BoNT/A (FIG. 18 B right graph), the level of protection was a function of the relative binding affinity or Kd of the VHH to BoNT/A shown in FIG. 18 A. Specifically the greatest protection at 1,000-fold BoNT/A LD₅₀ was observed in subjects administered the VHH mixture containing ciA-B5, ciA-C2, and ciA-H7, which had the strongest BoNT/A affinity (i.e., lowest Kd value of 0.06±0.07; FIG. 18A and FIG. 18B right graph). The least extent of protection was observed in subjects administered the VHH mixture containing ciA-B5, ciA-C2, and ciA-H11 (weakest BoNT/A affinity and highest Kd value of 4.3±0.09; FIG. 18A and FIG. 18B right graph), in which survival was comparable to that of control subjects not administered VHH monomers.

Correlating the Kd values with antitoxin-toxin binding and affinities, it was observed that the lower Kd value was a measure of the greater the respective toxin affinity and the greater the antitoxin efficacy in vivo of the VHH. VHH ciA-H7 was observed to have the lowest Kd and the strongest binding affinity to BoNT/A, and was observed to have greater antitoxin efficacy than other VHH compositions identified in FIG. 18 A. Thus, sub-nanomolar affinities or Kd values for the tagged toxin binding agents was determined to be an important factor in identifying the VHH with greatest antitoxin efficacy and most efficacy to protect subjects from toxin-associated pathology and death.

### Example 17

### Antitoxin VHHs Heterodimers

A resulting multimeric binding protein molecule was obtained by engineering and expressing two anti-BoNT/A VHHs as a heterodimer, and this composition was found to bind to two different sites on the toxin and yield an improved toxin affinity. Examples herein analyzed the role of epitopic tags on the heterodimer and the role of the amount of the tagging of the heterodimer compared to the clearing antibody with respect to increasing antitoxin efficacy of the heterodimer.

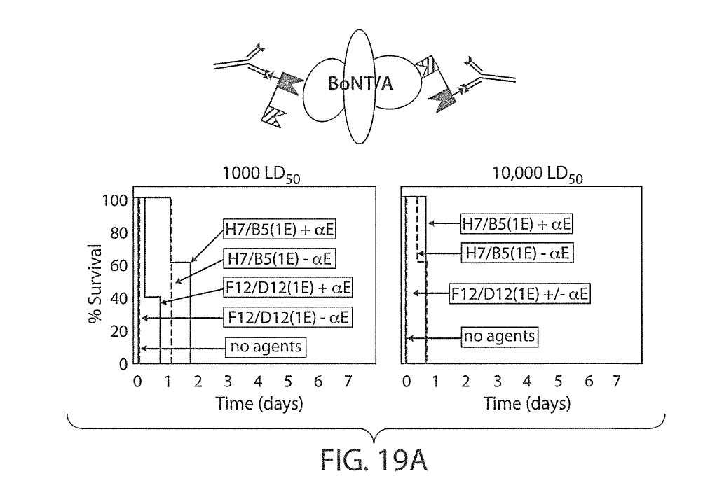

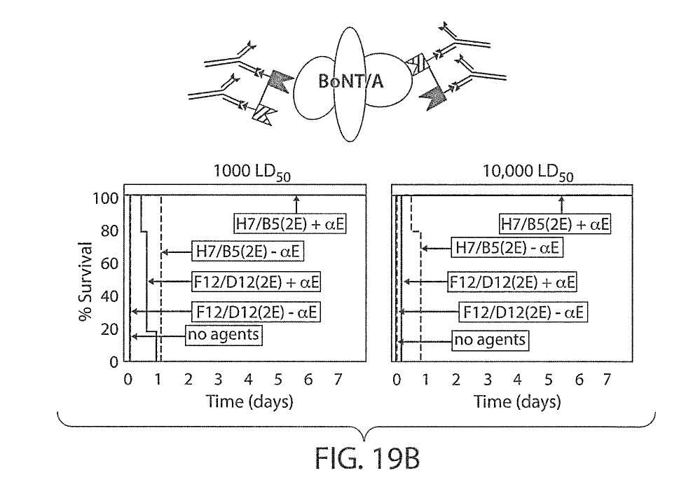

VHH heterodimers were engineered to contain an epitopic tag for decoration of BoNT/A with two anti-tag clearing antibodies (FIG. 19A top drawing). Survival and protection of subjects was analyzed following challenge with each of 100-fold and 1000-fold the LD₅₀ of BoNT/A (FIG. 19A bottom left and right graphs). Data show that administering heterodimer containing two strongly neutralizing VHHs, ciA-B5 and ciA-H7, resulted in greater antitoxin efficacy as measured by longer survival of subjects than administering heterodimers containing two weak or non-neutralizing VHHs, ciA-D12 and ciA-F12 (FIG. 19A bottom left and right graphs).

Presence of second copy of the epitopic tag to the heterodimers compared to one epitopic tag was observed to promote toxin decoration with four clearing antibodies and to yield greater clearing efficacy (FIG. 19B top drawing). All (100%) of subjects survived a challenge with either 1000-fold or 10,000-fold the LD₅₀ of BoNT/A and co-administration of ciA-B5/ciA-H7 heterodimer having two epitopic tags and anti-tag clearing antibody (FIG. 19B bottom graphs).

To further analyze whether two or more epitopic tags improved heterodimer antitoxin efficacy, two sets of anti-BoNT/A VHH heterodimers were constructed in which the two VHHs in the heterodimers were either non-neutralizing (ciA-D12/F12) or potent toxin neutralizing agents (ciA-B5/H7). The two different VHH heterodimers were engineered to contain either one or two copies of the epitopic tag (E-tag) and these proteins were expressed and characterized. SPR analysis determined that the heterodimer affinities were in the range of 10 picomolar to 100 picomolar which was significantly greater than the affinities of the component monomers (FIG. 15 and Table 5).

The antitoxin efficacies of the single tagged heterodimers administered to mouse subjects after challenge with 1000-fold LD₅₀ of BoNT/A (FIG. 19 A bottom left graph) were observed to be similar efficacies observed after administering a mixture of the two corresponding monomers only (FIG. 16 A bottom right graph). Administering the non-neutralizing single-tagged heterodimer, ciA-D12/F12(1E), resulted in no protection from challenge with 1000-fold LD₅₀ of BoNT/A in the absence of clearing antibody, and only slightly delayed death in the presence of clearing antibody (FIG. 19A bottom left graph). The toxin neutralizing single-tagged heterodimer, ciA-B5/H7(1E), delayed death in mice exposed to 1000 LD₅₀ BoNT/A for one to two days in the absence of clearing antibody and efficacy was only slightly improved by the addition of clearing antibody (FIG. 19A bottom left graph).

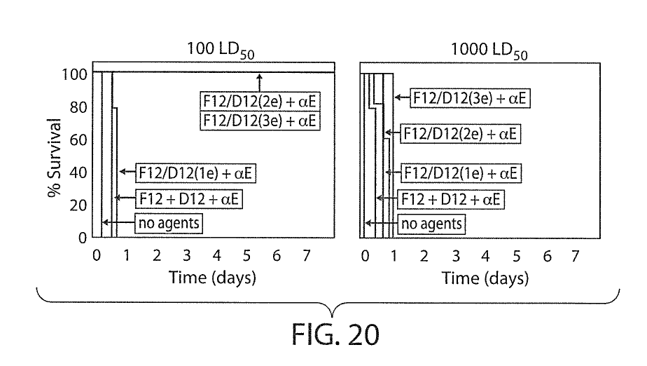

Improved antitoxin efficacy was observed in subjects administered a heteromultimeric agent having a second copy of the epitopic tag, with either of the non-neutralizing and neutralizing anti-BoNT/A VHH heterodimers in which the heterodimer agent was co-administered with clearing antibody. Without being limited by any particular theory or mechanism of action, it is here envisioned that component binding regions in a ‘double-tagged heterodimer’ bind at two sites on the toxin and each bound heterodimer decorates toxin with two clearing antibodies, resulting in decoration of the toxin with at least four clearing antibodies (FIG. 19B top drawing) which Examples herein show had increased clearance. Administering non-neutralizing double-tagged heterodimer containing ciA-D12/F12(2E) resulted in virtually no antitoxin efficacy in subjects in the absence of clearing antibody at both 1000-fold and 10,000-fold the LD₅₀ of BoNT/A (FIG. 19 B bottom left and right graphs). In the presence of clearing antibody, ciA-D12/F12(2E) heterodimer fully protected subjects (100% survival) from 100-fold BoNT/A LD₅₀ and delayed death about one day in subjects receiving 1000-fold BoNT/A LD₅₀ compared to control subjects administered no agents (FIG. 19B bottom right graph and FIG. 20 left graph). Thus the presence of a second epitopic tag attached to the heterodimer dramatically improved the antitoxin efficacy.

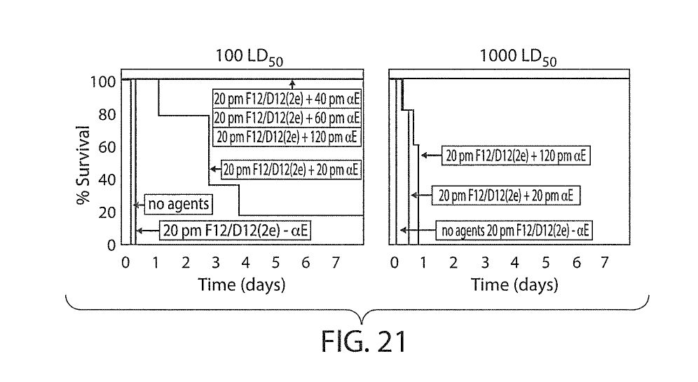

Non-neutralizing heterodimer, ciA-D12/F12, with either one, two or three epitopic tags was analyzed for antitoxin efficacy in the presence of clearing antibody (FIG. 20). The single-tagged heterodimer protected subjects only slightly from toxin challenge of 100-fold the LD₅₀ of BoNT/A. Subjects challenged with double-tagged heterodimers or triple-tagged heterodimers were fully protected from a challenge of 100-fold the LD₅₀ of BoNT/A (FIG. 20 left graph). Only little improvement in antitoxin efficacy was observed with the triple-tagged heterodimers compared to the double-tagged heterodimers, consistent with the observation that near maximal clearance was achieved by decorating the target with four clearing antibodies. A titration of the clearing antibody administered with the double-tagged ciA-D12/F12 heterodimer demonstrated that maximal antitoxin efficacy against each of 100-fold and 1,000-fold the LD₅₀ of BoNT/A was achieved with the number of clearing antibody molecules (measured in picomoles) administered in an amount approximately equivalent to the number of epitopic tags (FIG. 21 left and right graphs).

An even more dramatic antitoxin effect was observed in cell culture intoxication assays using the double-tagged heterodimer, ciA-B5/H7(2E), in which both of the component anti-BoNT/A VHHs individually possess potent neutralizing activity (FIG. 15). In the absence of clearing antibody, the double-tagged ciA-B5/H7(2E) heterodimer produced the same antitoxin efficacy as the equivalent single-tagged heterodimer (compare FIG. 19A bottom left and right graphs to FIG. 19 B bottom left and right graphs). In the presence of clearing antibody, the neutralizing double-tagged heterodimer at 40 picomoles (pmoles) was observed to be a highly potent antitoxin that fully protected cells from lethality when co-administered with 10,000-fold the LD₅₀ of BoNT/A, i.e., the total amount was about 3 pmoles.

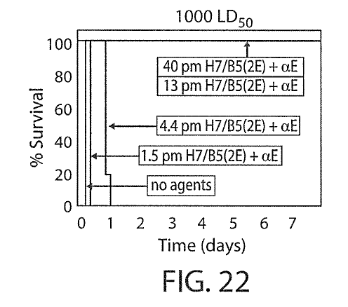

A dose-response assay was performed in mouse subjects with double-tagged ciA-B5/H7(2E) heterodimer co-administered with 1000-fold the LD₅₀ of BoNT/A (FIG. 22). It was observed that each of 40 pmoles and 13 pmoles of double-tagged ciA-B5/H7(2E) heterodimer completely protected the subjects after exposure to 1000-fold the LD₅₀ of BoNT/A. A dose of 4 pmoles ciA-B5/H7(2E) heterodimer had the same protective efficacy for 1,000-fold the LD₅₀ of BoNT/A as a dose of 40 pmoles did with 10,000-fold the LD₅₀ of BoNT/A (FIG. 15B and FIG. 22). These data show that co-administering about a fifteen-fold molar excess of the double-tagged heterodimer binding agent with the clearing antibody was sufficient to effectively neutralize and/or clear substantially all (greater than 99.99%) of the BoNT/A.

### Example 18

### Recombinant Antitoxin Efficacy in a Clinically Relevant Post-Intoxication Assay

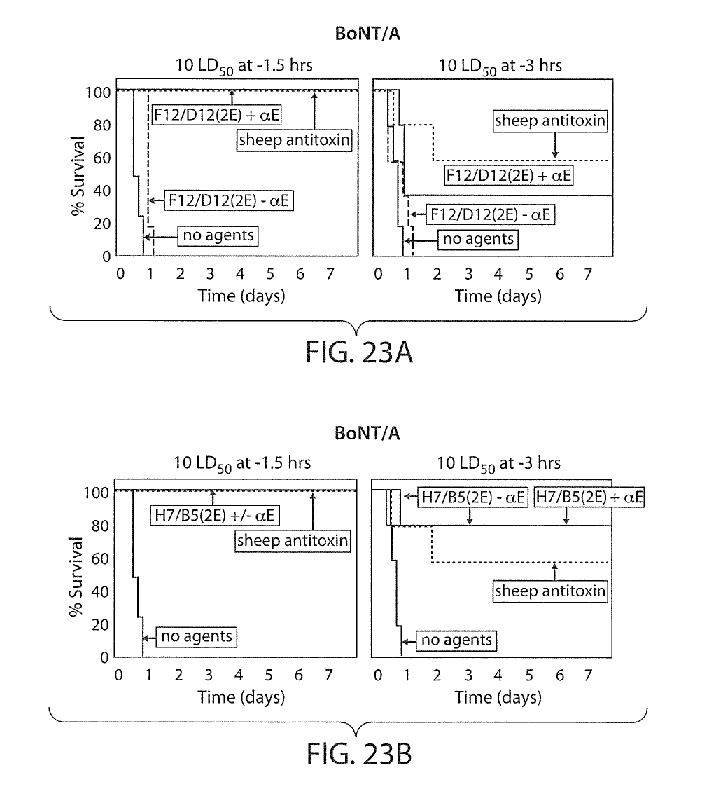

Sensitive quantification of antitoxin efficacy was achieved using assays in which a varied dose of toxin is co-administered with antitoxin agents were observed to permit. To more accurately reflect the typical clinical situation, antitoxin agents were tested in an assay of greater clinical relevance by administering to mouse subjects ten-fold the LD₅₀ of BoNT/A intraperitoneally, and at 1.5 hours and three hours afterwards, administering neutralizing heterodimer antitoxin agents intravenously with and without the anti-tag clearing antibody. Different sets of anti-BoNT/A VHH heterodimers were tested: a heterodimer containing non-neutralizing double-tagged ciA-D12/F12(2E), and a heterodimer containing neutralizing double-tagged ciA-H7/B5(2E) heterodimer (FIG. 23A-FIG. 23B). A potent sheep anti-BoNT/A serum was used as a control in the assay at a dose demonstrated to protect 100% of mice from lethality given 10,000-fold the LD₅₀ of BoNT/A.

The non-neutralizing ciA-D12/F12(2E) heterodimer was observed to have little or no antitoxin efficacy in absence of clearing antibody following administration either 1.5 hours or three hours after BoNT/A challenge (FIG. 23A left and right graphs). However, ciA-D12/F12(2E) heterodimer administered with clearing antibody displayed an efficacy nearly equivalent to the positive control sheep antiserum (FIG. 23B left and right graphs). These results show that toxin clearance alone is sufficient to protect mice from a low dose BoNT challenge, even when administered 1.5 or three hours post-exposure to toxin.

Surprisingly the neutralizing ciA-H7/B5(2E) heterodimer was observed to be as highly effective as an antitoxin in this assay, in the presence or even absence of clearing antibody (FIG. 23 B). The double-tagged toxin neutralizing heterodimer administered 1.5 hours after toxin challenge with ten-fold the LD₅₀ of BoNT/A resulted in an antitoxin efficacy equivalent to the sheep serum polyclonal antitoxin. It was observed that following challenge at 10 BoNT/A LD₅₀ for 1.5 hours, subjects administered ciA-H7/B5(2E) heterodimer absent anti-tag clearing survived fully (100% survival; FIG. 23B left graph). The survival for subjects administered ciA-H7/B5(2E) heterodimer was comparable to subjects administered sheep antitoxin (FIG. 23B left graph).

Data show that administration after three hours after toxin challenge at ten-fold the LD₅₀ of BoNT/A, of the neutralizing ciA-H7/B5(2E) heterodimer resulted in greater subject survival (80%) than of the sheep serum polyclonal antitoxin (60% survival; FIG. 23 B right graph). Most important, the extent of survival of subjects using neutralizing ciA-H7/B5(2E) heterodimer was the same with or without clearing antibody (FIG. 23B right graph).

These data show that BoNT neutralization was sufficient for full antitoxin efficacy in a clinically relevant post-intoxication (post-exposure to toxin) assay with low dose toxin challenge. A single recombinant multimeric binding protein with potent toxin neutralization properties was as effective as antitoxin sera in a model of a typical clinical situation involving toxin exposure and subsequent treatment at a later time point.

### Example 19

### Antitoxin Efficacy of a Double-Tagged Heterodimer Targeting Botulinum Toxin, BoNT/B

Double-tagged VHH heterodimer antitoxins that specifically recognized and bound unique epitopes on BoNT/B holotoxin (FIG. 13B) were identified and expressed. Two of the VHHs, ciB-A11 And ciA-B5, were observed to be the most effective antitoxins of those obtained from monomer pool assays, and were engineered and expressed as double-tagged heterodimer, ciB-A11/B5(2E).

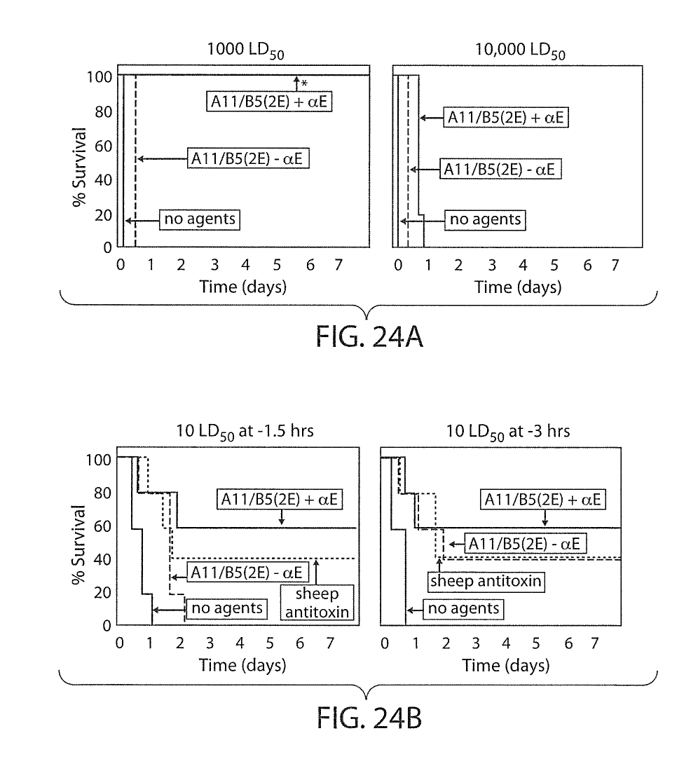

Subjects were exposed to either 1,000-fold (FIG. 24 A left graph) or 10,000-fold (FIG. 24A right graph) BoNT/B LD₅₀, and were administered a ciB-A11 And ciB-B5 heterodimer with (+αE) or without (−αE) anti-tag clearing antibody. Control subject were exposed only to toxin (no therapeutic agents, vix, no heterodimer binding proteins). Data show that in the presence of clearing antibody the ciB-A11/B5(2E) heterodimer fully protected subjects challenged with 1000-fold the LD₅₀ of BoNT/B (FIG. 24A left graph) and extended the life of subjects challenged with 10,000-fold the LD₅₀ of BoNT/B (FIG. 24A right graph).

Analysis was performed to determine whether the ciB-A11 And ciA-B5 double tagged heterodimer was effective to treat subjects in a BoNT/B post-exposure in vivo model.

Subjects were exposed intravenously to 10 LD₅₀ of BoNT/A, and then administered 1.5 hours or three hours afterward either: ciB-A11 And ciA-B5 double tagged heterodimeric protein with or without clearing antibody, or a sheep antitoxin serum. Control subjects were exposed to 10 LD₅₀ of BoNT/B only (no heterodimeric binding protein agents administered). See FIG. 24 B left and right graphs. Data show 60% of subjects administered ciB-A11/B5 double tagged heterodimer with anti-tag antibody survived 1.5 hours and three hours after BoNT/B exposure, and which is 20% more of the subjects that survived than those treated with sheep antitoxin at each of the time points (FIG. 24B left and right graphs). It was observed that subjects administered A11/B5 double tagged heterodimer binding protein only (without anti-tag antibody three hours after BoNT/B exposure) survived as long as subjects administered sheep antitoxin (FIG. 24B right graph).

Results herein from these clinically relevant post-intoxication assays showed that ciB-A11/B5 heterodimer with or without clearing antibody was as effective as sheep anti-BoNT/B serum in protecting subjects from death caused by BoNT/B holotoxin exposure.

### Example 20

### VHH Monomers Protect CT26 Cells from TcdA

Cells of murine colorectal cancer cell line CT26 were exposed to TcdA (2 ng/ml) for 24 hours and were then administered a VHH monomer specific to TcdA (A3H, SEQ ID NO 61; AUG, SEQ ID NO:63; AC1, SEQ ID NO: 62; AE1, SEQ ID NO: 64; AH3, SEQ ID NO: A1; or AA6, SEQ ID NO: 60). Controls cells were exposed to TcdA and no therapeutic VHH monomer was administered. Percentage cell rounding was monitored using a phase contrast microscope. Control cells administered only TcdA showed extensive cell rounding and distorted cell morphology associated with TcdA toxin exposure.

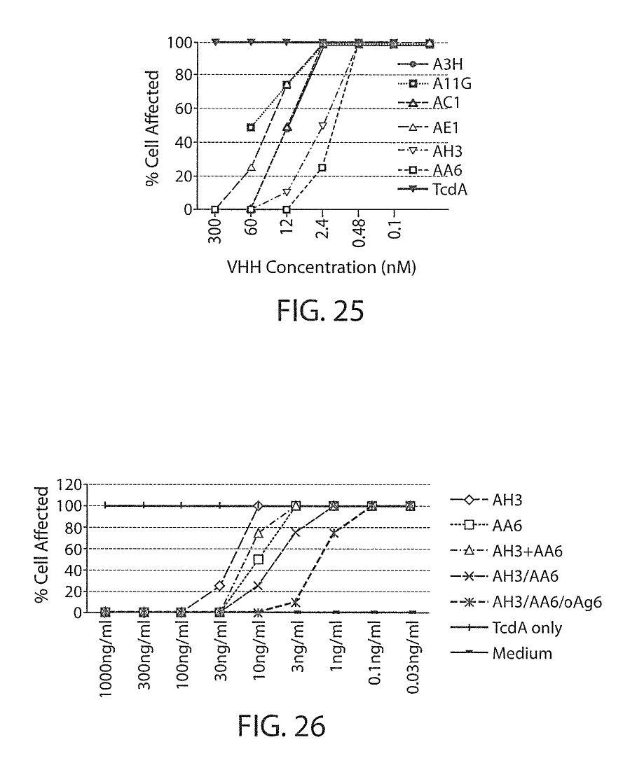

It was observed that each of the VHH monomers reduced the percentage of affected cells and protected the cells from the pathological effects of TcdA exposure (FIG. 25). In order of greatest VHH monomer activity to the weakest VHH monomer activity, the greatest activity was observed for AA6, followed AH3, AC1, A3H, AE1, and A116 respectively. It was observed that VHH monomers AA6 and AH3 neutralized TcdA and protected 50% of cells from toxin cytotoxicity at VHH concentrations less than about 10 nM, and thus were considered to have strong TcdA neutralizing activity.

### Example 21

### Multimeric Binding Proteins Protect Cells from TcdA

CT26 cells were exposed to TcdA (2 ng/ml) and then administered a concentration (0.1 nM, 0.48 nM, 2.4 nM, 12 nM, 60 nM, or 300 nM) of each of VHH monomers: A3H, A11G, AC1, AE1, AH3, or AA6. Control cells were exposed to toxin only. The strength of each neutralizing VHH activity was observed by analyzing extent of protection of cells from the toxin by VHH monomers. Percentage of cell rounding (% cell affected) caused by TcdA was monitored using a phase contrast microscope (FIG. 25). Thus, the strongest VHH produced the greatest protection at the lowest concentration. The VHHs were identified in the following order of efficacy: AA6 as the strongest therapeutic agent, followed by AH3, AC1, AE1, A11G, and then A3H as weakest therapeutic agent.

To determine whether VHH monomers or VHH multimers effectively neutralized TcdA, CT26 cells were exposed for 24 hours to TcdA (2 ng/ml) and to a different concentration (0.03 ng/mL, 0.1 ng/mL, 1 ng/mL, 3 ng/mL, 10 ng/mL, 30 ng/mL, 100 ng/mL, 300 ng/mL, or 1000 ng/mL) of VHH monomers (AH3 or AA6), VHH heterodimer containing AH3 and AA6, or a homodimer of the heterodimer containing the heterodimer of AH3 and AA6 and fused to an artificial homodimerization domain called oAgBc (Ah3/AA6/oAgBc; SEQ ID NO: 95). The oAgBc domain peptide has amino acid sequence TSPSTVRLESRVRELEDRLEELRDELERAERRANEMSIQLDEC (SEQ ID NO: 94) that binds to proteins having the same sequence to form homodimers. The cysteine (three letter amino acid abbreviation Cys or one letter C) at the carboxyl end of oAgBc becomes oxidized forming a covalent disulfide linkage between the two protein molecules to stabilize the homodimer (dimerizing sequence). Thus the AH3/AA6 heterodimer per se forms a homodimer containing two copies of AH3/AA6 joined by the oAgBc dimerization domain (SEQ ID NO: 95). Control cells were exposed to toxin only and not to VHH agents. The percentage of cell rounding (% cell affected) was monitored using a phase contrast microscope (FIG. 26).

Data show that each of the VHH monomers, AH3/AA6 heterodimer and AH3/AA6/oAgBc heterodimer/homodimer neutralized TcdA and protected the CT26 cells from the toxin (FIG. 26), in contrast to control cells contacted with toxin only showed extensive toxin mediated-cell rounding. The AH3/AA6/oAgBc heterodimer/homodimer displayed greatest activity to neutralize and protect cells compared to the AH3/AA6 heterodimer, AH3 monomer, and AA6 monomer respectively. The AH3/AA6/oAgBc heterodimer/homodimer displayed about three-fold stronger neutralizing activity for TcdA and protection of the cells than the AH3/AA6 heterodimer alone, and about ten-fold better activity and protection than the VHH monomers (AH3 and AA6 respectively).

### Example 22

### Heterodimer Binding Proteins Protect Cells from TcdA and TcdB

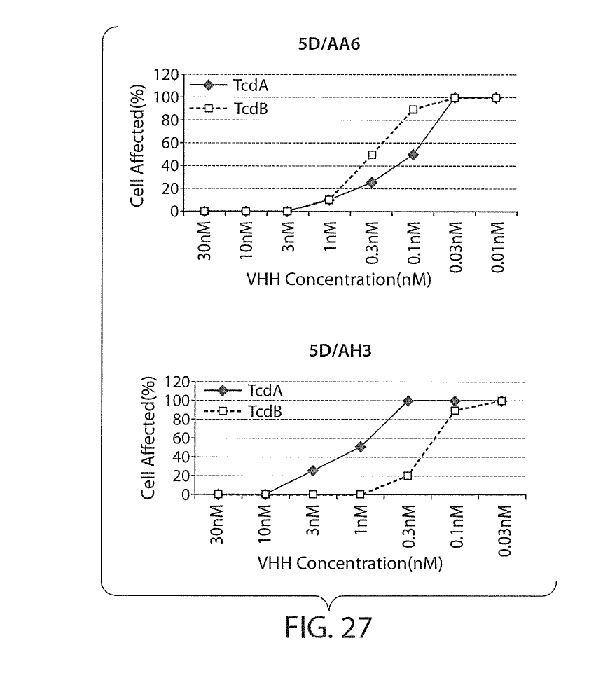

To determine activity of VHH heterodimers to neutralize both TcdA and TcdB, CT26 cells were exposed overnight to TcdA (2 ng/ml) or TcdB (0.1 ng/ml), and then treated with a heterodimer composition containing VHH 5D and VHH AA6 (FIG. 27 left graph) or with a heterodimer composition containing VHH 5D and VHH AH3 (FIG. 27 right graph). Each heterodimer was engineered to contain a VHH (5D) that strongly neutralized TcdB (FIG. 13) and to contain also a VHH (AA6 or AH3) that strongly neutralized TcdA (FIG. 25). The percentage of cell rounding (% cell affected) was monitored using a phase contrast microscope (FIG. 27).

Data show that each of the 5D/AA6 heterodimer and the 5D/AH3 heterodimer neutralized both TcdA and TcdB (FIG. 27). It was observed that 5D/AA6 heterodimer was about five-fold more effective for neutralizing TcdA than the 5D/AH3 heterodimer. Thus, the relative neutralization strength of each heterodimer (FIG. 27) corresponded to the relative neutralization strength of each corresponding AA6 monomer and AH3 monomer shown in FIGS. 25-26.

It was observed that the 5D/AA6 heterodimer was about three-fold or four-fold more effective for neutralizing TcdB than the 5D/AH3 heterodimer. At a concentration of about 0.2 nM of administered 5D/AA6 heterodimer, 50% of cells were protected, compared to about 1 nM of 5D/AH3 heterodimer required for this same level of protection. Without being limited by any particular theory or mechanism of action, it is here envisioned that the relative greater TcdA neutralization ability of the AA6 binding region compared to AH3 binding region resulted in a synergistically greater ability of the respective heterodimer to neutralize TcdB. The increased toxin neutralization for 5D/AA6 for TcdB is presumably caused by amino acid sequences in TcdA and TcdB that are similar and are neutralized effectively by the AA6 component of the heterodimer compared to the AH3 component of the heterodimer.

### Example 23

### 5D/AA6 Heterodimer Protected Subjects from C. difficile Infection

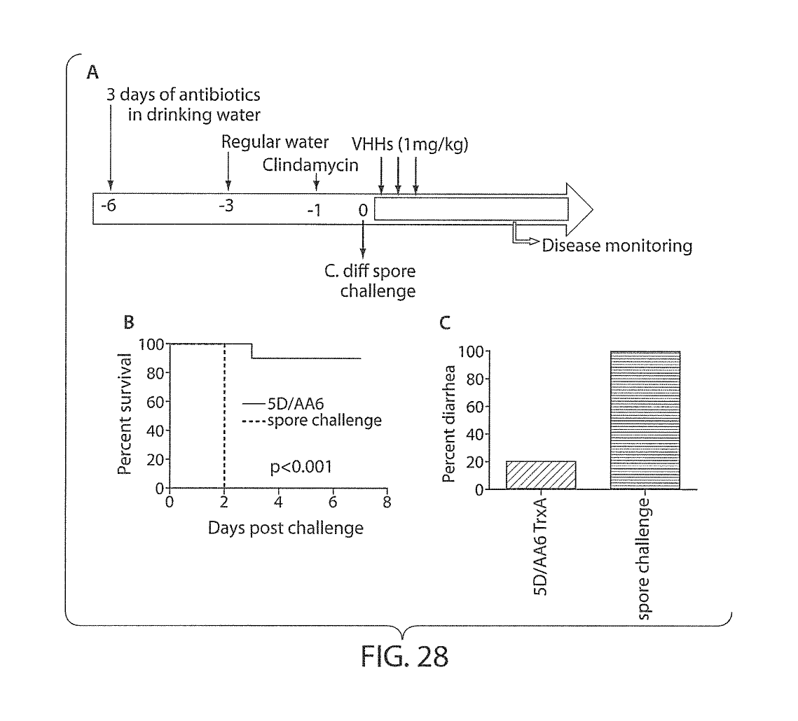

To determine whether a single heterodimer could neutralize both TcdA and TcdB and protect mice from oral C. difficile spore challenge, a protocol for a clinically relevant mouse C. difficile infection model (Chen et al. 2008 Gastroenterology 135: 1984-1992) was performed as shown in FIG. 28. Groups of mice (ten mice/group) were treated to obtain a model of C. difficile associated diarrhea by administration for three days with antibiotics in drinking water of the subjects, and then two days later by intraperitoneal administration of a single dose clindamycin before challenge with spores of a C. difficile strain expressing both TcdA and TcdB (10⁶ spores/subject) on day zero (FIG. 28A). To induce more severe and fulminant disease, steroid dexamethasone was supplied to the subjects in drinking water from day −6 (100 mg/mL) to day zero (Sun et al. 2001 Infection and Immunity 79: 2556-2864). Subjects were injected intraperitoneally with VHH heterodimer containing 5D and AA6 (1 mg/kg) three times (at six hours, 16 hours, and 24 hours following the C. difficile inoculation/challenge). Control subjects were received PBS instead of the VHH heterodimer. Subjects were monitored hours and days following the VHH injection.

Data show that 100% of control subjects administered toxin died within two days of toxin challenge (FIG. 28B) and suffered diarrhea (FIG. 28C). In contrast, only 20% of subjects administered 5D/AA6 heterodimer developed diarrhea and 90% survived for eight days of the timecourse (FIG. 28B and FIG. 28C). Thus, 5D/AA6 heterodimer protected subjects from both TcdA and TcdB spore challenge in a clinically relevant mouse C. difficile infection model.

### Example 24

### Recombinant Multimeric Binding Proteins Neutralize a Plurality of Disease Agents

Effectiveness of the antitoxin treatment using multimeric binding proteins is analyzed by determining ability of the binding proteins to bind to and neutralize a disease agent target.

Recombinant heteromultimeric neutralizing binding protein containing multiple binding regions with or without epitopic tags are produced. The binding regions are not identical and each binding region has affinity to specifically bind a non-overlapping portion of a disease agent: TcdA toxin, TcdB toxin, and a Shiga toxin. The genes encoding proteins are multimerized to form different heteromultimeric binding proteins using the oAgBc dimerization domain (SEQ ID NO: 94) shown in Example 21.

Subjects are exposed to a mixture of disease agents (TcdA toxin, TcdB toxin, Shiga toxin and a norovirus), and then are administered each of the heteromultimeric binding proteins, or a mixture of monoclonal antibodies specific for one of TcdA, TcdB, Shiga Toxin 1, and the norovirus. Control subjects are administered the mixture of disease agents only (no multimeric binding proteins). Subjects are monitored for indicia of exposure to the pathogenic molecules such as diarrhea, fever, tachycardia, respiratory distress, and death.

Meyer-Kaplan plots quantifying survival of subjects are prepared and weeks later surviving subjects are sacrificed to analyze tissue and cell morphology. A surprising synergistic protective effect is observed for subjects administered the multimeric binding proteins with or without epitopic tags. Data show that subjects administered the multimeric binding proteins survive longer and have little or no indicia of exposure to the mixture of disease agents compared results for subjects administered monoclonal antibodies to each disease agent and for control subjects administered only disease agents. Subject administered heteromultimeric binding proteins specific for disease agents do not experience diarrhea, fever or other indicia of exposure to the disease agents. Tissues from subjects administered multimeric binding proteins show normal cell appearance without signs of cell rounding or cell lysis caused by either TcdA, TcdB, Shiga Toxin 1, and the norovirus. The multimeric binding proteins neutralize each of these disease agents. Control subjects have diarrhea, and tissues excised from the intestinal systems show indicia of colitis and extensive internal bleeding.

The multimeric binding protein specific for a mixture of bacterial toxins and a viral infectious agent neutralize each of the disease agents and protected the cells from the subjects from cytotoxicity and cell lysis.

### Example 25

### Materials and Methods

Purified, catalytically inactive mutant forms of full-length recombinant disease agent (shiga toxin, anthrax protective antigen, ricin A chain toxin, or ricin B chain) were obtained. Shiga toxins were obtained from Phoenix Lab at Tufts Medical Center. Purified anti-Stx1 monoclonal antibody (mAb) 4D3, anti-Stx2 mAbs 3D1 And 5C12, and recombinant Stx1B chain and recombinant Stx2 A and B chain were kindly provided by Dr. Abhineet Sheoran. Stx1 And Stx2 toxoids were prepared by formalin inactivation of the holotoxins and then dialyzed. Reagents for Western blotting were purchased from KPL Inc. (Gaithersburg, Md.). Antibodies used were anti-E-tag mAb (Phadia; Uppsala, Sweden); HRP-anti-E-tag mAb (Bethyl Laboratories Inc.; Montgomery, Tex.); HRP-anti-M13 Ab (GE Healthcare; Woburn, Mass.).

### Example 26

### VHHs that Bind Shiga Toxins

VHH binding agents were produced, purified and were screened to identify those that specifically bind to Shiga toxins. It was observed that one resulting VHH, JET-H12, bound specifically to both Shiga-like toxin Stx1 And Stx2. Another VHH, JFG-H6, was observed to bind specifically to Stx2 (See FIG. 29 A-B). The amino acid sequences and nucleotide sequences for JET-H12 and JFG-H6 were determined and are shown below:

JET-H12
(SEQ ID NO: 96)
QVQLVETGGGLVQAGDPLRLSCVASGRTVSRYDKAWFRQAPGKEREFVAGISWNG
DTKIYADSVKGRFTISRENSRDTLDLQIDNLKPEDTAAYYCAVGIAGVQSMARMLG
VRYWGQGTQVTVSSEPKTPKPQ;
(SEQ ID NO: 97)
CAGGTGCAGCTCGTGGAGACGGGGGGAGGATTGGTGCAGGCTGGGGACCCTCT
GAGACTCTCCTGTGTAGCCTCTGGACGCACCGTCAGTCGCTATGACAAGGCCTG
GTTCCGCCAGGCTCCAGGGAAGGAGCGTGAGTTTGTAGCAGGAATTAGCTGGA
ACGGCGATACAAAAATTTATGCAGACTCCGTGAAGGGCCGATTCACCATCTCCA
GAGAGAACTCCAGGGATACACTGGATCTGCAAATTGACAACCTGAAACCTGAG
GACACGGCCGCGTATTACTGTGCGGTCGGAATTGCGGGTGTTCAGAGTATGGCG
CGTATGCTCGGAGTGCGCTACTGGGGCCAGGGGACCCAGGTCACCGTCTCCTCA
GAACCCAAGACACCAAAACCACAA;
JFG-H6
(SEQ ID NO: 98)
QVQLVETGGGLVQPGGSLRLSCAASGFSLDPYVIGWFRQAPGKEREGVSCITSRAAS
RTSVDSVNERFTISRDNAKNTVDLHINNLKPEDSGVYYCAAVPPAKPLFSLCRSLP
AKYDYWGQGTQVTVSSAHHSEDPS;
(SEQ ID NO: 99)
CAGGTGCAGCTCGTGGAGACGGGGGGAGGCTTGGTGCAGCCTGGGGGGTCTCT
GAGACTCTCCTGTGCAGCCTCTGGTTTCAGTTTGGACCCTTATGTGATAGGATGG
TTCCGGCAGGCCCCAGGGAAGGAGCGTGAGGGGGTCTCATGTATTACGAGTAG
GGCTGCTAGTCGAACGTCTGTAGACTCCGTGAACGAGCGATTCACCATCTCCAG
AGACAACGCCAAGAATACGGTCGATCTACACATCAATAACCTGAAACCTGAGG
ACTCGGGCGTTTATTACTGTGCAGCGGTCCCCCCTGCCAAATTACCACTTTTCAG
CCTATGTCGCTCCCTGCCAGCAAAGTATGACTACTGGGGCCAGGGGACCCAGGT
CACCGTCTCCTCAGCGCACCACAGCGAAGACCCCTCG;

### Example 27

### VHHs that Bind Anthrax Protective Antigen

VHH binding agents were produced, purified and identified that are specific to anthrax protective antigen (PA) positive VHHs (See FIG. 29A-FIG. 29B). It was observed that the following VHHs specifically bind anthrax PA: JHD-B6, JHE-D9, JIJ-A12, JIJ-B8, JIJ-C11, JIJ-D3, JIJ-E9, JIJ-F11, JIK-B8, JIK-B1, JIK-B12, and JIK-F4. The amino acid sequence and nucleotide sequence of each of these VHHs were determined and are shown below:

JHD-B6
(SEQ ID NO: 100)
QVQLVESGGGLVQPGGSLRLSCAASGSSFSRYAMRWYRQAPGKQRELVANINSRG
TSNYADSVKGRFTISRDNAKNTVYLQMNSLKPEDTAVYYCNAEWLGRSEPSWGQG
TQVTVSSEPKTPKPQ
(SEQ ID NO: 101)
CAGGTGCAGCTCGTGGAGTCGGGGGGAGGCTTGGTGCAGCCTGGGGGGTCTCTG
AGACTCTCCTGTGCAGCCTCTGGAAGTAGCTTCAGTAGATATGCCATGCGCTGG
TACCGCCAGGCTCCAGGGAAGCAGCGCGAGTTGGTCGCAAACATTAATAGTCGT
GGTACCTCAAACTATGCAGACTCCGTGAAGGGCCGATTCACCATCTCCAGAGAC
AACGCCAAGAACACGGTGTATCTGCAAATGAACAGCCTGAAACCTGAAGACAC
GGCCGTCTATTATTGTAATGCAGAGTGGTTGGGACGATCGGAGCCTTCCTGGGG
CCAGGGGACCCAGGTCACCGTCTCCTCGGAACCCAAGACACCAAAACCACAA
JHE-D9
(SEQ ID NO: 102)
QVQLVESGGGLVQPGGSLRLSCAASGFIFSLYTMRWHRQAPGKERELVATITSATGI
TNYADSVKGRFIISRDDAKKTGYLQMNSLKPEDTAVYYCNAVRTTVSRDYWGQGT
QVTVSSEPKTPKPQ
(SEQ ID NO: 103)
CAGGTGCAGCTCGTGGAGTCAGGAGGAGGCTTGGTGCAGCCTGGGGGGTCTCTG
AGACTCTCCTGTGCAGCCTCTGGATTCATTTTCAGTCTTTATACCATGAGGTGGC
ACCGCCAGGCTCCAGGGAAGGAGCGCGAGTTGGTCGCGACTATTACTAGTGCTA
CTGGTATTACAAACTATGCAGACTCCGTGAAGGGCCGATTCATCATCTCCAGAG
ACGATGCCAAGAAGACGGGGTATCTGCAAATGAACAGCCTGAAACCTGAGGAC
ACGGCCGTGTATTACTGTAATGCAGTCCGCACTACCGTGTCACGAGACTACTGG
GGCCAGGGGACCCAGGTCACCGTCTCCTCAGAACCCAAGACACCAAAACCACA
A
JIJ-A12
(SEQ ID NO: 104)
QVQLVESGGGLVQPGGSLRLSCAASGIIFSIYTMGWYRQAPGKQRELVAAIPSGPSA
NATDSVGGRFTITRDNAENTVYLQMNDLKPEDTAVYYCNARRGPGIKNYWGQGT
QVTVSSEPKTPKPQ
(SEQ ID NO: 105)
CAGGTGCAGCTCGTGGAGTCTGGGGGAGGCTTGGTGCAGCCTGGGGGGTCTCTG
AGACTCTCCTGTGCAGCCTCTGGAATCATCTTCAGTATCTATACCATGGGCTGGT
ACCGCCAGGCTCCAGGGAAGCAGCGCGAATTGGTCGCAGCTATACCTAGTGGTC
CTAGCGCAAACGCTACAGACTCCGTGGGGGGCCGATTCACCATCACCAGAGAC
AACGCCGAGAACACGGTGTATCTGCAAATGAACGACCTGAAACCTGAGGACAC
GGCCGTCTATTACTGTAATGCTCGGCGGGGTCCGGGTATCAAAAACTACTGGGG
CCAGGGGACCCAGGTCACCGTCTCCTCAGAACCCAAGACACCAAAACCACAA
JIJ-B8
(SEQ ID NO: 106)
QVQLVESGGGLVQPGGSLSVSCAASGSIARPGAMAWYRQAPGKERELVASITPGGL
TNYADSVTGRFTISRDNAKRTVYLQMNSLQPEDTAVYYCHARIIPLGLGSEYRDHW
GQGTQVTVSSAHHSEDPS
(SEQ ID NO: 107)
CAGGTGCAGCTCGTGGAGTCCGGGGGCGGCTTGGTGCAGCCCGGGGGGTCTCTG
AGTGTCTCCTGTGCAGCCTCTGGAAGCATCGCAAGACCAGGTGCCATGGCCTGG
TACCGCCAGGCTCCAGGGAAGGAGCGCGAGTTGGTCGCGTCTATTACGCCTGGT
GGTCTTACAAACTATGCGGACTCCGTGACGGGCCGATTCACCATTTCCAGAGAC
AACGCCAAGAGGACGGTGTATCTGCAGATGAACAGCCTCCAACCCGAGGACAC
GGCCGTCTATTACTGTCATGCACGAATAATTCCCCTAGGACTTGGGTCCGAATA
CAGGGACCACTGGGGCCAGGGGACTCAGGTCACCGTCTCCTCAGCGCACCACA
GCGAAGACCCCTCG
JIJ-C11
(SEQ ID NO: 108)
QVQLVETGGGLVQPGGSLGLSCVVASGRSINNYGMGWYRQAPGKQRELVAQISSG
GTTNYAGSVEGRFTISRDNVKKMVYLQMNSLKPEDTAVYYCNSLLRTFSWGQGTQ
VTVSSAHHSEDPS
(SEQ ID NO: 109)
CAGGTGCAGCTCGTGGAGACGGGGGGAGGCTTGGTGCAGCCTGGGGGGTCTCT
GGGACTCTCCTGTGTAGTCGCCTCTGGAAGAAGCATCAATAATTATGGCATGGG
CTGGTACCGCCAGGCTCCAGGGAAGCAGCGCGAGTTGGTCGCGCAAATTAGTA
GTGGTGGTACCACAAATTATGCAGGCTCCGTAGAGGGCCGATTCACCATCTCCA
GAGACAACGTCAAGAAAATGGTGTATCTTCAAATGAACAGCCTGAAACCTGAG
GACACGGCCGTCTATTACTGTAATTCACTGCTCCGAACTTTTTCCTGGGGCCAGG
GGACCCAGGTCACCGTCTCCTCGGCGCACCACAGCGAAGACCCCTCG
JIJ-D3
(SEQ ID NO: 110)
QVQLVETGGLVQPGGSLRLSCAASGLTFSSTAMAWFRQAPGKEREFVARISGAGITI
YYSDSVKDRFTISRNNVENTVYLQMNSLKTEDTAVYYCAARRNTYTSDYNIPARYP
YWGQGTQVTVSSEPKTPKPQ
(SEQ ID NO: 111)
CAGGTGCAGCTCGTGGAGACCGGGGGGTTGGTGCAGCCTGGGGGCTCCCTGCG
ACTCTCCTGTGCAGCCTCCGGACTCACCTTCAGTAGCACTGCCATGGCCTGGTTC
CGCCAGGCTCCAGGGAAGGAGCGTGAGTTTGTAGCACGTATTAGCGGGGCTGGT
ATTACGATCTACTATTCGGACTCCGTGAAGGACCGATTCACCATCTCCAGAAAC
AACGTCGAGAACACGGTGTATTTGCAAATGAACAGCCTGAAAACTGAGGACAC
GGCCGTTTACTACTGTGCAGCAAGACGGAATACTTACACTAGCGACTATAACAT
ACCCGCCCGGTATCCCTACTGGGGCCAGGGGACCCAGGTCACCGTCTCCTCAGA
ACCCAAGACACCAAAACCACAA
JIJ-E9
(SEQ ID NO: 112)
QVQLVETGGLVQPGGSLRLSCAASRSTTATIYSMNWYRQAPGKQRELVAGMTSDG
QTNYATSVKGRFFISRDNAKNTVYLLMNSLKLEDTAVYYCYVKPWRLQGWDYWG
QGTQVTVSSEPKTPKPQ
(SEQ ID NO: 113)
CAGGTGCAGCTCGTGGAGACGGGGGGCTTGGTGCAGCCTGGGGGGTCTCTGAG
ACTCTCCTGTGCAGCCTCTAGAAGCACGACGGCCACAATTTATAGTATGAACTG
GTACCGCCAGGCTCCAGGGAAGCAGCGCGAGTTGGTCGCGGGTATGACTAGTG
ATGGTCAGACAAACTATGCAACCTCCGTGAAGGGCCGATTCACCATCTCCAGAG
ACAACGCCAAGAACACGGTATATTTGCTAATGAACAGCCTGAAACTTGAGGAC
ACGGCCGTCTATTATTGTTATGTAAAACCATGGAGACTACAAGGTTGGGACTAC
TGGGGCCAGGGGACCCAGGTCACCGTCTCCTCAGAACCCAAGACACCAAAACC
ACAA
JIJ-F11
(SEQ ID NO: 114)
QVQLVESGGGLVQPGGSLRLSCAAPESIVNSRTMAWYRQAPGKQRERVATITTAGS
PNYADSVKGRFAISRDNAKNTVYLQMNSLKPEDTAVYYCNTLLSTLPYGQGTQVT
VSSAHHSEDPS
(SEQ ID NO: 115)
CAGGTGCAGCTCGTGGAGTCGGGCGGCGGCTTGGTGCAGCCTGGGGGGTCTCTG
AGACTCTCCTGTGCAGCCCCTGAAAGCATCGTCAATAGCAGAACCATGGCCTGG
TACCGCCAGGCTCCAGGAAAGCAGCGCGAAAGGGTCGCCACTATTACTACTGCT
GGTAGCCCAAATTATGCAGACTCTGTGAAGGGCCGATTCGCCATCTCCAGAGAC
AACGCCAAGAACACGGTATATCTGCAAATGAACAGCCTGAAACCTGAGGACAC
GGCCGTCTATTACTGCAATACACTTCTCAGCACCCTTCCCTATGGCCAGGGGACC
CAGGTCACCGTCTCCTCGGCGCACCACAGCGAAGACCCCTCG;
JIK-B8
(SEQ ID NO: 116)
QVQLVESGGGLVQPGGSLGLSCVVASERSINNYGMGWYRQAPGKQRELVAQISSG
GTTNYADSVEGRFTISRDNVKKMVHLQVNSLKPEDTAVYYCNSLLRTFSWGQGTQ
VTVSSEPKTPKPQ
(SEQ ID NO: 117)
CAGGTGCAGCTCGTGGAGTCGGGCGGAGGCTTGGTGCAGCCTGGGGGGTCTCTG
GGACTCTCCTGTGTAGTCGCCTCTGAAAGAAGCATCAATAATTATGGCATGGGC
TGGTACCGCCAGGCTCCAGGGAAGCAGCGCGAGTTGGTCGCGCAAATTAGTAGT
GGTGGTACCACAAATTATGCAGACTCCGTAGAGGGCCGATTCACCATCTCCAGA
GACAACGTCAAGAAAATGGTGCATCTTCAAGTGAACAGCCTGAAACCTGAGGA
CACGGCCGTCTATTACTGTAATTCGCTACTCCGAACTTTTTCCTGGGGCCAGGGG
ACCCAGGTCACCGTCTCCTCGGAACCCAAGACACCAAAACCACAA
JIK-B10
(SEQ ID NO: 118)
QVQLVETGGGLVQPGGSLRLSCAASGFTFSSYRMSWYRQAAGKERDVVATITANG
VPTGYADSVMGRFTISRDNAKNTVYLEMNSLNPEDTAVYYCNAPRLHTSVGYWG
QGTQVTVSSEPKTPKPQ
(SEQ ID NO: 119)
CAGGTGCAGCTCGTGGAGACGGGAGGAGGCTTGGTGCAGCCTGGGGGGTCTCT
GAGACTCTCCTGTGCAGCCTCTGGATTCACCTTCAGTAGTTATCGCATGAGCTGG
TACCGGCAGGCTGCAGGGAAGGAGCGCGACGTGGTCGCAACAATTACTGCTAA
TGGTGTTCCCACAGGCTATGCAGACTCCGTGATGGGCCGATTCACCATTTCCAG
AGACAATGCCAAGAACACGGTGTATCTGGAAATGAACAGCCTGAATCCTGAGG
ACACGGCCGTGTATTACTGTAACGCGCCCCGTTTGCATACATCTGTAGGCTACTG
GGGCCAGGGGACCCAGGTCACCGTCTCCTCAGAACCCAAGACACCAAAACCAC
AA
JIK-B12
(SEQ ID NO: 120)
QVQLVESGGGLVQAGNSLRLSCTASGVIFSIYTMGWFRQAPGKEREFVAAIGVADG
TALVADSVTGRFTISRDNAKNTVYLHMNSLKPEDTAVYSCAAYLSPRVQSPYITDS
RYQLWGQGTQVTVSSEPKTPKPQ
(SEQ ID NO: 121)
CAGGTGCAGCTCGTGGAGTCGGGAGGAGGATTGGTGCAGGCTGGGAACTCTCT
GAGACTCTCCTGTACGGCCTCTGGTGTGATCTTCTCTATCTATACCATGGGCTGG
TTCCGCCAGGCTCCAGGGAAGGAGCGTGAGTTTGTAGCAGCGATAGGGGTGGCT
GATGGTACCGCACTTGTGGCAGACTCCGTGACGGGCCGATTCACCATCTCCAGA
GACAACGCCAAGAACACCGTTTATCTGCATATGAACAGCCTGAAGCCTGAGGAC
ACGGCCGTCTATTCCTGTGCAGCGTATCTTAGCCCCCGTGTCCAATCCCCCTACA
TAACTGACTCCCGGTATCAACTCTGGGGCCAGGGGACCCAGGTCACCGTCTCCT
CAGAACCCAAGACACCAAAACCACAA
JIK-F4
(SEQ ID NO: 122)
TGGGLVQAGGSLRLSCAASGRYAMGWFRQAPGKEREFVATISRSGAIREYADSVK
GRFTISRDGAENTVYLEMNSLKPDDTAIYVCAEGRGATFNPEYAYWGQGTQVTVSS
AHHSEDPS
(SEQ ID NO: 123)
CAGGTGCAGCTCGTGGAGACTGGGGGAGGATTGGTGCAGGCTGGGGGCTCTCT
GAGGCTCTCCTGTGCAGCCTCTGGACGCTATGCCATGGGCTGGTTCCGCCAGGC
TCCAGGGAAGGAGCGTGAATTTGTAGCGACTATTAGCCGGAGTGGTGCTATCAG
AGAGTATGCAGACTCCGTGAAGGGCCGATTCACCATCTCCAGAGACGGCGCCG
AGAACACGGTGTATCTGGAAATGAACAGCCTGAAACCTGACGACACGGCCATrr
ATGTCTGTGCAGAAGGACGAGGGGCGACATTCAACCCCGAGTATGCTTACTGGG
GCCAGGGGACCCAGGTCACCGTCTCCTCAGCGCACCACAGCGAAGACCCCTCG

### Example 28

### VHHs that Bind to Ricin Toxin (A Chain)

VHH binding agents were produced, purified and identified that are specific to ricin toxin A chain (RTA; see FIG. 29 A-B). The following VHHs were determined to specifically bind RTA: JIV-F5, JIV-F6, JIV-G12, JIY-A7, JIY-D9, JIY-D10, JIY-E1, JIY-E3, JIY-E5, JIY-F10 and JIY-G11. The amino acid sequence and nucleotide sequence of each of these VHHs were determined and are shown below:

JIV-F5
(SEQ ID NO: 124)
QVQLVESGGGLVQPGGSLRLSCAASGFTLDDYAIGWFRQVPGKEREGVACVKDGS
TYYADSVKGRFTISRDNGAVYLQMNSLKPEDTAVYYCASRPCFLGVPLIDFGSWGQ
GTQVTVSSEPKTPKPQ
(SEQ ID NO: 125)
CAGGTGCAGCTCGTGGAGTCGGGCGGAGGCTTGGTGCAGCCTGGGGGGTCTCTG
AGACTCTCCTGTGCAGCCTCTGGATTCACTTTGGATGATTATGCCATAGGCTGGT
TCCGCCAGGTCCCAGGGAAGGAGCGTGAGGGGGTCGCATGTGTTAAAGATGGT
AGTACATACTATGCAGACTCCGTGAAGGGCCGATTCACCATCTCCAGAGACAAC
GGCGCGGTGTATCTGCAAATGAACAGCCTGAAACCTGAGGACACAGCCGTTTAT
TACTGTGCATCCAGGCCCTGCTTTTTGGGTGTACCACTTATTGACTTTGGTTCCT
GGGGCCAGGGGACCCAGGTCACCGTCTCCTCGGAACCCAAGACACCAAAACCA
CAA
JIV-F6
(SEQ ID NO: 126)
QVQLVESGGGLVQAGGSLRLSCATSGGTFSDYGMGWFRQAPGKEREFVAAIRRNG
NGGNGIEYADSVKGRFTISRDNAKNTVHLQMNSLTPEDTAVYYCAASISGYAYNTI
ERYNYWGQGTQVTVSSEPKTPKPQ
(SEQ ID NO: 127)
CAGGTGCAGCTCGTGGAGTCAGGGGGAGGATTGGTGCAGGCTGGGGGCTCTCT
GAGACTCTCCTGCGCAACCECTGGCGGCACCTTCAGTGACTATGGAATGGGCTG
GTTCCGCCAGGCTCCAGGGAAGGAGCGTGAGETTGTAGCAGCTATTAGGCGGAA
TGGTAATGGCGGTAATGGCATTGAATATGCAGACTCCGTGAAGGGCCGATTCAC
CATCTCCAGAGACAACGCCAAGAACACGGTGCATCTACAAATGAACAGCCTGA
CACCTGAGGACACGGCCGTTTATTACTGTGCAGCGTCAATATCGGGATACGCTT
ATAACACAATTGAAAGATATAACTACTGGGGCCAGGGAACCCAGGTCACCGTCT
CCTCAGGAACCCAAGACACCAAAACCACAA
JIV-G12
(SEQ ID NO: 128)
QVQLVESGGGLVQAGGSLSLSCAASGGDFSRNAMAWFRQAPGKEREFVASINWTG
SGTYYLDSVKGRFTISRDNAKNALYLQMNNLKPEDTAVYYCARSTVFAEITGLAGY
QSGSYDYWGQGTQVTVSSEPKTPKPQ
(SEQ ID NO: 129)
CAGGTGCAGCTCGTGGAGTCCGGCGGAGGATTGGTGCAGGCGGGGGGCTCTCT
GAGTCTCTCCTGTGCAGCCTCTGGAGGTGACTTCAGTAGGAATGCCATGGCCTG
GTTCCGTCAGGCTCCAGGGAAGGAGCGTGAATTTGTAGCATCTATTAACTGGAC
TGGTAGTGGCACATATTATCTAGACTCCGTGAAGGGCCGATTCACCATCTCCAG
AGACAACGCCAAGAACGCCCTGTATCTGCAAATGAACAACCTGAAACCTGAGG
ACACGGCCGTTTATTACTGTGCACGCTCCACGGTGTTTGCCGAAATTACAGGCTT
AGCAGGCTACCAGTCGGGATCGTATGACTACTGGGGCCAGGGGACCCAGGTCA
CCGTCTCCTCAGAACCCAAGACACCAAAACCACAA
JIY-A7
(SEQ ID NO: 130)
QVQLVETGGGTVQTGGSLRLSCSASGGSFSRNAMGWFRQAPGKEREFVAAINWSA
SSTYYRDSVKGRFTVSRDNAKNTVYLHLNSLKLEDTAAYYCAGSSVYAEMPYADS
VKATSYNYWGQGTQVTVSSEPKTPKPQ
(SEQ ID NO: 131)
CAGGTGCAGCTCGTGGAGACCGGCGGAGGAACGGTGCANACTGGGGGCTCTCT
GAGACTCTCCTGTTCAGCCTCTGGCGGCTCCTTCAGTAGGAATGCCATGGGCTG
GTTCCGCCAGGCTCCAGGGAAGGAGCGTGAATTTGTAGCAGCTATTAACTGGAG
TGCCTCTAGTACTTATTATAGAGACTCCGTGAAGGGACGATTCACCGTCTCCAG
AGACAACGCCAAGAACACGGTGTATCTGCATTTGAACAGCCTGAAACTTGAGG
ACACGGCCGCGTATTACTGTGCTGGAAGCTCGGTGTATGCAGAAATGCCGTACG
CCGACTCTGTCAAGGCAACTTCCTATAACTACTGGGGCCAGGGGACCCAGGTCA
CCGTCTCCTCAGAACCCAAGACACCAAAACCACAA
JIY-D9
(SEQ ID NO: 132)
QVQLVETGGGLVQAGGSLRLPCSFSGFPFDNYFVGWFRQAPGKEREGVSCISSSDGS
TYYADSVKGRFTISRDNAKNTVYLQMNSLKPEDTAVYYCGADFLTPHRCPALYDY
WGQGTQVTVSSAHHSEDPS
(SEQ ID NO: 133)
CAGGTGCAGCTCGTGGAGACCGGGGGAGGCTTGGTGCAGGCTGGGGGGTCTCT
GAGACTCCCCTGTTCATTCTCTGGATTCCCTTTCGATAATTATTTCGTAGGCTGG
TTCCGCCAGGCCCCAGGGAAGGAGCGTGAGGGGGTCTCATGTATTAGTAGTAGT
GATGGTAGCACATACTATGCAGACTCCGTGAAGGGCCGGTTCACCATCTCCAGA
GACAACGCCAAGAACACGGTGTATCTGCAAATGAACAGTCTGAAACCTGAGGA
TACGGCCGTTTATTACTGTGGAGCAGATTTCCTCACCCCACATAGGTGTCCAGCC
TTATATGACTACTGGGGCCAGGGGACCCAGGTCACCGTCTCCTCAGCGCACCAC
AGCGAAGACCCCTCG
JIY-D10
(SEQ ID NO: 134)
QVQLVESGGGLVQPGGSLRLHCAASGSIASIYRTCWYRQGTGKQRELVAAITSGGN
TYYADSVKGRFTISRDNAKNTIDLQMNSLKPEDTAVYYCNADEAGIGGFNDYWGQ
GTQVTVSSAHHSEDPS
(SEQ ID NO: 135)
CAGGTGCAGCTCGTGGAGTCTGGTGGAGGCTTGGTGCAGCCTGGGGGGTCTCTG
AGACTCCACTGTGCAGCCTCTGGAAGCATCGCCAGTATCTATCGCACGTGCTGG
TACCGCCAGGGCACAGGGAAGCAGCGCGAGTTGGTCGCAGCCATTACTAGTGG
TGGTAACACATACTATGCGGACTCCGTFAAGGGCCGATTCACCATCTCCAGAGA
CAACGCCAAAAACACAATCGATCTGCAAATGAACAGCCTGAAACCTGAGGACA
CGGCCGTCTATTACTGTAATGCAGACGAGGCGGGGATCGGGGGATTTAATGACT
ACTGGGGCCAGGGGACCCAGGTCACCGTCTCCTCAGCGCACCACAGCGAAGAC
CCCTCG
JIY-E1
(SEQ ID NO: 136)
QVQLVESGGGLVQAGGSLRLSCAASGRTFSRSSMGWFRQAPGKEREFVASIVWAD
GTTLYGDSVKGRFTVSRDNVKNMVYLQMNNLKPEDTALYYCADNKFVRGLVAVR
AIDYDYWGQGTQVTVSSEPKTPKPQ
(SEQ ID NO: 137)
CAGGTGCAGCTCGTGGAGTCGGGGGGAGGATTGGTGCAGGCTGGGGGCTCTCT
GAGACTCTCCTGTGCAGCCTCTGGACGCACCTTCAGTCGCAGTTCCATGGGCTG
GTTCCGCCAGGCTCCAGGGAAGGAGCGTGAATTCGTTGCGTCCATTGTCTGGGC
TGATGGTACGACGTTGTATGGAGACTCCGTAAAGGGCCGATTCACCGTCTCCAG
GGACAACGTCAAGAACATGGTGTATCTACAAATGAACAACCTGAAACCTGAGG
ACACGGCCCTTTATTACTGTGCGGACAATAAATTCGTCCGTGGATTAGTGGCTGT
CCGTGCGATAGATTATGACTACTGGGGCCAGGGGACCCAGGTCACCGTCTCGTC
AGAACCCAAGACACCAAAACCACAA
JIY-E3
(SEQ ID NO: 138)
QVQLVESGGLVQAGGSLRLSCAASGRADIIYAMGWFRQAPGKEREFVAAVDWSGG
STYYADSVKGRFTISRDNAKNSVYLQMNSLKPEDTAVYYCAARRSWYRDALSPSR
VYEYDYWGQGTQVTVSSEPKTPKPQ
(SEQ ID NO: 139)
CAGGTGCAGCTCGTGGAGTCGGGAGGATTGGTGCAGGCTGGAGGCTCTCTGAG
ACTCTCCTGCGCAGCCTCTGGACGCGCCGACATAATCTATGCCATGGGCTGGTT
CCGCCAGGCTCCAGGGAAGGAGCGTGAGTTTGTAGCGGCAGTAGACTGGAGTG
GTGGTAGCACATACTATGCAGACTCCGTGAAGGGCCGATTCACCATCTCCAGAG
ACAACGCCAAGAACTCGGTGTATCTGCAAATGAACAGCCTGAAACCTGAGGAC
ACGGCCGTTTATTACTGTGCAGCCCGAAGGAGCTGGTACCGAGACGCGCTATCC
CCCTCCCGGGTGTATGAATATGACTACTGGGGCCAGGGGACCCAGGTCACCGTC
TCCTCAGAACCCAAGACACCAAAACCACAA
JIY-E5
(SEQ ID NO: 140)
QVQLVETGGGLVQPGGSLTLSCAGSGGTLEHYAIGWFRQAPGKEHEWLVCNRGEY
GSTVYVDSVKGRFTASRDNAKNTVYLQLNSLKPDDTGIYYCVSGCYSWRGPWGQ
GTQVTVSSAHHSEDPS
(SEQ ID NO: 141)
CAGGTGCAGCTCGTGGAGACGGGAGGAGGCTTGGTGCAGCCTGGGGGGTCTCT
GACACTCTCCTGTGCAGGCTCCGGTGGCACTTTGGAACATTATGCTATAGGCTG
GTTCCGCCAGGCCCCTGGGAAAGAGCATGAGTGGCTCGTATGTAATAGAGGTGA
ATATGGGAGCACTGTCTATGTAGACTCCGTGAAGGGCCGATTCACCGCCTCCAG
AGACAACGCCAAGAACACGGTGTATCTGCAATTGAACAGTCTGAAACCTGACG
ACACAGGCATTTATTACTGTGTATCGGGATGTTACTCCTGGCGGGGTCCCTGGG
GCCAGGGGACCCAGGTCACCGTCTCCTCGGCGCACCACAGCGAAGACCCCTCG
JIY-F10
(SEQ ID NO: 142)
QVQLVESGGGLVQPGGSLKLSCRASGSIVSIYAVGWYRQAPGKQRELLAAITTDGS
TKYSDSVKGRFTISRDNAKNIVYLQMNNLKPEDIAIYSCIGDAAGWGDQYYWGQ
GTQVTVSSEPKTPKPQ
(SEQ 1D NO: 143)
CAGGTGCAGCTCGTGGAGTCTGGGGGAGGTTTGGTGCAGCCTGGGGGGTCTCTG
AAACTCTCCTGTAGAGCCTCTGGAAGCATAGTCAGTATCTATGCCGTGGGCTGG
TACCGCCAGGCTCCAGGGAAGCAGCGCGAGTTGCTCGCGGCTATCACTACTGAT
GGTAGCACGAAGTACTCAGACTCCGTGAAGGGCCGATTCACCATCTCCCGAGAC
AACGCCAAGAACACGGTATATCTGCAAATGAACAACCTCAAACCTGAGGACAC
GGCCATCTATTCCTGTATCGGGGACGCGGCGGGTTGGGGCGACCAATACTACTG
GGGCCAGGGGACCCAGGTCACCGTCTCCTCAGAACCCAAGACACCAAAACCAC
AA
JIY-G11
(SEQ ID NO: 144)
QVQLVESGGGLVQAGGSLRLSCAASGSIVNFETMGWYRQAPGKERELVATITNEGS
SNYADSVKGRFTISGDNAKNTVSLQMNSLKPEDTAVYYCSATFGSRWPYAHSDHW
GQGTQVTVSSEPKTPKPQ
(SEQ ID NO: 145)
CAGGTGCAGCTCGTGGAGTCAGGCGGAGGCTTGGTGCAGGCTGGGGGGTCTCTG
AGACTCTCCTGTGCAGCCTCTGGAAGCATCGTCAATTTCGAAACCATGGGCTGG
TACCGCCAGGCTCCAGGGAAGGAGCGCGAGTTGGTCGCAACTATTACTAATGAA
GGTAGTTCAAACTATGCAGACTCCGTGAAGGGCCGATTCACCATCTCCGGAGAC
AACGCCAAGAACACGGTGTCCCTGCAAATGAACAGCCTGAAACCTGAGGACAC
GGCCGTCTACTACTGTTCGGCGACGTTCGGCAGTAGGTGGCCGTACGCCCACAG
TGATCACTGGGGCCAGGGGACCCAGGTCACCGTCTCCTCAGAACCCAAGACACC
AAAACCACAA

### Example 29

### VHHs Specific for Ricin Toxin (B Chain)

VHH binding agents were produced, purified and identified that are specific to ricin toxin B chain (See FIG. 29A-FIG. 29B). VHHs that specifically bind to RTB were observed as follows: JIW-B1, JIW-C12. JIW-D12, JIW-G5, JIW-G10, JIZ-B7, JIZ-B9, JIZ-D8, and JIZ-G4. The amino acid sequences and encoding nucleotide sequences for each of these VHHs were determined and are shown below:

JIW-B1
(SEQ ID NO: 146)
QVQLVETGGALVHTGGSLRLSCEVSGSTFSSYGMAWYRQAPGEQRKWVAGIMPD
GTPSYVNSVKGRFTISRDNAKNSVYLHMNNLRPEDTAVYYCNQWPRTMPDANWG
RGTQVTVSSEPKTPKPQ
(SEQ ID NO: 147)
CAGGTGCAGCTCGTGGAGACGGGCGGAGCATTGGTGCACACTGGGGGTTCTCTG
AGACTCTCCTGCGAAGTCTCCGGAAGCACCTTCAGTAGCTATGGCATGGCCTGG
TACCGCCAAGCTCCAGGCGAGCAGCGTAAGTGGGTCGCAGGTATTATGCCGGAT
GGTACTCCAAGCTATGTAAACTCCGTGAAGGGCCGATTCACCATCTCCAGAGAC
AACGCCAAGAACTCGGTGTATCTGCACATGAACAACCTGAGGCCTGAAGACAC
GGCCGTCTATTATTGCAACCAATGGCCGCGCACGATGCCTGACGCGAACTGGGG
CCGGGGGACCCAGGTCACCGTCTCCTCAGAACCCAAGACACCAAAACCACAA
JIW-C12
(SEQ ID NO: 148)
QVQLVETGGSLRLTCVTSGSTFNNPAITWYRQPPGKQREWVASLRSGDGPVYRESV
KGRFTIFRDNATDALYLRMNSLKPEDTAVYHCNTASPASWLDWGQGTQVTVSSEP
KTPKPQ
(SEQ ID NO: 149)
CAGGTGCAGCTCGTGGAGACTGGGGGGTCTCTGAGGCTCACCTGTGTAACCTCT
GGAAGCACCTTCAATAATCCTGCCATAACCTGGTACCGCCAGCCTCCAGGGAAG
CAGCGTGAGTGGGTCGCAAGTCTTCGTAGTGGTGATGGTCCAGTATATAGGGAA
TCCGTGAAGGGCCGATTCACCATTTTTAGAGACAACGCCACGGACGCGCTGTAT
CTGCGGATGAATAGCCTGAAACCTGAGGACACGGCCGTCTATCACTGTAACACC
GCCTCACCTGCTAGTTGGCTGGACTGGGGCCAGGGGACCCAGGTCACTGTCTCC
TCAGAACCCAAGACACCAAAACCACAA
JIW-D12
(SEQ ID NO: 150)
QVQLVETGGGLVQPGGSLRLSCATSGFPFSTERMSWVRQAPGKGLEWVSGITEGGE
TTLAAPSVKGRFNISRDNARNILYLQMNSLKPEDAAVYYCFRGVFFRTSFPPELARG
QGTQVTVSSEPKTPKPQ
(SEQ ID NO: 151)
CAGGTGCAGCTCGTGGAGACGGGAGGAGGATTGGTGCAACCTGGGGGTTCTCT
GAGACTCTCTTGTGCAACCTCTGGATTCCCCTTCAGTACGGAGCGTATGAGCTG
GGTCCGCCAGGCTCCAGGAAAGGGGCTCGAGTGGGTCTCAGGTATTACTGAGG
GTGGTGAAACCACTCTCGCGGCACCCTCCGTGAAGGGCCGATTCAACATCTCCA
GAGACAACGCCAGGAATATCCTATATCTACAGATGAATTCCTTGAAACCTGAGG
ACGCGGCCGTTTACTATTGTTTTAGAGGTGTTTTTTTTAGAACGAGTTTTCCTCCC
GAACTCGCGCGGGGCCAGGGGACCCAGGTCACCGTCTCCTCAGAACCCAAGAC
ACCAAAACCACAA
JIW-G5
(SEQ ID NO: 152)
QVQLVESGGGLVQAGGSLRLSCAASGSAVSDSFSTYAISWHRQAPGKQREWIAGIS
NRGATSYRDSVKGRFTISRDNAKNTVYLQMNNLKPEDTGVYYCEPWPREGLGGGQ
GTQVTVSSEPKTPKPQ
(SEQ ID NO: 153)
CAGGTGCAGCTCGTGGAGTCGGGCGGAGGCTTGGTGCAGGCAGGGGGGTCTTT
GAGACTCTCCTGTGCAGCCTCTGGAAGCGCCGTCAGTGACAGCTTCAGTACCTA
TGCCATCTCCTGGCACCGCCAGGCTCCAGGGAAGCAGCGTGAGTGGATCGCAGG
TATTAGTAATCGTGGTGCGACAAGCTATAGAGACTCCGTGAAGGGCCGATTCAC
CATCTCCAGAGACAACGCCAAGAACACGGTATATCTGCAAATGAACAACCTGA
AACCTGAGGACACGGGCGTCTATTATTGTGAGCCATGGCCACGCGAAGGACTTG
GGGGGGGCCAGGGGACTCAGGTCACCGTCTCCTCAGAACCCAAGACACCAAAA
CCACAA
JIW-G10
(SEQ ID NO: 154)
QVQLVESGGGSVQTGGSLTLSCVVSGSTFSDYAVAWYRQVPGKSRAWVAGVSTTG
STSYTDSVRGRFTISRDNHKKTVYLSMNSLKPEDTGIYYCNLWPFTNPPSWGQGTQ
VTVSSAHHSEDPS
(SEQ ID NO: 155)
CAGGTGCAGCTCGTGGAGTCGGGGGGAGGCTCGGTGCANACTGGGGGGTCTCT
GACACTCTCCTGTGTAGTCTCTGGAAGTACCTTCAGTGACTATGCGGTGGCCTGG
TACCGCCAGGTTCCAGGCAAATCGCGTGCGTGGGTCGCGGGTGTTAGTACTACT
GGCTCGACATCTTATACAGACTCCGTGAGGGGCCGGTTCACCATCTCCAGAGAC
AACCACAAGAAGACGGTGTATCTTTCAATGAACAGCCTGAAACCTGAGGACAC
GGGCATCTATTACTGCAACTTATGGCCGTTCACAAATCCTCCTTCCTGGGGCCAG
GGAACCCAAGTCACCGTTTCCTCGGCGCACCACAGCGAAGACCCCTCG
JIZ-B7
(SEQ ID NO: 156)
QVQLVESGGAVVQPGGSLRLSCATSGFTFSDDRMSWARQAPGKGLEWVSGISTASE
GFATLYAPSVKGRFTISRDNAKHMLYLQMDTLKPEDTAVYYCLRGVFFRTNIPPEV
LRGQGTQVTVSSAHHSEDPS
(SEQ ID NO: 157)
CAGGTGCAGCTCGTGGAGTCTGGAGGAGCCGTGGTGCAACCTGGGGGTTCTCTG
AGACTCTCCTGTGCAACCTCTGGATTCACCTTCAGTGACGATCGTATGAGCTGG
GCCCGCCAGGCTCCAGGAAAGGGGCTCGAGTGGGTCTCAGGTATTAGTACTGCT
AGTGAAGGTTTTGCTACACTCTACGCACCCTCCGTGAAGGGCCGATTCACCATC
TCCAGAGACAACGCCAAGCATATGCTGTATCTGCAAATGGATACCTTGAAACCT
GAGGACACGGCCGTGTATTACTGTTTAAGAGGGGTTTTTTTTAGAACGAACATT
CCTCCCGAGGTACTGCGGGGCCAGGGGACCCAGGTCACCGTCTCCTCAGCGCAC
CACAGCGAAGACCCCTCG
JIZ-B9
(SEQ ID NO: 158)
QVQLVETGGDLVQPGGSLRLSCAASGSSFSRAAVGWYRQAPGKEREWVARLASGD
MTDYTESVRGRFTISRDNAKHTVYLQMDNLKPEDTAVYYCKARIPPYYSIEYWGK
GTRVTVSSEPKTPKPQ
(SEQ ID NO: 159)
CAGGTGCAGCTCGTGGAGACGGGGGGAGACTTGGTGCANCCTGGGGGGTCTCT
GAGACTCTCCTGTGCAGCCTCTGGAAGCTCCTTCAGCCGCGCTGCCGTGGGCTG
GTACCGTCAGGCTCCAGGAAAGGAGCGTGAGTGGGTCGCACGTCTCGCGAGTG
GTGATATGACGGACTATACCGAGTCCGTGAGGGGCCGATTCACTATCTCCAGAG
ACAACGCCAAGCACACGGTGTATCTGCAAATGGACAACCTGAAACCTGAGGAC
ACGGCCGTCTACTATTGTAAGGCCAGGATACCCCCTTATTACTCTATAGAGTACT
GGGGCAAAGGGACCCGGGTCACCGTCTCCTCANAACCCAAGACACCAAAACCA
CAA
JIZ-D8
(SEQ ID NO: 160)
QVQLVETGGGLVQAGGSLRLSCVVSSPLFNLYDMAWYRQAPGNQRELVAGILTDG
RATYSDSVKGRFTISRNNLINTVFLQMSSLKPEDTAVYYCNRKNSIYWDSWGQGT
QVTVSSEPKTPKPQ
(SEQ ID NO: 161)
CAGGTGCAGCTCGTGGAGACAGGTGGAGGCTTGGTGCAGGCTGGGGGGTCTCT
GAGACTCTCCTGTGTAGTATCTAGTCCCCTGTTCAATCTTTACGACATGGCCTGG
TATCGCCAGGCTCCAGGGAATCAGCGTGAGTTGGTCGCAGGCATCTTGACTGAT
GGTCGCGCAACATATTCAGACAGCGTGAAGGGCCGATTCACCATTTCCAGAAAC
AACCTGACGAACACGGTGTTTTTACAAATGAGCAGCCTGAAACCTGAGGACACG
GCCGTCTATTATTGTAATAGAAAGAATAGTATCTACTGGGATTCCTGGGGCCAG
GGGACCCAGGTCACCGTCTCCTCGGAACCCAAGACACCAAAACCACAA
JIZ-G4
(SEQ ID NO: 162)
QVQLVESGGGLVQAGGSLRLSCVASGLTFSRYGMGWFRQAPGQERVVVSVISPDG
GSAYYADSVKGRFTISRDNAKNTVYLQMSTLRFEDTGVYYCTAGPRNGATTVLRP
GDYDYWGQGTQVTVSSEPKTPKPQ
(SEQ ID NO: 163)
CAGGTGCAGCTCGTGGAGTCGGGGGGAGGATTGGTGCAGGCTGGGGGCTCTCT
GAGACTCTCCTGCGTAGCCTCTGGACTCACCTTCAGTCGCTATGGCATGGGCTG
GTTCCGCCAGGCTCCAGGACAGGAGCGTGTAGTCGTATCAGTTATTAGTCCCGA
CGGTGGTAGCGCATACTACGCAGACTCCGTGAAGGGCCGATTCACCATCTCCAG
AGACAACGCCAAGAACACGGTGTATCTGCAAATGAGCACCCTGAGATTTGAGG
ACACGGGCGTTTATTATTGTACAGCAGGGCCCCGGAATGGAGCGACTACAGTCC
TCCGGCCAGGGGATTATGACTACTGGGGCCAGGGGACCCAGGTCACTGTCTCCT
CAGAACCCAAGACACCAAAACCACAA

### Example 30

### VHH Binding Proteins Bind to Neutralized Toxin-Disease Agents

Effectiveness of the antitoxin treatment using VHH binding proteins composed of SEQ ID NOs: 96-163 (FIG. 29A and FIG. 29B) were analyzed to determine ability of the binding proteins to bind to and neutralize a toxin disease agent target. Data show that the VHH effectively bound to and neutralized Stx1, Stx2, anthrax toxins, RTA, and RTB.

### Example 31

### Recombinant Multimeric Binding Proteins Neutralize a Plurality of Disease Agents

Recombinant heteromultimeric neutralizing binding proteins containing multiple binding regions composed of any of SEQ ID NOs: 96-163 are produced. At least two of the binding regions are not identical and each binding region has affinity to specifically bind a non-overlapping portion of a disease agent associated with toxin proteins produced by bacteria or plants such as a Shiga toxin, a ricin toxin (e.g., RTA and RTB), and anthrax toxin.

Subjects are exposed to one or more of Shiga toxin, ricin toxin A chain, and ricin toxin B chain, and then are administered each of the heteromultimeric binding proteins. Control subjects are administered the one or more disease agents only (no multimeric binding proteins). Subjects are monitored for indicia of exposure to the pathogenic molecules such as diarrhea, fever, tachycardia, respiratory distress, and death.

Subject administered heteromultimeric binding proteins specific for disease agents are observed to have little or no indicia of exposure to the one or more disease agents. In vitro analysis of each of cell, blood and tissue samples from the subjects show that the multimeric binding proteins neutralize each of these disease agents in the samples. Control subjects show indicia of being exposed to the disease agents (e.g., diarrhea, internal bleeding, and cell lysis). Thus, the recombinant heteromultimeric neutralizing binding proteins are found to be effective inhibitors of the toxin disease agents.

### Example 32

### VHHs that Bind and Neutralize Plant Toxins

Methods as described in Examples herein using phage libraries are used to produce and identify VHHs that specifically bind and neutralize plant toxins. The VHHs specifically neutralize each of the following plant toxins: Akar saga (Abrus precatorius), Deathcamas, Amianthium Angel's Trumpet (Brugmansia), Angel Wings (Caladium), Anticlea, Autumn crocus (Colchicum autumnale), Azalea (Rhododendron), Bittersweet nightshade (Solanum dulcamara), Black hellebore (Helleborus niger), Black locust (Robinia pseudoacacia), Black nightshade (Solanum nigrum), Bleeding heart (Dicentra cucullaria), Blind-your-eye mangrove (Excoecaria agallocha), Blister Bush (Peucedanum galbanum), Bloodroot (Sanguinaria canadensis), Blue-green algae (Cyanobacteria), Bobbins (Arum maculatum), Bracken (Pteridium aquilinum), Broom (Cytisus scoparius), calabar bean (Physostigma venenosum), castor bean, Christmas rose (Helleborus niger), Columbine (Aquilegia), Corn cockle (Agrostemma githago), corn lily (veratrum), cowbane (Cicuta), cows and bulls (Arum maculatum), crab's eye (Abrus precatorius), cuckoo-pint (Arum maculatum), daffodil (Narcissus), Darnel (Lolium temulentum), Deadly nightshade (Atropa belladonna), Devils and angels (Arum maculatum), False acacia (Robinia pseudoacacia), False hellebore (Veratrum), Foxglove (Digitalis purpurea), Frangipani (Plumeria), Doll's eyes (Actaea pachypoda), Dumbcane (Dieffenbachia), Dutchman's breeches (Dicentra cucullaria), Elder/Elderberry (Sambucus), Giant hogweed (Heracleum mantegazzianum), Giddee giddee, Gifblaar (Dichapetalum cymosum), Greater celandine (Chelidonium majus), Gympie gympie (Dendrocnide moroides), Heart of Jesus (Caladium), hemlock (Conium maculatum), hemlock water-dropwort (Oenanthe crocata), henbane (Hyoscyamus niger), Horse chestnut (Aesculus hippocastanum), Holly (Ilex aquifolium), Hyacinth (Hyacinthus orientalis), Indian licorice, Jack in the pulpit, Jamestown weed, jequirity, Jerusalem cherry, Jimson weed, John Crow bead, Jumbie bead, Lily of the Valley, Lords and Ladies, Madiera winter cherry, Mayapple, Meadow saffron, Milky mangrove, Monkshood, Moonseed, Passion flower, Plumeria, Poison hemlock, Poison ivy, Poison oak, Poison parsnip, Poison sumac, Poison ryegrass, Pokeweed, Precatory bean, Privet, ragwort, redoul, River poison tree, Robinia pseudoacacia (also known as black locust and false acacia), Rosary pea, Sosnowsky's Hogweed, Spindle tree, Starch-root, Stenanthium, Stinging tree, Stinkweed, Strychnine tree, Suicide tree (Cerbera odollam), thorn apple, Toxicoscordion, Wake robin, Water hemlock, White baneberry, White snakeroot, Wild arum, Winter cherry, Wolfsbane, Yellow Jessamine, Yew, and Zigadenus.

### Example 33

### Immunoassay Using VHHs to Detect Toxin

Immunoassay are performed using VHH camelids to detect toxin in samples. Each of toxin-specific VHHs SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162 are separately incubated in buffer in wells of a plastic microtiter plate. The VHH camelids are incubated for sufficient time and under conditions such that the VHHs are adsorbed to the surface of the well. Control cells are incubated with buffer only.

A of diluated aliquots of a sample containing either a Shiga toxin, a B. anthracis toxin, a ricin A chain toxin, or a ricin B chain toxin are incubated in duplicate in the VHH-coated wells and control wells, such that the VHH in the VHH-coated wells specifically bind to the toxin, thereby retaining the toxin in the well. Wells are washed to remove toxin that is not specifically bound to the VHH camelids.

A polyclonal antibody with enzymes or dye molecules attached to the polyclonal antibody is contacted to the wells, thereby forming an toxin antigen ‘sandwich’ between the VHH camelids and the polyclonal antibody. The enzymes or dye molecules attached to the polyclonal antibodies generate a color signal proportional to the amount of target toxin present in the sample added to the wells of the plate. It is observed that the toxin-specific VHHs specifically bound the respective toxin, such that SEQ ID NO: 96 and SEQ ID NO: 98 specifically bind the Shiga toxin, and SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 specifically bind the anthrax toxin, and SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 specifically bind the ricin A chain toxin, and wherein SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 specifically bind the ricin B chain toxin.

### Example 34

### Immunofluorescence Staining Using the Toxin-Specific VHHs

Subconfluent test cells on coverslips are treated with toxin (either a Shiga toxin, a B. anthracis toxin, a ricin A chain toxin, or a ricin B chain toxin) alone or toxin in the presence of the toxin-specific VHHs (specific VHHs SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162). The cells are fixed with paraformaldehyde, followed by permeabilization in a permeabilizing buffer. For immunocomplex or toxin staining, cells are incubated with fluorochrome-conjugated anti-VHH, or polyclonal rabbit anti-toxin serum (prepared herein by methods known to one of skill in the art of antibody production), followed by fluorochrome-conjugated anti-rabbit-IgG. Cells are counterstained with 4′,6-diamidino-2-phenylindole (DAPI) and imaged using a confocal microscope. Surface binding of toxin-specific VHHs to cells is examined by flow cytometry.

Data from the immunofluorescence staining that the toxin-specific VHHs specifically bind the respective toxin, and the toxin-specific VHHs are effective for detecting the toxin, such that SEQ ID NO: 96 and SEQ ID NO: 98 specifically bind the Shiga toxin, and SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, and SEQ ID NO: 122 specifically bind the anthrax toxin, and SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144 specifically bind the ricin A chain toxin, and wherein SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, and SEQ ID NO: 162 specifically bind the ricin B chain toxin.

Test cells are incubated with toxin alone, toxin/toxin-specific VHH, or toxin/non-specific VHH, followed by phycoerythrin-conjugated anti-VHH staining. Cells are subsequently analyzed by cell sorting using a FACS Calibur flow cytometer. Data show that the VHHs effectively detect the location and relative abundance of specific Shiga toxin proteins, anthrax proteins, and ricin toxin proteins.

### Example 35

### Toxins and Reagents

O157:H7 Stx1 purified from cell lysates of Stx1-producing E. coli HB101-H19B (Jacewicz M S, et al. 1999 Infect Immun 67: 1439-1444) and 0157:117 Stx2 from culture supernatants of Stx2-producing E. coli C600W (Ibid; Donohue-Rolfe A, 1989 Infect Immun 57: 3888-3893) were obtained from Phoenix Lab at Tufts Medical Center. The toxins were dissolved at 1 mg/ml in phosphate buffered saline (PBS), aliquoted and stored at −80° C. Reagents for western blotting were purchased from KPL. Antibodies used were anti-E-tag mAb (Phadia); HRP-anti-E-tag mAb (Bethyl Labs); and HRP-anti-M13 Ab (GE Healthcare).

### Example 36

### Preparation of Stx Reagents for Immunization

Intact Stx1B-subunit (Stx1B) and Stx2 A unit (Stx2A) and Stx2 B subunit (Stx2B) were produced as recombinant proteins in E. coli. The DNAs encoding the subunits (GenBank accession nos. M19473.1 And EF441614.1) were amplified by PCR and ligated into pET-25B in frame with C-terminal His tags, and plasmids were confirmed by sequencing. Expression and purification of recombinant Stx subunits was performed as described for VHH expression (Tremblay J M, et al. Toxicon 56(6): 990-998) The purified proteins were dialyzed with PBS, sterilized using 0.22 μm filter, and stored at −70° C. Stx1 And Stx2 toxoids were prepared by formalin inactivation of the holotoxins followed by dialysis with PBS and stored at −70° C.

### Example 37

### Alpaca Immunization and VHH-Display Library Preparation

An alpaca was immunized by four successive multi-site subcutaneous injections at 3 week intervals using an immunogen comprising of 50 μg of Stx1 toxoid and 50 μg of Stx2 toxoid in alum/CpG adjuvant. The serum of the alpaca at the completion of the immunization process contained Ab titers for Stx1 of approximately 1:10,000 and for Stx2 of approximately 1:100,000. Six days after the final boost, blood from the alpaca was obtained for lymphocyte preparation and a VHH-display phage library was prepared from the immunized alpaca. (Ibid., and Maass D R, et al. 2007 Int J Parasitol 37: 953-962) More than 106 independent clones were prepared from B cells of the alpaca successfully immunized with each of the immunogens.

### Example 38

### ELISAs and Western Blots

Capture ELISAs were performed by coating plates with 0.5 μg/ml of 4D3 mAb for Stx1 And 3D1 mAb for Stx2. See Donohue-Rolfe A, et al. 1989 Infect Immun 57: 3888-3893. The plates were blocked and then incubated with 0.3 μg/ml of Stx1 or Stx2. For standard ELISAs, plates were coated with 1.5 μg/ml of Stx1 or Stx2, or 2 μg/ml Stx subunits. Test VHH agents were serially diluted, incubated for 1 hour at room temperature (RT), washed and bound VHH agent were detected with HRP-anti-E-tag. Bound HRP was detected using the TMB kit (Sigma) and values were plotted as a function of the input VHH concentration. EC50 values were estimated from these plots as the VHH concentration that produced a signal equal to 50% of the peak binding signal. Competition ELISAs were performed as described in Mukherjee J, et al. 2012 PLoS ONE 7: e29941. Western blots to identify Stx subunit recognition of the purified VHHs were performed. See Ibid.

### Example 39

### Anti-Stx VHH Identification and Preparation

About 2×10⁶ independent clones were prepared from B cells of the alpaca successfully immunized with the Stx immunogens. Panning, phage recovery and clone fingerprinting were performed much as described in Mukherjee J. et al. 2012 PLoS ONE 7: e29941; Tremblay J M, et al. 2010 Toxicon. 56(6): 990-998; Maass D R, et al. 2007 Int J Parasitol 37: 953-962 with the following variations. Separate panning processes were performed for Stx1 And Stx2. Panning for each toxin was initially performed using toxin coated onto plastic (Nunc Immuno) and later another panning process was performed using the toxins captured onto plastic with a mAb. For each process, three cycles of panning were performed in which two cycles were performed at ‘low stringency’ and third cycle was performed at ‘high stringency’. For low stringency panning, the two Stx toxins were coated directly onto plastic wells at 10 μg/ml or captured to plastic with 5 μg/ml of capture mAb followed by 1.5 μg/ml of toxin. These Stx coated wells were incubated for 1 hr with about 10¹² input phage followed by 15 rapid washes, a 15 min wash and elution of bound phage. For high stringency panning, the two Stx toxins were coated onto plastic wells at 0.5 μg/ml or captured to plastic with 5 μg/ml of capture mAb followed by 0.15 μg/ml of toxin. Wells were incubated for 10 min with about 10¹⁰ input phage followed by 15 rapid washes, a final wash of 1 hr and elution.

After plating the phage from the third panning cycle, individual colonies were picked and grown overnight at 37° C. in 96-well plates. A replica plate was then prepared by transferring 2 μl of culture to another 96-well plate containing 180 μl of culture medium. After 4 hours of incubation at 37° C., IPTG was added to 3 mM in all wells and incubation continued at 30° C. overnight. Bacteria were pelleted by centrifugation at 1000×g and 50 μl of the supernatant was screened for Stx-binding soluble VHH by ELISA as described herein.

For each panning regimen, about 10-20% of VHH clones were observed to be positives for binding to Stx1 And Stx2 based on ELISA signals at least 2× the signal of negative controls. About 100 positives for each toxin were selected for DNA fingerprinting. For the DNA fingerprinting, the VHH coding region was amplified from each of the clones by PCR and separately digested with HaeIII, BsaJ1 or BstN1. The products of the digests were resolved on gels to identify clones with distinctive digestion products. Eighteen DNA fingerprints were identified among the VHHs selected as positives for Stx1 And twenty-five DNA fingerprints were identified among the VHHs selected as positives for Stx2.

Clones from each group of clones with substantially identical or nearly identical DNA fingerprints were selected for DNA sequence analysis of the VHH coding region. Clones selected for sequencing were those from each fingerprint group that produced the strongest ELISA signals. DNA sequences of the VHH coding regions were obtained and analyzed by phylogenetic tree analysis to identify closely-related VHHs likely to have common B cell clonal origins. Phylogenetic trees were obtained using Accelrys Gene 2.0 software following alignment of only the VHH amino acid sequences encoded internal to the PCR primers which were employed to amplify the VHH coding DNAs from alpaca B cells (i.e. primer binding sites and hinge regions were excluded). Based on this analysis, VHHs that appeared to be unrelated to any other VHH were selected for protein expression. In addition, some VHHs that produced particularly strong signals on ELISA but were distantly-related to other VHHs, and VHHs that appeared to have interesting properties such as cross-specificity to both Stxs, were also selected for protein expression.

Expression and purification of VHHs in E. coli as recombinant thioredoxin (Trx) fusion proteins containing hexahistidine was performed as described in examples herein. (Tremblay J M, et al. 2010 Toxicon. 56(6): 990-998) VHH heteromultimer were engineered such that all VHHs were in the same reading frame separated by DNA encoding a 15 amino acid flexible spacer ((GGGGS)3). Monomer VHHs were expressed with a carboxyl terminal E-tag epitope. Heteromultimer VHHs were engineered to contain a second copy of the E-tag in frame between the Trx and VHH domains. (Mukherjce J. et al. 2012 PLoS ONE 7: e29941) Competition analysis was performed as described in examples herein to identify VHHs that may bind to identical or overlapping epitopes.

### Example 40

### Kinetic Analysis by Surface Plasmon Resonance

Assessment of the kinetic parameters of the VHHs was performed using a ProteOn XPR36 Protein Interaction Array System (Bio-Rad, Hercules, Calif.) after Stx1 or Stx2 were immobilized by amine coupling chemistry using the manufacturer's recommended protocol. GLH (high protein immobilization capacity) chip surface was activated with a mixture of 0.4 M EDC (1-ethyl-3-(3-dimethylaminopropyl)carbodiimide) and then 0.1 M sulfo-NHS (N-hydroxysulfosuccinimide) was injected for 300 s at 30 μL/min, Stx1 or Stx2 was immobilized by passing a 90 or 30 μg/mL solution of the protein, respectively, at pH 5 over the surface for 150 s at 25 μL/min. The surface was deactivated with a 30 μL/min injection of 1 M ethanolamine for 300 s. A concentration series for each VHH (between 1.5625 nM and 400 nM, optimized for each antibody fragment) was passed over the surface at 100 μL/min for 60 s, and dissociation was recorded for 600 s or 1200 s. The surface was then regenerated with a 30 s injection of 50 mM HCl at 50 μL/min. Running buffer used for these examples was 10 mM Hepes, pH 7.4, 150 mM NaCl, 0.005% Tween-20. Data was evaluated with ProteOn Manager software (version 3.1.0.6) using the Langmuir interaction model. Data herein are values that are the mean of at least four runs.

### Example 41

### Cell-Based Shiga Toxin Neutralization Assay

Stx neutralization by VHH-based agents was assessed as previously described (Sheoran A S, et al. 2005 Infect Immun 73: 4607-4613) with the following modifications. Vero cells (ATCC# CCL-81) were cultured in 96-well plates in 100 μl of Minimum Essential Medium (Mediatech Inc), supplemented with 10% FBS (HyClone). The cells were plated at about 10,000 cells/well the day prior to the assay. Stx doses were determined by performing a dose-response assay with each batch of toxin. Serial dilutions of Stx were added to wells of near confluent Vero cells, cultured 48 hours and stained with Crystal Violet. The Stx dose selected for neutralization assays was the minimum dose that caused greater than 90% cell death based on reduced well staining (A590). Typically these doses were approximately 0.1 ng/well (about 15 pM) for Stx1 And approximately 0.25 ng/well (about 35 pM) for Stx2. Control wells containing dilutions of toxin were included in each assay to assess that the toxin potency on the cells. Serial dilutions of various test antitoxin agents were generated in culture medium, combined with toxin and incubated for 1 hr at 37° C. Toxin-only control wells were included. Vero cell media was removed and replaced with the mixture of test agents and toxin, then cultured 48 hours prior to staining and reading of absorbance at 590 nm. IC50 estimates were assessed as the agent concentration that produced a signal that was 50% of the difference between the peak signal and the baseline signal from wells having no agent.

### Example 42

### In Vivo Mouse Assay of Shiga Toxin Lethality

Female CD1 mice, 15-17 g each, (Charles River Labs) were weighed and sorted into groups of five mice each to minimize inter-group weight variation. The minimum lethal dose (MLD) of Stx1 And Stx2 was determined from dose-response studies. For evaluation of test agents, a dose of 1.25× the MLD was utilized: 60 ng Stx2/mouse or 1.25 μg Stx1/mouse. Solutions of test agents and Stx were prepared at twice the final concentration required and then 600 μl of test agent and 600 μl of the selected Stx in PBS were combined, resulting in the final desired concentration of each component. Following incubation at room temperature for 30 minutes, 200 μl of the mixture was administered by intravenous tail vein injection at time 0 to mice in groups of five. Mice were monitored 4-6 times each day and individually scored for overall disposition, presence of central nervous system signs (trembling, ataxia, paralysis, opisthotonos), activity level, and mortality. Mice that were moribund or exhibiting central nervous system signs were euthanized. The time to death was determined for each mouse. No relapse was found to occur through 18 days in VHH- or mAb-treated mice that survived the lethal dose of Stx1 or Stx2 in an early study, so surviving mice in subsequent studies were typically euthanized alter about one week.

### Example 43

### Tissue Evaluation by Light Microscopy

Following euthanasia, right and left kidneys from each mouse were harvested, fixed in 10% neutral buffered formalin, dehydrated, paraffin-embedded, sectioned at 3 μm, stained with hematoxylin and eosin, and evaluated by board certified veterinary pathologists (RP and GB) blinded to the treatment groups. Tubular lesions were quantified by counting the number of affected tubules in 6 random 20× fields in the cortex and corticomedullary junctions from the left and right kidneys.

### Example 44

### Identification and Binding Properties of VHHs Recognizing Stx1 and/or Stx2

Heavy-chain-only Vh (VHH) binding agents were obtained from a VHH-display phage library representing the VHH repertoire of an alpaca immunized with both Stx1 And Stx2 immunogens. Eighteen clearly distinct Stx1-binding VHHs and 25 Stx2-binding VHHs were identified using DNA fingerprinting. Coding DNA analysis of the Stx-binding VHHs (See FIG. 38) identified numerous unique VHHs and a large group of related VHHs (See FIG. 39). The group of related VHHs contained clones selected on both Stx1 And Stx2, including some that were virtually identical (e.g. Stx-F1 And Stx-H3). These results indicated that VHH members or this group recognize both Stx toxins. Eleven members of the large homology group, and each of the unique Stx1- and Stx2-selected VHHs (all the VHHs in FIG. 38), were expressed as soluble proteins and purified for further characterization.

Anti-Stx VHH binding to Stx1 And Stx2 was assessed by dilution ELISA and representative results are shown in FIGS. 30 and 31. The ELISA results confirmed that all 11 members of the large VHH homology group (FIG. 38) recognized both Stx1 And Stx2, although with wide variation in the relative EC50s for the two toxins. The two VHHs in this homology group having the lowest EC50 for both Stx1 And Stx2 (Stx-A4, Stx-A5) were selected for further study. All of the remaining VHHs were highly specific for either Stx1 or Stx2. The two Stx1-specific VHHs with the lowest EC50 (Stx1-A9, Stx1-D4) and the six Stx2-specific VHHs with lowest the EC50 (Stx2-A6, Stx2-D2, Stx2-D10, Stx2-G1, Stx2-G9, Stx2-H6) were selected for further study.

Selected Stx-binding VHHs were further characterized for affinity and subunit recognition. Binding affinities (KD) were determined by performing surface plasmon resonance (SPR). These data correlated well with EC50 values (Table 6) and confirmed the Stx cross-specificity of Stx-A4 and Stx-A5. Several VHHs displayed KD values in the sub-nanomolar range indicating very high affinity. Western blot analysis (FIG. 40) detected binding to Stx1 And Stx2 subunits following SDS-PAGE and specificity matched the ELISA and SPR data. Surprisingly, all VHHs were observed to recognize the Stx. B subunits except for Stx1-D4 which recognizes the Stx1A subunit. Despite the high affinity of these VHHs for native Stxs, binding to the denatured Stx1/2 B subunits on the Western blots was generally poor, consistent with these VHHs recognizing conformationally-sensitive epitopes. Stx subunit binding for the VHHs as reported in Table 6 was confirmed by ELISAs using purified recombinant Stx subunits.

TABLE 6
Properties of VHHs recognizing Stx1 and/or Stx2

| VHH | | | | Sub- | K_{D} (nM)^{b} | K_{D} (nM)^{b} | EC₅₀ (nM)^{c} | IC₅₀ (nM)^{d} | EC₅₀ (nM)^{c} | IC₅₀ (nM)^{d} | Gen- |
|---|---|---|---|---|---|---|---|---|---|---|---|
| name | Clone | Protein | Specificity | unit^{a} | Stx1 | Stx2 | Stx1 | Stx1 | Stx2 | Stx2 | bank |
| Stx1-A9 | JFA-26 | JET-A9 | Stx1 | B | 7.6 ± 0.9 | NB | 10 | 10 | >1000 | >1000 | |
| Stx1-D4 | JGL-8 | JGG-D4 | Stx1 | A | 0.128 ± 0.006 | NB | 0.5 | >1000 | >1000 | >1000 | |
| Stx-A4 | JFL-17 | JFD-A4 | Stx1/Stx2 | B | 7.2 ± 0.8 | 12 ± 4 | 30 | >330 | 10 | 50 | |
| Stx-A5 | JFL-29 | JFD-A5 | Stx1/Stx2 | B | 12.5 ± 0.9 | 7.7 ± 0.5 | 15 | 100 | 1 | 10 | |
| Stx2-A6 | JFA-31 | JEU-A6 | Stx2 | B | NB | 5 ± 2 | >1000 | ND | 1 | 5 | |
| Stx2-D2 | JFA-36 | JEU-D2 | Stx2* | B | NB | 7.0 ± 0.9 | >1000 | ND | 2 | 20 | |
| Stx2-D10 | JFL-47 | JEN-D10 | Stx2 | B | NB | 0.21 ± 0.01 | >1000 | >1000 | 0.3 | 0.7 | |
| Stx2-G1 | JGL-34 | JGH-G1 | Stx2 | B | NB | 0.023 ± 0.003 | >1000 | >1000 | 0.1 | 0.04 | |
| Stx2-G9 | JGL-40 | JGH-G9 | Stx2* | B | NB | 19 ± 2 | >1000 | >1000 | 2 | 3 | |
| Stx2-H6 | JFL-88 | JFG-H6 | Stx2 | B | NB | 0.41 ± 0.01 | >1000 | >1000 | 0.5 | 1 | |

^{a}Subunit assessed by Western blot
^{b}K_{D} assessed by SPR
^{c}EC₅₀s assessed by dilution ELISAs (see FIGs. 31, 32)
^{d}IC₅₀s assessed by cell assays (see FIGs. 33, 34)
*Slight cross-reactivity to Stx1

Competition ELISAs indicated that VHHs binding to the B subunit, including both the Stx-specific VHHs and the cross-specific VHHs, displayed ability to compete for the binding of the other VHHs recognizing the same toxinotype. VHHs with a lower KD for Stx were stronger competitors than other VHHs. The results indicate that VHHs recognizing the Stx B subunit bound at the same or overlapping epitopes, or induced conformational changes that reduced binding by other VHHs.

### Example 45

### Binding Properties of Stx-Binding VHH Heterodimers

The examples herein show that linking of two toxin-neutralizing VHHs into heterodimers that also contain two epitopic tags (referred to herein as VHH-based neutralizing agent or VNA) neutralized the toxin target and protected animals from intoxication. Antitoxin protection, especially with high dose challenge, was enhanced by co-administration of an anti-tag effector antibody (efAb) See Mukherjee J. et al. 2012 PLoS ONE 7: e29941. As shown in FIG. 32A, the double-tagged heterodimer directs four efAb molecules to the toxin leading to clearance from the serum. See Sepulveda J, et al. 2010 Infect Immun 78: 756-763. To test this strategy with Shiga toxins, several heterodimeric VNAs were generated by fusing different combinations of two VHHs that target Stx1 And/or Stx2. The five heterodimeric VNAs with highest affinities for Stx1 And Stx2 are listed in Table 7. The binding properties of these VNAs were assessed by dilution ELISAs (FIGS. 30 and 31) and EC50's were estimated (Table 7).

TABLE 7
Properties of VHH heteromultimers recognizing Stxl and/or Stx2

| Heteromulti- | | | | | K_{D} (nM)^{a} | K_{D}(nM)^{b} | EC₅₀ (nM)^{b} | IC₅₀ (nM)^{c} | EC₅₀ (nM)^{b} | IC₅₀ (nM)^{c} |
|---|---|---|---|---|---|---|---|---|---|---|
| mer name | Clone | VHH 1 | VHH 2 | VHH 3 | Stx1 | Stx2 | Stx1 | Stx1 | Stx2 | Stx2 |
| A5/A4 | JFX-10 | Stx-A5 | Stx-A4 | none | 0.74 ± 0.04 | 0.9 ± 0.1 | 0.4 | 0.3 | 0.3 | 0.05 |
| A9/A4 | JFX-27 | Stx1-A9 | Stx-A4 | none | 0.50 ± 0.03 | 80 ± 20 | 0.5 | 0.05 | 50 | >100 |
| A9/D4 | JGX-2 | Stx1-A9 | Stx1-D4 | none | 1.2 ± 0.4 | NB | 0.6 | 1 | ND | >100 |
| A5/D10 | JFX-16 | Stx-A5 | Stx2-D10 | none | 9.2 ± 0.8 | 0.20 ± 0.01 | 30 | 50 | 0.8 | 0.02 |
| G1/D10 | JGX-19 | Stx2-G1 | Stx2-D10 | none | NB | 0.004 ± 0.005 | >1000 | >100 | 0.3 | 0.04 |
| A9/A5/D10 | JFZ-29 | Stx1-A9 | Stx-A5 | Stx2-D10 | 0.71 ± 0.03 | 0.7 ± 0.1 | 0.3 | 0.08 | 0.3 | 0.03 |
| A9/A5/G1 | JHO02 | Stx1-A9 | Stx-A5 | Stx2-G1 | 0.46 ± 0.02 | 0.09 ± 0.02 | 0.5 | 0.05 | 0.3 | 0.04 |

^{a}K_{D} assessed by SPR
^{b}EC₅₀s assessed by dilution ELISAs (see FIGs. 31, 32)
^{c}IC₅₀s assessed by cell assays (see FIGs. 33, 34)

Heterodimeric VNAs were observed to display substantially lower EC50 and KD values compared with either VHH alone (Table 7), indicating that linking the VHHs together improves target affinity. Enhanced binding affinities were unambiguous when the two component VHHs had lower target affinity, such as with the Stx cross-specific VHHs, Stx-A4 and Stx-A5. In this case, monomer VHHs displayed EC50 and KD values in the range of 1-30 nM for both Stx1 And Stx2 while the A5/A4 heterodimer displayed sub-nanomolar values, about 10× improvements. Similar improvements were observed with other heterodimers (e.g. A9/A4 and G1/D10) when both VHH components recognized the same Stx toxinotype.

### Example 46

### Shiga Toxin Neutralization Properties of VHH-Based Agents Recognizing Stx1 and/or Stx2

Stx1- and Stx2-binding VHHs were assessed for their toxin neutralization potency in a cell-based assay. Dilution assays are shown in FIG. 33 for Stx1 and in FIG. 34 for Stx2. The results, including the IC50 estimates from serial dilution assays, are summarized in Tables 6 and 7. VHHs in Table 6, except Stx1-D4 (FIG. 33C), displayed ability to neutralize one or both of the Stxs (FIGS. 33 and 34). None of the VHHs tested showed neutralizing activity on a Stx for which no binding was detected by ELISA or SPR. VNAs with the lowest ELISA EC50 displayed the lowest cell-based neutralizing IC50, indicating that toxin affinity plays an important role in neutralization. The cross-specific VHH monomers Stx-A4 and A5 displayed substantially higher IC50s than EC50s and were poor toxin neutralizers as monomers. The Stx1- or Stx2-specific VNAs generally displayed IC50s that were equal or slightly lower than the EC50s and were as low as 100 picomolar for Stx2-specific VHH Stx2-G1.

Stx neutralization by the heterodimeric VNAs listed in Table 7 was assessed by comparing their IC50s with equimolar pools of their two component monomers. As shown in FIGS. 33 and 34, neutralization potency of monomer pools was observed to be about the same or less than that of the most potent monomer in the pool. In contrast, linking VHHs into a heterodimeric VNA almost always improved neutralization potency. The neutralization potency of heterodimeric VNA was greater in the poorly neutralizing Stx cross-specific VHHs, Stx-A4 and StxA5, for which the A4/A5 heterodimer potency on both Stx1 And Stx2 was 100 fold greater than the pool of monomer VHH components (FIGS. 33A and 34A, Tables 6 and 7). Similar major improvements in potency of heterodimeric VNAs compared to monomer pools were observed with Stx-A4 and Stx1-A9 (FIG. 33B), and with Stx-A5 and Stx2-D10 (FIG. 34C). A heterodimer joining the non-neutralizing VHH, Stx1-D4 and the neutralizing VHH, Stx1-A9, was substantially more potent at neutralizing Stx1 than was an equimolar treatment with Stx1-D4 and Stx1-A9 monomers, indicating that the improvement in affinity afforded by the A9/D4 heterodimer vs Stx1-A9 monomer (Tables 6 and 7) is sufficient to improve the neutralizing potency.

Only one heterodimeric VNA, G1/D10, did not achieve greater Stx neutralization potency than the component monomers (FIG. 34B). The neutralizing IC50 of the monomer, Stx2-G1, at 40 pM, is approximately the same as the Stx2 concentration (35 pM) used in the cell-based assay. Since neutralization is expected to require at least a 1:1 stoichiometric ratio of agent:toxin, further improvement in potency may not be possible even if higher affinity VHHs are identified (Table 6 and 7). By this analysis, many of the VHH-based anti-Stx VNAs in Table 7 were found to be effective at Stx neutralization when combined at equimolar ratios to the toxin target (e.g. A9/A4 with Stx1 And for A5/A4, A5/D10 and G1/D10 with Stx2).

### Example 47

### VHH Heteromultimers that Recognize Both Shiga Toxins Stx1 and Stx2

An ideal antitoxin agent for the Shiga toxins would be a single protein capable of neutralizing both Stx1 And Stx2. Because some neutralizing VHHs were cross-specific for both Stx1 And Stx2, VNAs were engineered that included one Stx cross-specific VHH and two neutralizing VHHs specific to either Stx1 or Stx2. Two such heterotrimeric VNAs were produced; one combining Stx1-A9, Stx-A5, and Stx2-D10 (A9/A5/D10) and another combining Stx1-A9, Stx-A5 and Stx2-G1 (A9/A5/G1). Each VHH in the VNAs were separated by a flexible spacer region (GGGGS)3 and a copy of the E-tag peptide was present at the amino and carboxyl sides of the VHH heterotrimer.

The Stx-binding properties of the two heterotrimer VNAs were characterized by ELISAs and neutralization assays. FIGS. 30D, 31C and 31D and Table 7 show that the VNAs have EC50 binding properties in the sub-nanomolar range for both toxins. The EC50 and KD values of the heterotrimeric VNAs (Table 7) were similar to those of corresponding heterodimer VNAs, indicating that the full binding function of all three VHHs in the heterotrimers were retained. Both Stx-binding heterotrimer VNAs also showed excellent neutralization properties against both Stx1 And Stx2 in cell-based assays (FIGS. 33D, 34C and 34D). In fact, the IC50 estimates for the heterotrimer VNAs were near the toxin concentrations for both Stx1 And Stx2, implying that each agent was able to neutralize both toxins when present at near equimolar concentrations to the toxins (Table 7) in these assays. Thus a single heterotrimer VNA consisting of a high affinity Stx1-binding VHH, a high affinity Stx2-binding VHH, and a moderate affinity Stx cross-specific VHH, is capable of potent neutralization of both Stx1 And Stx2.

### Example 48

### Protection from Shiga Toxin Intoxication in Mice Using VHH-Based Antitoxin Agents

Stx1- and Stx2-binding monomer VHHs and heteromultimeric VNAs were tested for the ability to protect mice from Stx lethality. For these examples, 40 pmoles of VHH or VNA was co-administered with toxin an amount of 1.25× the minimal lethal dose (MLD) of Stx1 was about 20 pmoles and for Stx2 the 1.25×MLD was about 1 pmole. As a result of the different Stx potencies, the doses of VHH-based agents used were about two-fold molar excess to Stx1 And about forty-fold excess to Stx2.

The Stx1-binding monomer VHHs in Table 6 were tested for in vivo efficacy. It was observed that none led to improved survival (see FIGS. 35A and 35B). To test whether this was due to the small molar excess employed, a series of two fold higher doses of Stx1-A9 was employed up to sixteen fold (640 pmoles). The data showed that this change led to no improvement in efficacy (FIG. 35A). Use of heterodimer or heterotrimer VNAs resulted in little to no extension in the time to death in mice intoxicated with Stx1 (See FIGS. 35B and 35C). No improvement was detected using a two-fold higher dose. To determine whether efficacy could be improved by promoting clearance of Stx1, the anti-E-tag efAb was co-administered with either of the two heterotrimer VNAs. An 80 pmole dose of this efAb was employed to provide sufficient Ab to bind to both copies of the tag present on each of the heterotrimer VNAs, thus leading to toxin decoration by up to four efAbs. Inclusion of the efAb resulted in complete protection of mice from clinical signs and death due to Stx1 (FIGS. 35C and 35D). Administration of efAb alone had no effect on the survival of mice given 1.25 MLD of Stx1 or Stx2.

With Stx2 intoxication, monomer neutralizing VHHs did not improve survival (See FIGS. 36A and 36B). A beneficial effect on survival was observed with heteromultimeric VNAs for Stx2-intoxicated mice (FIGS. 36B and 36D), however, these mice had signs of intoxication (lethargy, dehydration, excessive urination). In contrast, clearance of the Stx2 from serum promoted by co-administering efAb with the VNA resulted in 100% survival in each group (e.g. FIGS. 36C and 36D) and no symptoms of intoxication were observed.

### Example 49

### Decorating Stx with efAb to Promote Clearance by Targeting the Pentameric B-Subunits

The Stxs consist of a single A-subunit and five B-subunits. VHHs that bind to the B-subunit thus have the potential to bind at five separate sites on each Stx molecule. If each VHH binds to a single efAb, the toxin could be decorated by a plurality of two to five Ab molecules (see FIG. 32B) which should be sufficient to promote serum clearance. In contrast, co-administering efAb with monomeric VHHs recognizing the pentameric B-subunit of Stx yielded substantial improvements in survival to toxin challenge. An example employing monomeric Stx2-D10 is shown in FIG. 36A. In the absence of efAb, the monomeric, toxin-neutralizing VHH delayed death for one to two days. In contrast, co-administration of efAb resulted in 100% survival. The same result was obtained in other examples testing two additional B subunit-binding, single-tagged monomeric VHHs, Stx2-G1 And Stx2-H6.

### Example 50

### Treatment with VNAs and efAb Protects Mice from Stx2 Induced Kidney Toxicity

Stx2 intoxicated mice that were treated only with the heteromultimer VNA, A9/A5/G1, and survived showed signs of kidney damage due to intoxication (lethargy, dehydration, excessive urination). The mice exposed with 1.25 MLD of Stx2 and were then treated with the A9/A5/G1 VNA alone. The kidneys were observed to have damage to distal tubular epithelial cells. Affected tubules demonstrated epithelial cell changes (apoptosis/necrosis, attenuation and restitution, hypertrophy, hyperplasia, and luminal dilation) and additional lesions (tubular atrophy/collapse, interstitial cell proliferation and early interstitial fibrosis).

In contrast, damaged tubules were significantly reduced in mice treated with A9/A5/G1 and the efAb (FIG. 37D). FIG. 37A shows control image from an untreated age- and sex-matched control kidney with no lesions, revealing minimal kidney damage with A9/A5/G1 and efAb (FIG. 37B), and severe distal tubular lesions in mice that received this VNA alone (FIG. 37C). The tubular epithelial lesions are consistent with the stereotypical reparative responses secondary to death of tubular epithelial cells due to Stx2 and with Stx2-induced tubular epithelial cell apoptosis. (Psotka M A, et al. 2009 Infect Immun 77: 959-969) These results indicate that A9/A5/G1 VNA and efAb treatment prevented kidney damage by promoting both toxin neutralization and clearance.

1. A pharmaceutical composition for treating a subject at risk for exposure to or exposed to at least one disease agent, the pharmaceutical composition comprising: at least one recombinant binding protein that neutralizes the disease agent and treats the subject for exposure to the disease agent, wherein the binding protein comprises at least one amino acid sequence selected from the group of:
(SEQ ID NO: 96)
QVQLVETGGGLVQAGDPLRLSCVASGRTVSRYDKAWFRQAPGKEREFVAGIS
WNGDTKIYADSVKGRFTISRENSRDTLDLQIDNLKPEDTAAYYCAVGIAGVQS
MARMLGVRYWGQGTQVTVSSEPKTPKPQ;
(SEQ ID NO: 98)
QVQLVETGGGLVQPGGSLRLSCAASGFSLDPYVIGWFRQAPGKEREGVSCITSR
AASRTSVDSVNERFTISRDNAKNTVDLHINNLKPEDSGVYYCAAVPPAKLPLFS
LCRSLPAKYDYWGQGTQVTVSSAHHSEDPS;
(SEQ ID NO: 100)
QVQLVESGGGLVQPGGSLRLSCAASGSSFSRYAMRWYRQAPGKQRELVANINS
RGTSNYADSVKGRFTISRDNAKNTVYLQMNSLKPEDTAVYYCNAEWLGRSEPS
WGQGTQVTVSSEPKTPKPQ;
(SEQ ID NO: 102)
QVQLVESGGGLVQPGGSLRLSCAASGFIFSLYTMRWHRQAPGKERELVATITSA
TGITNYADSVKGRFIISRDDAKKTGYLQMNSLKPEDTAVYYCNAVRTTVSRDY
WGQGTQVTVSSEPKTPKPQ;
(SEQ ID NO: 104)
QVQLVESGGGLVQPGGSLRLSCAASGIIFSIYTMGWYRQAPGKQRELVAAIPSG
PSANATDSVGGRFTITRDNAENTVYLQMNDLKPEDTAVYYCNARRGPGIKNY
WGQGTQVTVSSEPKTPKPQ;
(SEQ ID NO: 106)
QVQLVESGGGLVQPGGSLSVSCAASGSIARPGAMAWYRQAPGKERELVASITP
GGLTNYADSVTGRFTISRDNAKRTVYLQMNSLQPEDTAVYYCHARIIPLGLGSE
YRDHWGQGTQVTVSSAHHSEDPS;
(SEQ ID NO: 108)
QVQLVETGGGLVQPGGSLGLSCVVASGRSINNYGMGWYRQAPGKQRELVAQI
SSGGTTNYAGSVEGRFTISRDNVKKMVYLQMNSLKPEDTAVYYCNSLLRTFSW
GQGTQVTVSSAHHSEDPS;
(SEQ ID NO: 110)
QVQLVETGGLVQPGGSLRLSCAASGLTFSSTAMAWFRQAPGKEREFVARISGA
GITIYYSDSVKDRFTISRNNVENTVYLQMNSLKTEDTAVYYCAARRNTYTSDYN
IPARYPYWGQGTQVTVSSEPKTPKPQ;
(SEQ ID NO: 112)
QVQLVETGGLVQPGGSLRLSCAASRSTTATIYSMNWYRQAPGKQRELVAGMTS
DGQTNYATSVKGRFTISRDNAKNTVYLLMNSLKLEDTAVYYCYVKPWRLQGW
DYWGQGTQVTVSSEPKTPKPQ;
(SEQ ID NO: 114)
QVQLVESGGGLVQPGGSLRLSCAAPESIVNSRTMAWYRQAPGKQRERVATITT
AGSPNYADSVKGRFAISRDNAKNTVYLQMNSLKPEDTAVYYCNTLLSTLPYGQ
GTQVTVSSAHHSEDPS;
(SEQ ID NO: 116)
QVQLVESGGGLVQPGGSLGLSCVVASERSINNYGMGWYRQAPGKQRELVAQIS
SGGTTNYADSVEGRFTISRDNVKKMVHLQVNSLKPEDTAVYYCNSLLRTFSWG
QGTQVTVSSEPKTPKPQ;
(SEQ ID NO: 118)
QVQLVETGGGLVQPGGSLRLSCAASGFTFSSYRMSWYRQAAGKERDVVATITA
NGVPTGYADSVMGRFTISRDNAKNTVYLEMNSLNPEDTAVYYCNAPRLHTSV
GYWGQGTQVTVSSEPKTPKPQ;
(SEQ ID NO: 120)
QVQLVESGGGLVQAGNSLRLSCTASGVIFSIYTMGWFRQAPGKEREFVAAIGVA
DGTALVADSVTGRFTISRDNAKNTVYLHMNSLKPEDTAVYSCAAYLSPRVQSP
YITDSRYQLWGQGTQVTVSSEPKTPKPQ;
(SEQ ID NO: 122)
TGGGLVQAGGSLRLSCAASGRYAMGWFRQAPGKEREFVATISRSGAIREYADS
VKGRFTISRDGAENTVYLEMNSLKPDDTAIYVCAEGRGATFNPEYAYWGQGTQ
VTVSSAHHSEDPS;
(SEQ ID NO: 124)
QVQLVESGGGLVQPGGSLRLSCAASGFTLDDYAIGWFRQVPGKEREGVACVKD
GSTYYADSVKGRFTISRDNGAVYLQMNSLKPEDTAVYYCASRPCFLGVPLIDFG
SWGQGTQVTVSSEPKTPKPQ;
(SEQ ID NO: 126)
QVQLVESGGGLVQAGGSLRLSCATSGGTFSDYGMGWFRQAPGKEREFVAAIRR
NGNGGNGIEYADSVKGRFTISRDNAKNTVHLQMNSLTPEDTAVYYCAASISGY
AYNTIERYNYWGQGTQVTVSSEPKTPKPQ;
(SEQ ID NO: 128)
QVQLVESGGGLVQAGGSLSLSCAASGGDFSRNAMAWFRQAPGKEREFVASIN
WTGSGTYYLDSVKGRFTISRDNAKNALYLQMNNLKPEDTAVYYCARSTVFAEI
TGLAGYQSGSYDYWGQGTQVTVSSEPKTPKPQ;
(SEQ ID NO: 130)
QVQLVETGGGTVQTGGSLRLSCSASGGSFSRNAMGWFRQAPGKEREFVAAIN
WSASSTYYRDSVKGRFTVSRDNAKNTVYLHENSLKLEDTAAYYCAGSSVYAE
MPYADSVKATSYNYWGQGTQVTVSSEPKTPKPQ;
(SEQ ID NO: 132)
QVQLVETGGGLVQAGGSLRLPCSFSGFPFDNYFVGWFRQAPGKEREGVSCISSS
DGSTYYADSVKGRFTISRDNAKNTVYLQMNSLKPEDTAVYYCGADFLTPHRCP
ALYDYWGQGTQVTVSSAHHSEDPS;
(SEQ ID NO: 134)
QVQLVESGGGLVQPGGSLRLHCAASGSIASIYRTCWYRQGTGKQRELVAAITSG
GNTYYADSVKGRFTISRDNAKNTIDLQMNSLKPEDTAVYYCNADEAGIGGFND
YWGQGTQVTVSSAHHSEDPS;
(SEQ ID NO: 136)
QVQLVESGGGLVQAGGSLRLSCAASGRTFSRSSMGWFRQAPGKEREFVASIVW
ADGTTLYGDSVKGRFTVSRDNVKNMVYLQMNNLKPEDTALYYCADNKFVRG
LVAVRAIDYDYWGQGTQVTVSSEPKTPKPQ;
(SEQ ID NO: 138)
QVQLVESGGLVQAGGSLRLSCAASGRADIIYAMGWFRQAPGKEREFVAAVDW
SGGSTYYADSVKGRFTISRDNAKNSVYLQMNSLKPEDTAVYYCAARRSWYRD
ALSPSRVYEYDYWGQGTQVTVSSEPKTPKPQ;
(SEQ ID NO: 140)
QVQLVETGGGLVQPGGSLTLSCAGSGGTLEHYAIGWFRQAPGKEHEWLVCNR
GEYGSTVYVDSVKGRFTASRDNAKNTVYLQLNSLKPDDTGIYYCVSGCYSWR
GPWGQGTQVTVSSAHHSEDPS;
(SEQ ID NO: 142)
QVQLVESGGGLVQPGGSLKLSCRASGSIVSIYAVGWYRQAPGKQRELLAAITTD
GSTKYSDSVKGRFTISRDNAKNTVYLQMNNLKPEDTAIYSCIGDAAGWGDQYY
WGQGTQVTVSSEPKTPKPQ;
(SEQ ID NO: 144)
QVQLVESGGGLVQAGGSLRLSCAASGSIVNFETMGWYRQAPGKERELVATITN
EGSSNYADSVKGRFTISGDNAKNTVSLQMNSLKPEDTAVYYCSATFGSRWPYA
HSDHWGQGTQVTVSSEPKTPKPQ;
(SEQ ID NO: 146)
QVQLVETGGALVHTGGSLRLSCEVSGSTFSSYGMAWYRQAPGEQRKWVAGIM
PDGTPSYVNSVKGRFTISRDNAKNSVYLHMNNLRPEDTAVYYCNQWPRTMPD
ANWGRGTQVTVSSEPKTPKPQ;
(SEQ ID NO: 148)
QVQLVETGGSLRLTCVTSGSTFNNPAITWYRQPPGKQREWVASLRSGDGPVYR
ESVKGRFTIFRDNATDALYLRMNSLKPEDTAVYHCNTASPASWLDWGQGTQV
TVSSEPKTPKPQ;
(SEQ ID NO: 150)
QVQLVETGGGLVQPGGSLRLSCATSGFPFSTERMSWVRQAPGKGLEWVSGITE
GGETTLAAPSVKGRENISRDNARNILYLQMNSLKPEDAAVYYCFRGVFFRTSFP
PELARGQGTQVTVSSEPKTPKPQ;
(SEQ ID NO: 152)
QVQLVESGGGLVQAGGSLRLSCAASGSAVSDSFSTYAISWHRQAPGKQREWIA
GISNRGATSYRDSVKGRFTISRDNAKNTVYLQMNNLKPEDTGVYYCEPWPREG
LGGGQGTQVTVSSEPKTPKPQ;
(SEQ ID NO: 154)
QVQLVESGGGSVQTGGSLTLSCVVSGSTFSDYAVAWYRQVPGKSRAWVAGVS
TTGSTSYTDSVRGRFTISRDNHKKTVYLSMNSLKPEDTGIYYCNLWPFTNPPSW
GQGTQVTVSSAHHSEDPS;
(SEQ ID NO: 156)
QVQLVESGGAVVQPGGSLRLSCATSGFTFSDDRMSWARQAPGKGLEWVSGIST
ASEGFATLYAPSVKGRFTISRDNAKHMLYLQMDTLKPEDTAVYYCLRGVFFRT
NIPPEVLRGQGTQVTVSSAHHSEDPS;
(SEQ ID NO: 158)
QVQLVETGGDLVQPGGSLRLSCAASGSSFSRAAVGWYRQAPGKEREWVARLA
SGDMTDYTESVRGRFTISRDNAKHTVYLQMDNLKPEDTAVYYCKARIPPYYSIE
YWGKGTRVTVSSEPKTPKPQ;
(SEQ ID NO: 160)
QVQLVETGGGLVQAGGSLRLSCVVSSPLFNLYDMAWYRQAPGNQRELVAGIL
TDGRATYSDSVKGRFTISRNNLTNTVFLQMSSLKPEDTAVYYCNRKNSIYWDS
WGQGTQVTVSSEPKTPKPQ;
and,
(SEQ ID NO: 162)
QVQLVESGGGLVQAGGSLRLSCVASGLTFSRYGMGWFRQAPGQERVVVSVISP
DGGSAYYADSVKGRFTISRDNAKNTVYLQMSTLRFEDTGVYYCTAGPRNGATT
VLRPGDYDYWGQGTQVTVSSEPKTPKPQ.

2. The pharmaceutical composition according to claim 1, wherein the pharmaceutical composition comprises a source of expression of at least one recombinant binding protein that neutralizes the disease agent to treat the subject for exposure to the disease agent, wherein the source of expression comprises at least one nucleotide sequence selected from the group of:
(SEQ ID NO: 97)
CAGGTGCAGCTCGTGGAGACGGGGGGAGGATTGGTGCAGGCTGGGGACCCT
CTGAGACTCTCCTGTGTAGCCTCTGGACGCACCGTCAGTCGCTATGACAAGG
CCTGGTTCCGCCAGGCTCCAGGGAAGGAGCGTGAGTTTGTAGCAGGAATTA
GCTGGAACGGCGATACAAAAATTTATGCAGACTCCGTGAAGGGCCGATTCA
CCATCTCCAGAGAGAACTCCAGGGATACACTGGATCTGCAAATTGACAACC
TGAAACCTGAGGACACGGCCGCGTATTACTGTGCGGTCGGAATTGCGGGTG
TTCAGAGTATGGCGCGTATGCTCGGAGTGCGCTACTGGGGCCAGGGGACCC
AGGTCACCGTCTCCTCAGAACCCAAGACACCAAAACCACAA;
(SEQ ID NO: 99)
CAGGTGCAGCTCGTGGAGACGGGGGGAGGCTTGGTGCAGCCTGGGGGGTCT
CTGAGACTCTCCTGTGCAGCCTCTGGTTTCAGTTTGGACCCTTATGTGATAG
GATGGTTCCGGCAGGCCCCAGGGAAGGAGCGTGAGGGGGTCTCATGTATTA
CGAGTAGGGCTGCTAGTCGAACGTCTGTAGACTCCGTGAACGAGCGATTCA
CCATCTCCAGAGACAACGCCAAGAATACGGTCGATCTACACATCAATAACC
TGAAACCTGAGGACTCGGGCGTTTATTACTGTGCAGCGGTCCCCCCTGCCAA
ATTACCACTTTTCAGCCTATGTCGCTCCCTGCCAGCAAAGTATGACTACTGG
GGCCAGGGGACCCAGGTCACCGTCTCCTCAGCGCACCACAGCGAAGACCCC
TCG;
(SEQ ID NO: 101)
CAGGTGCAGCTCGTGGAGTCGGGGGGAGGCTTGGTGCAGCCTGGGGGGTCT
CTGAGACTCTCCTGTGCAGCCTCTGGAAGTAGCTTCAGTAGATATGCCATGC
GCTGGTACCGCCAGGCTCCAGGGAAGCAGCGCGAGTTGGTCGCAAACATTA
ATAGTCGTGGTACCTCAAACTATGCAGACTCCGTGAAGGGCCGATTCACCAT
CTCCAGAGACAACGCCAAGAACACGGTGTATCTGCAAATGAACAGCCTGAA
ACCTGAAGACACGGCCGTCTATTATTGTAATGCAGAGTGGTTGGGACGATC
GGAGCCTTCCTGGGGCCAGGGGACCCAGGTCACCGTCTCCTCGGAACCCAA
GACACCAAAACCACAA;
(SEQ ID NO: 103)
CAGGTGCAGCTCGTGGAGTCAGGAGGAGGCTTGGTGCAGCCTGGGGGGTCT
CTGAGACTCTCCTGTGCAGCCTCTGGATTCATTTTCAGTCTTTATACCATGAG
GTGGCACCGCCAGGCTCCAGGGAAGGAGCGCGAGTTGGTCGCGACTATTAC
TAGTGCTACTGGTATTACAAACTATGCAGACTCCGTGAAGGGCCGATTCATC
ATCTCCAGAGACGATGCCAAGAAGACGGGGTATCTGCAAATGAACAGCCTG
AAACCTGAGGACACGGCCGTGTATTACTGTAATGCAGTCCGCACTACCGTGT
CACGAGACTACTGGGGCCAGGGGACCCAGGTCACCGTCTCCTCAGAACCCA
AGACACCAAAACCACAA;
(SEQ ID NO: 105)
CAGGTGCAGCTCGTGGAGTCTGGGGGAGGCTTGGTGCAGCCTGGGGGGTCT
CTGAGACTCTCCTGTGCAGCCTCTGGAATCATCTTCAGTATCTATACCATGG
GCTGGTACCGCCAGGCTCCAGGGAAGCAGCGCGAATTGGTCGCAGCTATAC
CTAGTGGTCCTAGCGCAAACGCTACAGACTCCGTGGGGGGCCGATTCACCA
TCACCAGAGACAACGCCGAGAACACGGTGTATCTGCAAATGAACGACCTGA
AACCTGAGGACACGGCCGTCTATTACTGTAATGCTCGGCGGGGTCCGGGTAT
CAAAAACTACTGGGGCCAGGGGACCCAGGTCACCGTCTCCTCAGAACCCAA
GACACCAAAACCACAA;
(SEQ ID NO: 107)
CAGGTGCAGCTCGTGGAGTCCGGGGGGCGGCTTGGTGCAGGCCCGGGGGGTCT
CTGAGTGTCTCCTGTGCAGCCTCTGGAAGCATCGCAAGACCAGGTGCCATGG
CCTGGTACCGCCAGGCTCCAGGGAAGGAGCGCGAGTTGGTCGCGTCTATTA
CGCCTGGTGGTCTTACAAACTATGCGGACTCCGTGACGGGCCGATTCACCAT
TTCCAGAGACAACGCCAAGAGGACGGTGTATCTGCAGATGAACAGCCTCCA
ACCCGAGGACACGGCCGTCTATTACTGTCATGCACGAATAATTCCCCTAGGA
CTTGGGTCCGAATACAGGGACCACTGGGGCCAGGGGACTCAGGTCACCGTC
TCCTCAGCGCACCACAGCGAAGACCCCTCG;
(SEQ ID NO: 109)
CAGGTGCAGCTCGTGGAGACGGGGGGAGGCTTGGTGCAGCCTGGGGGGTCT
CTGGGACTCTCCTGTGTAGTCGCCTCTGGAAGAAGCATCAATAATTATGGCA
TGGGCTGGTACCGCCAGGCTCCAGGGAAGCAGCGCGAGTTGGTCGCGCAAA
TTAGTAGTGGTGGTACCACAAATTATGCAGGCTCCGTAGAGGGCCGATTCAC
CATCTCCAGAGACAACGTCAAGAAAATGGTGTATCTTCAAATGAACAGCCT
GAAACCTGAGGACACGGCCGTCTATTACTGTAATTCACTGCTCCGAACTTTT
TCCTGGGGCCAGGGGACCCAGGTCACCGTCTCCTCGGCGCACCACAGCGAA
GACCCCTCG;
(SEQ ID NO: 111)
CAGGTGCAGCTCGTGGAGACCGGGGGGTTGGTGCAGCCTGGGGGCTCCCTG
CGACTCTCCTGTGCAGCCTCCGGACTCACCTTCAGTAGCACTGCCATGGCCT
GGTTCCGCCAGGCTCCAGGGAAGGAGCGTGAGTTTGTAGCACGTATTAGCG
GGGCTGGTATTACGATCTACTATTCGGACTCCGTGAAGGACCGATTCACCAT
CTCCAGAAACAACGTCGAGAACACGGTGTATTTGCAAATGAACAGCCTGAA
AACTGAGGACACGGCCGTTTACTACTGTGCAGCAAGACGGAATACTTACAC
TAGCGACTATAACATACCCGCCCGGTATCCCTACTGGGGCCAGGGGACCCA
GGTCACCGTCTCCTCAGAACCCAAGACACCAAAACCACAA;
(SEQ ID NO: 113)
CAGGTGCAGCTCGTGGAGACGGGGGGCTTGGTGCAGCCTGGGGGGTCTCTG
AGACTCTCCTGTGCAGCCTCTAGAAGCACGACGGCCACAATTTATAGTATGA
ACTGGTACCGCCAGGCTCCAGGGAAGCAGCGCGAGTTGGTCGCGGGTATGA
CTAGTGATGGTCAGACAAACTATGCAACCTCCGTGAAGGGCCGATTCACCA
TCTCCAGAGACAACGCCAAGAACACGGTATATTTGCTAATGAACAGCCTGA
AACTTGAGGACACGGCCGTCTATTATTGTTATGTAAAACCATGGAGACTACA
AGGTTGGGACTACTGGGGCCAGGGGACCCAGGTCACCGTCTCCTCAGAACC
CAAGACACCAAAACCACAA;
(SEQ ID NO: 115)
CAGGTGCAGCTCGTGGAGTCGGGCGGCGGCTTGGTGCAGCCTGGGGGGTCT
CTGAGACTCTCCTGTGCAGCCCCTGAAAGCATCGTCAATAGCAGAACCATG
GCCTGGTACCGCCAGGCTCCAGGAAAGCAGCGCGAAAGGGTCGCCACTATT
ACTACTGCTGGTAGCCCAAATTATGCAGACTCTGTGAAGGGCCGATTCGCCA
TCTCCAGAGACAACGCCAAGAACACGGTATATCTGCAAATGAACAGCCTGA
AACCTGAGGACACGGCCGTCTATTACTGCAATACACTTCTCAGCACCCTTCC
CTATGGCCAGGGGACCCAGGTCACCGTCTCCTCGGCGCACCACAGCGAAGA
CCCCTCG;
(SEQ ID NO: 117)
CAGGTGCAGCTCGTGGAGTCGGGCGGAGGCTTGGTGCAGCCTGGGGGGTCT
CTGGGACTCTCCTGTGTAGTCGCCTCTGAAAGAAGCATCAATAATTATGGCA
TGGGCTGGTACCGCCAGGCTCCAGGGAAGCAGCGCGAGTTGGTCGCGCAAA
TTAGTAGTGGTGGTACCACAAATTATGCAGACTCCGTAGAGGGCCGATTCAC
CATCTCCAGAGACAACGTCAAGAAAATGGTGCATCTTCAAGTGAACAGCCT
GAAACCTGAGGACACGGCCGTCTATTACTGTAATTCGCTACTCCGAACTTTT
TCCTGGGGCCAGGGGACCCAGGTCACCGTCTCCTCGGAACCCAAGACACCA
AAACCACAA;
(SEQ ID NO: 119)
CAGGTGCAGCTCGTGGAGACGGGAGGAGGCTTGGTGCAGCCTGGGGGGTCT
CTGAGACTCTCCTGTGCAGCCTCTGGATTCACCTTCAGTAGTTATCGCATGA
GCTGGTACCGGCAGGCTGCAGGGAAGGAGCGCGACGTGGTCGCAACAATTA
CTGCTAATGGTGTTCCCACAGGCTATGCAGACTCCGTGATGGGCCGATTCAC
CATTTCCAGAGACAATGCCAAGAACACGGTGTATCTGGAAATGAACAGCCT
GAATCCTGAGGACACGGCCGTGTATTACTGTAACGCGCCCCGTTTGCATACA
TCTGTAGGCTACTGGGGCCAGGGGACCCAGGTCACCGTCTCCTCAGAACCC
AAGACACCAAAACCACAA;
(SEQ ID NO: 121)
CAGGTGCAGCTCGTGGAGTCGGGAGGAGGATTGGTGCAGGCTGGGAACTCT
CTGAGACTCTCCTGTACGGCCTCTGGTGTGATCTTCTCTATCTATACCATGGG
CTGGTTCCGCCAGGCTCCAGGGAAGGAGCGTGAGTTTGTAGCAGCGATAGG
GGTGGCTGATGGTACCGCACTTGTGGCAGACTCCGTGACGGGCCGATTCACC
ATCTCCAGAGACAACGCCAAGAACACCGTTTATCTGCATATGAACAGCCTG
AAGCCTGAGGACACGGCCGTCTATTCCTGTGCAGCGTATCTTAGCCCCCGTG
TCCAATCCCCCTACATAACTGACTCCCGGTATCAACTCTGGGGCCAGGGGAC
CCAGGTCACCGTCTCCTCAGAACCCAAGACACCAAAACCACAA
(SEQ ID NO: 123)
CAGGTGCAGCTCGTGGAGACTGGGGGAGGATTGGTGCAGGCTGGGGGCTCT
CTGAGGCTCTCCTGTGCAGCCTCTGGACGCTATGCCATGGGCTGGTTCCGCC
AGGCTCCAGGGAAGGAGCGTGAATTTGTAGCGACTATTAGCCGGAGTGGTG
CTATCAGAGAGTATGCAGACTCCGTGAAGGGCCGATTCACCATCTCCAGAG
ACGGCGCCGAGAACACGGTGTATCTGGAAATGAACAGCCTGAAACCTGACG
ACACGGCCATTTATGTCTGTGCAGAAGGACGAGGGGCGACATTCAACCCCG
AGTATGCTTACTGGGGCCAGGGGACCCAGGTCACCGTCTCCTCAGCGCACC
ACAGCGAAGACCCCTCG;
(SEQ ID NO: 125)
CAGGTGCAGCTCGTGGAGTCGGGCGGAGGCTTGGTGCAGCCTGGGGGGTCT
CTGAGACTCTCCTGTGCAGCCTCTGGATTCACTTTGGATGATTATGCCATAG
GCTGGTTCCGCCAGGTCCCAGGGAAGGAGCGTGAGGGGGTCGCATGTGTTA
AAGATGGTAGTACATACTATGCAGACTCCGTGAAGGGCCGATTCACCATCTC
CAGAGACAACGGCGCGGTGTATCTGCAAATGAACAGCCTGAAACCTGAGGA
CACAGCCGTTTATTACTGTGCATCCAGGCCCTGCTTTTTGGGTGTACCACTTA
TTGACTTTGGTTCCTGGGGCCAGGGGACCCAGGTCACCGTCTCCTCGGAACC
CAAGACACCAAAACCACAA;
(SEQ ID NO: 127)
CAGGTGCAGCTCGTGGAGTCAGGGGGAGGATTGGTGCAGGCTGGGGGCTCT
CTGAGACTCTCCTGCGCAACCTCTGGCGGCACCTTCAGTGACTATGGAATGG
GCTGGTTCCGCCAGGCTCCAGGGAAGGAGCGTGAGTTTGTAGCAGCTATTA
GGCGGAATGGTAATGGCGGTAATGGCATTGAATATGCAGACTCCGTGAAGG
GCCGATTCACCATCTCCAGAGACAACGCCAAGAACACGGTGCATCTACAAA
TGAACAGCCTGACACCTGAGGACACGGCCGTTTATTACTGTGCAGCGTCAAT
ATCGGGATACGCTTATAACACAATTGAAAGATATAACTACTGGGGCCAGGG
AACCCAGGTCACCGTCTCCTCAGGAACCCAAGACACCAAAACCACAA;
(SEQ ID NO: 129)
CAGGTGCAGCTCGTGGAGTCCGGCGGAGGATTGGTGCAGGCGGGGGGCTCT
CTGAGTCTCTCCTGTGCAGCCTCTGGAGGTGACTTCAGTAGGAATGCCATGG
CCTGGTTCCGTCAGGCTCCAGGGAAGGAGCGTGAATTTGTAGCATCTATTAA
CTGGACTGGTAGTGGCACATATTATCTAGACTCCGTGAAGGGCCGATTCACC
ATCTCCAGAGACAACGCCAAGAACGCCCTGTATCTGCAAATGAACAACCTG
AAACCTGAGGACACGGCCGTTTATTACTGTGCACGCTCCACGGTGTTTGCCG
AAATTACAGGCTTAGCAGGCTACCAGTCGGGATCGTATGACTACTGGGGCC
AGGGGACCCAGGTCACCGTCTCCTCAGAACCCAAGACACCAAAACCACAA;
(SEQ ID NO: 131)
CAGGTGCAGCTCGTGGAGACCGGCGGAGGAACGGTGCANACTGGGGGCTCT
CTGAGACTCTCCTGTTCAGCCTCTGGCGGCTCCTTCAGTAGGAATGCCATGG
GCTGGTTCCGCCAGGCTCCAGGGAAGGAGCGTGAATTTGTAGCAGCTATTA
ACTGGAGTGCCTCTAGTACTTATTATAGAGACTCCGTGAAGGGACGATTCAC
CGTCTCCAGAGACAACGCCAAGAACACGGTGTATCTGCATTTGAACAGCCT
GAAACTTGAGGACACGGCCGCGTATTACTGTGCTGGAAGCTCGGTGTATGC
AGAAATGCCGTACGCCGACTCTGTCAAGGCAACTTCCTATAACTACTGGGGC
CAGGGGACCCAGGTCACCGTCTCCTCAGAACCCAAGACACCAAAACCACAA;
(SEQ ID NO: 133)
CAGGTGCAGCTCGTGGAGACCGGGGGAGGCTTGGTGCAGGCTGGGGGGTCT
CTGAGACTCCCCTGTTCATTCTCTGGATTCCCTTTCGATAATTATTTCGTAGG
CTGGTTCCGCCAGGCCCCAGGGAAGGAGCGTGAGGGGGTCTCATGTATTAG
TAGTAGTGATGGTAGCACATACTATGCAGACTCCGTGAAGGGCCGGTTCAC
CATCTCCAGAGACAACGCCAAGAACACGGTGTATCTGCAAATGAACAGTCT
GAAACCTGAGGATACGGCCGTTTATTACTGTGGAGCAGATTTCCTCACCCCA
CATAGGTGTCCAGCCTTATATGACTACTGGGGCCAGGGGACCCAGGTCACC
GTCTCCTCAGCGCACCACAGCGAAGACCCCTCG;
(SEQ ID NO: 135)
CAGGTGCAGCTCGTGGAGTCTGGTGGAGGCTTGGTGCAGCCTGGGGGGTCT
CTGAGACTCCACTGTGCAGCCTCTGGAAGCATCGCCAGTATCTATCGCACGT
GCTGGTACCGCCAGGGCACAGGGAAGCAGCGCGAGTTGGTCGCAGCCATTA
CTAGTGGTGGTAACACATACTATGCGGACTCCGTTAAGGGCCGATTCACCAT
CTCCAGAGACAACGCCAAAAACACAATCGATCTGCAAATGAACAGCCTGAA
ACCTGAGGACACGGCCGTCTATTACTGTAATGCAGACGAGGCGGGGATCGG
GGGATTTAATGACTACTGGGGCCAGGGGACCCAGGTCACCGTCTCCTCAGC
GCACCACAGCGAAGACCCCTCG;
(SEQ ID NO: 137)
CAGGTGCAGCTCGTGGAGTCGGGGGGAGGATTGGTGCAGGCTGGGGGCTCT
CTGAGACTCTCCTGTGCAGCCTCTGGACGCACCTTCAGTCGCAGTTCCATGG
GCTGGTTCCGCCAGGCTCCAGGGAAGGAGCGTGAATTCGTTGCGTCCATTGT
CTGGGCTGATGGTACGACGTTGTATGGAGACTCCGTAAAGGGCCGATTCAC
CGTCTCCAGGGACAACGTCAAGAACATGGTGTATCTACAAATGAACAACCT
GAAACCTGAGGACACGGCCCTTTATTACTGTGCGGACAATAAATTCGTCCGT
GGATTAGTGGCTGTCCGTGCGATAGATTATGACTACTGGGGCCAGGGGACC
CAGGTCACCGTCTCGTCAGAACCCAAGACACCAAAACCACAA;
(SEQ ID NO: 139)
CAGGTGCAGCTCGTGGAGTCGGGAGGATTGGTGCAGGCTGGAGGCTCTCTG
AGACTCTCCTGCGCAGCCTCTGGACGCGCCGACATAATCTATGCCATGGGCT
GGTTCCGCCAGGCTCCAGGGAAGGAGCGTGAGTTTGTAGCGGCAGTAGACT
GGAGTGGTGGTAGCACATACTATGCAGACTCCGTGAAGGGCCGATTCACCA
TCTCCAGAGACAACGCCAAGAACTCGGTGTATCTGCAAATGAACAGCCTGA
AACCTGAGGACACGGCCGTTTATTACTGTGCAGCCCGAAGGAGCTGGTACC
GAGACGCGCTATCCCCCTCCCGGGTGTATGAATATGACTACTGGGGCCAGG
GGACCCAGGTCACCGTCTCCTCAGAACCCAAGACACCAAAACCACAA;
(SEQ ID NO: 141)
CAGGTGCAGCTCGTGGAGACGGGAGGAGGCTTGGTGCAGCCTGGGGGGTCT
CTGACACTCTCCTGTGCAGGCTCCGGTGGCACTTTGGAACATTATGCTATAG
GCTGGTTCCGCCAGGCCCCTGGGAAAGAGCATGAGTGGCTCGTATGTAATA
GAGGTGAATATGGGAGCACTGTCTATGTAGACTCCGTGAAGGGCCGATTCA
CCGCCTCCAGAGACAACGCCAAGAACACGGTGTATCTGCAATTGAACAGTC
TGAAACCTGACGACACAGGCATTTATTACTGTGTATCGGGATGTTACTCCTG
GCGGGGTCCCTGGGGCCAGGGGACCCAGGTCACCGTCTCCTCGGCGCACCA
CAGCGAAGACCCCTCG;
(SEQ ID NO: 143)
CAGGTGCAGCTCGTGGAGTCTGGGGGAGGTTTGGTGCAGCCTGGGGGGTCT
CTGAAACTCTCCTGTAGAGCCTCTGGAAGCATAGTCAGTATCTATGCCGTGG
GCTGGTACCGCCAGGCTCCAGGGAAGCAGCGCGAGTTGCTCGCGGCTATCA
CTACTGATGGTAGCACGAAGTACTCAGACTCCGTGAAGGGCCGATTCACCA
TCTCCCGAGACAACGCCAAGAACACGGTATATCTGCAAATGAACAACCTCA
AACCTGAGGACACGGCCATCTATTCCTGTATCGGGGACGCGGCGGGTTGGG
GCGACCAATACTACTGGGGCCAGGGGACCCAGGTCACCGTCTCCTCAGAAC
CCAAGACACCAAAACCACAA;
(SEQ ID NO: 145)
CAGGTGCAGCTCGTGGAGTCAGGCGGAGGCTTGGTGCAGGCTGGGGGGTCT
CTGAGACTCTCCTGTGCAGCCTCTGGAAGCATCGTCAATTTCGAAACCATGG
GCTGGTACCGCCAGGCTCCAGGGAAGGAGCGCGAGTTGGTCGCAACTATTA
CTAATGAAGGTAGTTCAAACTATGCAGACTCCGTGAAGGGCCGATTCACCA
TCTCCGGAGACAACGCCAAGAACACGGTGTCCCTGCAAATGAACAGCCTGA
AACCTGAGGACACGGCCGTCTACTACTGTTCGGCGACGTTCGGCAGTAGGT
GGCCGTACGCCCACAGTGATCACTGGGGCCAGGGGACCCAGGTCACCGTCT
CCTCAGAACCCAAGACACCAAAACCACAA;
(SEQ ID NO: 147)
CAGGTGCAGCTCGTGGAGACGGGCGGAGCATTGGTGCACACTGGGGGTTCT
CTGAGACTCTCCTGCGAAGTCTCCGGAAGCACCTTCAGTAGCTATGGCATGG
CCTGGTACCGCCAAGCTCCAGGCGAGCAGCGTAAGTGGGTCGCAGGTATTA
TGCCGGATGGTACTCCAAGCTATGTAAACTCCGTGAAGGGCCGATTCACCAT
CTCCAGAGACAACGCCAAGAACTCGGTGTATCTGCACATGAACAACCTGAG
GCCTGAAGACACGGCCGTCTATTATTGCAACCAATGGCCGCGCACGATGCCT
GACGCGAACTGGGGCCGGGGGACCCAGGTCACCGTCTCCTCAGAACCCAAG
ACACCAAAACCACAA;
(SEQ ID NO: 149)
CAGGTGCAGCTCGTGGAGACTGGGGGGTCTCTGAGGCTCACCTGTGTAACCT
CTGGAAGCACCTTCAATAATCCTGCCATAACCTGGTACCGCCAGCCTCCAGG
GAAGCAGCGTGAGTGGGTCGCAAGTCTTCGTAGTGGTGATGGTCCAGTATA
TAGGGAATCCGTGAAGGGCCGATTCACCATTTTTAGAGACAACGCCACGGA
CGCGCTGTATCTGCGGATGAATAGCCTGAAACCTGAGGACACGGCCGTCTA
TCACTGTAACACCGCCTCACCTGCTAGTTGGCTGGACTGGGGCCAGGGGACC
CAGGTCACTGTCTCCTCAGAACCCAAGACACCAAAACCACAA;
(SEQ ID NO: 151)
CAGGTGCAGCTCGTGGAGACGGGAGGAGGATTGGTGCAACCTGGGGGTTCT
CTGAGACTCTCTTGTGCAACCTCTGGATTCCCCTTCAGTACGGAGCGTATGA
GCTGGGTCCGCCAGGCTCCAGGAAAGGGGCTCGAGTGGGTCTCAGGTATTA
CTGAGGGTGGTGAAACCACTCTCGCGGCACCCTCCGTGAAGGGCCGATTCA
ACATCTCCAGAGACAACGCCAGGAATATCCTATATCTACAGATGAATTCCTT
GAAACCTGAGGACGCGGCCGTTTACTATTGTTTTAGAGGTGTTTTTTTTAGA
ACGAGTTTTCCTCCCGAACTCGCGCGGGGCCAGGGGACCCAGGTCACCGTCT
CCTCAGAACCCAAGACACCAAAACCACAA;
(SEQ ID NO: 153)
CAGGTGCAGCTCGTGGAGTCGGGCGGAGGCTTGGTGCAGGCAGGGGGGTCT
TTGAGACTCTCCTGTGCAGCCTCTGGAAGCGCCGTCAGTGACAGCTTCAGTA
CCTATGCCATCTCCTGGCACCGCCAGGCTCCAGGGAAGCAGCGTGAGTGGA
TCGCAGGTATTAGTAATCGTGGTGCGACAAGCTATAGAGACTCCGTGAAGG
GCCGATTCACCATCTCCAGAGACAACGCCAAGAACACGGTATATCTGCAAA
TGAACAACCTGAAACCTGAGGACACGGGCGTCTATTATTGTGAGCCATGGC
CACGCGAAGGACTTGGGGGGGGCCAGGGGACTCAGGTCACCGTCTCCTCAG
AACCCAAGACACCAAAACCACAA;
(SEQ ID NO: 155)
CAGGTGCAGCTCGTGGAGTCGGGGGGAGGCTCGGTGCANACTGGGGGGTCT
CTGACACTCTCCTGTGTAGTCTCTGGAAGTACCTTCAGTGACTATGCGGTGG
CCTGGTACCGCCAGGTTCCAGGCAAATCGCGTGCGTGGGTCGCGGGTGTTA
GTACTACTGGCTCGACATCTTATACAGACTCCGTGAGGGGCCGGTTCACCAT
CTCCAGAGACAACCACAAGAAGACGGTGTATCTTTCAATGAACAGCCTGAA
ACCTGAGGACACGGGCATCTATTACTGCAACTTATGGCCGTTCACAAATCCT
CCTTCCTGGGGCCAGGGAACCCAAGTCACCGTTTCCTCGGCGCACCACAGCG
AAGACCCCTCG;
(SEQ ID NO: 157)
CAGGTGCAGCTCGTGGAGTCTGGAGGAGCCGTGGTGCAACCTGGGGGTTCT
CTGAGACTCTCCTGTGCAACCTCTGGATTCACCTTCAGTGACGATCGTATGA
GCTGGGCCCGCCAGGCTCCAGGAAAGGGGCTCGAGTGGGTCTCAGGTATTA
GTACTGCTAGTGAAGGTTTTGCTACACTCTACGCACCCTCCGTGAAGGGCCG
ATTCACCATCTCCAGAGACAACGCCAAGCATATGCTGTATCTGCAAATGGAT
ACCTTGAAACCTGAGGACACGGCCGTGTATTACTGTTTAAGAGGGGTTTTTT
TTAGAACGAACATTCCTCCCGAGGTACTGCGGGGCCAGGGGACCCAGGTCA
CCGTCTCCTCAGCGCACCACAGCGAAGACCCCTCG;
(SEQ ID NO: 159)
CAGGTGCAGCTCGTGGAGACGGGGGGAGACTTGGTGCANCCTGGGGGGTCT
CTGAGACTCTCCTGTGCAGCCTCTGGAAGCTCCTTCAGCCGCGCTGCCGTGG
GCTGGTACCGTCAGGCTCCAGGAAAGGAGCGTGAGTGGGTCGCACGTCTCG
CGAGTGGTGATATGACGGACTATACCGAGTCCGTGAGGGGCCGATTCACTA
TCTCCAGAGACAACGCCAAGCACACGGTGTATCTGCAAATGGACAACCTGA
AACCTGAGGACACGGCCGTCTACTATTGTAAGGCCAGGATACCCCCTTATTA
CTCTATAGAGTACTGGGGCAAAGGGACCCGGGTCACCGTCTCCTCANAACC
CAAGACACCAAAACCACAA;
(SEQ ID NO: 161)
CAGGTGCAGCTCGTGGAGACAGGTGGAGGCTTGGTGCAGGCTGGGGGGTCT
CTGAGACTCTCCTGTGTAGTATCTAGTCCCCTGTTCAATCTTTACGACATGGC
CTGGTATCGCCAGGCTCCAGGGAATCAGCGTGAGTTGGTCGCAGGCATCTTG
ACTGATGGTCGCGCAACATATTCAGACAGCGTGAAGGGCCGATTCACCATTT
CCAGAAACAACCTGACGAACACGGTGTTTTTACAAATGAGCAGCCTGAAAC
CTGAGGACACGGCCGTCTATTATTGTAATAGAAAGAATAGTATCTACTGGG
ATTCCTGGGGCCAGGGGACCCAGGTCACCGTCTCCTCGGAACCCAAGACAC
CAAAACCACAA;
and,
(SEQ ID NO: 163)
CAGGTGCAGCTCGTGGAGTCGGGGGGAGGATTGGTGCAGGCTGGGGGCTCT
CTGAGACTCTCCTGCGTAGCCTCTGGACTCACCTTCAGTCGCTATGGCATGG
GCTGGTTCCGCCAGGCTCCAGGACAGGAGCGTGTAGTCGTATCAGTTATTAG
TCCCGACGGTGGTAGCGCATACTACGCAGACTCCGTGAAGGGCCGATTCAC
CATCTCCAGAGACAACGCCAAGAACACGGTGTATCTGCAAATGAGCACCCT
GAGATTTGAGGACACGGGCGTTTATTATTGTACAGCAGGGCCCCGGAATGG
AGCGACTACAGTCCTCCGGCCAGGGGATTATGACTACTGGGGCCAGGGGAC
CCAGGTCACTGTCTCCTCAGAACCCAAGACACCAAAACCACAA.

3. The pharmaceutical composition according to claim 1, wherein the binding protein comprises a heteromultimeric neutralizing binding protein having a plurality of binding regions, wherein the binding regions are not identical and each binding region has affinity to specifically bind a non-overlapping portion of the disease agent.

4. The pharmaceutical composition according to claim 1, wherein the binding protein further comprises at least one of a tag that is an epitope that is specifically bound by an antibody, and a linker that separates the binding regions, wherein the linker comprises at least one selected from the group of: a peptide, a protein, a sugar, and a nucleic acid.

5. The pharmaceutical composition according to claim 1, wherein the disease agent is a toxin, wherein the toxin is a plant lectin, wherein the plant lectin is at least one selected from the group of: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein or, wherein the toxin is a bacterial toxin, wherein the bacterial toxin is produced by at least one bacterial species selected from the group of: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovate; Pseudomonas for example P. aeruginosa; Neisseria for example N. gonorrhoeae; Escherichia coli for example E. coli O157:H7; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

6. The pharmaceutical composition according to claim 1, wherein the at least one disease agent comprises a plurality of non-identical disease agents, and the binding protein binds to the plurality of the disease agents, thereby neutralizing the disease agents.

7. The pharmaceutical composition according to claim 1, wherein the binding protein comprises an amino acid sequence that is substantially identical to at least one of SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, wherein substantially identical is having at least 50% identity, 60% identity, at least 65% identity, at least 70% identity, at least 75% identity, at least 80% identity, at least 85% identity, at least 90% identity, or and at least 95% identity to the amino acid sequence of SEQ ID NOs: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162.

8. The pharmaceutical composition according to claim 2, wherein the source of expression of the binding protein is selected from the group of: a nucleic acid vector with a gene encoding the binding protein; a viral vector encoding the binding protein; and the binding protein expressed directly from naked nucleic acid.

9. The pharmaceutical composition according to claim 2, wherein the source of expression of the binding protein comprises an nucleotide sequence that is substantially identical to at least one of SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163, wherein substantially identical is having at least 50% identity, 60% identity, at least 65% identity, at least 70% identity, at least 75% identity, at least 80% identity, at least 85% identity, at least 90% identity, or and at least 95% identity to the nucleotide sequence of SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163.

10. A method for treating a subject at risk for exposure to or exposed to at least one disease agent, the method comprising:
administering to the subject at least one binding protein including at least one binding region having an amino acid sequence selected from: SEQ ID NO: 96, SEQ ID NO: 98, SEQ ID NO:100, SEQ ID NO:102, SEQ ID NO:104, SEQ ID NO:106, SEQ ID NO:108, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, and SEQ ID NO:162, wherein the binding protein neutralizes the disease agent thereby treating the subject for the exposure.

11. The method according to claim 10, wherein the binding protein comprises at least one linker selected from the group of: a peptide, a protein, a sugar, or a nucleic acid.

12. The method according to claim 10, wherein the disease agent is a toxin, wherein the toxin is a plant lectin, wherein the plant lectin is at least one selected from the group of: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein or, wherein the toxin is a bacterial toxin, wherein the bacterial toxin is produced by at least one bacterial species selected from the group of: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovate; Pseudomonas for example P. aeruginosa; Neisseria for example N. gonorrhoeae; Escherichia coli for example E. coli O157:117; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

13. The method according to claim 10 further comprising observing or detecting neutralization of the disease agent by the binding protein and/or survival of the subject; or identifying a reduction or remediation in at least one pathology symptom associated with the disease agent.

14. The method according to claim 10, wherein the disease agent comprises a plurality of disease agents, and the method comprises prior to administering, engineering the binding protein to bind to a feature of each of the plurality of the disease agents.

15. The method according to claim 14, wherein the feature is non-identical.

16. The method according to claim 14, wherein the feature is identical.

17. A method for treating a subject at risk for exposure to or exposed to at least one disease agent, the method comprising:
administering to the subject a source of expression of a binding protein having a nucleotide sequence encoding the binding protein, wherein the nucleotide sequence comprises at least one selected from the group consisting of: a naked nucleic acid vector, bacterial vector, and a viral vector, wherein the nucleotide sequence comprises at least one selected from the group of: SEQ ID NO: 97, SEQ ID NO: 99, SEQ ID NO:101, SEQ ID NO:103, SEQ ID NO:105, SEQ ID NO:107, SEQ ID NO:109, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, and SEQ ID NO:163; and,
measuring neutralizing by the binding protein of the disease agent, thereby treating the subject for the exposure.

18. The method according to claim 17, wherein the binding protein comprises at least one linker selected from the group of: a peptide, a protein, a sugar, or a nucleic acid.

19. The method according to claim 17, wherein the disease agent is a toxin, wherein the toxin is a plant lectin, wherein the plant lectin is at least one selected from the group of: bean protein for example a castor bean protein (for example a ricin toxin), a jequirity (Abrus precatorius) bean protein, a jack bean (Concanavalia ensiformis) protein, a French bean (for example a phytohaerno glutinin toxin), or a soybean protein; a flower protein such as a mistletoe (Viscum album) protein, a sweet clover protein, or a snowdrop protein; a pea protein; a grain protein (for example protein in wheat, wheat germ, quinoa, rice, buckwheat, oats, rye, barley, millet and corn); and a peanut protein or, wherein the toxin is a bacterial toxin, wherein the bacterial toxin is produced by at least one bacterial species selected from the group of: Bacillus for example B. anthracis; Clostridium for example C. tetani, C. difficile, and C. perfringens; Corynebacterium for example C. diphtheriae; Bordetella for example B. pertussis; Mycobacterium for example M. tuberculosis; Salmonella for example S. enterica; Staphylococcus for example S. aureus and S. epidermis; Streptococcus for example S. pneumoniae and S. mutans; Treponema for example T. pallidum; Plasmodium for example P. falciparum, P. vivax, P. malariae, and P. ovale; Pseudomonas for example P. aeruginosa; Neisseria for example N. gonorrhoeae; Escherichia coli for example E. coli O157:H7; Shigella for example S. enteritis and S. flexneri; Campylobacter for example C. jejuni; Yersinia for example Y. pseudotuberculosis and Y. pestis; Listeria for example L. monocytogenes; Vibrio for example V. cholerae; and the like.

20. The method according to claim 17 further comprising observing or detecting neutralizing the disease agent by the binding protein and/or viability of the subject; or identifying a reduction or remediation in at least one pathology symptom associated with the disease agent.

21. The method according to claim 17, wherein the disease agent comprises a plurality of disease agents, and the method comprises prior to administering, engineering the binding protein to bind to a feature of each of the plurality of the disease agents.

22. The method according to claim 21, wherein the feature is non-identical.

23. The method according to claim 21, wherein the feature is identical.

24. The method according to claim 17, wherein measuring comprises detecting a reduced amount of degree of at least one of: bacterial titer in a tissue or bodily fluid, fever, inflammation, pain, diarrhea, bleeding, tissue discoloration, clotting, and tachycardia.

25-34. (canceled)
